(19) United States
(12) Patent Application Publication
(10) Pub. No.: US 2025/0257408 A1
(43) Pub. Date: Aug. 14, 2025

(54) CLONAL HEMATOPOIESIS AS A BIOMARKER

(71) Applicant: UNIVERSITY HEALTH NETWORK, Toronto (CA)

(72) Inventors: Filio BILLIA, Toronto (CA); John DICK, Toronto (CA); Robert VANNER, Toronto (CA); Jessie MEDEIROS, Brampton (CA); Fernando SCOLARI, Rio Grande do Sul (BR); Sagi ABELSON, Toronto (CA)

(21) Appl. No.: 18/859,154

(22) PCT Filed: Apr. 24, 2023
(86) PCT No.: PCT/CA2023/050553

§ 371 Date: Oct. 22, 2024

Related U.S. Application Data
(60) Provisional application No. 63/333,628, filed on Apr. 22, 2022.

(51) Int. Cl. C12Q 1/6886; C12Q 1/6869; G16B 20/20; G16H 50/30

(52) U.S. Cl. CPC C12Q 1/6886; C12Q 1/6869; G16B 20/20; G16H 50/30; C12Q 2600/106; C12Q 2600/118; C12Q 2600/156

(57) ABSTRACT

There is described herein methods predicting the risk of various disease condition by measuring clonal hematopoiesis in a patient, probes used to make such measurement and methods for treatment or preventive treatment of the disease condition.

### RELATED APPLICATIONS

This application claims priority to U.S. Provisional Application No. 63/333,628 filed on Apr. 22, 2022, which is hereby incorporated by reference in its entirety.

### FIELD OF THE INVENTION

The invention relates to the measurement of clonal haematopoiesis for the purposes of assessing the risk associated with various disease states, particularly relating to solid organs and/or cancer.

### BACKGROUND OF THE INVENTION

Clonal haematopoiesis (CH) is the acquisition of mutations in hematopoietic stem cells that results in selective clonal expansion leading to enhanced systemic inflammation.⁵ While early studies had shown that at least 10% of individuals harbour CH mutations in their seventies,⁵ more sensitive techniques have demonstrated that CH mutations are ubiquitous in healthy middle-age adults.^{6,7} Despite these mutations increasing the risk of haematological cancer development, progression to malignancy is low.⁸

Interestingly, CH is also associated with elevated cardiovascular risk;^{5,9,10} more than most traditional risk factors.¹¹ The presence of CH mutations is linked to a 25% increase in the incidence of de novo heart failure (HF) and a 2-fold increase in mortality in patients with pre-existing HF.^{9,12} In animal models of HF, mice harbouring CH mutations developed adverse left ventricular (LV) remodelling with fibrosis and a concomitant increase in IL-6 and IL-1ß.¹³

However, the incidence of CH in more specific cardiac diseases, specific cancers and diseases in other organs, and its impact in clinical outcomes, remains unknown.

For example, cardiogenic shock (CS) is a life-threatening condition with markedly reduced cardiac output resulting in multi-organ failure.¹ Despite increasing recognition of CS and implementation of intensive therapies, morbidity and mortality remain exceedingly high.² The evolving epidemiological descriptions of CS patients illustrate that there is a large variability in the underlying aetiology, response to medical therapy, and outcomes.^{1,3} The association of a systemic inflammatory response syndrome with worse outcomes in CS³ could be a key to understanding the heterogeneity in the natural history of CS and uncover new pathways to target for treatment.⁴

Further, orthotopic heart transplant (OHT) is the treatment of choice for patients with advanced heart failure to improve quality of life and survival.^{s1} In the United States, over 80,000 HT were performed in the past three decades and over 5,000 yearly worldwide.^{s2, s3} However, despite the improvement in donor and recipient pre-transplant care, post-transplant survival is affected by a number of complications including infections due to long-term immunosuppression therapy, cancer development and cardiac allograft vasculopathy (CAV).^{s4, s5} OHT recipients require extensive monitoring after transplantation with endomyocardial biopsies (EMB) for rejection vigilance, viral detection for opportunistic infection, echocardiograms and coronary angiography for allograft function and CAV assessment to instill early treatment and improve survival.⁴ Despite this intensive care, new non-invasive surveillance methods that could improve morbidity and reduce mortality are still warranted.^{s6} The increased inflammatory cytokine/chemokine production linked to CH could also affect outcomes in OHT recipients, but this association has never been evaluated.^{s8, s15}

There remains a need to study the link between CH and various disease states, including those listed above.

### SUMMARY OF THE INVENTION

We hypothesized that the enhanced inflammatory environment associated with CH may play a significant role in various disease outcomes and its related complications. Therefore, we evaluated the prevalence of CH in patients with various cardiac diseases, solid organ diseases and specific cancers.

In an aspect, there is provided a method of predicting the risk of a disease condition of a solid organ in a patient, the method comprising: receiving a sample from the patient containing hematopoietic stem cells; sequencing the sample to detect a degree of clonal hematopoiesis; comparing the degree of clonal hematopoiesis in the patient to a control degree; and determining the patient is at an elevated risk of the disease condition if the degree of clonal hematopoiesis in the patient is higher than the control degree in a statistically significant manner.

In an aspect, there is provided a kit comprising a library of probes library comprising at least 50%, 60%, 70%, 80%, 90%, 95%, 98% or 99% of the probes listed in Table A.

In an aspect, there is provided a method of predicting the benefit of immunotherapy in a patient with melanoma, the method comprising: receiving a sample from the patient containing hematopoietic stem cells; sequencing the sample to detect a degree of clonal hematopoiesis in TET2; comparing the degree of TET2 clonal hematopoiesis in the patient to a control degree; and determining the patient would benefit from immunotherapy if the degree of TET2 clonal hematopoiesis in the patient is higher than the control degree in a statistically significant manner. Preferably, the method further comprises treating the patient with immunotherapy.

In an aspect, there is provided a method of predicting metastatic risk in a patient with a non-hematological cancer, the method comprising: receiving a sample from the patient containing hematopoietic stem cells; sequencing the sample to detect a degree of clonal hematopoiesis in TET2; comparing the degree of TET2 clonal hematopoiesis in the patient to a control degree; and determining the patient is at a lower risk of metastasis if the degree of TET2 clonal hematopoiesis in the patient is higher than the control degree in a statistically significant manner. Preferably, the method further comprises treating the patient with a treatment and monitoring regimen reflective of a low risk of metastasis.

### BRIEF DESCRIPTION OF FIGURES

These and other features of the preferred embodiments of the invention will become more apparent in the following detailed description in which reference is made to the appended drawings wherein:

FIG. 1. Study design. Cardiogenic shock patients were screened for eligibility and those with available biospecimen and consent were included. Ambulatory heart failure patients were screened on a digital database and those with complete data for age, sex, ejection fraction and aetiology of heart failure, available biospecimen and consent were included. Cardiogenic shock and ambulatory heart failure patients were matched on a 1:1 ratio for age, sex, ejection fraction, and aetiology. Ambulatory heart failure and cardiogenic shock patients were sequenced for clonal haematopoiesis related genes and its prevalence was estimated. A survival analysis was performed in each group. A survival analysis for specific genes mutations and a cytokine profile were assessed in cardiogenic shock patients.

FIG. 2. Somatic variant characteristics in cardiogenic shock cohorts and ambulatory heart failure. Panel A shows the number of mutations for the 10 most frequently mutated genes according to each cohort. Panel B shows the number of mutations per patient in those where a clonal haematopoiesis mutation was identified. Panel C shows the variant allele frequency boxplot for patients with a mutation with a VAF≥2%. No differences were observed between cohorts (P=0.87). Panel D shows the number of patients with CH mutation according to age in both cohorts. Filled columns represent those with CH mutations, and unfilled those without.

FIG. 3. Survival according to clonal haematopoiesis and specific gene mutations. Panels A, B and C shows the reduced survival in CS patients with CH mutations in 30-day, 90-day and 3-year survival respectively. Panels D, E and F shows the reduced survival in CS patients according to specific CH-related mutations (DNMT3A, ASLX1 and TET2) in 30-day, 90-day and 3-year survival respectively. All figures represents VAD/OHT censored survival analysis.

FIG. 4. Inflammatory cytokines in CS patients with TET2 and ASXL1 mutations. Panel A shows the differences in SCD40L, IFNγ, IL-4, TNF-α and abundance in plasma of patients with or without TET2 mutations of variant allele frequency ≥2%. Panel B shows the differences in CCL7 in patients with or without ASXL1 mutations with a variant allele frequency ≥2% (P=0.03).

FIG. 5. Somatic clonal hematopoiesis gene mutations prevalence and characteristics in orthotopic heart transplant recipients. Panel A shows the frequency of CH in OHT recipients. Panel B shows the number of mutations according to genes related to CH. Panel C shows the number of patients with one, two, three, five and six CH mutations. Panel D shows the number of patients with CH mutations according to age. (CH, clonal hematopoiesis; OHT, orthotopic heart transplant).

FIG. 6. Survival analysis in orthotopic heart transplant recipients according to the presence of clonal hematopoiesis gene mutations. (CH, clonal hematopoiesis) FIG. 7. Somatic mutations related to clonal hematopoiesis in patients with hypertrophic cardiomyopathy. Panel A shows the number of mutations in the most common affected genes in the cohort. Panel B shows the number of mutations per patients in those with clonal hematopoiesis. Panel C shows the number of patients with clonal hematopoiesis according to the decade of the assessment. (CH, clonal hematopoiesis; HCM, hypertrophic cardiomyopathy).

FIG. 8. Survival in patients with hypertrophic cardiomyopathy stratified according to the presence of clonal hematopiesis. Panel A shows the survival according to the presence of clonal hematopoiesis among HCM patients. Panel B shows the survival according to the presence of clonal hematopiesis in the DNMT3A, TET2, and ASXL1 genes among HCM patients. Panel C shows the survival according to the presence of clonal hematopoiesis among HCM patients with sarcomeric mutations. Panel D shows the survival according to the presence of clonal hematopiesis in the DNMT3A, TET2, and ASXL1 genes among HCM patients with sarcomeric mutations. (CH, clonal hematopoiesis; HCM, hypertrophic cardiomyopathy).

FIG. 9. Troponin I, cytokines and chemokines levels among HCM patients with sarcomeric mutations according to the presence of clonal hematopoiesis. (CH, clonal hematopoiesis; HCM, hypertrophic cardiomyopathy).

FIG. 10. TET2-mutant clonal hematopoiesis is associated with clinical benefit from immunotherapy in melanoma. A) 569 patients with melanoma, bladder cancer, renal cell carcinoma (RCC), or non-small cell lung cancer (NSCLC) treated with immune-checkpoint blockade were screened for clonal hematopoiesis using publicly-available exome sequencing, with at least one mutation of variant allele frequency (VAF)>0.02 detected in 74 patients (datasets EGAD00001006632, SRP064805, SRP067938, SRP072934, SRP090294, SRP095809, SRP115658, SRP128156). B) TET2-mutant clonal hematopoiesis is associated with significantly higher odds of clinical benefit 6 months after receiving immunotherapy in melanoma patients (Odds Ratio=5.98), but not bladder cancer, renal cell carcinoma, or non-small cell lung cancer patients (p value from Firth's multivariate logistic regression adjusted for patient age, sex, study, and immune checkpoint). C) In an animal model of immunotherapy, mice with Tet2-mutant hematopoiesis—mimicking TET2-mutant clonal hematopoiesis—show enhanced response to PD-1 immune checkpoint blockade, while isotype control (ISO) treated tumours show identical growth kinetics (*p<0.05 by Mann-Whitney Test).

FIG. 11. TET2-mutant clonal hematopoiesis is associated with lower risk of metastatic disease in patients with non-hematological cancers. A) 16,744 patients with metastatic or non-metastatic solid tumours from Nguyen et al. Cell, 2022 were tested for clonal hematopoiesis in Bolton et al. Nature Genetics, 2020 using the MSK-IMPACT targeted sequencing panel. CH mutations with variant allele frequency of at least 0.02 were detected in 19.5% of patients. B) The 5 most commonly detected clonal hematopoiesis mutations from A) are shown. C) Exposure to TET2-mutant CH is associated with lower risk of having metastatic cancer in patients from A) as assessed by multivariate logistic regression adjusted for age, sex, smoking status, and chemotherapy treatment. D) Exposure to TET2-CH is associated with significantly lower risk of having metastases in patients from A) with Non-Small Cell Lung and Breast Cancer, with a trend towards lower risk of metastases in colorectal and bladder cancer, as assessed by multivariate logistic regression adjusted for age, sex, smoking status, and chemotherapy treatment. Patients in A) with TET2-mutant CH have significantly few metastases detected on clinical imaging (E) and have fewer overall sites/organs involved with metastatic disease (F), p values from Wilcoxon Rank Sum Test.

FIG. 12. Clonal hematopoiesis prevalence among the 782 transplant recipients.

FIG. 13. Clonal hematopoiesis prevalence according to transplanted organ.

FIG. 14. Clonal hematopoiesis mutated genes among the 782 transplant recipients.

FIG. 15. Clonal hematopoiesis mutated genes among the 127 heart transplant recipients.

FIG. 16. Clonal hematopoiesis mutated genes among the 90 lung transplant recipients.

FIG. 17. Clonal hematopoiesis mutated genes among the 189 kidney transplant recipients.

FIG. 18. Clonal hematopoiesis mutated genes among the 374 liver transplant recipients.

FIG. 19. Mortality according to the transplanted organ.

FIG. 20. Mortality according to clonal hematopoiesis.

FIG. 21. Mortality according to clonal hematopoiesis by transplanted recipients.

### DETAILED DESCRIPTION

In the following description, numerous specific details are set forth to provide a thorough understanding of the invention. However, it is understood that the invention may be practiced without these specific details.

As noted above, we investigated the role that the enhanced inflammatory environment associated with CH may play in various disease outcomes and its related complications.

In one example, we studied cardiogenic shock (CS) and its association with variable systemic inflammation and whether it may be responsible for the patient heterogeneity and the exceedingly high mortality rate. Cardiovascular events have been associated with clonal haematopoiesis (CH) where specific gene mutations in hematopoietic stem cells lead to clonal expansion and the development of inflammation. This study aims to assess the prevalence of CH and its association with survival in a population of CS patients in a quaternary centre.

We compared the frequency of CH mutations among 341 CS patients and 345 ambulatory heart failure (HF) matched for age, sex, ejection fraction, and HF aetiology. The association of CH with survival and levels of circulating inflammatory cytokines was analysed. We detected 266 CH mutations in 149 of 686 (22%) patients. CS patients had a higher prevalence of CH-related mutations than HF patients (OR 1.5; 95% Cl 1.0-2.1, P=0.02) and was associated with decreased survival (30-days: HR 2.7; 95% Cl 1.3-5.7, P=0.006; 90-days: HR 2.2; 95% Cl 1.3-3.9, P=0.003; and 3-years: HR 1.7; 95% Cl 1.1-2.8, P=0.01). TET2 or ASXL1 mutations were associated with lower survival in CS patients at all-time points (P≤0.03). CS patients with TET2 mutations had higher circulating levels of SCD40L, IFNγ, IL-4, and TNFα (P≤0.04), while those with ASXL1 mutations had decreased levels of CCL7 (P=0.03).

CS patients have high frequency of CH, notably mutations in TET2 and ASXL1. This was associated with reduced survival and dysregulation of circulating inflammatory cytokines in those CS patients with CH.

Novel risk stratification and non-invasive surveillance methods are also needed in orthotopic heart transplant (OHT) to reduce morbidity and mortality post-transplant, and this was thus the focus of another example. The purpose of this study was to investigate the association between CH and OHT. Blood samples were collected from 127 OHT recipients. Error-corrected sequencing was used to detect CH-associated mutations. We evaluated the association between CH and acute cellular rejection, CMV infection, cardiac allograft vasculopathy (CAV), malignancies, and survival. CH mutations were detected in 26 (20.5%) patients, mostly in DNMT3A, ASXL1, and TET2. Patients with CH showed a higher frequency of CAV grade 2 or 3 (0% vs. 18%, P<0.001). Moreover, a higher mortality rate was observed in patients with CH [11 (42%) vs. 15 (15%), P=0.008] with an adjusted hazard ratio of 2.9 (95% CI, 1.4-6.3; P=0.003). CH was not associated with acute cellular rejection, CMV infection or malignancies. The prevalence of CH in OHT recipients is higher than previously reported for the general population of the same age group, with an associated higher prevalence of CAV and mortality.

Similar studies were conducted with respect to hypertrophic cardiomyopathy, lung transplant, immunotherapy benefit in myleloma and metatstic risk of a non-hematological cancer.

Accordingly, in an aspect, there is provided a method of predicting the risk of a disease condition of a solid organ in a patient, the method comprising: receiving a sample from the patient containing hematopoietic stem cells; sequencing the sample to detect a degree of clonal hematopoiesis; comparing the degree of clonal hematopoiesis in the patient to a control degree; and determining the patient is at an elevated risk of the disease condition if the degree of clonal hematopoiesis in the patient is higher than the control degree in a statistically significant manner.

As used herein, the term “control” refers to a specific value or dataset that can be used as a reference to classify a measured value e.g. the wild type or frequency of mutations in a cohort. A person skilled in the art will appreciate that the comparison between the measurement in the test sample and the reference values in the control will depend on the control used.

As used herein “hematopoietic stem cell” refers to cells capable of developing into any blood cell, including mature myeloid and/or lymphoid cells. These cells are typically bone marrow, liver, spleen or cord blood in origin. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells.

The term “sample” as used herein refers to any fluid, cell or tissue sample from a subject that can be assayed for the mutations in hematopoietic stem cells described herein.

In some embodiments, the degree of clonal hematopoiesis is measured using a variant allele frequency of mutations determined to be associated with clonal hematopoiesis. Preferably, the variant allele frequency (VAF) is ≥2%. Further preferably, the VAF is ≥5%.

In some embodiments, the following genes are sequenced in the sample: TET2, DNMT3A, and ASXL1, and optionally one or more of, but preferably all of, BCOR, BRAF, CALR, CBL, CEBPA, EZH2, FLT3A, GATA1, GATA2, GNAS, IDH1, IDH2, JAK2, KIT, KRAS, MPL, NRAS, PHF6, PPM1D, PTPN11, RAD21, RUNX1, SETBP1, SF3B1, SMC1A, SMC3, SRSF2, STAG2, TP53, U2AF1, WT1, and ZRSR2.

In some embodiments, the sequencing is performed using single-molecule molecular inversion probes (smMIPs). The smMIPs technique is an assay that combines single molecule tagging with multiplex targeted capture to enable practical and highly sensitive detection of low-frequency or subclonal variation.

In some embodiments, the mutations associated with clonal hematopoiesis are detectable by the probes listed in Table A.

In some embodiments, the mutations associated with clonal hematopoiesis are detected using a library comprising at least 50%, 60%, 70%, 80%, 90%, 95%, 98% or 99% of the probes listed in Table A.

In some embodiments, the mutations associated with clonal hematopoiesis are detected using a library consisting of at least 50%, 60%, 70%, 80%, 90%, 95%, 98% or 99% of the probes listed in Table A.

In some embodiments, the mutations associated with clonal hematopoiesis are detected using a library consisting of substantially all of the probes listed in Table A.

In some embodiments, the mutations associated with clonal hematopoiesis are detected using a library consisting of the probes listed in Table A.

In some embodiments, the solid organ is a heart.

In some embodiments, the disease condition is cardiogenic shock. In some embodiments, an elevated risk of cardiogenic shock is associated with an elevated risk of death. Preferably, the method further comprises treating or preventatively treating the patient for cardiogenic shock.

In some embodiments, the disease condition is an adverse outcome after orthotopic heart transplant (OHT). In some embodiments, the adverse outcome is an elevated risk of mortality and/or elevated risk of cardiac allograft vasculopathy. Preferably, the method further comprises treating or preventatively treating the patient for cardiac allograft vasculopathy.

In some embodiments, the disease condition is hypertrophic cardiomyopathy. Preferably, the method further comprises treating or preventatively treating the patient for hypertrophic cardiomyopathy.

In some embodiments, the solid organ is a lung. In some embodiments, the disease condition is an adverse outcome, including death, after lung transplant. Preferably, the method further comprises treating or preventatively treating the patient for the adverse outcome after lung transplant.

In an aspect, there is provided a kit comprising a library of probes library comprising at least 50%, 60%, 70%, 80%, 90%, 95%, 98% or 99% of the probes listed in Table A.

In some embodiments, the kit comprises a library consisting of at least 50%, 60%, 70%, 80%, 90%, 95%, 98% or 99% of the probes listed in Table A.

In some embodiments, the kit comprises a library consisting of substantially all of the probes listed in Table A.

In some embodiments, the kit comprises a library consisting of the probes listed in Table A.

In an aspect, there is provided a method of predicting the benefit of immunotherapy in a patient with melanoma, the method comprising: receiving a sample from the patient containing hematopoietic stem cells; sequencing the sample to detect a degree of clonal hematopoiesis in TET2; comparing the degree of TET2 clonal hematopoiesis in the patient to a control degree; and determining the patient would benefit from immunotherapy if the degree of TET2 clonal hematopoiesis in the patient is higher than the control degree in a statistically significant manner. Preferably, the method further comprises treating the patient with immunotherapy.

In an aspect, there is provided a method of predicting metastatic risk in a patient with a non-hematological cancer, the method comprising: receiving a sample from the patient containing hematopoietic stem cells; sequencing the sample to detect a degree of clonal hematopoiesis in TET2; comparing the degree of TET2 clonal hematopoiesis in the patient to a control degree; and determining the patient is at a lower risk of metastasis if the degree of TET2 clonal hematopoiesis in the patient is higher than the control degree in a statistically significant manner. Preferably, the method further comprises treating the patient with a treatment and monitoring regimen reflective of a low risk of metastasis.

The advantages of the present invention are further illustrated by the following examples. The examples and their particular details set forth herein are presented for illustration only and should not be construed as a limitation on the claims of the present invention.

### Example 1

### Materials and Methods

### Patient Selection

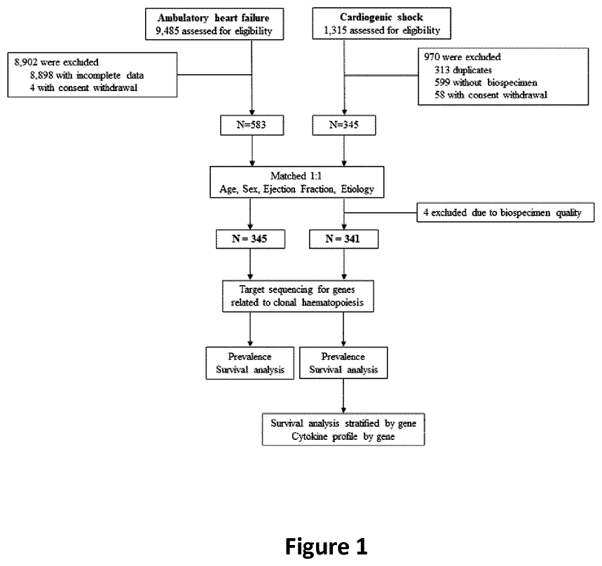

We performed a retrospective study to evaluate the prevalence of CH in CS patients and compared it to matched stable ambulatory HF patients. Stable ambulatory HF patients were chosen as controls as there is a known relationship with CH. We screened 1,315 consecutive patient admission with CS admitted to the cardiac intensive care unit (CICU) at the Toronto General Hospital (Ontario, Canada) from January 2014 to December 2020, identifying 341 patients with suitable biospecimens and consent. CS was diagnosed based on international consensus.¹⁴ Only the first CICU admission was considered for inclusion into the study. We also screened 9,485 ambulatory HF patients with no previous history of CS, orthotopic heart transplant (OHT), durable ventricular assist device (VAD), or HF admission in the 12 months prior to biospecimen collection and clinical evaluation (N=583). We included all patients aged ≥18 years, with biospecimens stored in the Peter Munk Cardiac Centre Cardiovascular Biobank. We excluded patients with incomplete digital records, those who withdrew consent during follow-up, or low biospecimen quality (FIG. 1).

### Definition of Clinical States of Enrolled Patients

Cardiogenic shock diagnosis was based on international consensus criteria¹⁴ which required a systolic blood pressure≤90 mmHg for more than 30 minutes, or the need for inotrope/vasopressor support, signs of end-organ failure (clammy skin, capillary filling time >3 seconds, urine output <0.5 mL/kg/h, lactate level >4 mmol/L), or a low cardiac output (<2.2 L/min/m² if receiving inotropes/vasopressors or <1.8 L/min/m² without inotropes/vasopressors). In contrast, we defined stable ambulatory HF as patients with HF, with no history of OHT or VAD, or HF admission in the 12 months up to the time-point of biospecimen collection.

### Data Collection

Clinical and laboratory data for CS patients were collected within the first 24 hours of CICU admission. The Society for Cardiovascular Angiography and Intervention (SCAI) CS stage¹⁴ was calculated at 24 hours after CICU admission. Data collected after this time were related to in-hospital outcomes such as use of mechanical circulatory support (MCS), renal replacement therapy, mechanical ventilation and death. Data for ambulatory HF patients were collected as close to the biospecimen collection date as possible. All study data were collected from electronic records and prior to CH sequencing.

### Follow-Up and Outcomes

All CS patients were followed until death or their last visit to our institution after hospital discharge. Follow-up time for the ambulatory HF group was defined from the time of biospecimen collection until the last visit to our institution or notice of death. As OHT and VAD substantially increase survival, a patient was right censored at the time of VAD, OHT or last follow-up, whichever was earlier. We reported event-free survival at 30-day, 90-day and 3-year. Similar to other studies,⁹ we defined CH-related mutations at a VAF cut-off ≥2%.

### Biospecimens and Analysis

Biospecimens were collected from patients during their admission to the CICU unit of the Toronto General Hospital with deferred consent. In the case that a patient, or their substitute decision-maker, denied consent at a later time point, the biospecimens were discarded. Biospecimens from ambulatory HF patients were obtained during routine heart function clinic visits at our institution. All samples were stored at −80° C. at the Peter Munk Cardiac Centre biobank.

For patients with CS, biospecimens were collected within a median of 10 (interquartile range, IQR 67) days from CS admission. In the ambulatory HF patient group, 89% of biospecimens were collected on the same day as clinical evaluation. We felt the timing of biospecimen collection was appropriate as changes in clonal haematopoiesis do not occur over days, but rather over years.

### Genetic Sequencing Procedures

Next-generation sequencing library construction was conducted with smMIPs.¹⁵

### Cytokine Analysis

To define whether cytokine levels can be potential markers of inflammation, the analysis was restricted to those samples obtained during the admission to the CICU.

### Statistical Analysis

All analyses on the frequency and associations with CH were performed with a variant allele frequency (VAF) cut-off ≥2%. For specific gene analysis, we compared patients with no mutation with those with a mutation in one of the three more common mutations (DNMT3A, TET2, or ASXL1) with a VAF ≥2% but with no overlap mutations. We also transformed VAF and cytokines levels using the logarithm function to reduce the distribution skewness.

A sample size of 345 patients for each group would detect a ratio of 1.5 in CH frequency between CS and ambulatory HF groups with a power of 87.5% and alpha of 0.05. There were 345 CS patients and 583 stable HF patients selected after initial screening. The CS and stable HF groups were one-to-one matched by age, sex, aetiology of HF, and ejection fraction. A nearest neighbour method was applied without replacement. After matching, there were 345 patients in each group. FIG. 1 summarizes sample selection.

All variables were tested for normality with histogram analysis and Shapiro-Wilk test. Normal and non-normal continuous variables are presented as mean±standard deviation or median (interquartile range), and categorical variables with frequencies. Between-cohort differences in continuous variables were evaluated with a Student's t-test or Mann-Whitney accordingly. Between-cohort differences in categorical variables were evaluated with a chi-square test. Logistic regression was performed to quantify the association of the prevalence of CH between CS and ambulatory heart failure patients in terms of odds ratios. Next, we separately characterized the 30-day, 90-day and 3-year event-free survival for each cohort using the Kaplan-Meier survival method. Furthermore, within each cohort, we contrasted event-free survival in patients with and without CH in terms of hazard ratios (HR) using Cox proportional hazards regression and evaluated the survival differences using log-rank tests. The HRs in this descriptive analysis were not adjusted for covariates. Subsequently, we also quantified the association between CS and mortality both with age only and with other clinically relevant covariates (i.e., age, sex, aetiology, use of mechanical circulatory support, creatinine, sodium, and white blood count). The model was validated by the proportional hazard assumption test based on Schoenfeld residual. Finally, as a sensitivity analysis, we repeated the previous analyses on all-cause mortality without right censoring patients at the time of OHT and VAD implantation. We described and explored the event-free survival and the levels of 48 cytokines in patients with a single gene mutation in the three most commonly mutated genes to patients without any mutations. We used a statistical significance of 0.05 for all analyses and a two-sided p-value. All analyses were performed using SPSS, version 25.0 (SPSS Inc., NY, USA).

### Results and Discussion

In this study, 341 patients were included in the CS group and 345 in the ambulatory HF group. Four (1%) patients were excluded from the CS group due to poor biospecimen quality (FIG. 1). All characteristics included in the case-control study were similar between groups (Table 1). Our study population consisted mostly of males with a predominance of non-ischemic cardiomyopathy. Patients with CS, however, had a lower body mass index, more frequent history of smoking, lower incidence of pre-existing chronic HF, higher admission serum creatinine and higher BNP when compared with ambulatory HF patients.

In the CS group, the most common aetiology was acute decompensation of chronic HF in 225 (66%) patients, followed by new onset HF in 93 (27%), myocardial infarction in 10 (3%) and myocarditis in 11 (3%). The majority were classified at SCAI stage D (285 patients, 83%) and pulmonary artery catheter was used in 178 (52%) patients confirming disease severity. Fifty-two (15%) patients required temporary MCS with intra-aortic balloon pump being the most commonly used (n=26, 8%), followed by extra-corporeal life support (n=18, 5%), and Impella Device® (Abiomed Inc., Massachusetts, USA) (n=8, 2%). Mechanical ventilation was needed in 63 (18%) patients and renal replacement therapy in 35 (10%). Clinical characteristics are summarized for both CS patients (Table 2) and ambulatory HF (data not shown). CS patients were followed for a median of 676 (1289) days, while ambulatory HF patients were followed for 1246 (884) days.

In the CS group, 104 (30%) underwent OHT, 79 (23%) required durable VAD, 132 (39%) died during follow-up with 59 (17%) patients dying during the index hospitalization and 73 (21%) after hospital discharge. In the ambulatory HF group, 19 (6%) patients had OHT, seven (2%) were referred for durable VAD, and 35 (10%) patients died.

### Prevalence and Distribution of CH Mutations in the Study Groups

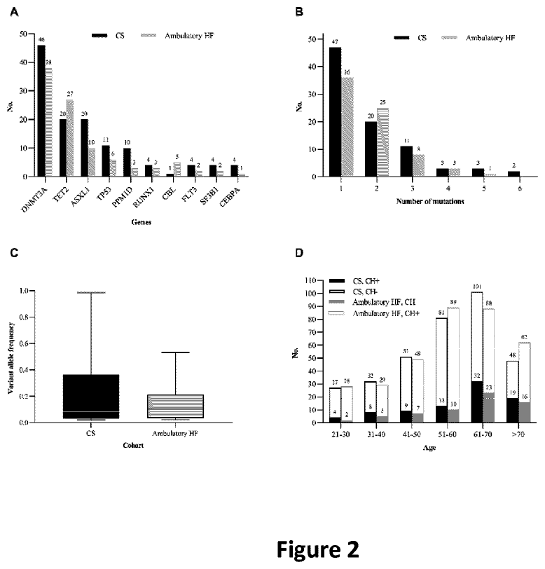

Overall, 422 mutations were identified in 28 genes, of which 266 (63%) mutations had a VAF ≥2% and were included in the analysis (data not shown), affecting 149 (22%) of the 686 patients. The most common mutation type was missense, followed by frameshift, nonsense and splice site (data not shown). The most mutated gene was DNMT3A in 84 (32%), followed by TET2 in 48 (18%), and ASXL1 in 30 (11%) patients, with a similar distribution in mutations observed in both the CS and ambulatory HF groups (FIG. 2A). Among all patients with CH, 83 (56%) had a single mutation, 35 (23%) had two and 31 (21%) had three or more (FIG. 2B). The VAF distribution was similar in both populations (FIG. 2C). As expected, an age-associated increase in CH prevalence (FIG. 2D) was observed in both groups; however, the overall frequency of CH was significantly higher in CS patients, as compared with ambulatory HF patients (25.2% versus 18.3%; odds ratio, 1.5; 95% Cl 1.0-2.1, P=0.02). CS patients with CH were older than those without mutations, and had a higher frequency of dyslipidaemia, lower sodium, and white blood count (Table 2). The frequency of CH in CS patients with previous HF was similar than those without out [24 (26%) vs. 24 (23%), P=0.54). Ambulatory HF patients with CH were older and more likely to have ischemic cardiomyopathy (data not shown).

### Association Between CH and Survival

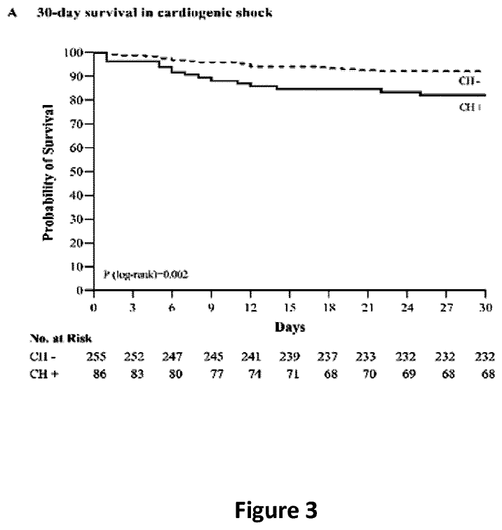

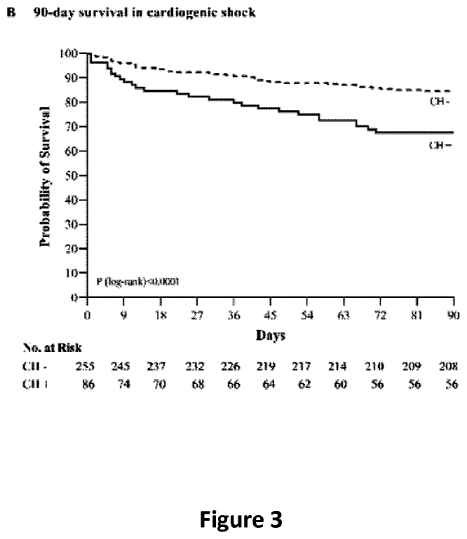

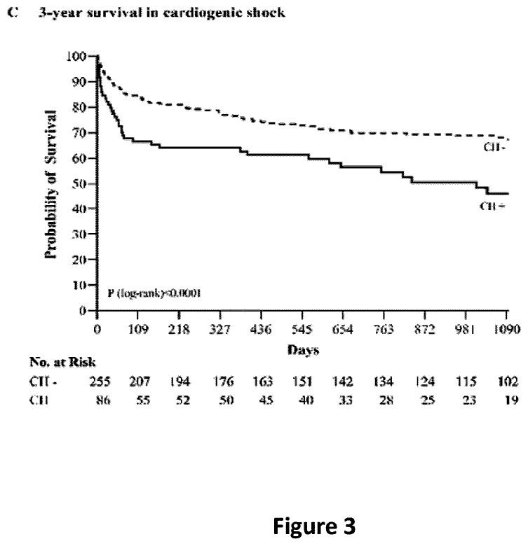

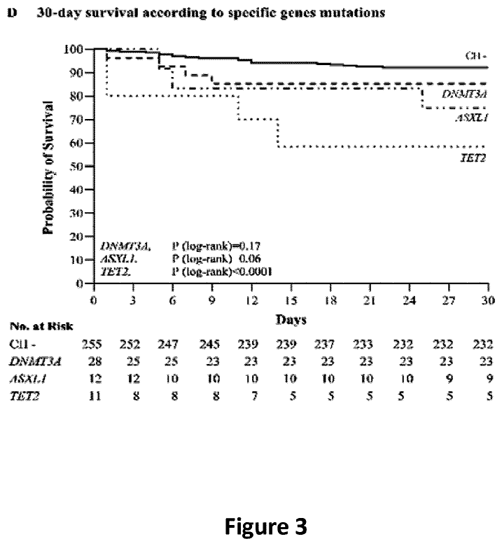

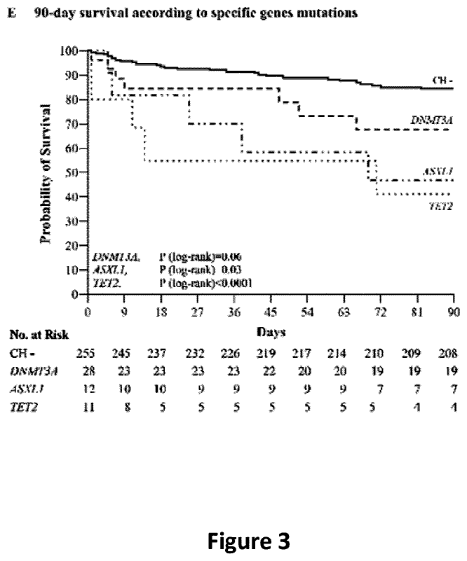

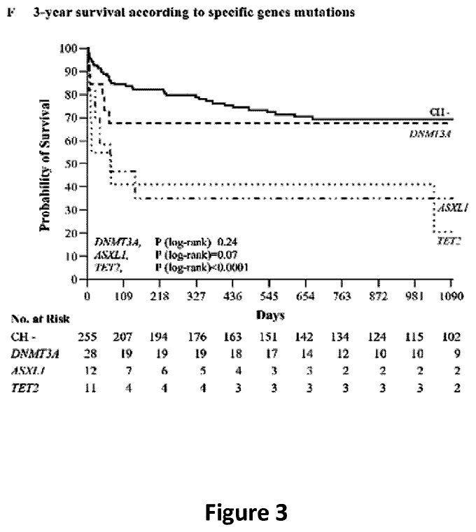

The presence of CH in the CS group was associated with an increased risk of death in a multivariable adjusted analysis for age, sex, aetiology of CS, use of mechanical circulatory support, creatinine, sodium, and white blood count and censored for VAD/OHT: 17 patients (20%) with CH died in the first 30-days compared with only 17 (7%) without mutations (hazard ratio, (HR), 2.7; 95% Cl 1.3 to 5.7, P=0.006; FIG. 3A). A similar ratio was seen at 90-days (HR, 2.2; 95% Cl 1.3 to 3.9, P=0.003; FIG. 3B). At 3 years, 36 patients (42%) with CH had died compared with 47 (18%) without detectable CH at initial admission for CS (HR, 1.7, 95% Cl 1.1 to 2.8, P=0.01; FIG. 3C). Similar results were observed with the unadjusted and age-adjusted analysis for both uncensored and VAD/OHT censored (data not shown). There were no differences in survival between CH and non-CH groups in the ambulatory HF patients uncensored or VAD/OHT censored (data not shown). In addition, when considering VAF as a continuous variable, there was no association with death. However, analysis of VAF categorized as ≥2 to <5% and ≥5% was associated with a higher risk of death in those with VAF ≥5%, at 90-days and 3-years. Increasing number of mutated genes was not associated with death (data not shown).

We then stratified the CS group according to the three most mutated genes (DNMT3A, TET2, and ASXL1, FIGS. 3D, 3E, 3F) and found an association for TET2 and ASXL1 mutations with decreased 30-day, 90-day, and 3-year survival compared with patients who had no CH mutations. Results for survival censored for VAD/OHT had similar results. DNMT3A mutations had no effect on survival at 30-day, 90-day or 3-year follow-up.

### Cytokine Expression in Cardiogenic Shock

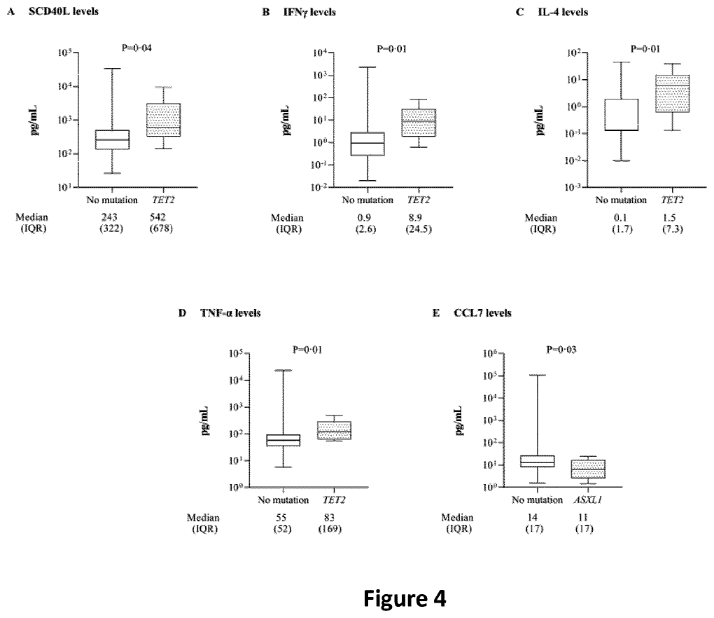

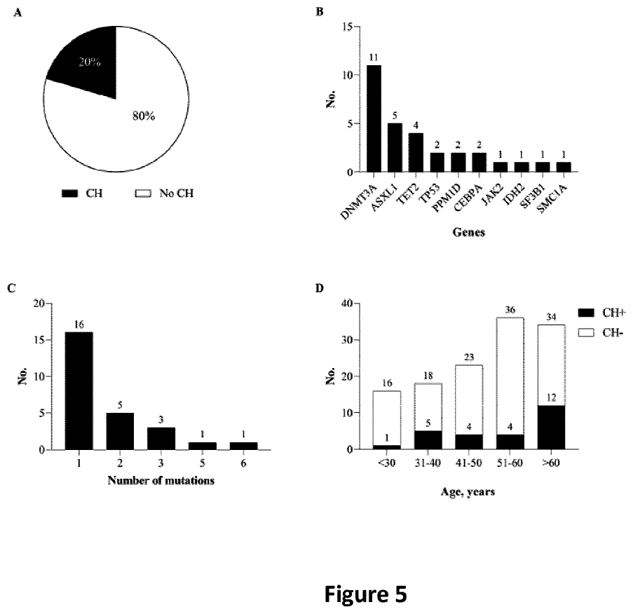

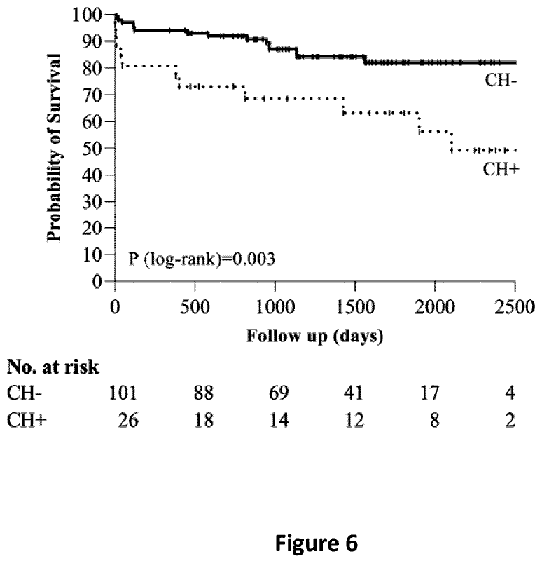

To assess changes in the inflammatory milieu associated with CH, we compared 48 cytokines in the plasma of CS patients with mutations in the three most mutated genes, to those without CH. CS patients with TET2 mutations exhibited higher circulating levels of SCD40L, IFNγ, IL-4, and TNFα (FIGS. 4A, 4B, 4C and 4D), and those with ASXL1 mutations had lower levels of CCL7 (FIG. 4E) compared with patients lacking CH mutations. There were no differences in the cytokine profiles of patients with DNMT3A mutations (data not shown).

### DISCUSSION

CH mutations are associated with increased risk for adverse cardiovascular events in specific populations of patients with atherosclerosis and heart failure.^{5,9,10} We found that patients with CS had a 1.5-fold higher prevalence of CH as compared to ambulatory HF patients, with an associated decrease in 30-day, 90-day, and 3-year survival. The observed prevalence of CH in ambulatory HF patients (18.3%) is consistent with other reported findings, emphasizing the comparability of the methodology applied.^{9,16} Importantly, our data shows that CH may be considered as risk factor for CS admission regardless of prior HF. Of note, mutations specifically in TET2 and ASXL1 impacted the prognosis of CS and were associated with distinct circulating inflammatory cytokine profiles, compared with those patients without CH mutations. While the retrospective nature of this study may have introduced a survival bias, as CS patients who did not survive the first hours of presentation were less likely to have biospecimens taken, our study likely underestimates the prevalence of CH in CS. To our knowledge, this is the first study to show the higher prevalence of CH and lower survival in patients admitted with CS.

DNMT3A was the most common mutated gene in both groups, followed by TET2 and ASXL1. These genes are epigenetic regulators with a role in altering DNA methylation to potentially promote stem cell self-renewal and clonal expansion.¹⁷ While DNMT3A is responsible for de novo DNA methylation, TET2 promotes demethylation.¹¹ ASXL1 has a role in chromatin regulation, promoting myeloid leukemogenesis.¹⁸ Although the mechanisms ascribed to these genes are still poorly understood, they likely boost systemic inflammation which may have an impact in CS.^{10,13,17} TET2 mutations were associated with a significant decrease in both short- and long-term survival in CS patients. These findings mirror previous work showing higher pathogenicity of mutations in TET2, compared to DNMT3A in the broader setting of HF.^{9,10,16,19} Additionally, ASXL1 mutations have been related to increased risk for myocardial infarction¹⁰ but its role in HF was unknown prior to our study. Here, we report a decrease of short- and long-term survival in ASXL1 carriers admitted with CS. DNMT3A mutations have been associated with HF hospitalization and death¹⁶, but this was not seen in our analysis. Mutations in DNMT3A may have lower pathogenicity in CS, explaining their higher frequency in this population.

CS is associated with acute systemic inflammation, which has been shown to increase mortality.^{4,20} Augmentation of this inflammatory state could explain the increased risk of death seen in CS patients with CH mutations. In our study, patients with CS harbouring TET2 mutations had elevated circulating levels of SCD40L, IFNγ, IL-4, and TNF-α. SCD40L has a pro-inflammatory, pro-coagulant function associated with cardiovascular events related to atherosclerosis.^{21,22} IL-4 leads to tissue macrophage accumulation,²³ and increases IFNγ expression, which has a key role in the adaptive immune response²⁴, and promotes myelopoiesis in response to inflammation^{24,25}. TNF-α is primarily produced by macrophages and can induce apoptosis in hematopoietic cells.^{26,27} Additionally, ASXL1 mutations in CS patients were associated with lower circulating levels of CCL7, a chemokine that is a potent chemo-attractant for myeloid cells.²⁸ The differential regulation of cytokines promoted by specific CH mutation could enhance the immune response leading to reduced survival in TET2 and ASXL1 mutation carriers. The lack of any dysregulation of circulating cytokines with DNMT3A mutations offers an explanation why survival was not affected in our CS patients.

Clinically, the increasing incidence of CS remains a major limitation in patient management.² The association of CH mutations with higher mortality could provide a new biomarker to help identify patients at elevated risk, establishing a new paradigm of risk assessment in CS. The fact that CH patients were older does not explain the dramatic increase in mortality in CH patients, as the age-adjusted analysis showed similar results. Also, similar age differences between study populations were described in previous studies showing the impact of CH in cardiovascular outcomes.^{9,19} Mechanistically, the increased risk due to CH likely occurs through augmentation of the acute inflammatory state in CS. The altered cytokine profile may provide potential therapeutic targets in these patients. Prior studies have shown that decreased IL-6 receptor activity improved outcomes in patients with CH.²⁹ The development of smMIP-sq for CH assessment in our study should overcome the barrier to clinical implementation of other sequencing methods.^{16,19} Furthermore, the 2% VAF cut-off remains controversial. VAF reflects the size of the expanded clone evaluated in the peripheral blood and it is reasonable to hypothesize that risk increases with an increase in VAF.^{11,16,17} However, we did not find a difference in the VAF between CS patients and ambulatory HF, and its use as a continuous variable was not associated with increased mortality in CS patients. We did find that CH-associated risk increased with a categorized higher VAF cutoff.¹⁶ This may be explained by the highly-skewed distribution of VAF.

CS patients had a 50% higher prevalence of CH mutations than stable ambulatory HF patients. These mutations were associated with a 2-fold reduction in survival of CS patients. Specifically, mutations in TET2 and ASXL1 genes were shown to be more lethal than DNMT3A in this context and were associated with an altered profile of circulating inflammatory cytokines that may suggest a mechanism for CH to affect patient outcomes.

### Example 2

### Materials and Methods

### Study Population

We performed a retrospective study of 127 patients that underwent OHT in the Toronto General Hospital from 2005 to 2021. Only patients older than 18 with available biospecimens and complete clinical evaluation were included. No patients with active malignancies were included because it is considered a criteria for heart transplant candidacy.

### Data Collection

The clinical chart and pre-transplant assessment of OHT recipients were reviewed from the patients' digital health records. We collected demographic data, medical history, laboratory assessment, date of OHT, transplant-related treatment at the time of the procedure and the immunosuppressive regimen at 1-yeart post-OHT, the occurrence and grade of acute cellular rejection episodes, CMV infection, CAV, any malignancy and death. All baseline characteristics regarding demographic and comorbidities were collected as close as possible to the biospecimens collection date because of its relationship with CH mutations.

### Follow-Up and Outcomes

Patients were followed from the time of OHT procedure to the last visit at our institution or death. The patients were evaluated for the following outcomes: the first occurrence of acute cellular rejection, CMV infection, de novo post-transplant malignancy, CAV, and death.

Cellular rejection episode was diagnosed and classified according to the International Society for Heart and Lung Transplantation (ISHLT).^{s4} A positive cellular rejection episode was defined as the occurrence of 2R and 3R classification in EMB performed routinely or due to clinical suspicion of cellular rejection. As a routine, patients are routinely evaluated for rejection in weeks 1-4, 6, 8, 10 and months 3-6, 9, 12, 18, 24. Gene-expression profiling was also used for rejection surveillance in low-risk patients at our center, with a gene-expression derived high-risk score verified by confirmatory EMB. CMV assessment was performed when infection was suspected or when prophylaxis was discontinued. CMV infection was defined as positive PCR in peripheral blood regardless of clinical symptoms. Post-OHT malignancy vigilance is performed routinely by our centre and the diagnosis is based on tissue biopsy showing malignant neoplastic cells. OHT recipients have coronary angiography performed with intravascular ultrasound at 3 months, 12 months, 1-year and 5-years post OHT, though this may be deferred if intercurrent illness or significant kidney disease is present. The findings are graded according to the ISHLT criteria for CAV and considered positive in the presence of CAV2 or CAV3.^{s4} In the survival analysis, we considered death from any cause in the end of follow-up.

### Genetic Sequencing Procedures

Peripheral blood samples were collected with patient consent during clinical visits to Toronto General Hospital and were stored in the Peter Munk Cardiac Centre biobank. 100 ng of DNA was used to construct sequencing libraries using single molecule Molecular Inversion Probes (smMIP) as previously described.^{s16} Paired-end 150 bp sequencing reads were generated using the Illumina Novaseq platform (Illumina Inc., California, USA).

### Statistical Analysis

All variables were tested for normality using the Shapiro-Wilk test. Normal and non-normal continuous variables are presented as mean±standard deviation or median (interquartile range). Difference among groups were evaluated with a Student's t-test or Mann-Whitney accordingly. Group differences in categorical variables were evaluated with a chi-square test or Fisher's exact test as appropriate. The hazard ratios (HR) for the event-free survival for OHT patients with or without CH was calculated using the Cox proportional hazards regression. An adjusted Cox proportional hazards regression including age, sex, primary heart failure diagnosis (ischemic, non-ischemic and congenital) and chronic kidney disease was also calculated to estimate the impact of CH on survival. The model was ascertained by the proportional hazard assumption test based on Schoenfeld residual and found to be valid. The survival function is represented graphically using Kaplan-Meier curves and compared according to the presence of CH mutations using log-rank test. We used a statistical significance of 0.05 for all analyses and a two-sided p-value. All analyses were performed using SPSS, version 25.0 (SPSS Inc., NY, USA).

### Results and Discussion

### Study Participants

Between 2005 and 2021, 589 patients underwent OHT at our institution. 127 patients (21.5%) were included in this study. 97 males and 30 females. Their mean age was 49±14 years. The most common comorbidities were prior smoking (n=34, 27%), hypertension (n=33, 26%), dyslipidemia (n=33, 26%) and type 2 diabetes (n=28, 22%). 90 patients (71%) were diagnosed with underlying non-ischemic HF prior to OHT while 28 patients (22%) had ischemic heart disease. Transplantation was conducted in 44 (35%) patients with a ventricular assist devices (VAD). All patients received induction therapy, with the vast majority (n-123, 97%) receiving rabbit anti-thymocyte globulin. The most common immunosuppressive therapy regimen at hospital discharge included tacrolimus (n=119, 94%), sodium mycophenolate (n=123, 97%) and corticosteroids (n=127, 100%). Table 3 summarizes the patient's baseline characteristics.

### Evaluation of CH in OHT Recipients

The targeted sequencing of CH genes was successfully performed in all patients. In 87 (68%) cases, samples were collected before the procedure with a median time from sample to procedure of 200 (IQR 428) days, and in 40 (32%) cases the sample was collected after the transplant, with a median time from the procedure to the sample collection of 114 (IQR 624) days. We observed 46 CH mutations in 26 (20%) OHT recipients (FIG. 1A) with a median variant allele frequency (VAF) of 8.6% (3.2-35.9). The most commonly affected genes were DNMT3A in 11 (9%), ASXL1 in 5 (4%), and TET2 in 4 (3%). TP53, PPM1D, and CEBPA affected two (2%) patients each, while JAK2, IDH2, SF3B1 and SMC1A were found in one (1%) patient each (FIG. 2B). Most patients (N=16, 62%) with CH had only one mutation (FIG. 3C). Clinical characteristics of patients with specific CH mutations were summarized (data not shown). The list of variants detected can be found in Table 5. Gene mutations and the VAF according to immunosuppressive regimen at hospital discharge were summarized (data not shown).

OHT patients with CH were older than those without mutation (54±13 vs 48±14 years, P=0.04) and were followed for a longer period after OHT (4.3±3.9 vs. 2.9±2.1, P=0.01). CH carriers also received an organ from EBV+ donor less frequently than those without CH [16 (62%) vs. 81 (80%), P=0.02]. At hospital discharge, CH patients were less likely to receive tacrolimus [22 (85%) vs. 97 (96%) P=0.03], and sodium mycophenolate [23 (89%) vs. 100 (99%), P=0.006]. There were no statistically significant differences in the prevalence of cardiovascular risk factors (diabetes, hypertension, BMI, smoking, dyslipidemia) or pre-OHT HF etiology between patients with and without CH (Table 3).

CH is Associated with Increase Mortality in the Setting of OHT

The mean follow-up post-OHT was 3.2±2.6 years. Primary graft dysfunction occurred in 19 (15%) with an in-hospital mortality post-OHT of 9%. During follow-up, 59 (46%) patients had CMV infection, 69 (54%) had at least one 2/3R acute cellular rejection episode, 11 (9%) had post-transplant malignancy, 4 (3%) CAV grade 2 or 3, and 26 (20%) died.

All four CAV diagnosis occurred OHT recipients with CH (18%; P<0.001), but no CAV was found among those without CH. The number of patients assessed for CAV with coronary angiogram was similar between patients with CH and those without CH CH [17 (65%). Vs. 68 (67%), P=0.85], and also time from OHT to first coronary angiogram 441 (1192) days vs. 399 (107) days, P=0.19. Patients with CH showed a higher mortality rate than those without CH [11 (42%) vs. 15 (15%), P=0.008], with an unadjusted hazard ratio (HR) of 3.1 (95% Cl 1.4-6.7), P=0.005, and an adjusted HR of 2.9 (95% Cl 1.4-6.3; P=0.003). The clinical characteristics and cause of death of patients with our without CH were summarized (data not shown). Because CH patients had a trend towards more Cyclosporine prescription at discharge and at the end of the first year after OHT, we performed an sensitivity analysis with age and type of immunosuppressive drugs in the of the 1^{st} year. The adjusted model showed that CH was associated with increased mortality with an HR of 5.9 (95% Cl 1.7-19.9), P=0.004. FIG. 2 shows the Kaplan-Meier survival analysis with an increased mortality in those with CH (P log-rank=0.003). The evaluation of outcomes according to specific gene mutation showed that more acute cellular rejection 2/3R were observed in those with DNMT3A, more CAV grade 2/3 in patients with DNMT3A and with TET2, and higher mortality in those with TET2 gene mutation (data not shown). We did not observe a higher mortality in patients with a VAF≥10% (n=14) in comparison to those with VAF<10% (N=113) [5 (35.7%) vs. 21 (18.6%), P=0.31]. Considering our results, we undertook a post-hoc power calculation. With a sample size of 127 patients and a hazard ratio of 3.1 for OHT survival, given an alpha of 0.05, we would have a power of 99.2% of detecting such a difference. No differences were observed between OHT patients with or without CH regarding CMV infection, 2/3R acute cellular rejection or post-transplant malignancy. Both cases of post-OHT malignancy in patients with CH were diagnosed with squamous cell carcinoma of the skin, and one also developed lung carcinoma. No patients developed PTLD. Table 4 summarizes outcomes in OHT recipients according to CH.

### DISCUSSION

CH has been linked to inflammatory conditions, increased mortality and incidence of cardiovascular diseases, but data in the setting of solid organ transplantation is scarce.^{s8, s17} We showed that the CH prevalence in OHT recipients is higher than expected for the same age in the general population. CH mutations are associated CAV and a 3-fold increase in mortality after OHT. These findings suggests a new biomarker in post-transplantation risk assessment.

The detection of CH is an exceedingly common feature of aging, with a prevalence of 10% by the 7^{th} decade of life and an overall prevalence of 4-5%.^{s7} In this study, 20% of OHT recipients were found to have a CH mutation in a younger population, mainly in DNMT3A, ASXL1 and TET2 genes, with most patients harboring a single mutation. Previously, HF patients were shown to have an 18.5% frequency in CH mutations at a median age of 65 years.^{s10, s14} Similarly, the median VAF in our cohort was 12%, which is much higher than the reported ≈4-6% in non-advanced HF cohorts.^{s10, s14} Additionally, CH patients have been consistently reported to be older than those without these mutation, similar to our study. While concerns can been raised about CH being a risk marker of aging instead of a disease-driving factor, several studies have shown that CH is independently associated with increased cardiovascular risk after adjusting for age.^{s10, s14} CH has also been shown to have a causal effect in mice harboring either Dnmt3a, Tet2, Jak2, Tp53, and Ppm1d mutations with adverse LV remodeling, lower LV ejection fraction and worsening degree of fibrosis post-myocardial infarction.^{s15, s18-s21}

CH mutations have been shown to be associated with atherosclerosis, myocardial infarction, stroke and HF.^{s8-s10, s14} A similar background of inflammation driving clinical outcomes would be expected in OHT recipients. In our study, CH was associated CAV grade 3. Experimental models have shown that Tet2-deficient mice have larger atherosclerotic plaque size and increased levels of several inflammatory cytokines.^{s8, s22} Higher levels of coronary artery disease have been observed in patients with CH.^{s8} The pathways involved in atherosclerosis development overlap with the inflammatory background of CH, but the pathophysiology of CAV comprises a complex interaction of immune and non-immune factors which contribute to a pro-inflammatory state and ultimately result in endothelial injury, vascular cell proliferation, fibrosis, and remodeling.^{s23-s25} The increased inflammatory cytokines expressed in patients with CH could have a role in CAV development, but further studies are needed to confirm this hypothesis, especially due to the low number of CAV diagnosis in the sample.

The complex relationship of immune phenomena and inflammation promoted by CH could be responsible for other outcomes in OHT recipients. The innate immune system can be activated by several different cytokines resulting in rejection episode^{s26, s27}, and activation of IL-1R pathway has a central role in ischemic reperfusion injury.^{s28} However, despite the overall higher proportion of CH patients experiencing acute cellular rejection, it failed to reach statistical significance. One possibility is that based on the 16% absolute increase observed, a sample size of over 400 patients would be required to reach statistical significance. CH has also been associated with the occurrence of infections and malignancies.^{s11, s12} We observed only a small number of such events which may explain the lack of association of CH with these outcomes. Nevertheless, CH was associated with a 3-fold increase in mortality in OHT recipients even after adjustment for confounding factors. Cardiovascular events in CH patients have been shown to be related with ischemic events and progression to heart failure, but these are not common in OHT recipients.^{s8, s10, s14} The cause of death in this study was related to allograft dysfunction, acute rejection episode, CAV, and septic shock. The mechanisms responsible for the observed high mortality rate remains unclear, yet previous work have shown that non-survivors after OHT were more likely to present with sustained inflammation.^{s29} Nonetheless, patients with CH were older, and, despite not being statistically significant, also had higher frequency of hypertension, dyslipidemia and smoking that could have an impact in our results.

In summary, we demonstrated that CH is associated with, and a potential risk factor, for CAV and mortality in OHT recipients. The complex interaction of the inflammatory cytokines promoted by CH and the immune system could drive several other potential outcomes such as rejection, infections and malignancies. A prior case series of graft versus host disease in transplanted liver patients showed that 71% had CH, highlighting the possible association with these somatic mutations to adverse outcomes.^{s30}

### Example 3

### Methods and Materials

We investigated a cohort of patients with hypertrophic cardiomyopathy (HCM) submitted to targeted sequencing for detecting CH. We included 799 patients who were ≥18 years of age, with a clinical diagnosis of HCM by current guidelines²², cardiac magnetic resonance imaging (MRI) and available biospecimens from the PMCC Biobank. We excluded patients with incomplete records, those that withdraw consent, or low biospecimen quality.

Among the 799 included patients, CH was found in 183 (22.9%). HCM patients with genotype-positive and CH were found to be more symptomatic and with a higher burden of fibrosis. CH was associated with major cardiovascular event (MACE) in HCM patients [adjusted HR of 3.46 (95% Cl 1.25-9.52; p=0.016)], with the highest risk among genotype-positive and DNMT3A, TET2 and ASXL1 mutated genes [adjusted HR of 7.23 (95% Cl 1.79-29.13) p=0.005]. Several cytokine and chemokines (IL-1ra, IL-6, IL-17F, TGFa, CCL21, CCL1, CCL8, and CCL17), and also troponin I were upregulated in those genotype-positive with CH. Fibrosis, a hallmark of HCM, was found to be increased in those with CH, as well as ABPR at exercise. CH was also associated with a higher mortality and major cardiovascular events (MACE). These results indicate that CH is frequent among HCM patients and associated with a worse clinical phenotype and outcomes.

### Definitions

HCM was defined as the presence of maximal LV wall thickness (MLVWT) ≥15 mm. Also included were patients with MLVWT ≥13 mm and a P/LP genetic variant or a family history of HCM in the absence of other causes for hypertrophy. MLVWT was defined as the higher LV wall measure on echocardiogram or cardiac MRI. The assessment of P/LP variants was conducted using a previously published strategy by our group²³. Evaluation for non-sustained ventricular tachycardia (NSVT) or abnormal blood pressure response (ABPR) at exercise were performed according to the attending clinician discretion. All patients with HCM underwent cardiac MRI with late gadolinium enhancement (LGE) for quantification of fibrosis. The LGE was assessed visually and quantified manually as previously validated⁸. LGE extent was defined as the LGE mass percentage of the total LV mass. We were able to quantify LGE in (84.1%) of patients with MRI. In the remaining 127 (15.9%) patients, we only included the qualitative measure of LGE as present or not.

Clonal hematopoiesis and cytokine assessment

The full method on CH and cytokines evaluation was performed substantially as described in earlier examples. Briefly, we used a single molecule molecular inversion probe (smMIP) method including 35 myeloid genes related to CH and smMIP-tools to call mutations and reported those with variant allele frequency (VAF), a measure of clone size, ≥2% for all analysis. The cytokine analysis was performed using a human cytokine/chemokine 71-plex assay with the Luminex™ 200 system by Eve Technologies Corp. (Alberta, Canada) and included brain natriuretic peptide (BNP) and cardiac troponin I (cTnI).

### Results and Discussion

### Clonal Hematopoiesis Prevalence and Characteristics

Overall characteristic of cohort is summarized in Table 7. The median time from biospecimen collection to the echocardiogram was 0 (0-16) days and median time from biospecimen to MRI assessment was 2.2 (0.2-5.6) years.

All patients had an assessment of CH performed and all samples passed quality control specifications. CH mutations were observed in 183 (22.9%) patients with a median VAF of 6.7 (2.8-40.8) %, being 136 (17.0%) in the three most common genes: DNMT3A in 70 (8.8%), TET2 in 51 (6.3%), and ASXL1 in 24 (3.0%), comprising 73.8% of all CH mutations. All other genes included in the smMIP panel were present in less than 1% of patients. Most patients (158, 19.8%) harbored a single mutation, while two mutations were found in 18 (2.3%) patients, 4 (0.5%) three mutations, and 3 (0.3%) four or more mutations. Among the 183 patients with CH, 135 (73.7%) were over 50 years old. However, among the 54 patients <30 years, CH was present in 14 (25.9%). In relation to the age of HCM diagnosis, 27 (14.8%) patients with CH were diagnosis before 30 years of age, while 62 (33.8%) within 31-50 years, and the majority, 94 (51.3%), were diagnosed after 50 years old. FIG. 7 summarizes CH mutations characteristics. Overall clinical characteristics of CH patients were similar to those without CH (Table 7).

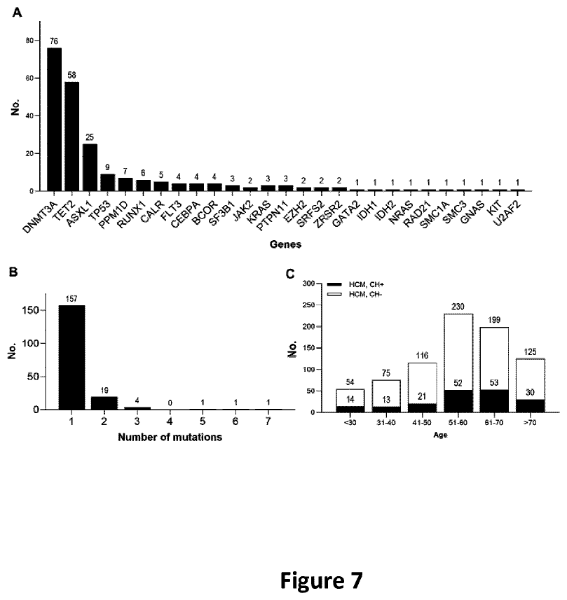

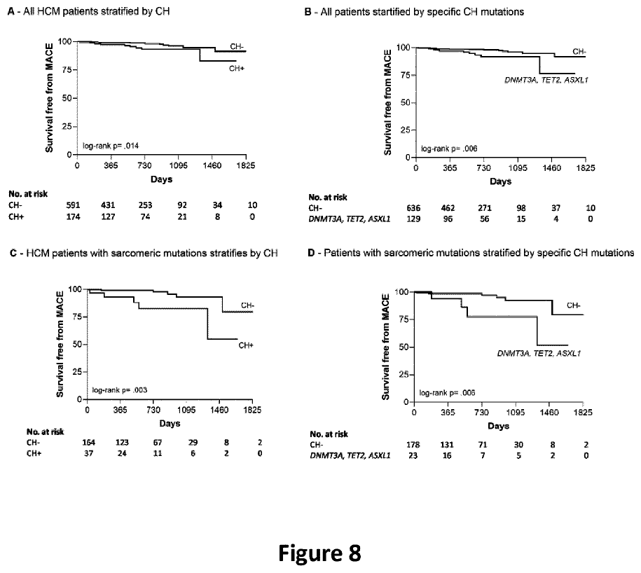

We then analyzed the association of CH with the HCM phenotype. No differences were found in those with or without CH on echocardiographic parameter such as MLVWT (18.3±4.2 mm vs. 18.8±5.1 mm, p=0.256), left atrium diameter (36.7±14.5 mm vs. 38.1±12.7 mm, p=0.208) and LVOT gradient (24.1±36.2 mmHg vs. 22.1±34.6 mmHg, p=0.491). Cardiac MRI parameters, including the presence of fibrosis evaluated by LGE, as well as other HCM features such as syncope, ABPR at exercise, among others were also similar between groups. However, apical aneurysm [33 (5.4%) vs. 14 (7.7) %, p=0.004] and death or need for orthotopic heart transplant was higher among patients with CH [4 (0.6%) vs. 6 (3.3%), p=0.005]. MACE was also more frequent among patients with CH [12 (2.0%) vs. 9 (5.2%), p=0.026], with an unadjusted HR of 2.72 (95% Cl 1.14-6.49; p=0.023), and adjusted HR of 3.46 (95% CI 1.25-9.52; p=0.016). CH patients showed a worse survival in comparison to those without CH as shown in the Kaplan-Meier curve (Log-Rank P=0.018) (FIG. 8A).

### Clonal Hematopoiesis Related to DNMT3A, TET2 and ASXL1

We evaluated whether somatic mutations in 3 genes (DNMT3A, TET2 and ASXL1) alone are sufficient to drive poor HCM patient outcome. We found that patients harboring DNMT3A, TET2 and ASXL1 mutations were older than their counterparts [55.3 (14.5%) vs. 58.1 (14.5%), p=0.042] and were more likely to have a family history of SCD [28 (4.2%) vs. 9 (6.7%), p=0.038]. As we noted in the wider CH analysis, these patients were more likely to have hypertension, a P/LP germline HCM related mutation, a pacemaker and treated with a non-dihydropyridine calcium channel blocker (Table 6).

While patients with DNMT3A, TET2 and ASXL1 CH mutations, exhibited a similar HCM phenotype to those without CH, patients with CH showed a higher mortality or need for orthotopic heart transplant [4 (0.6%) vs. 6 (4.4%), p<0.0001] and MACE [13 (2.0%) vs. 8 (6.2%), p=0.008]. The unadjusted HR for MACE was 3.19 (95% Cl 1.32-7.73; p=0.010) and adjusted HR 3.97 (95% Cl 1.39-11.28; p=0.010). FIG. 8B shows the Kaplan-Meir curve with worse survival for patients with CH (Log-Rank p=0.006).

Clonal Hematopoiesis in HCM Patients with a Sarcomeric Mutation

HCM patients harboring a sarcomeric gene mutation are associated with early age at diagnosis, higher MLVWT and worse outcomes²⁵. Because this subtype of HCM patients have a distinct clinical profile, we sought to investigate if there could be a distinct interaction with CH. The overall characteristics shown among those with or without CH were recorded (data not shown). A trend of a higher burden of symptoms (NYHA class II to IV) [15 (40.5%) versus 47 (28.0%), p=0.132] among those with CH, but it was not statistically significant. However, CH was associated with a higher burden of fibrosis as reflected by LGE in >15% of the LV mass [11 (29.7%) versus 26 (15.3%), p=0.044], with an odds ratio of 2.32 (95% Cl 1.00-5.38, p=0.048), there was a trend towards a higher amount of fibrosis in the LV of those with CH [15.6% (10.4-24.6)] in comparison to those without CH [12.2% (7.7-17.6)], p=0.068. CH patients also showed a higher frequency of ABPR at exercise [25 (29.8%) vs. 10 (62.5%), p=0.012]. Finally, CH was associated with worse outcomes with higher mortality or orthotopic heart transplant [3 (8.1%) versus 2 (1.2%), (p=0.013)]. This was also observed for MACE in patients with CH [5 (13.5%) versus 5 (3.0%), p=0.008], with an unadjusted HR of 5.28 (95% Cl 1.51-18.4, p=0.009), and adjusted HR of 6.89 (95% CI 1.78-26.6, p=0.005). FIG. 8C shows the Kaplan-Meier survival curve showing a worse survival for those with CH (log-rank p=0.003).

We then stratified the HCM phenotype and clinical outcomes in those with sarcomeric mutations and specifically the presence of DNMT3A, TET2, and ASXL1 mutations. Baseline characteristics was similar between groups. The amount of fibrosis assessed by LGE was higher in those with CH [21.2% (13.7-41.8) versus 12.2% (7.7-17.5), p=0.014], whereas LGE≥15% was not statistically different in HCM patients with and without CH [7 (35.0%) vs. 30 (18.3%), p=0.09]. However, CH patients showed a higher frequency of abnormal blood pressure response (ABPR) with exercise than those without CH [8 (66.7%) vs. 27 (30.7%), p=0.014). Finally, mortality or orthotopic heart transplant was more common in the CH group than in those without CH [3 (13.0%) vs. 2 (1.1%), p<0.0001], as well as MACE [4 (17.4%) vs. 6 (3.2%), p=0.004]. MACE was associated with CH with an unadjusted HR of 5.04 (95% Cl 1.39-18.28) p=0.014 and adjusted HR of 7.23 (95% Cl 1.79-29.13) p=0.005. FIG. 8D shows the Kaplan-Meier survival curve showing a worse survival for those with CH (log-rank p=0.007).

### Biomarkers and Cytokines/Chemokines

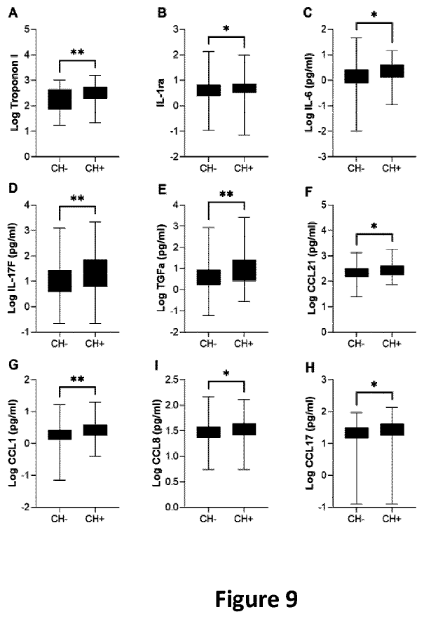

A panel of 71 cytokines and chemokines, BNP and cardiac troponin I were evaluated in patients with a sarcomeric mutation and levels were compared among those with (N=37) or without CH (N=169). It was observed that troponin I (p=0.008), IL-1ra (p=0.037), IL-6 (p=0.028), IL-17F (p=0.006), TGFa (p=0.005), CCL21 (p=0.036), CCL1 (p=0.002), CCL8 (p=0.036), and CCL17 (p=0.047) were all upregulated in patients with CH. FIG. 9 shows levels measured among those with and without CH. We then sought to investigate if specific CH mutations were associated with specific cytokines/chemokines. In HCM patients with sarcomeric mutations, those with CH due to DNMT3A (N=16) mutations showed higher levels of IL-9 (p=0.031) and CXCL12 (p=0.046). TET2 CH carriers (N=6) showed higher levels of troponin I (p=0.013), IL-5 (p=0.012), IL-10 (p=0.010), CXCL10 (p=0.001), CXCL9 (p=0.012), CCL4 (p=0.037), VEFG-A (p=0.041), CCL21 (p=0.013), CXCL13 (BCAp=0.012) and CCL1 (p=0.007). Finally, CH due to ASXL1 (N=2) did not show any differences in comparison to those without CH.

### Discussion

In this study, we showed that CH prevalence among HCM patients is higher than described in the general population. Fibrosis, a known SCD risk factor, was more prevalent among CH patients with HCM sarcomeric gene mutations. We also observed higher burden of symptoms and higher frequency of ABPR during exercise among CH patients. Finally, CH was associated with higher mortality or orthotopic heart transplant and MACE. Several cytokines, chemokines and troponin were differently expressed in HCM patients with CH, highlighting a plausible causal relationship with the phenotype and outcomes. This is the first cohort in which there is an association between CH prevalence, HCM phenotype and clinical outcomes.

To our knowledge, this is the first study to evaluate the prevalence of CH in patients with HCM. In this study, we found that 22.9% of HCM patients harbored CH.

When restricting analysis to the three specific genes DNMT3A, TET2 and ASXL1, we observed that 17% of HCM patients harbored CH mutations.

Fibrosis was more common in those patients with HCM who have a sarcomeric mutation and specific DNMT3A, TET2 and ASXL1 mutations. This is an important finding as fibrosis is linked to SCD in patients with HCM⁴⁰. In addition, ABPR at exercise, a known marker of SCD risk and worse outcomes⁴¹⁻⁴³, was also more frequent among those with CH and HCM with sarcomeric mutations. This result potentially illustrates that CH could affect the HCM phenotype and promote adverse outcomes in patients with HCM.

We showed that, in subsets of HCM patients and evaluating for overall CH or specific CH mutations, the mortality or need for orthotopic heart transplant is increased among CH patients even when adjusting for confounding factors. Moreover, condensing important major cardiovascular events in HCM, such as stroke, sudden cardiac death, appropriate ICD shock, death or orthotopic heart transplant, CH increases its risk in all subsets of patients, reaching the highest risk among those with sarcomeric mutations and with specific CH genes. Our results show that CH is a new risk factor among HCM patients. In fact, HCM patients with sarcomeric mutations with CH on the most common genes showed the worse survival (17%), contrasting to recent cohorts showing that HCM patients have a low mortality.⁹ Our results do not show a clear evidence of which mechanisms CH increase mortality in HCM, but its strong association with ageing¹³ leads us to hypothesize that the epigenic ageing and the inflammatory milieu may have a causal relationship with the reduced survival.

Among HCM with sarcomeric mutations, CH was associated with several inflammatory cytokines and chemokines such as IL-1ra, IL-6, IL-17F, TGFa, CCL21, CCL1, CCL8, and CCL17, but also with troponin I, a marker of myocyte injury that is associates with clinical outcomes in HCM.⁴⁷ However, specific CH mutations may have distinct prognosis and inflammatory profiles.^{14,34} We observed that DNMT3A driven CH was associated with IL-9 and CXCL12. TET2 driven CH was associated with the higher number of differently expressed cytokines/chemokines including troponin I among all tested genes. DNMT3A was associated with LV remodeling and worse outcomes, but the high frequency CH mediated by this gene in the population could be explained by its low lethality and lower burden of inflammation, introducing a survival bias.^{11,19,28,29,34} Cardiac aging processes mediated by TET2 have shown to produce an enhanced inflammatory background that could be related to hypertrophy and fibrosis, but the worse prognosis associated with TET2 could also explain its lower frequency in studies.^{11,17,29} ASXL1 carriers did not show different levels of expressed cytokines, chemokines, BNP or Troponin I. We believe that the low number of patients with this specific mutation could explain the results.

### Example 4

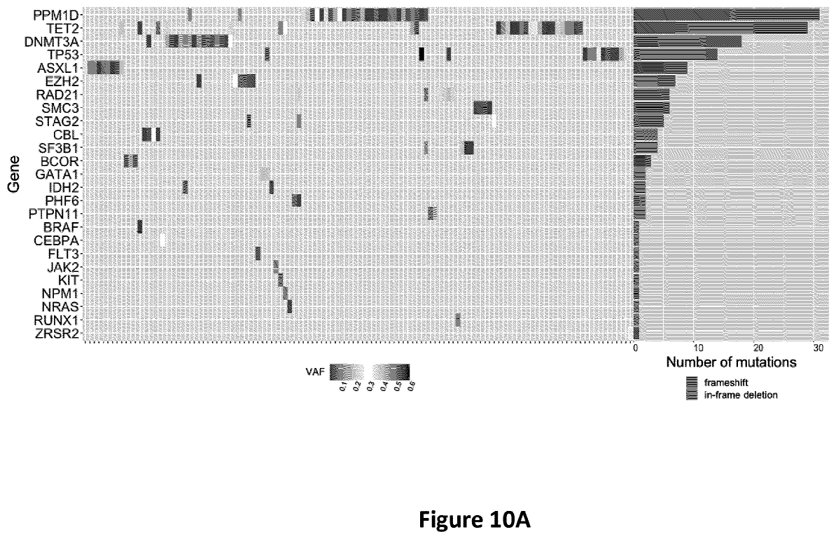

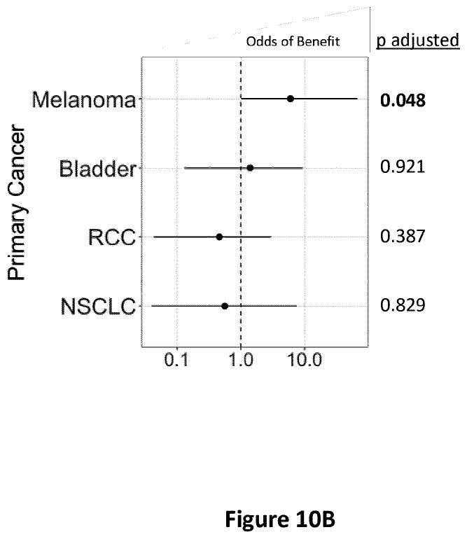

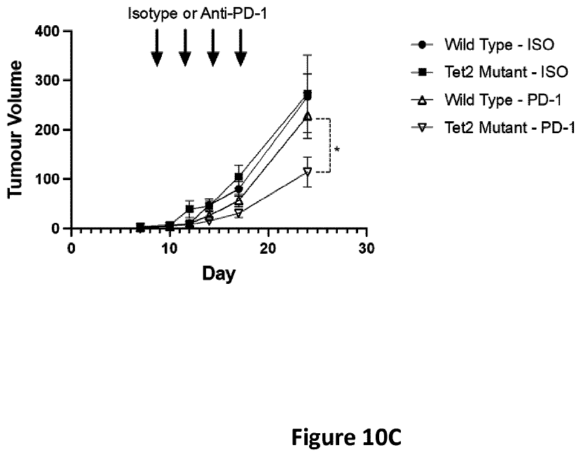

We also investigated TET2 mutant clonal hematopoiesis and its association with the benefit of immunotherapy, as well as metastasis of non-hematological cancers. Referring to FIG. 10, TET2-mutant clonal hematopoiesis is associated with clinical benefit from immunotherapy in melanoma. FIG. 10A shows 569 patients with melanoma, bladder cancer, renal cell carcinoma (RCC), or non-small cell lung cancer (NSCLC) treated with immune-checkpoint blockade were screened for clonal hematopoiesis using publicly-available exome sequencing, with at least one mutation of variant allele frequency (VAF)>0.02 detected in 74 patients (datasets EGAD00001006632, SRP064805, SRP067938, SRP072934, SRP090294, SRP095809, SRP115658, SRP128156). FIG. 10B shows TET2-mutant clonal hematopoiesis is associated with significantly higher odds of clinical benefit 6 months after receiving immunotherapy in melanoma patients (Odds Ratio=5.98), but not bladder cancer, renal cell carcinoma, or non-small cell lung cancer patients (p value from Firth's multivariate logistic regression adjusted for patient age, sex, study, and immune checkpoint). FIG. 10C shows in an animal model of immunotherapy, mice with Tet2-mutant hematopoiesis—mimicking TET2-mutant clonal hematopoiesis—show enhanced response to PD-1 immune checkpoint blockade, while isotype control (ISO) treated tumours show identical growth kinetics (*p<0.05 by Mann-Whitney Test).

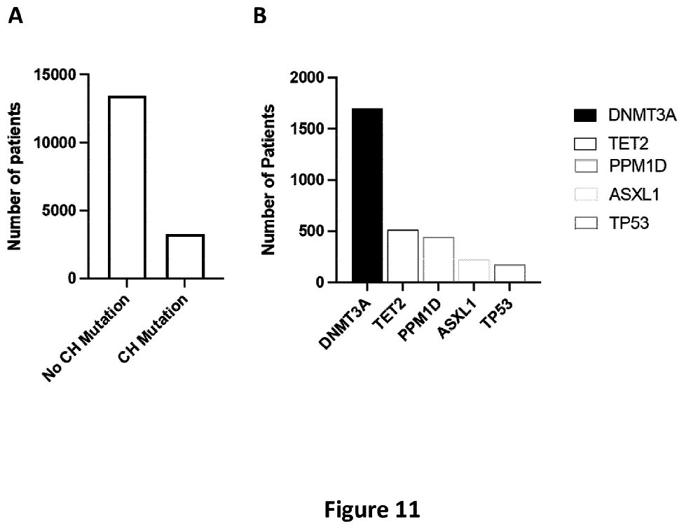

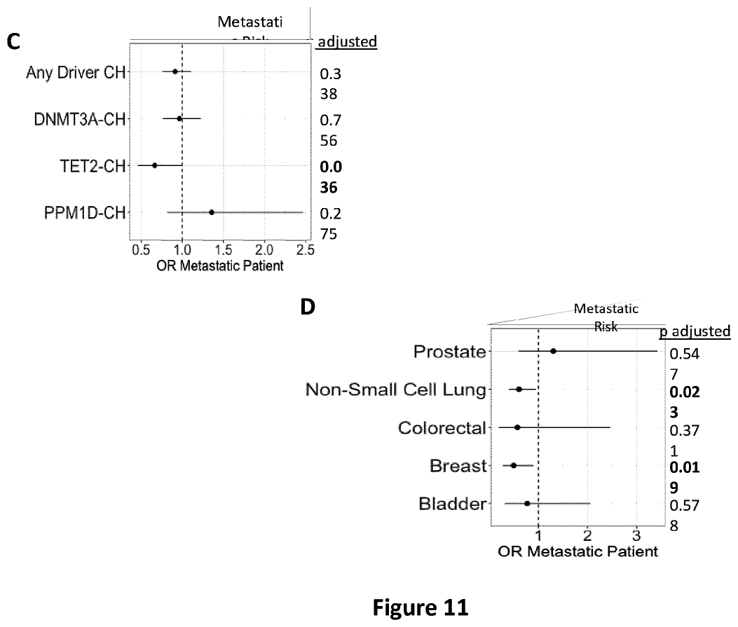

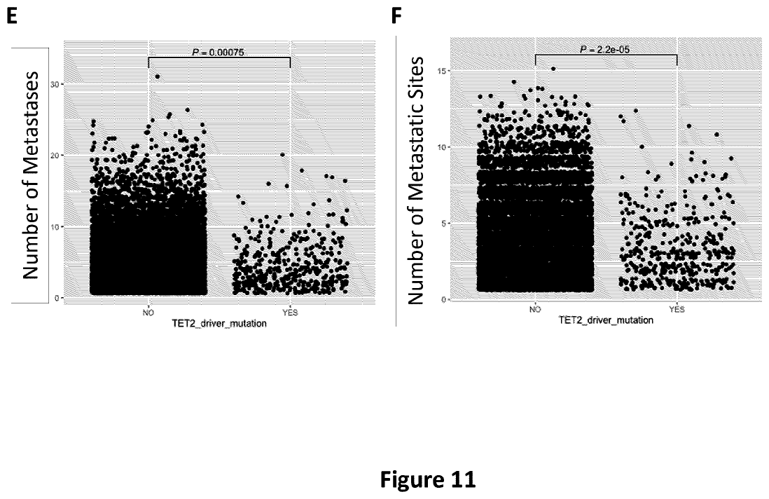

Referring to FIG. 11, TET2-mutant clonal hematopoiesis is associated with lower risk of metastatic disease in patients with non-hematological cancers. FIG. 11A shows 16,744 patients with metastatic or non-metastatic solid tumours from Nguyen et al. Cell, 2022 were tested for clonal hematopoiesis in Bolton et al. Nature Genetics, 2020 using the MSK-IMPACT targeted sequencing panel. CH mutations with variant allele frequency of at least 0.02 were detected in 19.5% of patients. FIG. 11B shows the 5 most commonly detected clonal hematopoiesis mutations from FIG. 11A are shown. FIG. 11C shows exposure to TET2-mutant CH is associated with lower risk of having metastatic cancer in patients from FIG. 11A as assessed by multivariate logistic regression adjusted for age, sex, smoking status, and chemotherapy treatment. FIG. 10D shows exposure to TET2-CH is associated with significantly lower risk of having metastases in patients from FIG. 11A with Non-Small Cell Lung and Breast Cancer, with a trend towards lower risk of metastases in colorectal and bladder cancer, as assessed by multivariate logistic regression adjusted for age, sex, smoking status, and chemotherapy treatment. Patients in FIG. 11A with TET2-mutant CH have significantly few metastases detected on clinical imaging (FIG. 11E) and have fewer overall sites/organs involved with metastatic disease (FIG. 11F), p values from Wilcoxon Rank Sum Test.

### Example 5

Applicant further sought to investigate the prevalence of CH among solid organ transplant (SOT) recipients (heart, lung, liver and kidney), study the association with specific CH-related genes and the impact on outcomes.

### Methods and Materials

### Study Population

We conducted a retrospective study of 1,500 patients who underwent SOT at the Toronto General Hospital between 2005 and 2021. The inclusion criteria were patients aged 18 years or older with available biospecimens and complete clinical evaluation. Patients with active malignancies were excluded, as it is a criterion for solid organ transplant candidacy.

### Data Collection

The electronic health records of the SOT recipients were reviewed to collect demographic data, medical history, laboratory assessments, date of SOT, transplant-related treatment at the time of the procedure, and the immunosuppressive regimen at one year post-SOT. Baseline characteristics, including demographic data and comorbidities, were collected as close as possible to the biospecimens collection date due to their relationship with CH mutations.

### Follow-Up and Outcomes

Patients were followed from the time of SOT procedure until the last visit at our institution or death. The following outcomes were evaluated: the first occurrence of acute cellular rejection, CMV infection, de novo post-transplant malignancy, CAV, and death.

### Genetic Sequencing

Peripheral blood samples from patients who had provided consent during their visits to Toronto General Hospital. These samples were stored in the biobank of the Peter Munk Cardiac Centre. To construct the sequencing libraries, 100 ng of DNA was utilized, and single molecule Molecular Inversion Probes (smMIP) were employed. The smMIPs were utilized to capture 35 genes known to be recurrently mutated in myeloid neoplasms. Sequencing was performed using the Illumina Novaseq platform, and an in-house computational pipeline was used to reduce artifacts and false-positive mutation calls. Alleles were filtered based on a P-value cut-off of 0.05 and were manually inspected based on several criteria, including base-pair change, annotation in COSMIC, minor allele frequency, and number of reads supporting the alternative allele. The resulting variants were analyzed and manually inspected to avoid selecting false positives.

### Statistical Analysis

The normality of all variables was assessed using the Shapiro-Wilk test, and both normal and non-normal continuous variables are reported as mean±standard deviation or median (interquartile range). Student's t-test or Mann-Whitney test were used to evaluate differences among groups depending on the normality of the data. Categorical variables were assessed using chi-square test or Fisher's exact test. All analyses were performed using SPSS, version 25.0 (SPSS Inc., NY, USA), with a statistical significance level of 0.05 and a two-sided p-value.

### Results and Discussion

Out of the 1,500 patients enrolled, 782 have been sequenced as of the current date. Among these patients, 127 (16.2%) underwent heart transplant, 90 (11.5%) underwent lung transplant, 189 (24.2%) underwent kidney transplant, 374 (47.8%) underwent liver transplant, and 2 (0.3%) underwent lung-liver transplant. The average age of the entire cohort was 54.2±12.7 years, with a majority of male patients (52.9%). The age distribution varied significantly across organ groups (p<0.0001), with lung-liver transplant patients being the youngest (47±12.7), followed by heart (49.2±14.0), kidney (53.7±13.3), lung (54.4±15.1) and liver (56.1±10.8) transplant patients. Sex also differed among the organ groups (p=0.05), with all lung-liver transplant recipients being male, and a majority of heart (76.4%), kidney (76.2%), liver (66.5%), and lung (58.9%) transplant recipients being male. For a summary of the overall cohort characteristics, refer to Table 8.

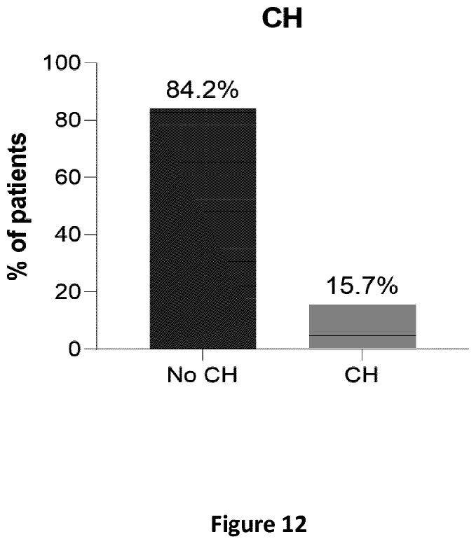

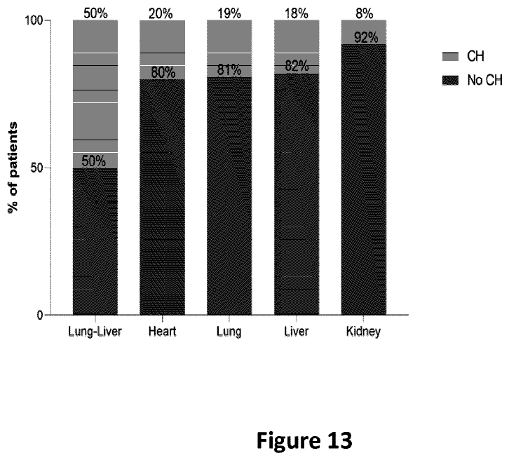

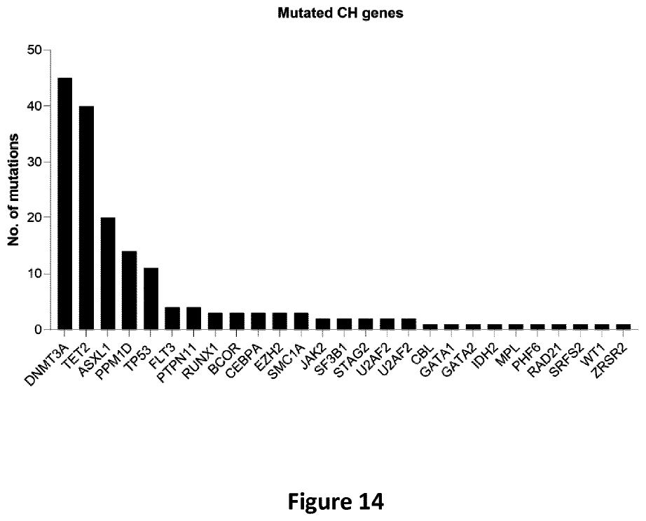

CH was observed in 123 (15.7%) patients. CH prevalence was similar across the organ groups (FIG. 12), except for kidney (8%) that was lower than heart (20%), lung (19%), and (liver 18%), p=0.007. Lung-liver showed a 50% of CH, but the low number of patients may explain this result.

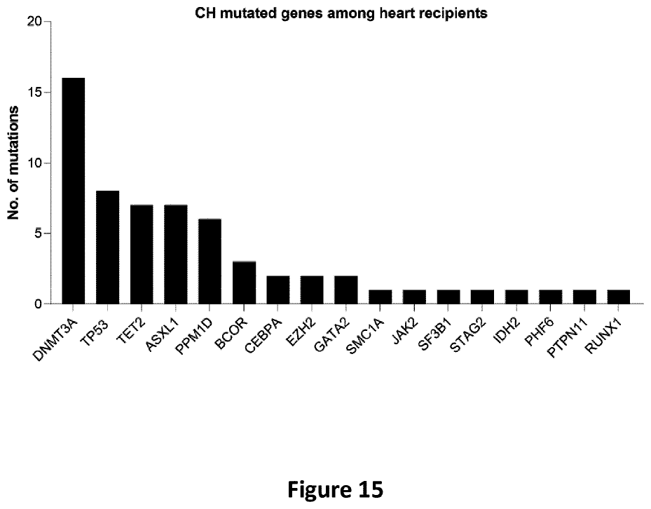

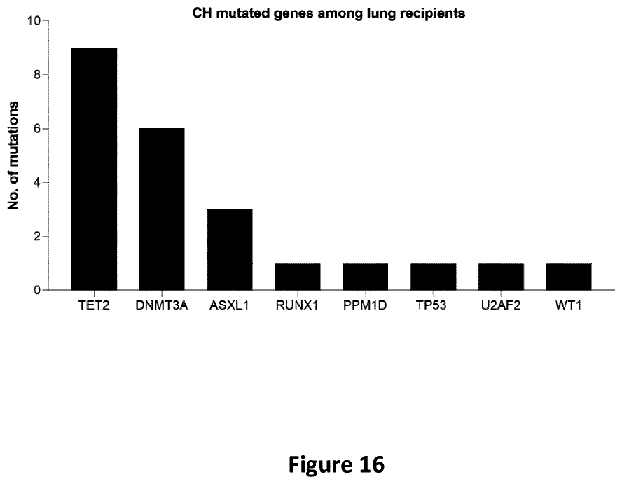

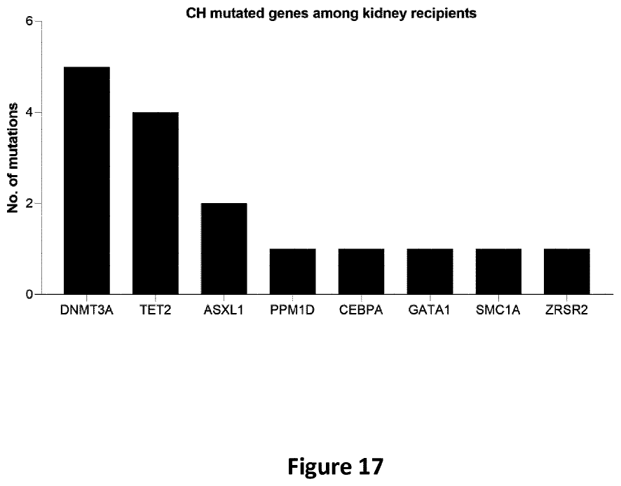

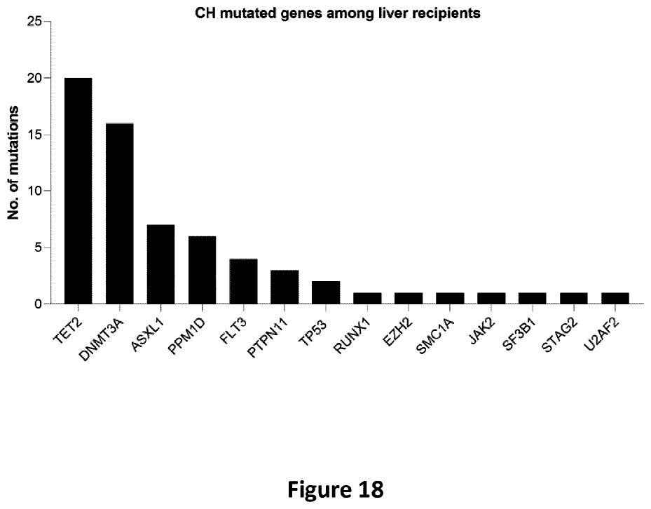

We than analyzed the specific mutated genes among patients with CH. In the literature, DNMT3A is the most common mutated gene in the general population, followed by TET2. [13] In our cohort, DNMT3A was the most commonly mutated gene (n=45, 5.7%), followed by TET2 (n=40, 5.1%) and ASXL1 (n=20, 2.5%). Notably, DNMT3A was the most commonly mutated gene among heart transplant recipients (FIG. 15) and among kidney recipients (FIG. 17), while TET2 was the most common mutated gene among lung (FIG. 16) and liver (FIG. 18) recipients. These findings suggest that TET2 may play a role in lung and liver end-stage disease, but further clinical validation is required.

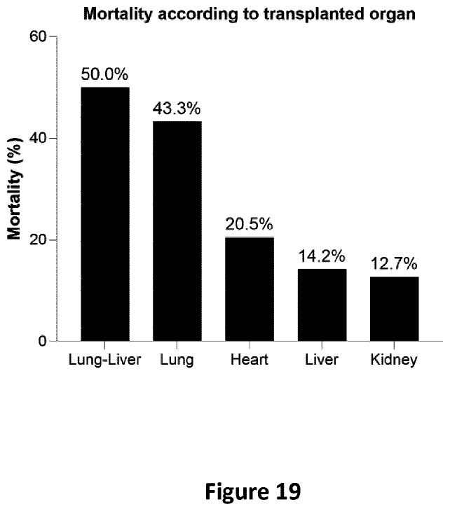

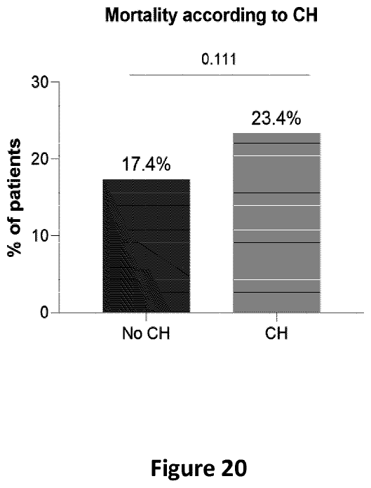

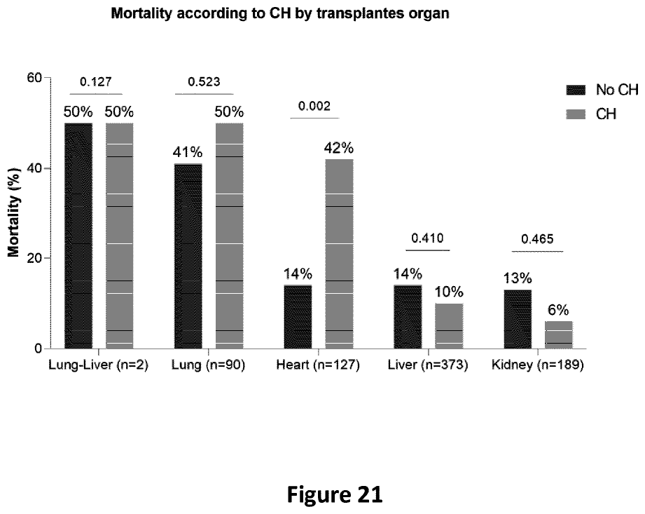

The overall mortality rate in our cohort was 18.3%. Mortality differed across the different SOT (p<0.0001), with the highest mortality observed in lung-liver recipients (50%), followed by lung (43.3%), heart (20.5%), liver (14.2%), and kidney (12.7%) recipients (FIG. 19). We observed a trend in mortality according to the presence of CH (17.4% vs. 23.4%) (FIG. 20). Among solid organ transplant recipients, those who received a heart transplant showed a higher mortality rate when developing CH (14% vs. 42%, p=0.002) with a trending shown for lung. CH did not affect the mortality of recipients of kidney or liver (FIG. 21).

The study partial results shows that CH was observed in 15.7% of patients, with similar prevalence across most organ groups except for kidney, which had a lower prevalence. DNMT3A was the most commonly mutated gene among heart and kidney transplant recipients, while TET2 was the most common in lung and liver recipients. Heart transplant recipients had a higher mortality rate when developing CH.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

TABLE 1
CHARACTERISTICS OF THE PATIENTS AT BASELINE^{A}

| | Cardiogenic Shock | | Ambulatory HF | | P |
|---|---|---|---|---|---|
| | (N = 341) | | (N = 345) | | value |
| Matched variables | | | | | |
| Age (years) | 55 ± 15 | | 57 ± 15 | | 0.25 |
| - median (range) | 58 | (19) | 58 | (19) | |
| Male sex | 249 | (73%) | 247 | (72%) | 0.64 |
| Heart failure aetiology | | | | | |
| Ischemic cardiomyopathy | 107 | (31%) | 106 | (31%) | 0.85 |
| Non-ischemic cardiomyopathy | 221 | (65%) | 225 | (65%) | 0.91 |
| Congenital disease | 13 | (4%) | 14 | (4%) | 0.86 |
| Ejection fraction (%) | 26 ± 12 | | 27 ± 10 | | 0.09 |
| Other baseline characteristics | | | | | |
| Body mass index (kg/m²)^{b} | 26 ± 6 | | 29 ± 6 | | <0.001 |
| Hypertension | 125 | (37%) | 109 | (32%) | 0.16 |
| Dyslipidaemia | 115 | (34%) | 122 | (35%) | 0.65 |
| Diabetes | 99 | (29%) | 78 | (23%) | 0.05 |
| Prior/current smoker | 101 | (30%) | 82 | (24%) | <0.001 |
| Prior history of cancer | 33 | (10%) | 41 | (12%) | 0.35 |
| Prior coronary revascularization | 69 | (20%) | 84 | (24%) | 0.19 |
| Previous cerebrovascular | 39 | (11%) | 33 | (10%) | 0.42 |
| disease/transient ischemic attack | | | | | |
| Atrial Fibrillation/Flutter | 122 | (36%) | 101 | (29%) | 0.06 |
| Chronic Heart Failure | 237 | (70%) | 345 | (100%) | <0.001 |
| Implantable cardioverter-defibrillator | 85 | (25%) | 106 | (31%) | 0.09 |
| Cardiac resynchronization therapy | 69 | (20%) | 70 | (20%) | 0.98 |
| White blood count (×10⁹/L) | 10.7 ± 6.6 | | 7.3 ± 2.4 | | <0.001 |
| Creatinine (μmol/L) | 179 ± 128 | | 110 ± 73 | | <0.001 |
| Sodium (mmol/L) | 135 ± 6 | | 138 ± 3 | | <0.001 |
| B-type natriuretic peptide (pg/mL) | 1,428 | (1730) | 168 | (337) | <0.001 |

^{a}Plus-minus are means ± SD.
^{b}The body-mass index is the weight in kilograms divided by the square of the height in meters.

TABLE 2
Characteristics of Cardiogenic Shock Patients^{a}

| | | | No clonal | | Clonal | | |
|---|---|---|---|---|---|---|---|
| | All cardiogenic shock | | haematopoiesis | | haematopoiesis | | P |
| | (N = 341) | | (N = 256) | | (N = 85) | | value |
| Age (year) | 55 ± 15 | | 54 ± 15 | | 60 ± 14 | | 0.002 |
| Male sex | 249 | (73%) | 188 | (73%) | 61 | (72%) | 0.76 |
| Body mass index (kg/m²)^{b} | 26 ± 6 | | 26 ± 6 | | 26 ± 6 | | 0.50 |
| Hypertension | 125 | (37%) | 87 | (34%) | 38 | (44%) | 0.09 |
| Dyslipidaemia | 115 | (34%) | 78 | (30%) | 37 | (44%) | 0.03 |
| Diabetes | 99 | (29%) | 72 | (28%) | 27 | (31%) | 0.57 |
| Prior/current smoker | 101 | (30%) | 75 | (29%) | 25 | (29%) | 0.89 |
| Prior history of cancer | 33 | (10%) | 25 | (10%) | 9 | (11%) | 0.86 |
| Prior coronary | 69 | (20%) | 52 | (20%) | 17 | (20%) | 0.90 |
| revascularization | | | | | | | |
| Previous cerebrovascular | 39 | (11%) | 27 | (11%) | 12 | (14%) | 0.39 |
| disease/transient ischemic | | | | | | | |
| attack | | | | | | | |
| Cardiogenic shock aetiology | | | | | | | |
| Acute decompensation of | 225 | (66%) | 168 | (66%) | 57 | (67%) | 0.94 |
| chronic heart failure | | | | | | | |
| New onset of heart | 93 | (27%) | 72 | (28%) | 21 | (25%) | 0.27 |
| failure | | | | | | | |
| Myocardial infarction | 10 | (3%) | 6 | (2%) | 4 | (5%) | 0.87 |
| Myocarditis | 11 | (3%) | 8 | (3%) | 3 | (4%) | 0.58 |
| SCAI classification^{c} | | | | | | | |
| B | 27 | (8%) | 21 | (8%) | 6 | (7%) | 0.73 |
| C | 27 | (8%) | 20 | (8%) | 7 | (8%) | 0.90 |
| D | 285 | (83%) | 213 | (83%) | 72 | (85%) | 0.74 |
| E | 2 | (1%) | 2 | (1%) | 0 | | 0.41 |
| Number of inotropes or | 1.7 ± 1.0 | | 1.6 ± 1.0 | | 1.8 ± 1.0 | | 0.24 |
| vasopressors | | | | | | | |
| Pulmonary artery catheter | 178 | (52%) | 134 | (52%) | 44 | (52%) | 0.92 |
| Temporary mechanical | | | | | | | |
| circulatory support | | | | | | | |
| Intra-aortic balloon pump | 26 | (8%) | 20 | (8%) | 6 | (7%) | 0.50 |
| Impella device ® | 8 | (2%) | 8 | (3%) | 0 | | 0.09 |
| Extra-corporeal life | 18 | (5%) | 16 | (6%) | 3 | (4%) | 0.59 |
| support | | | | | | | |
| Mechanical ventilation | 63 | (18%) | 45 | (18%) | 18 | (21%) | 0.45 |
| Renal replacement therapy | 35 | (10%) | 24 | (9%) | 11 | (13%) | 0.34 |
| White blood count (×10⁹/L) | 7.3 ± 2.4 | | 11.1 ± 7.2 | | 9.4 ± 4.3 | | 0.01 |
| Creatinine (□mol/L) | 110 ± 73 | | 176 ± 129 | | 188 ± 124 | | 0.44 |
| Sodium (mmol/L) | 138 ± 3 | | 135 ± 6 | | 133 ± 5 | | 0.02 |
| Peak lactate (mmol/L) | 3.4 ± 3.2 | | 3.4 ± 3.1 | | 3.4 ± 3.1 | | 0.86 |
| B-type natriuretic peptide | 1428 | (1730) | 1446 | (1712) | 1350 | (1737) | 0.42 |
| (pg/mL) | | | | | | | |

^{a}Plus-minus are mean ± SD.
^{b}The body-mass index is the weight in kilograms divided by the square of the height in metres.
^{c}Patients were classified according to the Society for Cardiovascular Angiography & Interventions. Briefly,
Class B are patients at beginning of shock; Class C is classic cardiogenic shock; Class D is deteriorating and failure to respond to initial interventions; and Class E is extreme shock on patients supported by multiple interventions who may be experiencing cardiac arrest and/or extracorporeal life support.

TABLE 3
Clinical characteristics of OHT recipients and according to CH status.

| | All | | No CH | | CH | | |
|---|---|---|---|---|---|---|---|
| Characteristics | (N = 127) | | (N = 101) | | (N = 26) | | P value |
| Age - year | 49 ± 14 | | 48 ± 14 | | 54 ± 13 | | 0.04 |
| Median (range) | 53 | (23) | 52 | (24) | 58 | (21) | 0.03 |
| Male sex - no. (%) | 97 | (77%) | 79 | (78%) | 18 | (69%) | 0.33 |
| Body mass index - kg/m²† | 25 ± 6 | | 25 ± 6 | | 25 ± 5 | | 0.97 |
| Hypertension - no. (%) | 33 | (26%) | 25 | (25%) | 8 | (31%) | 0.53 |
| Dyslipidemia - no. (%) | 33 | (26%) | 24 | (24%) | 9 | (35%) | 0.26 |
| Diabetes - no. (%) | 28 | (22%) | 23 | (23%) | 5 | (19%) | 0.69 |
| Prior smoker - no. (%) | 34 | (27%) | 25 | (25%) | 9 | (35%) | 0.31 |
| Prior neoplasia- no. (%) | 8 | (6%) | 8 | (8%) | None | | 0.20 |
| Heart failure etiology- no. (%) | | | | | | | 0.41 |
| Ischemic cardiomyopathy | 28 | (22%) | 20 | (20%) | 8 | (31%) | |
| Non-ischemic | 90 | (71%) | 73 | (72%) | 17 | (65%) | |
| cardiomyopathy- no. (%) | | | | | | | |
| Congenital disease- no. | 9 | (7%) | 8 | (8%) | 1 | (4%) | |
| (%) | | | | | | | |
| Previous VAD - no. (%) | 44 | (35%) | 37 | (37%) | 7 | (27%) | 0.35 |
| White blood count (×10⁹/L) | 8.8 ± 4.4 | | 7.9 ± 4.5 | | 6.9 ± 3.5 | | 0.11 |
| Sodium (mmol/L) | 134 ± 5 | | 136 ± 5 | | 135 ± 6 | | 0.53 |
| Creatinine (mmol/L) | 117 | (92) | 122 | (86) | 116 | (127) | 0.65 |
| Induction therapy | | | | | | | |
| Thymoglobulin | 123 | (97%) | 97 | (96%) | 26 | (100%) | 0.30 |
| Basiliximab | 4 | (3%) | 4 | (4%) | None | | |
| Primary graft dysfunction | 19 | (15%) | 17 | (17%) | 2 | (8%) | 0.24 |
| In-hospital death post OHT | 12 | (9%) | 10 | (10%) | 2 | (8%) | 0.73 |
| MCS post-OHT | 18 | (14%) | 17 | (17%) | 1 | (4%) | 0.09 |
| Follow-up from OHT, years | 3.2 ± 2.6 | | 2.9 ± 2.1 | | 4.3 ± 3.9 | | 0.01 |
| Donor CMV+ | 57 | (46%) | 45 | (46%) | 12 | (46%) | 0.98 |
| Recipient CMV+ | 80 | (65%) | 63 | (64%) | 17 | (65%) | 0.91 |
| Donor EBV+ | 97 | (14%) | 81 | (80%) | 16 | (62%) | 0.02 |
| Recipient EBV+ | 115 | (93%) | 89 | (91%) | 26 | (100%) | 0.10 |
| Immunosuppressive therapy | | | | | | | |
| at discharge post-OHT | | | | | | | |
| Tacrolimus | 119 | (94%) | 97 | (96%) | 22 | (85%) | 0.03 |
| Sodium mycophenolate | 123 | (97%) | 100 | (99%) | 23 | (89%) | 0.006 |
| Steroids | 127 | (100%) | 101 | (100%) | 26 | (100%) | — |
| MTOR inhibitor | 1 | (1%) | None | | 1 | (4%) | 0.04 |
| Cyclosporine | 6 | (5%) | 3 | (3%) | 3 | (12%) | 0.06 |
| Azathioprine | 1 | (1%) | None | | 1 | (4%) | 0.04 |
| Immunosuppressive therapy | | | | | | | |
| at 1 year after OHT | | | | | | | |
| Tacrolimus | 80 | (86%) | 64 | (87%) | 16 | (84%) | 0.79 |
| Sodium mycophenolate | 71 | (77%) | 58 | (80%) | 13 | (68%) | 0.30 |
| Steroids | 87 | (94%) | 68 | (82%) | 19 | (100%) | 0.19 |
| MTOR inhibitor | 17 | (19%) | 11 | (15%) | 6 | (32%) | 0.09 |
| Cyclosporine | 8 | (9%) | 5 | (7%) | 3 | (16%) | 0.21 |
| Azathioprine | 2 | (2%) | 1 | (1%) | 1 | (5%) | 0.30 |

CH, clonal hematopoiesis; CMV, cytomegalovirus; EBV, Epstein-barr virus; HF, heart failure MCS, mechanical circulatory support; OHT, orthotopic heart transplant; VAD, ventricular assist device; VAF, variant allele frequency;

TABLE 4
Clinical outcomes in patients with OHT according to CH mutations

| | All | | No CH | | CH | | P- |
|---|---|---|---|---|---|---|---|
| | (N = 127) | | (N = 101) | | (N = 26) | | value |
| CMV infection | 59 | (46%) | 48 | (47.5%) | 11 | (42.3%) | 0.63 |
| Acute cellular rejection | 69 | (54%) | 51 | (53%) | 18 | (69%) | 0.14 |
| 2/3R | | | | | | | |
| Post-transplant | 11 | (9%) | 9 | (9%) | 2 | (8%) | 0.84 |
| malignancy | | | | | | | |
| CAV grade 2 or 3 | 4 | (3%) | None | | 4 | (18%) | <0.001 |
| Mortality | 26 | (20%) | 15 | (15%) | 11 | (42%) | 0.008 |

CAV, cardiac allograft vasculopathy; CH, clonal hematopoiesis; CMV, cytomegalovirus

TABLE 5
List of clonal hematopoiesis mutations detected among orthotopic heart transplant recipients.

| | | | Start | End | | Variant |
|---|---|---|---|---|---|---|
| Sample ID | Gene | Chromosome | position | position | Variant type | type |
| [symbol] CHCVD_0004 | CEBPA | chr19 | 33792312 | 33792312 | Missense_Variant | SNV |
| [symbol] CHCVD_0006A | ASXL1 | chr20 | 31023894 | 31023894 | Missense_Variant | SNV |
| [symbol] CHCVD_0010B | DNMT3A | chr2 | 25463562 | 25463562 | Frame_Shift_Del | indel |
| [symbol] CHCVD_0010B | DNMT3A | chr2 | 25467086 | 25467086 | Frame_Shift_Del | indel |
| [symbol] CHCVD_0010B | FLT3 | chr13 | 28608064 | 28608064 | Missense_Variant | SNV |
| [symbol] CHCVD_0011 | DNMT3A | chr2 | 25463187 | 25463187 | Missense_Variant | SNV |
| [symbol] CHCVD_0014 | ASXL1 | chr20 | 31024221 | 31024221 | Missense_Variant | SNV |
| [symbol] CHCVD_0017 | ASXL1 | chr20 | 31022344 | 31022344 | Missense_Variant | SNV |
| [symbol] CHCVD_0017 | BCOR | chrX | 39933074 | 39933074 | Missense_Variant | SNV |
| [symbol] HCVD_0017 | DNMT3A | chr2 | 25470612 | 25470612 | Missense_Variant | SNV |
| [symbol] CVD_0017 | SF3B1 | chr2 | 198266552 | 198266552 | Missense_Variant | SNV |
| [symbol] HCVD_0017 | TP53 | chr17 | 7577127 | 7577127 | Stop_Gained | SNV |
| [symbol] HCVD_0029 | DNMT3A | chr2 | 25469086 | 25469086 | Missense_Variant | SNV |
| [symbol] CHCVD_0038 | DNMT3A | chr2 | 25468174 | 25468174 | Missense_Variant | SNV |
| [symbol] CHCVD_0038 | TP53 | chr17 | 7578371 | 7578371 | Missense_Variant | SNV |
| ARCHCVD_0038 | TP53 | chr17 | 7578380 | 7578380 | Missense_Variant | SNV |
| ARCHCVD_0041 | TP53 | chr17 | 7578380 | 7578380 | Missense_Variant | SNV |
| ARCHCVD_0043 | IDH2 | chr15 | 90631946 | 90631946 | Missense_Variant | SNV |
| ARCHCVD_0046 | DNMT3A | chr2 | 25470590 | 25470590 | Missense_Variant | SNV |
| ARCHCVD_0055 | TET2 | chr4 | 106158134 | 106158134 | Missense_Variant | SNV |
| ARCHCVD_0060 | ASXL1 | chr20 | 31023667 | 31023667 | Missense_Variant | SNV |
| ARCHCVD_0060 | TET2 | chr4 | 106158560 | 106158560 | Missense_Variant | SNV |
| ARCHCVD_0096 | TET2 | chr4 | 106157326 | 106157326 | Stop_Gained | SNV |
| ARCHCVD_0100 | DNMT3A | chr2 | 25463568 | 25463568 | Missense_Variant | SNV |
| ARCHCVD_0100 | PTPN11 | chr12 | 112910785 | 112910785 | Missense_Variant | SNV |
| ARCHCVD_0114 | DNMT3A | chr2 | 25467491 | 25467491 | Missense_Variant | SNV |
| [symbol] CHCVD_0117 | DNMT3A | chr2 | 25467124 | 25467124 | Frame_Shift_Ins | indel |
| [symbol] CHCVD_0117 | PPM1D | chr17 | 58740549 | 58740549 | Frame_Shift_Del | indel |
| [symbol] CHCVD_0117 | PPM1D | chr17 | 58740604 | 58740607 | Frame_Shift_Del | indel |
| [symbol] CHCVD_0117 | PPM1D | chr17 | 58740714 | 58740714 | Frame_Shift_Del | indel |
| [symbol] CHCVD_0117 | PPM1D | chr17 | 58740732 | 58740732 | Frame_Shift_Del | indel |
| [symbol] CHCVD_0117 | TP53 | chr17 | 7577526 | 7577526 | Missense_Variant | SNV |
| [symbol] CHCVD_0128 | DNMT3A | chr2 | 25469633 | 25469633 | Missense_Variant | SNV |
| [symbol] CHCVD_0128 | TP53 | chr17 | 7577556 | 7577556 | Missense_Variant | SNV |
| [symbol] CHCVD_0147 | ASXL1 | chr20 | 31023190 | 31023190 | Missense_Variant | SNV |
| [symbol] CHCVD_0149 | DNMT3A | chr2 | 25463196 | 25463197 | Frame_Shift_Del | indel |
| [symbol] CHCVD_0165 | TP53 | chr17 | 7577120 | 7577120 | Missense_Variant | SNV |
| [symbol] CHCVD_0166 | DNMT3A | chr2 | 25463236 | 25463238 | Frame_Shift_Del | indel |
| [symbol] HCVD_0166 | DNMT3A | chr2 | 25463586 | 25463586 | Missense_Variant | SNV |
| [symbol] HCVD_0166 | DNMT3A | chr2 | 25464455 | 25464455 | Missense_Variant | SNV |
| [symbol] HCVD_0170 | DNMT3A | chr2 | 25463583 | 25463583 | Missense_Variant | SNV |
| [symbol] HCVD_0200 | PHF6 | chrX | 133549136 | 133549136 | Stop_Gained | SNV |
| [symbol] CHCVD_0210 | SMC1A | chrX | 53431967 | 53431967 | Stop_Gained | SNV |
| [symbol] CHCVD_0227 | DNMT3A | chr2 | 25462078 | 25462078 | Missense_Variant | SNV |
| ARCHCVD_0227 | PPM1D | chr17 | 58740522 | 58740522 | Frame_Shift_Del | indel |
| ARCHCVD_0241 | RUNX1 | chr21 | 36252975 | 36252975 | Missense_Variant | SNV |
| ARCHCVD_0296 | DNMT3A | chr2 | 25458637 | 25458637 | Stop_Gained | SNV |
| ARCHCVD_0331 | DNMT3A | chr2 | 25470583 | 25470583 | Stop_Gained | SNV |
| ARCHCVD_0398 | ASXL1 | chr20 | 31022341 | 31022341 | Missense_Variant | SNV |
| ARCHCVD_0456 | GATA2 | chr3 | 128200154 | 128200154 | Missense_Variant | SNV |
| ARCHCVD_0492 | TET2 | chr4 | 106157803 | 106157810 | Frame_Shift_Del | indel |
| ARCHCVD_0563 | BCOR | chrX | 39933121 | 39933121 | Missense_Variant | SNV |
| ARCHCVD_0563 | EZH2 | chr7 | 148506185 | 148506185 | Missense_Variant | SNV |
| ARCHCVD_0568 | EZH2 | chr7 | 148504787 | 148504787 | Missense_Variant | SNV |
| ARCHCVD_0568 | STAG2 | chrX | 123220456 | 123220456 | Missense_Variant | SNV |
| [symbol] CHCVD_0585 | TP53 | chr17 | 7578278 | 7578278 | Missense_Variant | SNV |
| [symbol] CHCVD_0603 | JAK2 | chr9 | 5073739 | 5073739 | Missense_Variant | SNV |
| [symbol] CHCVD_0614 | TET2 | chr4 | 106197039 | 106197039 | Missense_Variant | SNV |
| [symbol] CHCVD_0629 | CEBPA | chr19 | 33792987 | 33792987 | Missense_Variant | SNV |
| [symbol] CHCVD_0636 | DNMT3A | chr2 | 25457243 | 25457243 | Missense_Variant | SNV |
| [symbol] CHCVD_0636 | DNMT3A | chr2 | 25469096 | 25469096 | Frame_Shift_Del | indel |
| [symbol] CHCVD_0636 | DNMT3A | chr2 | 25469162 | 25469162 | Stop_Gained | SNV |
| [symbol] CHCVD_0644 | TET2 | chr4 | 106155429 | 106155429 | Missense_Variant | SNV |
| [symbol] CHCVD_0657 | BCOR | chrX | 39933039 | 39933039 | Frame_Shift_Del | indel |
| [symbol] CHCVD_0673 | PPM1D | chr17 | 58740698 | 58740698 | Stop_Gained | SNV |
| [symbol] CHCVD_0680 | ASXL1 | chr20 | 31021109 | 31021109 | Missense_Variant | SNV |

| | Ref. | Alt. | Protein | Alt. | Ref. | |
|---|---|---|---|---|---|---|
| Sample ID | Allele | Allele | change | reads | reads | VAF |
| [symbol] CHCVD_0004 | T | A | p.Thr337Ser | 442 | 656 | 0.67378049 |
| [symbol] CHCVD_0006A | G | A | p.Asp1127Asn | 2559 | 13221 | 0.19355571 |
| [symbol] CHCVD_0010B | C | − | p.Gly707fs | 791 | 32884 | 0.02405425 |
| [symbol] CHCVD_0010B | G | − | p.Arg597fs | 277 | 92757 | 0.0029863 |
| [symbol] CHCVD_0010B | T | A | p.Lys634Asn | 19623 | 42835 | 0.45810669 |
| [symbol] CHCVD_0011 | A | G | p.Ile769Thr | 496 | 16037 | 0.03092848 |
| [symbol] CHCVD_0014 | T | G | p.Ser1236Ala | 6179 | 37380 | 0.1653023 |
| [symbol] CHCVD_0017 | G | A | p.Gly610Asp | 1265 | 7311 | 0.17302695 |
| [symbol] CHCVD_0017 | A | G | p.Trp509Arg | 745 | 46356 | 0.01607127 |
| [symbol] HCVD_0017 | G | A | p.Arg288Trp | 1350 | 4998 | 0.27010804 |
| [symbol] CVD_0017 | C | T | p.Ala762Thr | 734 | 24252 | 0.03026555 |
| [symbol] HCVD_0017 | C | A | p.Glu271* | 619 | 6642 | 0.09319482 |
| [symbol] HCVD_0029 | G | A | p.Arg458Trp | 416 | 4097 | 0.10153771 |
| [symbol] CHCVD_0038 | T | C | p.Asn501Ser | 9671 | 29430 | 0.32861026 |
| [symbol] CHCVD_0038 | C | G | p.Gly187Arg | 133 | 17741 | 0.00749676 |
| ARCHCVD_0038 | C | G | p.Asp184His | 133 | 17740 | 0.00749718 |
| ARCHCVD_0041 | C | G | p.Asp184His | 142 | 20782 | 0.00683284 |
| ARCHCVD_0043 | T | C | p.Asn136Ser | 2076 | 5343 | 0.38854576 |
| ARCHCVD_0046 | A | T | p.Leu295Gln | 570 | 8500 | 0.06705882 |
| ARCHCVD_0055 | C | T | p.Pro1012Leu | 3565 | 9098 | 0.39184436 |
| ARCHCVD_0060 | G | A | p.Arg1051His | 841 | 9827 | 0.08558054 |
| ARCHCVD_0060 | A | G | p.Asn1154Ser | 2104 | 7665 | 0.27449446 |
| ARCHCVD_0096 | C | T | p.Gln743* | 200 | 28409 | 0.00704002 |
| ARCHCVD_0100 | A | G | p.Ile705Thr | 375 | 12290 | 0.03051261 |
| ARCHCVD_0100 | G | A | p.Arg265Gln | 238 | 21688 | 0.01097381 |
| ARCHCVD_0114 | C | T | p.Asp529Asn | 170 | 10755 | 0.0158066 |
| [symbol] CHCVD_0117 | T | + | p.Tyr584fs | 1599 | 36285 | 0.0440678 |
| [symbol] CHCVD_0117 | G | − | p.Ile486fs | 77 | 24796 | 0.00310534 |
| [symbol] CHCVD_0117 | AACA | − | p.Asn505fs | 70 | 29465 | 0.0023757 |
| [symbol] CHCVD_0117 | A | − | p.Glu540fs | 362 | 22820 | 0.01586328 |
| [symbol] CHCVD_0117 | T | − | p.Leu546fs | 262 | 22819 | 0.01148166 |
| [symbol] CHCVD_0117 | A | T | p.Leu252His | 278 | 15061 | 0.01845827 |
| [symbol] CHCVD_0128 | G | A | p.Arg379Cys | 4474 | 62094 | 0.07205205 |
| [symbol] CHCVD_0128 | C | T | p.Cys242Tyr | 422 | 40218 | 0.01049281 |
| [symbol] CHCVD_0147 | C | A | p.Ser892Tyr | 228 | 36353 | 0.00627183 |
| [symbol] CHCVD_0149 | TT | − | p.Lys766fs | 1027 | 37205 | 0.02760382 |
| [symbol] CHCVD_0165 | C | G | p.Arg273Pro | 4705 | 12830 | 0.36671863 |
| [symbol] CHCVD_0166 | A | − | p.Trp753fs | 1226 | 55482 | 0.02209726 |
| [symbol] HCVD_0166 | C | T | p.Gly699Asp | 658 | 13363 | 0.04924044 |
| [symbol] HCVD_0166 | G | C | p.Asp686Glu | 145 | 12401 | 0.01169261 |
| [symbol] HCVD_0170 | G | A | p.Pro700Leu | 172 | 15467 | 0.01112045 |
| [symbol] HCVD_0200 | C | T | p.Arg274* | 895 | 62494 | 0.01432137 |
| [symbol] CHCVD_0210 | G | A | p.Arg725* | 697 | 13878 | 0.05022338 |
| [symbol] CHCVD_0227 | G | A | p.Pro777Ser | 547 | 19125 | 0.02860131 |
| ARCHCVD_0227 | A | − | p.Asn477fs | 728 | 13283 | 0.0548069 |
| ARCHCVD_0241 | C | T | p.Leu129Leu | 203 | 40261 | 0.0050421 |
| ARCHCVD_0296 | G | A | p.Gln846* | 196 | 7986 | 0.02454295 |
| ARCHCVD_0331 | C | T | p.Trp297* | 152 | 14952 | 0.01016586 |
| ARCHCVD_0398 | C | T | p.Thr609Ile | 5571 | 12858 | 0.43327112 |
| ARCHCVD_0456 | C | G | p.Arg384Thr | 127 | 47555 | 0.00267059 |
| ARCHCVD_0492 | AGAA | − | p.Asn903fs | 240 | 70735 | 0.00339295 |
| | ACCA | | | | | |
| ARCHCVD_0563 | C | T | p.Gly493Asp | 58 | 29158 | 0.00198916 |
| ARCHCVD_0563 | C | G | p.Glu725Gln | 59 | 24362 | 0.0024218 |
| ARCHCVD_0568 | G | C | p.Ala736Gly | 134 | 25452 | 0.00526481 |
| ARCHCVD_0568 | G | C | p.Trp1038Leu | 99 | 40520 | 0.00244324 |
| [symbol] CHCVD_0585 | G | C | p.Pro191Ala | 118 | 31392 | 0.00375892 |
| [symbol] CHCVD_0603 | C | A | p.His606Gln | 9294 | 25742 | 0.36104421 |
| [symbol] CHCVD_0614 | C | G | p.Ser1791Cys | 146 | 29756 | 0.00490657 |
| [symbol] CHCVD_0629 | G | T | p.Pro112Thr | 128 | 130 | 0.98461538 |
| [symbol] CHCVD_0636 | G | A | p.Arg882Cys | 6936 | 287680 | 0.02411012 |
| [symbol] CHCVD_0636 | G | − | p.Lys456fs | 659 | 100672 | 0.00654601 |
| [symbol] CHCVD_0636 | G | C | p.Tyr432* | 897 | 91092 | 0.00984719 |
| [symbol] CHCVD_0644 | G | T | p.Lys110Asn | 476 | 19776 | 0.02406958 |
| [symbol] CHCVD_0657 | G | − | p.Asn520fs | 461 | 41910 | 0.01099976 |
| [symbol] CHCVD_0673 | A | T | p.Lys535* | 3230 | 36839 | 0.08767882 |
| [symbol] CHCVD_0680 | T | A | p.Ser370Thr | 12555 | 20108 | 0.62437836 |

[symbol]

indicates data missing or illegible when filed

TABLE 6
Clinical phenotype and outcomes according to CH among HCM patients with sarcomeric mutations

| | All-CH | | | | | DNMT3A, TET2, ASXL1 | | | | |
|---|---|---|---|---|---|---|---|---|---|---|
| | No CH | | CH | | | No CH | | CH | | |
| | (N = 169) | | (N = 37) | | p | (N = 182) | | (N = 23) | | p |
| Echocardiogram | | | | | | | | | | |
| MLVWT, mean (SD), mm | 17.3 | (4.6) | 17.6 | (5.1) | 0.765 | 17.4 | (4.7) | 17.5 | (4.9) | 0.874 |
| LA diameter, mm | 3.9 | (1.2) | 3.8 | (1.1) | 0.651 | 3.9 | (1.2) | 3.7 | (1.4) | 0.642 |
| LAVi, ml/m² | 38.5 | (30.8) | 41.3 | (20.7) | 0.599 | 39.5 | (30.2) | 35.6 | (20.3) | 0.547 |
| LV EF, % | 58.2 | (15.3) | 62.4 | (6.2) | 0.106 | 58.5 | (14.8) | 63.0 | (7.3) | 0.156 |
| LVOT maximal gradient, mm Hg | 17.2 | (26.6) | 22.5 | (40.3) | 0.316 | 17.6 | (27.4) | 22.6 | (43.5) | 0.442 |
| SAM, No. (%) | 76 | (45.0) | 21 | (56.8) | 0.193 | 84 | (45.9) | 13 | (56.5) | 0.336 |
| Moderate-severe MR, No. (%) | 7 | (4.1) | 1 | (2.7) | 0.681 | 8 | (4.4) | 0 | | 0.306 |
| Cardiac MRI | | | | | | | | | | |
| LV mass, g | 137.3 | (52.9) | 143.1 | (61.4) | 0.601 | 137.8 | (52.9) | 142.3 | (65.5) | 0.728 |
| LV mass index, g/m² | 72.1 | (25.5) | 74.7 | (27.2) | 0.622 | 72.5 | (25.3) | 73.1 | (30.2) | 0.920 |
| MLVWT, mm | 18.9 | (6.4) | 19.2 | (5.6) | 0.814 | 19.0 | (6.4) | 19.0 | (5.8) | 0.980 |
| LV EF, % | 60.8 | (8.3) | 62.1 | (6.2) | 0.390 | 61.0 | (8.2) | 61.1 | (5.8) | 0.979 |
| LGE, No (%) | 134 | (79.3) | 27 | (73.0) | 0.400 | 144 | (78.7) | 17 | (73.9) | 0.601 |
| LGE >15%, No (%) | 26 | (17.7) | 11 | (33.3) | 0.044 | 30 | (18.8) | 7 | (35.0) | 0.090 |
| LV mass % of LGE¹, % | 12.2 | (7.7-17.6) | 15.6 | (10.4-24.6) | 0.068 | 12.2 | (7.7-17.5) | 21.2 | (13.7-41.8) | 0.014 |
| Apical Aneurysm, No (%) | 1 | (0.6) | 1 | (2.7) | 0.145 | 2 | (1.1) | 0 | | 0.613 |
| Syncope, No (%) | 6 | (3.6) | 3 | (8.1) | 0.219 | 7 | (3.8) | 2 | (8.7) | 0.281 |
| ABPR at exercise, No. (%) (85/15) | 25 | (29.8) | 10 | (62.5) | 0.012 | 27 | (30.7) | 8 | (66.7) | 0.014 |
| NVST, No. (%) (145/21) | 52 | (37.7) | 11 | (39.3) | 0.873 | 56 | (37.6) | 7 | (41.2) | 0.772 |
| Stroke, No. (%) | 2 | (1.2) | 2 | (5.4) | 0.092 | 3 | (1.6) | 1 | (4.3) | 0.375 |
| Appropriate ICD shock, No. (%) | 2 | (1.2) | 0 | | 0.506 | 2 | (1.1) | 0 | | 0.614 |
| Cardiac arrest, No. (%) | 0 | | 0 | | n/a | 0 | | 0 | | n/a |
| Death or orthotopic heart transplant, No. (%) | 2 | (1.2) | 3 | (8.1) | 0.013 | 2 | (1.1) | 3 | (13.0) | <0.0001 |
| MACE, No. (%) | 5 | (3.0) | 5 | (13.5) | 0.008 | 6 | (3.4) | 4 | (17.4) | .004 |

¹Quantified LGE >5%.
ABPR, abnormal blood pressure response;
EF, ejection fraction;
LA, left atrium diameter;
LAVi, left atrium volume index;
ICD, implantable cardioverter defibrillator;
LGE, late gadolinium enhancement;
LV, left ventricle;
LVOT, left ventricular outflow tract;
MACE, major cardiovascular events;
MLVWT, maximal left ventricular wall thickness;
MR, mitral regurgitation;
MRI, magnetic resonance imaging;
NSVT, non-sustained ventricular tachycardia;
SAM, systolic anterior motion;

TABLE 7
Overall characteristics of the HCM cohort and between those with or without CH.

| | All | | No CH | | CH | | |
|---|---|---|---|---|---|---|---|
| | (N = 799) | | (N = 616) | | (N = 183) | | P |
| Age, mean (SD), years | 55.7 | (14.6) | 55.3 | (14.5) | 57.2 | (14.8) | 0.126 |
| Age at diagnosis, mean (SD), years | 47.8 | (15.8) | 47.3 | (15.6) | 49.6 | (16.3) | 0.089 |
| Male sex, No. (%) | 547 | (68.5) | 424 | (68.8) | 123 | (67.2) | 0.679 |
| Body mass index¹, mean (SD), kg/m² | 29.0 | (6.9) | 29.0 | (7.1) | 29.2 | (6.2) | 0.647 |
| Hypertension, No. (%) | 315 | (39.4) | 233 | (37.8) | 82 | (44.8) | 0.093 |
| Diabetes, No. (%) | 104 | (13.0) | 78 | (12.6) | 26 | (14.2) | 0.863 |
| Prior/current smoker, No. (%) | 155 | (19.3) | 119 | (19.3) | 36 | (19.6) | 0.637 |
| Coronary artery disease, No. (%) | 73 | (9.1) | 61 | (9.9) | 12 | (6.5) | 0.166 |
| Atrial fibrillation, No. (%) | 143 | (17.8) | 107 | (17.3) | 36 | (19.6) | 0.493 |
| Genetic testing, No. (%) | 712 | (89.1) | 554 | (89.9) | 158 | (86.3) | 0.170 |
| Pathogenic/Likely pathogenic variant, No. (%) | 206 | (25.8) | 169 | (30.5) | 37 | (23.4) | 0.083 |
| MYH7, No. (%) | 53 | (25.7) | 43 | (25.6) | 10 | (27.0) | 0.664 |
| MYBPC3, No. (%) | 127 | (61.6) | 106 | (63.1) | 21 | (56.8) | |
| Other, No. (%) | 26 | (12.6) | 20 | (11.8) | 6 | (16.2) | |
| Family history of HCM, No. (%) | 251 | (31.4) | 194 | (31.5) | 57 | (31.1) | 0.087 |
| Family history of SCD, No. (%) | 37 | (4.6) | 26 | (4.2) | 1 | (5%) | 0.30 |
| Implantable cardioverter defibrillator, No. (%) | 111 | (13.9) | 89 | (14.4) | 22 | (12.0) | 0.405 |
| Pacemaker, No. (%) | 13 | (1.6) | 6 | (1.0) | 7 | (3.8) | 0.007 |
| NYHA II-IV, No. (%) | 235 | (29.4) | 182 | (29.5) | 53 | (28.9) | 0.803 |
| Beta-blocker, No. (%) | 421 | (52.7) | 321 | (52.1) | 100 | (54.6) | 0.547 |
| Non-dihydropyridine calcium channel blocker, No. | 116 | (14.5) | 77 | (12.5) | 39 | (21.3) | 0.003 |
| (%) | | | | | | | |
| Disopyridine, No (%) | 67 | (8.4) | 51 | (8.3) | 16 | (8.7) | 0.842 |
| Diuretic, No (%) | 81 | (10.1) | 59 | (9.5) | 22 | (12.0) | 0.303 |
| Septal reduction therapy, No. (%) | 76 | (9.5) | 53 | (8.6) | 23 | (12.5) | 0.130 |

Abbreviations: CH, clonal hematopoiesis; HCM, hypertrophic cardiomyopathy; SCD, sudden cardiac death
¹Body mass index calculated as weigh (kg)/height²(m)

TABLE 8
Clinical characteristics of OHT recipients
and according to CH status.

| | All | | No CH | | CH | | |
|---|---|---|---|---|---|---|---|
| Characteristics | (N = 782) | | (N = 659) | | (N = 123) | | P value |
| Age - year | 54.2 ± 12.7 | | 54.1 ± 12.8 | | 54.4 ± 12.3 | | 0.795 |
| Median (range) | | | 57 | (48-63) | 58 | (46-63) | |
| Male sex | 52.9% | | 68% | | 67.3% | | 0.882 |
| Organ | | | | | | | |
| Heart | 127 | (16.2%) | 101 | (15.3%) | 26 | (21.1%) | 0.007 |
| Lung | 90 | (11.5%) | 73 | (11.1%) | 17 | (13.8%) | |
| Kidney | 189 | (24.2%) | 174 | (26.4%) | 15 | (12.2%) | |
| Liver | 374 | (47.8%) | 310 | (47.0%) | 64 | (52%) | |
| Lung-Liver | 2 | (0.3%) | 1 | (0.2%) | 1 | (0.8%) | |

TABLE A
SmMIP Probe List

| | | | ext_ | ext_ | ext_ | | | lig_ | lig_ |
|---|---|---|---|---|---|---|---|---|---|
| | logistic_ | | probe_ | probe_ | probe_ | _ | SEQ | probe | probe_ |
| >mip_key | score | chr | start | stop | copy | ext_probe_sequence | ID | stop | copy |
| 1: 43814899-43815068/26, 18/− | 0.78875 | 1 | 43815043 | 43815068 | 1 | TGGTCCACCGCCAGTCTCCTGCCTGG | 1 | 43814916 | 1 |
| 1: 115256459-115256628/21, 23/+ | 0.981109 | 1 | 115256459 | 115256479 | 1 | GATGGCAAATACACAGAGGAA | 2 | 115256628 | 1 |
| 1: 115258635-115258804/21, 23/− | 0.951804 | 1 | 115258784 | 115258804 | 1 | CAACAGGTTCTTGCTGGTGTG | 3 | 115258657 | 1 |
| 2: 25457124-25457293/22, 22/+ | 0.833844 | 2 | 25457124 | 25457145 | 2 | TACCTCAGTTTGCCCCCATGTC | 4 | 25457293 | 1 |
| 2: 25457152-25457321/20, 24/− | 0.839643 | 2 | 25457302 | 25457321 | 2 | TCACCCTGCCCTCTCTGCCT | 5 | 25457175 | 1 |
| 2: 25458549-25458718/20, 24/− | 0.96996 | 2 | 25458699 | 25458718 | 2 | TCCTCTTTTCTCCTCTTCAT | 6 | 25458572 | 1 |
| 2: 25459749-25459918/21, 23/− | 0.860238 | 2 | 25459898 | 25459918 | 1 | GTTATCCAGGTTTCTGTTGTT | 7 | 25459771 | 1 |
| 2: 25461967-25462136/22, 22/− | 0.90969 | 2 | 25462115 | 25462136 | 1 | GCTGTTTCATGCTCCTCCTTGG | 8 | 25461988 | 1 |
| 2: 25463123-25463292/20, 24/+ | 0.881523 | 2 | 25463123 | 25463142 | 1 | AGGTAGAAGCCATTAGTGAG | 9 | 25463292 | 1 |
| 2: 25463185-25463354/20, 24/− | 0.871458 | 2 | 25463335 | 25463354 | 2 | GCCCCAGCTGATGGCTTTCT | 10 | 25463208 | 1 |
| 2: 25463484-25463653/20, 24/+ | 0.917457 | 2 | 25463484 | 25463503 | 1 | GTGGAGGGGACAGGATGGTA | 11 | 25463653 | 1 |
| 2: 25464510-25464629/20, 24/+ | 0.808999 | 2 | 25464510 | 25464529 | 1 | TCCTCACACACCTCCGAGGC | 12 | 25464629 | 1 |
| 2: 25464407-25464566/24, 20/− | 0.825581 | 2 | 25464543 | 25464566 | 1 | GCTGAAGGACTTGGGCATTCAGGT | 13 | 25464426 | 1 |
| 2: 25466743-25466912/20, 24/− | 0.909729 | 2 | 25466893 | 25466912 | 1 | GCTGGGTGGGAGCTTGGGAC | 14 | 25466766 | 1 |
| 2: 25467012-25467163/20, 24/+ | 0.81207 | 2 | 25467012 | 25467031 | 1 | CCCAGCACTCACAAATTCCT | 15 | 25467163 | 1 |
| 2: 25466991-25467110/22, 22/− | 0.807386 | 2 | 25467089 | 25467110 | 1 | AAGGGTACCTACGGGCTGCTGC | 16 | 25467012 | 1 |
| : 25467117-25467237/22, 22/− | 0.81286 | 2 | 25467216 | 25467237 | 1 | GTCTCCTCTGCTCACTGGGTCT | 17 | 25467138 | 1 |
| 2: 25467383-25467552/23, 21/− | 0.896693 | 2 | 25467530 | 25467552 | 1 | GTGGTTTCTGACCCTTCCCGCTG | 18 | 25467403 | 1 |
| 2: 25468100-25468249/20, 24/+ | 0.927609 | 2 | 25468100 | 25468119 | 1 | TCCTGGGTGGGTGTGCTCCT | 19 | 25468249 | 1 |
| 2: 25468075-25468224/23, 21/− | 0.855948 | 2 | 25468202 | 25468224 | 1 | CATCCCCTCCCTCTGCTTTCCAG | 20 | 25468095 | 1 |
| 2: 25468817-25468986/24, 20/+ | 0.721162 | 2 | 25468817 | 25468840 | 1 | CTTCCTAAGTGCCTCTGCTACTCT | 21 | 25468986 | 1 |
| 2: 25468993-25469162/20, 24/+ | 0.826636 | 2 | 25468993 | 25469012 | 2 | TCCTGGTGCCACCCTCTCCA | 22 | 25469162 | 1 |
| 2: 25469087-25469256/24, 20/+ | 0.953003 | 2 | 25469087 | 25469110 | 1 | GGGCTTTTTGGCTGGTGGAGGTGG | 23 | 25469256 | 1 |
| 2: 25469510-25469676/20, 24/+ | 0.700311 | 2 | 25469510 | 25469529 | 1 | TAGGGCCAGAAGGCTGGAAG | 24 | 25469676 | 1 |
| 2: 25469385-25469551/22, 22/− | 0.787747 | 2 | 25469530 | 25469551 | 1 | TGATTGAATGGGCCCTGGGGGG | 25 | 25469406 | 1 |
| 2: 25469926-25470095/23, 21/+ | 0.871049 | 2 | 25469926 | 25469948 | 1 | GACCTCGTAGATGGCTTTGCGGT | 26 | 25470095 | 1 |
| 2: 25469859-25470028/22, 22/− | 0.898607 | 2 | 25470007 | 25470028 | 1 | GGTGTGTGTTGAGAAGCTGATG | 27 | 25469880 | 1 |
| 2: 25470436-25470599/22, 22/+ | 0.833016 | 2 | 25470436 | 25470457 | 1 | CTACTGCCAAACCCCACAACTT | 28 | 25470599 | 1 |
| 2: 25470523-25470685/23, 21/− | 0.911389 | 2 | 25470663 | 25470685 | 1 | ATGGGGGGATCAGGGTGGCAGGG | 29 | 25470543 | 1 |
| 2: 25470913-25471078/26, 18/+ | 0.711861 | 2 | 25470913 | 25470938 | 1 | TCTGGCTCGTCATCGCCTGCTTTGGT | 30 | 25471078 | 1 |
| 2: 198266544-198266713/20, 24/+ | 0.804959 | 2 | 198266544 | 198266563 | 1 | GTAGTTGGCATATTCTGCAT | 31 | 198266713 | 1 |
| 2: 198266417-198266586/23, 21/− | 0.890112 | 2 | 198266564 | 198266586 | 1 | TGGGTATCTTATTCCTCTTATGG | 32 | 198266437 | 1 |
| 2: 198266730-198266899/20, 24/− | 0.956695 | 2 | 198266880 | 198266899 | 1 | TAGGTAATGTTGGGGCATAG | 33 | 198266753 | 1 |
| 2: 198267419-198267588/22, 22/+ | 0.934101 | 2 | 198267419 | 198267440 | 1 | GGGCAATAAAGAAGGAATGCCC | 34 | 198267588 | 1 |
| 2: 198267315-198267484/21, 23/− | 0.947509 | 2 | 198267464 | 198267484 | 1 | CGTAACACAACAGCTAGAGCT | 35 | 198267337 | 1 |
| 2: 209113063-209113232/24, 20/+ | 0.895571 | 2 | 209113063 | 209113086 | 1 | CAAAATCACATTATTGCCAACATG | 36 | 209113232 | 1 |
| 3: 128200021-128200186/21, 23/− | 0.950888 | 3 | 128200166 | 128200186 | 1 | TCTGAAAACTGGTGGTTGCCT | 37 | 128200043 | 1 |
| 3: 128200661-128200783/24, 20/+ | 0.899098 | 3 | 128200661 | 128200684 | 1 | CATTGTGCAGCTTGTAGTAGAGGC | 38 | 128200783 | 1 |
| 3: 128202705-128202871/20, 24/− | 0.848532 | 3 | 128202852 | 128202871 | 2 | TGTCTCTCCCTGTTCCCCTG | 39 | 128202728 | 1 |
| 4: 55589697-55589866/23, 21/+ | 0.941587 | 4 | 55589697 | 55589719 | 1 | GTTGCTGAGGTTTTCCAGCACTC | 40 | 55589866 | 1 |
| 4: 55599221-55599390/20, 24/− | 0.944773 | 4 | 55599371 | 55599390 | 1 | GCAGGACTGTCAAGCAGAGA | 41 | 55599244 | 1 |
| 4: 106155115-106155234/21, 23/+ | 0.928618 | 4 | 106155115 | 106155135 | 1 | ACCAACCATGTTGAGGGCAAC | 42 | 106155234 | 1 |
| 4: 106155243-106155367/20, 24/+ | 0.976973 | 4 | 106155243 | 106155262 | 1 | AAATGGAGACACCAAGTGGC | 43 | 106155367 | 1 |
| 4: 106155161-106155308/20, 24/− | 0.963051 | 4 | 106155289 | 106155308 | 1 | TGGCTTCCCTTCATACAGGG | 44 | 106155184 | 1 |
| 4: 106155408-106155577/22, 22/+ | 0.954287 | 4 | 106155408 | 106155429 | 1 | CTCTGGGCTCCTTCAGATCAAG | 45 | 106155577 | 1 |
| 4: 106155297-106155466/23, 21/− | 0.967386 | 4 | 106155444 | 106155466 | 1 | GTCTTTCTCCATTAGCCTTTTGG | 46 | 106155317 | 1 |
| 4: 106155628-106155777/18, 26/+ | 0.954069 | 4 | 106155628 | 106155645 | 1 | CCAGAGCTTCAGATTCTG | 47 | 106155777 | 1 |
| 4: 106155497-106155666/20, 24/− | 0.964361 | 4 | 106155647 | 106155666 | 1 | ACTTTTCCCCTCCTGCTCAT | 48 | 106155520 | 1 |
| 4: 106155827-106155996/20, 24/+ | 0.947632 | 4 | 106155827 | 106155846 | 2 | AGAAAACCACATCTCACATA | 49 | 106155996 | 1 |
| 4: 106155708-106155877/20, 24/− | 0.944218 | 4 | 106155858 | 106155877 | 1 | ACTCATTAGTAGCCTGACTG | 50 | 106155731 | 1 |
| 4: 106156035-106156184/25, 19/+ | 0.927737 | 4 | 106156035 | 106156059 | 1 | TACCTGTTCCTTTCAGAAACCAGAA | 51 | 106156184 | 2 |
| 4: 106155947-106156116/22, 22/− | 0.98082 | 4 | 106156095 | 106156116 | 1 | GTTATTTTCTGCAGGAGATGGG | 52 | 106155968 | 1 |
| 4: 106156251-106156420/22, 22/+ | 0.902481 | 4 | 106156251 | 106156272 | 1 | CTCAGTGTTCACTAAGGATTCC | 53 | 106156420 | 1 |
| 4: 106156143-106156312/25, 19/− | 0.903718 | 4 | 106156288 | 106156312 | 1 | GCAATTGTGATGGTGGTGGTGGTGT | 54 | 106156161 | 1 |
| 4: 106156470-106156639/22, 22/+ | 0.946747 | 4 | 106156470 | 106156491 | 1 | ACCACCTTCCCAGAGTCCTAAT | 55 | 106156639 | 1 |
| 4: 106156348-106156517/20, 24/− | 0.958324 | 4 | 106156498 | 106156517 | 1 | GAAGGGCTGCATACATGTGT | 56 | 106156371 | 1 |
| 4: 106156694-106156863/20, 24/+ | 0.907492 | 4 | 106156694 | 106156713 | 1 | TGATGAGAAACAAAGAGCAA | 57 | 106156863 | 1 |
| : 106156579-106156748/20, 24/− | 0.965115 | 4 | 106156729 | 106156748 | 1 | CGTGTTTGCTCCTTGTCTCG | 58 | 106156602 | 1 |
| 4: 106156916-106157085/23, 21/+ | 0.951204 | 4 | 106156916 | 106156938 | 1 | CTGGAAATTCCAACATGCCTGGG | 59 | 106157085 | 1 |
| 4: 106156809-106156978/21, 23/− | 0.972677 | 4 | 106156958 | 106156978 | 1 | GCTGTGTTGTTTTCTGGGTGT | 60 | 106156831 | 1 |
| 4: 106157149-106157318/21, 23/+ | 0.947906 | 4 | 106157149 | 106157169 | 1 | CAACAAAGAGCAGATTCCCAA | 61 | 106157318 | 1 |
| 4: 106157036-106157205/20, 24/− | 0.962137 | 4 | 106157186 | 106157205 | 1 | GTGCTGTTTCAACACTGGGG | 62 | 106157059 | 1 |
| 4: 106157399-106157568/18, 26/+ | 0.930675 | 4 | 106157399 | 106157416 | 1 | ATGATCAGCAAAGAGAAG | 63 | 106157568 | 1 |
| 4: 106157269-106157438/18, 26/− | 0.968587 | 4 | 106157421 | 106157438 | 1 | TTAGTCTGGCCAAAGAAT | 64 | 106157294 | 1 |
| 4: 106157597-106157766/21, 23/+ | 0.921979 | 4 | 106157597 | 106157617 | 1 | CTTGTTCAAACAATACACACC | 65 | 106157766 | 1 |
| 4: 106157482-106157651/20, 24/− | 0.953483 | 4 | 106157632 | 106157651 | 1 | GGATGTGTAGTCTGTTCTTT | 66 | 106157505 | 1 |
| 4: 106157839-106158008/21, 23/+ | 0.951954 | 4 | 106157839 | 106157859 | 1 | CTTGCTCAGCAAAGGTACTTG | 67 | 106158008 | 1 |
| 4: 106157718-106157892/22, 22/− | 0.959552 | 4 | 106157871 | 106157892 | 1 | AGGCACAGGAAAAACATTTGCA | 68 | 106157739 | 1 |
| 4: 106158007-106158176/18, 26/+ | 0.886265 | 4 | 106158007 | 106158024 | 1 | ACTGAGTCTTGCCATAGT | 69 | 106158176 | 1 |
| 4: 106157931-106158100/23, 21/− | 0.983485 | 4 | 106158078 | 106158100 | 1 | TGTTTTCTGGTGGTGCTGTGTGC | 70 | 106157951 | 1 |
| 4: 106158223-106158378/21, 23/+ | 0.956903 | 4 | 106158223 | 106158243 | 1 | GACCATAAGGCTCTTACTCTC | 71 | 106158378 | 1 |
| 4: 106158127-106158296/22, 22/− | 0.950701 | 4 | 106158275 | 106158296 | 1 | GTCAAAACTGTGACTGGCCCTG | 72 | 106158148 | 1 |
| 4: 106158415-106158589/20, 24/+ | 0.882262 | 4 | 106158415 | 106158434 | 1 | GAGTCACCTTCCAAATTACT | 73 | 106158589 | 1 |
| 4: 106158315-106158478/20, 24/− | 0.979383 | 4 | 106158459 | 106158478 | 1 | GAGTCTTGACAGGTGTATCC | 74 | 106158338 | 1 |
| 4: 106162450-106162619/20, 24/+ | 0.88601 | 4 | 106162450 | 106162469 | 1 | TAGTATAATTGAGGTCTAAA | 75 | 106162619 | 1 |
| 4: 106163956-106164125/22, 22/+ | 0.980266 | 4 | 106163956 | 106163977 | 4 | TTGTATGTGTGTGTGTTTCTGT | 76 | 106164125 | 1 |
| 4: 106164761-106164930/21, 23/+ | 0.926757 | 4 | 106164761 | 106164781 | 1 | TGTGTTTGGTGCGGGAGCGAG | 77 | 106164930 | 1 |
| 4: 106164674-106164843/20, 24/− | 0.873669 | 4 | 106164824 | 106164843 | 1 | CGGGATTCCTTCCCACACCA | 78 | 106164697 | 1 |
| 4: 106164852-106165021/21, 23/− | 0.899251 | 4 | 106165001 | 106165021 | 1 | CACGCTGAACTCTCTTCCTTT | 79 | 106164874 | 1 |
| 4: 106180720-106180889/24, 20/+ | 0.846203 | 4 | 106180720 | 106180743 | 1 | TGCACAGCCTATATAATGCTATCC | 80 | 106180889 | 1 |
| 4: 106180786-106180955/20, 24/− | 0.931831 | 4 | 106180936 | 106180955 | 1 | AGCGATTATACATCAGGAAG | 81 | 106180809 | 1 |
| 4: 106182895-106183049/19, 25/+ | 0.697024 | 4 | 106182895 | 106182913 | 1 | AGAATTATTCACTTTATAC | 82 | 106183049 | 1 |
| 4: 106190771-106190940/20, 24/+ | 0.930131 | 4 | 106190771 | 106190790 | 1 | AATATGAACACAGAGCACCA | 83 | 106190940 | 1 |
| 4: 106190690-106190859/21, 23/− | 0.810744 | 4 | 106190839 | 106190859 | 1 | AGCACAGAAGTCCAAACATGC | 84 | 106190712 | 1 |
| 4: 106193771-106193940/22, 22/+ | 0.917879 | 4 | 106193771 | 106193792 | 1 | GGATGAGCAGCTTCACGTTCTG | 85 | 106193940 | 1 |
| 4: 106193686-106193855/20, 24/− | 0.956667 | 4 | 106193836 | 106193855 | 3 | TTTTTTCTCCTCCTGAGCTT | 86 | 106193709 | 1 |
| 4: 106193946-106194115/20, 24/+ | 0.908902 | 4 | 106193946 | 106193965 | 1 | GAAGCCAAGAAAGCTGCAGC | 87 | 106194115 | 1 |
| 4: 106193871-106194040/22, 22/− | 0.986116 | 4 | 106194019 | 106194040 | 1 | TGTTTTGTACGTGATGGGGCTG | 88 | 106193892 | 1 |
| 4: 106196177-106196316/24, 20/+ | 0.915559 | 4 | 106196177 | 106196200 | 1 | TTTCTGTTCTCTCTTACCCTGTCC | 89 | 106196316 | 1 |
| 4: 106196361-106196530/21, 23/+ | 0.913618 | 4 | 106196361 | 106196381 | 1 | CCACCAATCCATACATGAGAC | 90 | 106196530 | 1 |
| 4: 106196262-106196431/20, 24/− | 0.95844 | 4 | 106196412 | 106196431 | 1 | GATATCTGAAGTGTGTGAAG | 91 | 106196285 | 1 |
| 4: 106196561-106196730/20, 24/+ | 0.846784 | 4 | 106196561 | 106196580 | 1 | CAATGCAATGGAAACCTATC | 92 | 106196730 | 1 |
| 4: 106196462-106196631/21, 23/− | 0.977129 | 4 | 106196611 | 106196631 | 1 | GGCTGAGACTGGGGAGAATAG | 93 | 106196484 | 1 |
| 4: 106196781-106196950/20, 24/+ | 0.893805 | 4 | 106196781 | 106196800 | 1 | GTTTCAGCAGTTGTACCATT | 94 | 106196950 | 1 |
| 4: 106196677-106196846/21, 23/− | 0.987067 | 4 | 106196826 | 106196846 | 1 | GAGTGGGATAAGGAGGCAATT | 95 | 106196699 | 1 |
| 4: 106196887-106197056/21, 23/+ | 0.931081 | 4 | 106196887 | 106196907 | 1 | ACCACCCAATCTGAGCAATCC | 96 | 106197056 | 1 |
| 4: 106196980-106197149/20, 24/+ | 0.893803 | 4 | 106196980 | 106196999 | 1 | CATGTTCAACAGCTCTCTTC | 97 | 106197149 | 1 |
| 4: 106197193-106197362/24, 20/+ | 0.908112 | 4 | 106197193 | 106197216 | 1 | CAACGATGAGGTCTGGTCAGACAG | 98 | 106197362 | 1 |
| : 106197071-106197240/18, 26/− | 0.907301 | 4 | 106197223 | 106197240 | 1 | TCAGGATCCAGAAAGCTC | 99 | 106197096 | 1 |
| 4: 106197400-106197569/20, 24/+ | 0.975927 | 4 | 106197400 | 106197419 | 1 | ACATGGCTTGGCTCTTTGGG | 100 | 106197569 | 1 |
| 4: 106197289-106197458/24, 20/− | 0.959564 | 4 | 106197435 | 106197458 | 1 | CTTCCTCTTTCTCACGGGCTTTTT | 101 | 106197308 | 1 |
| 4: 106197523-106197692/20, 24/− | 0.923275 | 4 | 106197673 | 106197692 | 1 | ACAAAAGGGGGTGATATCAT | 102 | 106197546 | 1 |
| 5: 170837475-170837644/22, 22/− | 0.943775 | 5 | 170837623 | 170837644 | 17 | GGACAGCCAGATATCAACTGTT | 103 | 170837496 | 1 |
| 7: 140453073-140453242/20, 24/+ | 0.916227 | 7 | 140453073 | 140453092 | 1 | ACCATCCACAAAATGGATCC | 104 | 140453242 | 1 |
| 7: 148504716-148504885/20, 24/+ | 0.944239 | 7 | 148504716 | 148504735 | 1 | AGGGGGGAGGAGGTAGCAGA | 105 | 148504885 | 1 |
| 7: 148506108-148506277/22, 22/− | 0.949232 | 7 | 148506256 | 148506277 | 1 | GCTCACTGACACCAGTGTGTCT | 106 | 148506129 | 1 |
| 7: 148506397-148506516/24, 20/+ | 0.969517 | 7 | 148506397 | 148506420 | 1 | CCTACCTTTTGCATAGCAGTTTGG | 107 | 148506516 | 1 |
| 7: 148507392-148507561/23, 21/− | 0.965315 | 7 | 148507539 | 148507561 | 1 | GTCAGGCTTGATCACCTTTATCC | 108 | 148507412 | 1 |
| 7: 148508689-148508858/20, 24/− | 0.923191 | 7 | 148508839 | 148508858 | 2 | TTTTCACCCTCCTTTTTTGA | 109 | 148508712 | 1 |
| 7: 148511097-148511266/23, 21/+ | 0.841123 | 7 | 148511097 | 148511119 | 1 | TTACTGTCCCAATGGTCAGCGGC | 110 | 148511266 | 1 |
| 7: 148510995-148511164/20, 24/− | 0.869647 | 7 | 148511145 | 148511164 | 1 | CTGGCTGTCCGAGAGTGTGA | 111 | 148511018 | 1 |
| 7: 148511955-148512124/20, 24/− | 0.889269 | 7 | 148512105 | 148512124 | 1 | CTCTAACCATGTTTACAACT | 112 | 148511978 | 1 |
| 7: 148512532-148512701/22, 22/− | 0.917806 | 7 | 148512680 | 148512701 | 1 | GCTCTCTGTTGGATTTGTAGCT | 113 | 148512553 | 1 |
| 7: 148516641-148516810/20, 24/− | 0.763635 | 7 | 148516791 | 148516810 | 1 | TATTAGATTCTTTGTTTCAT | 114 | 148516664 | 1 |
| 7: 148523639-148523808/20, 24/+ | 0.843345 | 7 | 148523639 | 148523658 | 1 | CAGATTTAGCATTTGGTCCA | 115 | 148523808 | 1 |
| 7: 148523519-148523688/20, 24/− | 0.900594 | 7 | 148523669 | 148523688 | 1 | ACTTCCTCCTGAATGTACCC | 116 | 148523542 | 1 |
| 7: 148526795-148526954/21, 23/− | 0.896884 | 7 | 148526934 | 148526954 | 1 | TTTTTTTCTTTTAGGTGGAAG | 117 | 148526817 | 1 |
| 8: 117864762-117864931/20, 24/− | 0.909931 | 8 | 117864912 | 117864931 | 1 | TTACACCGCTTGTACCAGAA | 118 | 117864785 | 1 |
| 8: 117864807-117864976/23, 21/− | 0.857987 | 8 | 117864954 | 117864976 | 17 | GAAATTTTTCTTTTTTTTTTTTT | 119 | 117864827 | 1 |
| 8: 117866456-117866633/21, 23/+ | 0.779265 | 8 | 117866456 | 117866476 | 1 | GAAGTAAAAATTCTGCAAACT | 120 | 117866633 | 1 |
| 8: 117866573-117866742/24, 20/− | 0.877384 | 8 | 117866719 | 117866742 | 1 | CAGGAAAATGATCTTATTTTTTAT | 121 | 117866592 | 1 |
| 8: 117868372-117868549/24, 20/− | 0.808009 | 8 | 117868526 | 117868549 | 31 | TTTTTTTTTTTTTTTTTTACAGTG | 122 | 117868391 | 1 |
| 9: 5069922-5070091/20, 24/− | 0.88135 | 9 | 5070072 | 5070091 | 1 | AAGGACAAAAAAGACAGTAA | 123 | 5069945 | 1 |
| 9: 5073712-5073881/20, 24/+ | 0.965559 | 9 | 5073712 | 5073731 | 1 | AGCAAGTATGATGAGCAAGC | 124 | 5073881 | 1 |
| 10: 112350147-112350316/21, 23/+ | 0.883234 | 10 | 112350147 | 112350167 | 1 | TTTTCATTTCTGACAACTTAC | 125 | 112350316 | 1 |
| 10: 112350217-112350386/21, 23/− | 0.943637 | 10 | 112350366 | 112350386 | 1 | TAAGCTATTCTTATTCCCTCT | 126 | 112350239 | 1 |
| 10: 112350706-112350875/22, 22/+ | 0.922256 | 10 | 112350706 | 112350727 | 1 | CCTGAGTACCAATAAAGATTTG | 127 | 112350875 | 1 |
| 10: 112350813-112350982/20, 24/− | 0.959726 | 10 | 112350963 | 112350982 | 1 | GTGTATAGTGCCTGGGCACT | 128 | 112350836 | 1 |
| 10: 112352791-112352960/22, 22/+ | 0.980092 | 10 | 112352791 | 112352812 | 1 | CTGTGTTGTGATCTCTCTGTTG | 129 | 112352960 | 1 |
| 10: 112352887-112353056/21, 23/− | 0.892519 | 10 | 112353036 | 112353056 | 1 | CGCCCAGCCAGATTATATGTT | 130 | 112352909 | 1 |
| 10: 112356126-112356295/20, 24/+ | 0.893075 | 10 | 112356126 | 112356145 | 1 | ACAGACCTATTACATATGTT | 131 | 112356295 | 1 |
| 11: 32413487-32413656/20, 24/+ | 0.897861 | 11 | 32413487 | 32413506 | 1 | GAAAAATAAATGTGAAGAAA | 132 | 32413656 | 1 |
| 11: 32414159-32414328/20, 24/− | 0.930567 | 11 | 32414309 | 32414328 | 1 | TCAAATAGAATATGTGTCTT | 133 | 32414182 | 1 |
| 11: 32417866-32418035/20, 24/+ | 0.938676 | 11 | 32417866 | 32417885 | 1 | GGTAAGCACACATGAAGGGG | 134 | 32418035 | 1 |
| 11: 32417752-32417921/20, 24/− | 0.91638 | 11 | 32417902 | 32417921 | 1 | TCTTGTACGGTCGGCATCTG | 135 | 32417775 | 1 |
| 11: 32421472-32421637/20, 24/+ | 0.93191 | 11 | 32421472 | 32421491 | 1 | TGGGGCCTGTCTGTGTGCTC | 136 | 32421637 | 1 |
| 11: 119148811-119148980/23, 21/+ | 0.851218 | 11 | 119148811 | 119148833 | 1 | CTGTTAACATTTATAATTGCAGT | 137 | 119148980 | 1 |
| 11: 119148895-119149064/20, 24/− | 0.92696 | 11 | 119149045 | 119149064 | 1 | TACCGAATTTTCCAAGGTTA | 138 | 119148918 | 1 |
| 11: 119149175-119149344/20, 24/+ | 0.895932 | 11 | 119149175 | 119149194 | 2 | AGTATTTTCAGATGCATCTG | 139 | 119149344 | 1 |
| 2: 25378504-25378673/20, 24/− | 0.911124 | 12 | 25378654 | 25378673 | 1 | GTACCTATGGTCCTAGTAGG | 140 | 25378527 | 1 |
| 12: 25380216-25380385/20, 24/− | 0.967652 | 12 | 25380366 | 25380385 | 1 | TGCACTGTAATAATCCAGAC | 141 | 25380239 | 1 |
| 12: 25398165-25398334/20, 24/− | 0.933725 | 12 | 25398315 | 25398334 | 1 | ATAAGGCCTGCTGAAAATGA | 142 | 25398188 | 1 |
| 12: 112888093-112888262/23, 21/− | 0.916866 | 12 | 112888240 | 112888262 | 3 | CCATTCTTCTCTTTTAATTGCCC | 143 | 112888113 | 1 |
| 12: 112910715-112910880/22, 22/+ | 0.894139 | 12 | 112910715 | 112910736 | 1 | CACGTAATAATATTGACTTTTC | 144 | 112910880 | 1 |
| 12: 112915411-112915580/20, 24/− | 0.966125 | 12 | 112915561 | 112915580 | 1 | GGCTAGAAATGTATGGTCAG | 145 | 112915434 | 1 |
| 12: 112926198-112926363/20, 24/− | 0.918085 | 12 | 112926344 | 112926363 | 2 | CATAAACTAAAAACAGAAAC | 146 | 112926221 | 1 |
| 12: 112926812-112926981/20, 24/+ | 0.957582 | 12 | 112926812 | 112926831 | 1 | GATGTTTCCTTCGTAGGTGT | 147 | 112926981 | 1 |
| 13: 28592560-28592729/20, 24/+ | 0.919768 | 13 | 28592560 | 28592579 | 1 | CACAAAATAGCCGTATAAAA | 148 | 28592729 | 1 |
| 13: 28602295-28602464/16, 29/+ | 0.881258 | 13 | 28602295 | 28602310 | 2 | TTTTCGTGGAAGTGGG | 149 | 28602464 | 1 |
| 13: 28607990-28608155/20, 24/− | 0.927129 | 13 | 28608136 | 28608155 | 1 | GCACGTACTCACCATTTGTC | 150 | 28608013 | 1 |
| 13: 28608205-28608374/20, 24/+ | 0.894916 | 13 | 28608205 | 28608224 | 1 | CATTCCATTCTTACCAAACT | 151 | 28608374 | 1 |
| 13: 28608170-28608339/20, 24/− | 0.920908 | 13 | 28608320 | 28608339 | 1 | GAAAGCCAGCTACAGATGGT | 152 | 28608193 | 1 |
| 13: 28608401-28608570/20, 24/+ | 0.891863 | 13 | 28608401 | 28608420 | 1 | AGAGGAAAGAATAATGAATT | 153 | 28608570 | 1 |
| 13: 28609586-28609755/21, 23/+ | 0.98033 | 13 | 28609586 | 28609606 | 1 | GTGGGGAATTCCTGATGGTGG | 154 | 28609755 | 1 |
| 13: 28609701-28609870/20, 24/+ | 0.946119 | 13 | 28609701 | 28609720 | 1 | ACCCTTTTATGGCTTCACTC | 155 | 28609870 | 1 |
| 13: 28610048-28610217/20, 24/+ | 0.916878 | 13 | 28610048 | 28610067 | 1 | AAGAGGCATCAATGTCCTTA | 156 | 28610217 | 1 |
| 15: 90631745-90631914/20, 24/+ | 0.801172 | 15 | 90631745 | 90631764 | 1 | AAGAGGATGGCTAGGCGAGG | 157 | 90631914 | 1 |
| 15: 90631845-90632014/20, 24/+ | 0.93551 | 15 | 90631845 | 90631864 | 1 | TGGTGATGGGCTTGGTCCAG | 158 | 90632014 | 1 |
| 17: 7572870-7573039/21, 23/+ | 0.981202 | 17 | 7572870 | 7572890 | 1 | AGGGGAGGGAGAGATGGGGGT | 159 | 7573039 | 1 |
| 17: 7573943-7574112/22, 22/− | 0.910933 | 17 | 7574091 | 7574112 | 1 | CAAACAATTGTAACTTGAACCA | 160 | 7573964 | 1 |
| 17: 7576792-7576961/24, 20/− | 0.847926 | 17 | 7576938 | 7576961 | 1 | TTCACTTTTATCACCTTTCCTTGC | 161 | 7576811 | 1 |
| 17: 7577075-7577244/24, 20/+ | 0.980151 | 17 | 7577075 | 7577098 | 1 | TTCTCTTCCTCTGTGCGCCGGTCT | 162 | 7577244 | 1 |
| 17: 7576963-7577132/21, 23/− | 0.924868 | 17 | 7577112 | 7577132 | 1 | GCTTTGAGGTGCGTGTTTGTG | 163 | 7576985 | 1 |
| 17: 7577438-7577602/20, 24/+ | 0.897643 | 17 | 7577438 | 7577457 | 1 | AGAGGCAAGCAGAGGCTGGG | 164 | 7577602 | 1 |
| 17: 7577529-7577693/22, 22/− | 0.959787 | 17 | 7577672 | 7577693 | 14 | AAAAAAAAAAAAAAAGGCCTCC | 165 | 7577550 | 1 |
| 17: 7578169-7578338/23, 21/+ | 0.893158 | 17 | 7578169 | 7578191 | 1 | AACCAGACCTCAGGCGGCTCATA | 166 | 7578338 | 1 |
| 17: 7578093-7578262/23, 21/− | 0.937736 | 17 | 7578240 | 7578262 | 1 | GAGTGGAAGGAAATTTGCGTGTG | 167 | 7578113 | 1 |
| 17: 7578393-7578562/18, 26/+ | 0.843508 | 17 | 7578393 | 7578410 | 1 | ATGGTGGGGGCAGCGCCT | 168 | 7578562 | 1 |
| 17: 7578334-7578453/18, 26/− | 0.833295 | 17 | 7578436 | 7578453 | 1 | CATGGCCATCTACAAGCA | 169 | 7578359 | 1 |
| 17: 7578469-7578628/26, 18/− | 0.890436 | 17 | 7578603 | 7578628 | 1 | CTGCCGTCTTCCAGTTGCTTTATCTG | 170 | 7578486 | 1 |
| 17: 7579238-7579407/20, 24/+ | 0.809488 | 17 | 7579238 | 7579257 | 1 | CAAACAAAAGAAATGCAGGG | 171 | 7579407 | 1 |
| 17: 7579298-7579467/21, 23/+ | 0.74921 | 17 | 7579298 | 7579318 | 1 | TCAGGGCAACTGACCGTGCAA | 172 | 7579467 | 1 |
| 17: 7579416-7579577/20, 24/+ | 0.784204 | 17 | 7579416 | 7579435 | 2 | AGGAGGGGGCTGGTGCAGGG | 173 | 7579577 | 1 |
| 17: 7579495-7579664/21, 23/− | 0.900055 | 17 | 7579644 | 7579664 | 2 | TGGGGACCTGGAGGGCTGGGG | 174 | 7579517 | 1 |
| 17: 7579802-7579971/24, 20/− | 0.864469 | 17 | 7579948 | 7579971 | 1 | TCATGCTGGATCCCCACTTTTCCT | 175 | 7579821 | 1 |
| 17: 58740425-58740594/22, 22/+ | 0.952631 | 17 | 58740425 | 58740446 | 1 | GCCTTCTCAGAGAATTTTTTAG | 176 | 58740594 | 1 |
| 17: 58740303-58740472/20, 24/− | 0.827005 | 17 | 58740453 | 58740472 | 1 | CTCTCGAGCTATCTCAGCTG | 177 | 58740326 | 1 |
| 17: 58740633-58740802/21, 23/+ | 0.979392 | 17 | 58740633 | 58740653 | 1 | TGAAGATGTCAACTCCTGGCC | 178 | 58740802 | 1 |
| 17: 58740506-58740675/21, 23/− | 0.853213 | 17 | 58740655 | 58740675 | 1 | TCAATTTCTTGGGCTTTCATT | 179 | 58740528 | 1 |
| 17: 58740833-58741002/20, 24/+ | 0.902941 | 17 | 58740833 | 58740852 | 1 | ATGCGACGCAGACTTAGGGG | 180 | 58741002 | 1 |
| 7: 58740728-58740897/21, 23/− | 0.938281 | 17 | 58740877 | 58740897 | 1 | GTTTTCCTGTGTTGATGAAGT | 181 | 58740750 | 1 |
| 17: 74732929-74733008/24, 20/+ | 0.924881 | 17 | 74732929 | 74732952 | 1 | GGTCCCCGGCGGCTGTGGTGTGAG | 182 | 74733008 | 1 |
| 18: 42531843-42532012/21, 23/+ | 0.911291 | 18 | 42531843 | 42531863 | 1 | CTCCCTAAAGGAAATCACGCT | 183 | 42532012 | 1 |
| 19: 13054585-13054754/20, 24/+ | 0.937952 | 19 | 13054585 | 13054604 | 1 | AAGAAGACAAGAAACGCAAA | 184 | 13054754 | 1 |
| 19: 13054480-13054649/24, 20/− | 0.794174 | 19 | 13054626 | 13054649 | 1 | ATCTTTGTCCTCATCATCCTCCTT | 185 | 13054499 | 1 |
| 19: 33792231-33792385/16, 27/− | 0.810719 | 19 | 33792370 | 33792385 | 12 | GAAGGTGCTGGAGCTG | 186 | 33792257 | 1 |
| 19: 33792335-33792504/20, 24/− | 0.830437 | 19 | 33792485 | 33792504 | 1 | AAGGCCAAGAAGTCGGTGGA | 187 | 33792358 | 1 |
| 19: 33792732-33792851/16, 26/+ | 0.652911 | 19 | 33792732 | 33792747 | 19 | GCGGGTGCGGGTGCGG | 188 | 33792851 | 1 |
| 19: 33792942-33793051/16, 24/− | 0.849634 | 19 | 33793036 | 33793051 | 2 | GGCCAAGGCGGCCGTG | 189 | 33792965 | 1 |
| 19: 33793045-33793154/16, 29/+ | 0.8992 | 19 | 33793045 | 33793060 | 10 | CTTGGCCTTCTCCTGC | 190 | 33793154 | 1 |
| 19: 33793184-33793288/16, 26/+ | 0.876116 | 19 | 33793184 | 33793199 | 12 | GGTGGGGGGGGAGGCT | 191 | 33793288 | 1 |
| 20: 31021052-31021221/21, 23/+ | 0.922229 | 20 | 31021052 | 31021072 | 1 | GGCCTGAAACTGATGGCTGTG | 192 | 31021221 | 1 |
| 20: 31021242-31021411/22, 22/+ | 0.940116 | 20 | 31021242 | 31021263 | 1 | CAGATCTCCGAACCAGAGCCAG | 193 | 31021411 | 1 |
| 20: 31021151-31021320/24, 20/− | 0.925719 | 20 | 31021297 | 31021320 | 1 | TTTGCATCCTTAGCAACCCCTGCT | 194 | 31021170 | 1 |
| 20: 31021437-31021606/20, 24/+ | 0.888392 | 20 | 31021437 | 31021456 | 1 | CCGAATTCCCAGTTGAGTCT | 195 | 31021606 | 1 |
| 20: 31021362-31021531/23, 21/− | 0.82328 | 20 | 31021509 | 31021531 | 1 | CAGGCTAGGAATTCTGTCTGGAG | 196 | 31021382 | 1 |
| 20: 31021534-31021703/20, 24/+ | 0.939897 | 20 | 31021534 | 31021553 | 1 | TCAGGAAACTGTGGATCAGG | 197 | 31021703 | 1 |
| 20: 31022183-31022352/22, 22/+ | 0.894333 | 20 | 31022183 | 31022204 | 1 | TAGGTCAGATCACCCAGTCAGT | 198 | 31022352 | 1 |
| 20: 31022389-31022533/16, 25/+ | 0.740488 | 20 | 31022389 | 31022404 | 2 | GAGGGGCGAGAGGTCA | 199 | 31022533 | 1 |
| 20: 31022292-31022441/20, 24/− | 0.670733 | 20 | 31022422 | 31022441 | 1 | TCCGATGGCAGTGGTGGCCG | 200 | 31022315 | 1 |
| 20: 31022643-31022812/21, 23/+ | 0.806603 | 20 | 31022643 | 31022663 | 1 | GGAACTGCCATGTCCAGAGCT | 201 | 31022812 | 1 |
| 20: 31022555-31022724/20, 24/− | 0.907715 | 20 | 31022705 | 31022724 | 1 | CTGTAGCCCTCTGTAGTAGG | 202 | 31022578 | 1 |
| 20: 31022881-31023050/22, 22/+ | 0.931489 | 20 | 31022881 | 31022902 | 1 | GTGAGTCTGGCACCACTTCCTG | 203 | 31023050 | 1 |
| 20: 31022782-31022951/23, 21/− | 0.91537 | 20 | 31022929 | 31022951 | 1 | ACCATTGTCTGCAGGAACGGTGG | 204 | 31022802 | 1 |
| 20: 31023086-31023255/21, 23/+ | 0.933626 | 20 | 31023086 | 31023106 | 1 | GCAGAACAGAGCATTTGATGA | 205 | 31023255 | 1 |
| 20: 31022984-31023153/23, 21/− | 0.932364 | 20 | 31023131 | 31023153 | 1 | TATCACTTTCCCTCATAGGAGGG | 206 | 31023004 | 1 |
| 20: 31023328-31023497/20, 24/+ | 0.949461 | 20 | 31023328 | 31023347 | 1 | CTGCATTGCCTGGGGATTTG | 207 | 31023497 | 1 |
| 20: 31023202-31023371/21, 23/− | 0.881999 | 20 | 31023351 | 31023371 | 1 | AGGATCTAGACCCTCCTCAGC | 208 | 31023224 | 1 |
| 20: 31023421-31023590/20, 24/− | 0.918557 | 20 | 31023571 | 31023590 | 1 | CTTTGTCACTGCAGCTTCTC | 209 | 31023444 | 1 |
| 20: 31023517-31023686/20, 24/− | 0.878099 | 20 | 31023667 | 31023686 | 1 | ATCTGTCCTTGTAACCAGAC | 210 | 31023540 | 1 |
| 20: 31023708-31023877/22, 22/+ | 0.887733 | 20 | 31023708 | 31023729 | 1 | TGGGTGTCTCGAGTATGTGCGG | 211 | 31023877 | 1 |
| 20: 31023619-31023788/20, 24/− | 0.904275 | 20 | 31023769 | 31023788 | 1 | GGCTCTTGGCTGGTACTCAG | 212 | 31023642 | 1 |
| 20: 31023826-31023995/20, 24/− | 0.897669 | 20 | 31023976 | 31023995 | 1 | GCCACTGTTTTTTCCAAGAC | 213 | 31023849 | 1 |
| 20: 31024004-31024173/20, 24/+ | 0.889572 | 20 | 31024004 | 31024023 | 1 | TGGAAGCAGCCCCAGTTCTT | 214 | 31024173 | 1 |
| 20: 31024067-31024236/21, 23/+ | 0.894101 | 20 | 31024067 | 31024087 | 1 | AGGCACTGGTCTTGCCAGGAT | 215 | 31024236 | 1 |
| 20: 31023931-31024100/20, 24/− | 0.934792 | 20 | 31024081 | 31024100 | 1 | CTGGGTGGCCTCAATCCTGG | 216 | 31023954 | 1 |
| 20: 31024183-31024352/20, 24/+ | 0.897143 | 20 | 31024183 | 31024202 | 1 | CTAGGAAACTGGAAGAAATG | 217 | 31024352 | 1 |
| 20: 31024301-31024470/24, 20/+ | 0.881533 | 20 | 31024301 | 31024324 | 1 | CCCAGGAGATCTTACTACCTCGAG | 218 | 31024470 | 1 |
| 20: 31024409-31024578/20, 24/+ | 0.868986 | 20 | 31024409 | 31024428 | 2 | AGGCCAAGGGAAGAAGCTTT | 219 | 31024578 | 1 |
| 20: 31024513-31024682/20, 24/+ | 0.949413 | 20 | 31024513 | 31024532 | 1 | TTCCCAGTGGGAAGTTGGGA | 220 | 31024682 | 1 |
| 20: 31024623-31024792/20, 24/+ | 0.95695 | 20 | 31024623 | 31024642 | 1 | GAGAAGACTTTTGTGGGGGG | 221 | 31024792 | 1 |
| 0: 31024736-31024905/23, 21/+ | 0.924311 | 20 | 31024736 | 31024758 | 1 | CTTGCCCTTCTGGAAATTACCCC | 222 | 31024905 | 1 |
| 20: 31024855-31025024/21, 23/+ | 0.905752 | 20 | 31024855 | 31024875 | 1 | TCCAACTGAGTTCCACCAGCT | 223 | 31025024 | 1 |
| 20: 31024965-31025134/20, 24/+ | 0.950131 | 20 | 31024965 | 31024984 | 1 | CACAGTGCATCACTTTCCTT | 224 | 31025134 | 1 |
| 20: 57484366-57484525/18, 26/− | 0.944333 | 20 | 57484508 | 57484525 | 1 | CAAGAAACCATGATCTCT | 225 | 57484391 | 1 |
| 21: 36164530-36164685/20, 24/+ | 0.734511 | 21 | 36164530 | 36164549 | 1 | GGTTCGGGAGGCTGGGGTTG | 226 | 36164685 | 1 |
| 21: 36164619-36164747/21, 23/+ | 0.781897 | 21 | 36164619 | 36164639 | 2 | ACCATGGAGAACTGGTAGGAG | 227 | 36164747 | 1 |
| 21: 36164705-36164815/20, 24/+ | 0.711885 | 21 | 36164705 | 36164724 | 1 | TTGCGACGAGCCGGGGTAGG | 228 | 36164815 | 1 |
| 21: 36164717-36164857/23, 21/+ | 0.828908 | 21 | 36164717 | 36164739 | 1 | GGGGTAGGGCGGCGGCAGGTAGG | 229 | 36164857 | 1 |
| 21: 36164802-36164956/19, 25/+ | 0.794232 | 21 | 36164802 | 36164820 | 1 | GTCGGGGAGTAGGTGAAGG | 230 | 36164956 | 1 |
| 21: 36171528-36171697/21, 23/− | 0.841039 | 21 | 36171677 | 36171697 | 1 | TCCATTGCCTCTCCTTCTGTG | 231 | 36171550 | 1 |
| 21: 36171646-36171815/22, 22/− | 0.879815 | 21 | 36171794 | 36171815 | 1 | CATTTTTTAAATCCCACCCCAC | 232 | 36171667 | 1 |
| 21: 36206622-36206777/23, 21/+ | 0.865415 | 21 | 36206622 | 36206644 | 1 | GGGAAGGTGTGTGCACATGGGGG | 233 | 36206777 | 1 |
| 21: 36206730-36206885/21, 23/+ | 0.735902 | 21 | 36206730 | 36206750 | 1 | GGGTTAAAGGCAGTGGAGTGG | 234 | 36206885 | 1 |
| 21: 36206799-36206954/23, 21/− | 0.791705 | 21 | 36206932 | 36206954 | 15 | TCTTCCCTCCCTCCTTCCCTCCC | 235 | 36206819 | 1 |
| 21: 36231732-36231901/22, 22/− | 0.873923 | 21 | 36231880 | 36231901 | 1 | TTTTGTTCTCTATCGTGTCCCC | 236 | 36231753 | 1 |
| 21: 36252801-36252970/23, 21/+ | 0.943161 | 21 | 36252801 | 36252823 | 1 | TGGGTTTGTTGCCATGAAACGTG | 237 | 36252970 | 1 |
| 21: 36252916-36253085/21, 23/− | 0.94378 | 21 | 36253065 | 36253085 | 1 | ATCACTACACAAATGCCCTAA | 238 | 36252938 | 1 |
| 21: 36259108-36259222/16, 29/+ | 0.817091 | 21 | 36259108 | 36259123 | 15 | CTGTCCTCCCACCACC | 239 | 36259222 | 2 |
| 21: 36259159-36259263/22, 22/+ | 0.706198 | 21 | 36259159 | 36259180 | 3 | GTCTTGTTGCAGCGCCAGTGCG | 240 | 36259263 | 1 |
| 21: 44514729-44514898/22, 22/− | 0.933507 | 21 | 44514877 | 44514898 | 2 | TTTCGCCGTGAGGAAGATGCGG | 241 | 44514750 | 1 |
| 21: 44524416-44524585/20, 24/+ | 0.94191 | 21 | 44524416 | 44524435 | 1 | CAAACAAACCTGGCTAAACG | 242 | 44524585 | 1 |
| X: 15833872-15834041/20, 24/+ | 0.979296 | X | 15833872 | 15833891 | 2 | AATGGAGCAGTGCAGGAGGG | 243 | 15834041 | 1 |
| X: 15833745-15833914/20, 24/− | 0.924197 | X | 15833895 | 15833914 | 2 | GCTTGCGTCAGGGTCATAGT | 244 | 15833768 | 1 |
| X: 15836657-15836826/21, 23/− | 0.93315 | X | 15836806 | 15836826 | 1 | AACAGAAACAGAACAACAAAC | 245 | 15836679 | 1 |
| X: 15838303-15838483/23, 21/+ | 0.712992 | X | 15838303 | 15838325 | 1 | GCATATCATTTGATTTTTGGTTT | 246 | 15838483 | 1 |
| X: 39911340-39911509/20, 24/+ | 0.940705 | X | 39911340 | 39911359 | 1 | GTACATGGTGGGTCCAGCTT | 247 | 39911509 | 1 |
| X: 39911542-39911711/21, 23/+ | 0.932675 | X | 39911542 | 39911562 | 1 | TTCTGCCTCTGCAATGGTGAC | 248 | 39911711 | 1 |
| X: 39911448-39911617/20, 24/− | 0.941736 | X | 39911598 | 39911617 | 1 | GAAATTGAAAATGTCCTCCC | 249 | 39911471 | 1 |
| X: 39914595-39914764/20, 24/+ | 0.919934 | X | 39914595 | 39914614 | 1 | AGACCATTTCTTGAACTTTG | 250 | 39914764 | 1 |
| X: 39914691-39914860/24, 20/− | 0.966337 | X | 39914837 | 39914860 | 1 | TGCCCAGCTTTGCCTGTTGCTTTT | 251 | 39914710 | 1 |
| X: 39932843-39933012/24, 20/+ | 0.82704 | X | 39932843 | 39932866 | 1 | TCCTGCTGGTTTTGGTGCCATCTG | 252 | 39933012 | 1 |
| X: 39933079-39933248/20, 24/+ | 0.945629 | X | 39933079 | 39933098 | 1 | GAGGGAGCAGTGCTGATGAT | 253 | 39933248 | 1 |
| X: 39932964-39933133/20, 24/− | 0.933519 | X | 39933114 | 39933133 | 1 | CTCCAGGAAATGGTTGTGCT | 254 | 39932987 | 1 |
| X: 48649464-48649633/16, 29/+ | 0.856993 | X | 48649464 | 48649479 | 14 | TTTCTGTGTCTGAGGA | 255 | 48649633 | 1 |
| X: 48649695-48649864/21, 23/+ | 0.897556 | X | 48649695 | 48649715 | 1 | TGGCCTACTACAGGGACGCTG | 256 | 48649864 | 1 |
| X: 48649576-48649745/20, 24/− | 0.823488 | X | 48649726 | 48649745 | 1 | TGGAGTTACCTGGGGAGTGT | 257 | 48649599 | 1 |
| X: 53431899-53432068/20, 24/+ | 0.892295 | X | 53431899 | 53431918 | 1 | TGTGTGGATGGCCTTTGGAG | 258 | 53432068 | 1 |
| X: 53432011-53432180/22, 22/+ | 0.897516 | X | 53432011 | 53432032 | 1 | ATCTGCAGTCCATGGGCCTGAG | 259 | 53432180 | 1 |
| X: 53432104-53432273/23, 21/+ | 0.897847 | X | 53432104 | 53432126 | 1 | GCTAGGTGGTAAGGTGGTGGCTG | 260 | 53432273 | 1 |
| X: 53432228-53432397/24, 20/+ | 0.904552 | X | 53432228 | 53432251 | 1 | TTTCTCATCCCAGCGCCGTGCCTT | 261 | 53432397 | 1 |
| X: 53432336-53432505/20, 24/+ | 0.904126 | X | 53432336 | 53432355 | 1 | GGGGGAAGAGAGAAGAGGGG | 262 | 53432505 | 1 |
| : 53432548-53432717/23, 21/+ | 0.873273 | X | 53432548 | 53432570 | 1 | ATCAATCACTAGCTTGGCCCCCT | 263 | 53432717 | 1 |
| X: 53432438-53432607/22, 22/− | 0.912547 | X | 53432586 | 53432607 | 1 | CAGGTGAAGCCTACAGATGAGA | 264 | 53432459 | 1 |
| X: 53432775-53432944/23, 21/+ | 0.945673 | X | 53432775 | 53432797 | 1 | GCCTGTCTTCTCCGAGTCCACAA | 265 | 53432944 | 1 |
| X: 53432662-53432831/22, 22/− | 0.898153 | X | 53432810 | 53432831 | 1 | ACCAAGGTTTTGGGCAAGAACA | 266 | 53432683 | 1 |
| X: 123181234-123181403/20, 24/+ | 0.95047 | X | 123181234 | 123181253 | 1 | TGGCACTAAATCTTAGCATT | 267 | 123181403 | 1 |
| X: 123181157-123181326/20, 24/− | 0.9185 | X | 123181307 | 123181326 | 1 | GCCTCTCATTGGCTCGTTTT | 268 | 123181180 | 1 |
| X: 123182833-123183002/20, 24/+ | 0.820783 | X | 123182833 | 123182852 | 1 | GAAATGATGTGTTTTTTTAC | 269 | 123183002 | 1 |
| X: 123184006-123184184/21, 23/+ | 0.907704 | X | 123184006 | 123184026 | 1 | TTATGCATCGTTTTTCCTTCC | 270 | 123184184 | 1 |
| X: 123184910-123185079/20, 24/+ | 0.833242 | X | 123184910 | 123184929 | 1 | GAAGTTGAAAATACATAGAG | 271 | 123185079 | 1 |
| X: 123185026-123185195/22, 22/+ | 0.858971 | X | 123185026 | 123185047 | 1 | AAGAGCTTAATTCCAAACTGGA | 272 | 123185195 | 1 |
| X: 123185116-123185285/20, 24/+ | 0.932372 | X | 123185116 | 123185135 | 5 | CTTTTCTCTTGCTTTCCTTT | 273 | 123185285 | 1 |
| X: 123189951-123190120/21, 23/− | 0.874373 | X | 123190100 | 123190120 | 1 | GCCTTAGAAAATGAGTAACAG | 274 | 123189973 | 1 |
| X: 123220363-123220532/20, 24/+ | 0.871879 | X | 123220363 | 123220382 | 1 | GTATCAAAGCTAACAGTTTC | 275 | 123220532 | 1 |
| X: 123220475-123220644/20, 24/− | 0.7772 | X | 123220625 | 123220644 | 1 | AAAGAGAATAAATTATATCC | 276 | 123220498 | 1 |
| X: 133511649-133511818/20, 24/+ | 0.966325 | X | 133511649 | 133511668 | 1 | TGTCAAGCTCAGTTGAACAG | 277 | 133511818 | 1 |
| X: 133511601-133511770/20, 24/− | 0.96135 | X | 133511751 | 133511770 | 1 | CGCTGCCACCTTCTGGTTTT | 278 | 133511624 | 1 |
| X: 133527602-133527771/20, 24/+ | 0.867927 | X | 133527602 | 133527621 | 1 | CTACCACTGTGCATTGCATG | 279 | 133527771 | 1 |
| X: 133527477-133527646/20, 24/− | 0.939432 | X | 133527627 | 133527646 | 1 | GGTTTCTCTCGTATTTGAGC | 280 | 133527500 | 1 |
| X: 133527898-133528017/22, 22/+ | 0.864925 | X | 133527898 | 133527919 | 1 | TTAGTTTGCTTACTAATTTTTG | 281 | 133528017 | 1 |
| X: 133547773-133547942/20, 24/+ | 0.896119 | X | 133547773 | 133547792 | 1 | GAAATGTTAAGTAAGCTTGA | 282 | 133547942 | 1 |
| X: 133547875-133548044/23, 21/− | 0.984411 | X | 133548022 | 133548044 | 17 | AAACAAACAACAAAAAAACAAAA | 283 | 133547895 | 1 |
| X: 133549006-133549180/27, 27/− | 0.875443 | X | 133549154 | 133549180 | 1 | TGTTGCATAACAAATATAAAAACTACA | 284 | 133549032 | |
| X: 133551196-133551365/22, 22/− | 0.876924 | X | 133551344 | 133551365 | 1 | GTTTAGGGAAATAGACAACTGT | 285 | 133551217 | 1 |

| | | mip_scan_ | |
|---|---|---|---|
| | SEQ | start_ | |
| lig_probe_sequence | ID | position | mip_name |
| TCAGACTTCGGCCCACCC | 286 | 43814917 | MPL_001_Myeloid_Panel |
| GTGTGGAGGGTAAGGGGGCAGGG | 287 | 115256480 | NRAS_001_Myeloid_Panel |
| GTAGCCCGCTGACCTGATCCTGT | 288 | 115258658 | NRAS_002_Myeloid_Panel |
| ACTGGGAAACCAAATACCCTGG | 289 | 25457146 | DNMT3A_001_Myeloid_Panel |
| GCTGAAGGAGTATTTTGCGTGTGT | 290 | 25457176 | DNMT3A_002_Myeloid_Panel |
| CCCGGGTTGTGCTGGCATCTGGCT | 291 | 25458573 | DNMT3A_003_Myeloid_Panel |
| GCAGGGAGAAGGAAGGGCAGGAT | 292 | 25459772 | DNMT3A_004_Myeloid_Panel |
| GCTCCTGGGCCTGGGGGGCTGT | 293 | 25461989 | DNMT3A_005_Myeloid_Panel |
| GCGCATCATGCAGGAGGCGGTAGA | 294 | 25463143 | DNMT3A_006_Myeloid_Panel |
| GCGTTAGTGACAAGAGGGACATCT | 295 | 25463209 | DNMT3A_007_Myeloid_Panel |
| GCTGTCCAGGGACAGAGGCAGACA | 296 | 25463504 | DNMT3A_008_Myeloid_Panel |
| TTGGAGCCATCTCCCTGGCACCCT | 297 | 25464530 | DNMT3A_009_Myeloid_Panel |
| GTCCATGCTGTGGGGCGCAG | 298 | 25464427 | DNMT3A_010_Myeloid_Panel |
| GTGAGGGGTGCAGGCCCAAGAGGT | 299 | 25466767 | DNMT3A_011_Myeloid_Panel |
| CTTCCTTAATGGCTGCCTGGGCAG | 300 | 25467032 | DNMT3A_012_Myeloid_Panel |
| GCCTGGGGCGCGGTCTCGAGCT | 301 | 25467013 | DNMT3A_013_Myeloid_Panel |
| CCCCTGGAACTGCTACATGTGC | 302 | 25467139 | DNMT3A_014_Myeloid_Panel |
| GCTGTTGTGGCCTCCAGTGGT | 303 | 25467404 | DNMT3A_015_Myeloid_Panel |
| GGTGAGTACCACCGAAGGGCCTCT | 304 | 25468120 | DNMT3A_016_Myeloid_Panel |
| TGCCGTTGAGGCCGGCCCTTC | 305 | 25468096 | DNMT3A_017_Myeloid_Panel |
| CCGCGCCTGCTCCTCGGATG | 306 | 25468841 | DNMT3A_018_Myeloid_Panel |
| CACATGTCCGTGTACACTTCTTTG | 307 | 25469013 | DNMT3A_019_Myeloid_Panel |
| TTCCCCCACCCTCCTTACAG | 308 | 25469111 | DNMT3A_020_Myeloid_Panel |
| GTGACACGCCAGGGTTGGGGTTGT | 309 | 25469530 | DNMT3A_021_Myeloid_Panel |
| GGCTGCCAAGGCCTCCACAGAG | 310 | 25469407 | DNMT3A_022_Myeloid_Panel |
| CACCCACCCCATGCCTTGCAA | 311 | 25469949 | DNMT3A_023_Myeloid_Panel |
| GCACTAGGAGGCCTGGAAGTTG | 312 | 25469881 | DNMT3A_024_Myeloid_Panel |
| TTCCCCCACACCAGCTCCCCAA | 313 | 25470458 | DNMT3A_025_Myeloid_Panel |
| GTGTCTTGGTGGATGACGGGC | 314 | 25470544 | DNMT3A_026_Myeloid_Panel |
| CGGGCCCCTGGTTTTCTT | 315 | 25470939 | DNMT3A_027_Myeloid_Panel |
| AGGTAATTGGTGGATTTACCTTTC | 316 | 198266564 | SF3B1_001_Myeloid_Panel |
| CTGGATATGTTTCATGGTTCT | 317 | 198266438 | SF3B1_002_Myeloid_Panel |
| GTGTTAAAGCCTTTATGGAAGGGT | 318 | 198266754 | SF3B1_003_Myeloid_Panel |
| GAAAGGACAGTCATGAGTTGGT | 319 | 198267441 | SF3B1_004_Myeloid_Panel |
| CTTATGGGCTGTGCCATCTTGCC | 320 | 198267338 | SF3B1_005_Myeloid_Panel |
| GGTGCCATTTGGTGATTTCC | 321 | 209113087 | IDH1_001_Myeloid_Panel |
| TGCAGGAGAAGTCATCCCCCTTC | 322 | 128200044 | GATA2_001_Myeloid_Panel |
| GGTGCCGGCTCTTCTGGCGG | 323 | 128200685 | GATA2_002_Myeloid_Panel |
| CACTCATCAAGCCCAAGCGAAGAC | 324 | 128202729 | GATA2_003_Myeloid_Panel |
| GTCCAGGAACTGAGCAGAGGT | 325 | 55589720 | KIT_001_Myeloid_Panel |
| GTGAATACACTATTAGGTTGGAGG | 326 | 55599245 | KIT_002_Myeloid_Panel |
| GCCCACTGCCTGAGAGAGCTCAT | 327 | 106155136 | TET2_001_Myeloid_Panel |
| GTAGAGGGTATTCCAAGTGTTTGC | 328 | 106155263 | TET2_002_Myeloid_Panel |
| GTTCTGTCTGGCAAATGGGAGGTG | 329 | 106155185 | TET2_003_Myeloid_Panel |
| TCTGTAGCCCAAGAAAATGCAG | 330 | 106155430 | TET2_004_Myeloid_Panel |
| CGACTATTCTGGCTTCCCTTC | 331 | 106155318 | TET2_005_Myeloid_Panel |
| TGGAACACACACATGGTGAACTCCTG | 332 | 106155646 | TET2_006_Myeloid_Panel |
| CATTTGGTTGACTGCTTTCACCTG | 333 | 106155521 | TET2_007_Myeloid_Panel |
| GTGAGTGAGGCCTGTGATGCTGAT | 334 | 106155847 | TET2_008_Myeloid_Panel |
| GCACCATTAGGCATTAGCACTGCC | 335 | 106155732 | TET2_009_Myeloid_Panel |
| CCAGCAGCAATTTGCAAGC | 336 | 106156060 | TET2_010_Myeloid_Panel |
| GCAGCTGGCTTTGGAGGCAGCT | 337 | 106155969 | TET2_011_Myeloid_Panel |
| CACCACCACTACCCCAACCAAA | 338 | 106156273 | TET2_012_Myeloid_Panel |
| TGAACAGAATTCTTCACCA | 339 | 106156162 | TET2_013_Myeloid_Panel |
| CCAATGTCAGAACACCTCAAGC | 340 | 106156492 | TET2_014_Myeloid_Panel |
| GCTTTTTCCTTCTGAAGGAAGCTG | 341 | 106156372 | TET2_015_Myeloid_Panel |
| CGTAATGAGGCATCACTGCCATCA | 342 | 106156714 | TET2_016_Myeloid_Panel |
| CCACAATGGAACAGTCATTGTCCC | 343 | 106156603 | TET2_017_Myeloid_Panel |
| CCCTCACACCAGGTGCACTTC | 344 | 106156939 | TET2_018_Myeloid_Panel |
| GGGATTCCGCTTGGTGAAAACGA | 345 | 106156832 | TET2_019_Myeloid_Panel |
| TCCCAGAGTTCACATCTCCCTCA | 346 | 106157170 | TET2_020_Myeloid_Panel |
| GGAACTGGAGATGTTGGTCCACTG | 347 | 106157060 | TET2_021_Myeloid_Panel |
| GAAATTCCCCTTATAGTCAGACCATG | 348 | 106157417 | TET2_022_Myeloid_Panel |
| GGTTGTGTTTGTGCTGCCTGTTTATG | 349 | 106157295 | TET2_023_Myeloid_Panel |
| GCTTTCAAGAACAGGAGCAGAAG | 350 | 106157618 | TET2_024_Myeloid_Panel |
| GACATTATGAGTCTCGAACTCGCT | 351 | 106157506 | TET2_025_Myeloid_Panel |
| CAGCAAACACAGCAACCCCAAAC | 352 | 106157860 | TET2_026_Myeloid_Panel |
| GTGAAGAAGATCTTGCTTTGGG | 353 | 106157740 | TET2_027_Myeloid_Panel |
| AATGTGCAGCAAAAGAGCATCATTGA | 354 | 106158025 | TET2_028_Myeloid_Panel |
| GCAGCATGCTTTTGAGTGTCC | 355 | 106157952 | TET2_029_Myeloid_Panel |
| CTTCTTCAGAAAAGACACCAACC | 356 | 106158244 | TET2_030_Myeloid_Panel |
| CACAGCTTGCAGGTGGATTCTC | 357 | 106158149 | TET2_031_Myeloid_Panel |
| CTTCAGATATGGGATTTTCCTTCT | 358 | 106158435 | TET2_032_Myeloid_Panel |
| GCTGGGGTGTGGCTATCAAGTTCT | 359 | 106158339 | TET2_033_Myeloid_Panel |
| GCAAAGGCACAGGGCAGATTAACG | 360 | 106162470 | TET2_034_Myeloid_Panel |
| GCCTTTGGTCTTAAATCTTGGG | 361 | 106163978 | TET2_035_Myeloid_Panel |
| CCAATCGCCGGTGTGCCTTGAAT | 362 | 106164782 | TET2_036_Myeloid_Panel |
| CCCCCCACCCCAACCAAAACAAAA | 363 | 106164698 | TET2_037_Myeloid_Panel |
| GCTCCGAGTAGAGTTTGTCAGCC | 364 | 106164875 | TET2_038_Myeloid_Panel |
| TTTGCCAGAAGCAAGATCCC | 365 | 106180744 | TET2_039_Myeloid_Panel |
| TTCTGGATCCAGCCCCTGACAGGC | 366 | 106180810 | TET2_040_Myeloid_Panel |
| GGCAGCAATTGTAACAACTTACTTG | 367 | 106182914 | TET2_041_Myeloid_Panel |
| GAGGACAGCTTAGCAGCTGTTGAG | 368 | 106190791 | TET2_042_Myeloid_Panel |
| GAAAACTCACTAGTATTTAGACC | 369 | 106190713 | TET2_043_Myeloid_Panel |
| GTCAAGACTTGCCGACAAAGGA | 370 | 106193793 | TET2_044_Myeloid_Panel |
| GTAAGACATTACAGCCTCAACTAC | 371 | 106193710 | TET2_045_Myeloid_Panel |
| GCATTTGTAGATAAATGTGTTGTG | 372 | 106193966 | TET2_046_Myeloid_Panel |
| GCCGAAAAGAACTCAGTACCTG | 373 | 106193893 | TET2_047_Myeloid_Panel |
| CCCCAGCAGCAGCAGCCACA | 374 | 106196201 | TET2_048_Myeloid_Panel |
| CCATGAACCCTTACCCTGGGCTT | 375 | 106196382 | TET2_049_Myeloid_Panel |
| GCTGGGGCTGTGGTGGCTGCTTCT | 376 | 106196286 | TET2_050_Myeloid_Panel |
| GCCAAGGTTTGGAAATAGCCAGAG | 377 | 106196581 | TET2_051_Myeloid_Panel |
| CCTGCAGCTTGAGATGAGGTGGA | 378 | 106196485 | TET2_052_Myeloid_Panel |
| GGTGAACATCATTCACCTTCTCAC | 379 | 106196801 | TET2_053_Myeloid_Panel |
| GTGTATGGATGGGTGGTAGACTG | 380 | 106196700 | TET2_054_Myeloid_Panel |
| GCTTTCCCACACAGCTAATGGGT | 381 | 106196908 | TET2_055_Myeloid_Panel |
| GTGATGCTAATGGTCAGGAAAAGC | 382 | 106197000 | TET2_056_Myeloid_Panel |
| TCACCCCACCAGGATCTCCC | 383 | 106197217 | TET2_057_Myeloid_Panel |
| GCAGTTCTATCATGGTTAAGAGCTGG | 384 | 106197097 | TET2_058_Myeloid_Panel |
| CAGAGCCCACTTACCTGCGTTTCA | 385 | 106197420 | TET2_059_Myeloid_Panel |
| GCAGCTCACGCTTTGCACAC | 386 | 106197309 | TET2_060_Myeloid_Panel |
| GAAGTTTCATGTGGCTCAGCAGGC | 387 | 106197547 | TET2_061_Myeloid_Panel |
| CCACAACACTTCATAGACATCA | 388 | 170837497 | NPM1_001_Myeloid_Panel |
| CAGTAGATCTCATTTTCCTATCAG | 389 | 140453093 | BRAF_001_Myeloid_Panel |
| CACAACAAAGCCTGCTGAAGATAG | 390 | 148504736 | EZH2_001_Myeloid_Panel |
| GGGCTTTTTCTACTGGATTGTG | 391 | 148506130 | EZH2_002_Myeloid_Panel |
| GTAAGCACAGCCCAGTGAAT | 392 | 148506421 | EZH2_003_Myeloid_Panel |
| GCTGGGAGGCAGTGAGTTCCT | 393 | 148507413 | EZH2_004_Myeloid_Panel |
| GGCACTGATAACCTGTATTCAGGT | 394 | 148508713 | EZH2_005_Myeloid_Panel |
| GGAGGTTCTTCACTCATCACC | 395 | 148511120 | EZH2_006_Myeloid_Panel |
| GTAGTTAGCTATTTAGTGATGCAA | 396 | 148511019 | EZH2_007_Myeloid_Panel |
| GCATGAGAACTAAATAGGTCTTTG | 397 | 148511979 | EZH2_008_Myeloid_Panel |
| GCTTTGTTTTCATTTGTTTTAG | 398 | 148512554 | EZH2_009_Myeloid_Panel |
| GCAGAGGGTACTTGAGAGGACTTT | 399 | 148516665 | EZH2_010_Myeloid_Panel |
| GTTTCTAAAAGGTTTCCATGTGTT | 400 | 148523659 | EZH2_011_Myeloid_Panel |
| CGTTTTCATTTTCTATCTTTGTTG | 401 | 148523543 | EZH2_012_Myeloid_Panel |
| GAGCCATATGCTTCTTCTCTTGG | 402 | 148526818 | EZH2_013_Myeloid_Panel |
| TTGTTCTGTTGGAATAGTTCCTTG | 403 | 117864786 | RAD21_001_Myeloid_Panel |
| GAGGACCAGCAACAGCAGCAT | 404 | 117864828 | RAD21_002_Myeloid_Panel |
| GAATAATCACTAAGTTGGGCTCT | 405 | 117866477 | RAD21_003_Myeloid_Panel |
| GGATCTGGCACCGCCCACCA | 406 | 117866593 | RAD21_004_Myeloid_Panel |
| CCTTTAGATTTATACAGCAT | 407 | 117868392 | RAD21_005_Myeloid_Panel |
| AGACTAGAAGGTTTGATTTATCTG | 408 | 5069946 | JAK2_001_Myeloid_Panel |
| CAGGATCACAGCTAGGTGTCAGTG | 409 | 5073732 | JAK2_002_Myeloid_Panel |
| CTTTCTACACATGCGTGGAAGTC | 410 | 112350168 | SMC3_001_Myeloid_Panel |
| GCTGGTTTATTCCTTTTCGACGG | 411 | 112350240 | SMC3_002_Myeloid_Panel |
| GTTAGATGTCAGGGATACAGCC | 412 | 112350728 | SMC3_003_Myeloid_Panel |
| AGTAACCTCTCCAGGAAGATTCAT | 413 | 112350837 | SMC3_004_Myeloid_Panel |
| CAGCTGGCCCGTGCTTTCACTA | 414 | 112352813 | SMC3_005_Myeloid_Panel |
| GAGTCTTTCCAAACACATGTTTG | 415 | 112352910 | SMC3_006_Myeloid_Panel |
| GCAAAGCTCAATGAAAACCTGCGC | 416 | 112356146 | SMC3_007_Myeloid_Panel |
| GCCTCGGCCCTAACAATGTGGGCA | 417 | 32413507 | WT1_001_Myeloid_Panel |
| GCTGTGTTCCCTTGGGCTAGGGTT | 418 | 32414183 | WT1_002_Myeloid_Panel |
| GTCTTGAGGGAGAGTGAGCACTGG | 419 | 32417886 | WT1_003_Myeloid_Panel |
| GCAAACATGGTTCAAGAGCTCCTT | 420 | 32417776 | WT1_004_Myeloid_Panel |
| AGGCTCAGTGTGGCTCACAGTCGC | 421 | 32421492 | WT1_005_Myeloid_Panel |
| GAGCCCTGTGGACACCTCATG | 422 | 119148834 | CBL_001_Myeloid_Panel |
| GTTGGAATGTGGAGCCCATCTCAC | 423 | 119148919 | CBL_002_Myeloid_Panel |
| GAGGCAAGGAGCAGAGGGAGCTCC | 424 | 119149195 | CBL_003_Myeloid_Panel |
| CACAGATCTGTTTTCTGCAAAATC | 425 | 25378528 | KRAS_001_Myeloid_Panel |
| GACTGGGGAGGGCTTTCTTTGTGT | 426 | 25380240 | KRAS_002_Myeloid_Panel |
| GCATATTACTGGTGCAGGACCATT | 427 | 25398189 | KRAS_003_Myeloid_Panel |
| GTCCATTGGAAAGGGAGGCAA | 428 | 112888114 | PTPN11_001_Myeloid_Panel |
| TCCGCTCAGTAATAGTCACTCT | 429 | 112910737 | PTPN11_002_Myeloid_Panel |
| GGTCACATAAGTCCTGGACTGCTT | 430 | 112915435 | PTPN11_003_Myeloid_Panel |
| GGACAACAGAATCATTCATGGGGG | 431 | 112926222 | PTPN11_004_Myeloid_Panel |
| GCGCAGGATTGAAGAAGAGCAGGT | 432 | 112926832 | PTPN11_005_Myeloid_Panel |
| GGCCAGGTCTCTGTGAACACACTG | 433 | 28592580 | FLT3_001_Myeloid_Panel |
| AAGGAGCATTAAAAATGTAAAACTCAAGT | 434 | 28602311 | FLT3_002_Myeloid_Panel |
| GTGGAAGGACAGCAACAAAGATGC | 435 | 28608014 | FLT3_003_Myeloid_Panel |
| GCTTCAGAGATGAAATGATGAGTC | 436 | 28608225 | FLT3_004_Myeloid_Panel |
| TCTGCAGCATTTCTTTTCCATTGG | 437 | 28608194 | FLT3_005_Myeloid_Panel |
| GCAACAAAAGAGTGTCACTCAGCG | 438 | 28608421 | FLT3_006_Myeloid_Panel |
| CTGCTCGACACCCACTGTCCAAA | 439 | 28609607 | FLT3_007_Myeloid_Panel |
| GCTGTCATCAGATTGGAAGTTAGG | 440 | 28609721 | FLT3_008_Myeloid_Panel |
| CGTGTGAAATAAGCTCACTGGCTG | 441 | 28610068 | FLT3_009_Myeloid_Panel |
| GCAGATGATGGGCTCCCGGAAGAC | 442 | 90631765 | IDH2_001_Myeloid_Panel |
| GAGATAATAGTGGTCCCACTGCAG | 443 | 90631865 | IDH2_002_Myeloid_Panel |
| GACAGAAGCAGGGAGGAGAGATG | 444 | 7572891 | TP53_001_Myeloid_Panel |
| GCTGGGAAGGAGCCAGGGGGGA | 445 | 7573965 | TP53_002_Myeloid_Panel |
| GTCTAACACTCAAAATGCCG | 446 | 7576812 | TP53_003_Myeloid_Panel |
| ACCAGGCTCCATCTACTCCC | 447 | 7577099 | TP53_004_Myeloid_Panel |
| CCAAGGGTGCAGTTATGCCTCAG | 448 | 7576986 | TP53_005_Myeloid_Panel |
| GTAGTGGATGGTGGTACAGTCAGA | 449 | 7577458 | TP53_006_Myeloid_Panel |
| GCGGCATGAACCGGAGGCCCAT | 450 | 7577551 | TP53_007_Myeloid_Panel |
| CCTGGGGACCCTGGGCAACCA | 451 | 7578192 | TP53_008_Myeloid_Panel |
| GTGAGCAGTAGGGGGGCTTTC | 452 | 7578114 | TP53_009_Myeloid_Panel |
| GTTGAGGGCAGGGGAGTACTGTAGGA | 453 | 7578411 | TP53_010_Myeloid_Panel |
| GGGCTGGAGAGACGACAGGGCTGGTT | 454 | 7578360 | TP53_011_Myeloid_Panel |
| TTCCACACCCCCGCCCGG | 455 | 7578487 | TP53_012_Myeloid_Panel |
| TTTCTGGGAAGGGACAGAAGATGA | 456 | 7579258 | TP53_013_Myeloid_Panel |
| GTAGGAGCTGCTGGTGCAGGGGC | 457 | 7579319 | TP53_014_Myeloid_Panel |
| GCATCAAATCATCCATTGCTTGGG | 458 | 7579436 | TP53_015_Myeloid_Panel |
| ACCCAGGTCCAGATGAAGCTCCC | 459 | 7579518 | TP53_016_Myeloid_Panel |
| AAGGGCAGGCCCACCACCCC | 460 | 7579822 | TP53_017_Myeloid_Panel |
| GCCTTCCAATTGGCCTTGTGCC | 461 | 58740447 | PPM1D_001_Myeloid_Panel |
| AAAAAATTTATCCCAGAACTCAAC | 462 | 58740327 | PPM1D_002_Myeloid_Panel |
| AGCCTGCAAGTCTCCCCACAACC | 463 | 58740654 | PPM1D_003_Myeloid_Panel |
| CAATTTTCTTCAAGTGGTTCTGG | 464 | 58740529 | PPM1D_004_Myeloid_Panel |
| GATGTTGAACTTTTTTTAAGGGGA | 465 | 58740853 | PPM1D_005_Myeloid_Panel |
| CGTCTATGCTTCTTCATCAGGGG | 466 | 58740751 | PPM1D_006_Myeloid_Panel |
| AGCTCGCGGCCGTCCAGCAC | 467 | 74732953 | SRSF2_001_Myeloid_Panel |
| TTTCTGCTCCCTGGACAACCCGG | 468 | 42531864 | SETBP1_001_Myeloid_Panel |
| GGCCTGCCTCCAGGGCTGGACTGA | 469 | 13054605 | CALR_001_Myeloid_Panel |
| CTTGCCCCCTGCCAGCCCTG | 470 | 13054500 | CALR_002_Myeloid_Panel |
| GCAACTGCGCGTGAGGCGCGCGGCTGT | 471 | 33792258 | CEBPA_001_Myeloid_Panel |
| TGACCGCCTGCGCAAGCGGGTGGA | 472 | 33792359 | CEBPA_002_Myeloid_Panel |
| GCGGGGCTCCTGCTTGATCACCAGCG | 473 | 33792748 | CEPBA_003_Myeloid_Panel |
| TCATGCCCGGGGGAGCGCACGGGC | 474 | 33792966 | CEBPA_004_Myeloid_Panel |
| GCTGATGTCGATGGACGTCTCGTGCTCGC | 475 | 33793061 | CEBPA_005_Myeloid_Panel |
| GCAGGTGGCTGCTCATCGGGGGCCGC | 476 | 33793200 | CEBPA_006_Myeloid_Panel |
| GCCACCCGACAGCGAGATGGGCA | 477 | 31021073 | ASXL1_001_Myeloid_Panel |
| TCCCCATCTGCCAGGCACATCC | 478 | 31021264 | ASXL1_002_Myeloid_Panel |
| CCTGGGACACACAAGCCACT | 479 | 31021171 | ASXL1_003_Myeloid_Panel |
| GAGCAGGCGGCCTCTGCATCCTTT | 480 | 31021457 | ASXL1_004_Myeloid_Panel |
| GCCCTGCTGGGTCAGTCTTAG | 481 | 31021383 | ASXL1_005_Myeloid_Panel |
| CACAGCCCACTAAAGAGGAGCCCA | 482 | 31021554 | ASXL1_006_Myeloid_Panel |
| CGGGGTTGGACTGGCGCCAGGA | 483 | 31022205 | ASXL1_007_Myeloid_Panel |
| GTGGTGATGGTGGTGAGGCCTGTGG | 484 | 31022405 | ASXL1_008_Myeloid_Panel |
| GGTGGGGATGATCCGGGGGCATAT | 485 | 31022316 | ASXL1_009_Myeloid_Panel |
| TGCCAGGCCTTGCCCCTACTGTC | 486 | 31022664 | ASXL1_010_Myeloid_Panel |
| GTACACTTTCCAGGGGTGCTCGGG | 487 | 31022579 | ASXL1_011_Myeloid_Panel |
| GGATCCTGTAAATGTGACCCCC | 488 | 31022903 | ASXL1_012_Myeloid_Panel |
| GCAAGGCCTGGCATGGCTGGT | 489 | 31022803 | ASXL1_013_Myeloid_Panel |
| GGTAGTGAAACAGCCCAAACCAG | 490 | 31023107 | ASXL1_014_Myeloid_Panel |
| GCTTGGCCAGTTCCTTTCTCT | 491 | 31023005 | ASXL1_015_Myeloid_Panel |
| GGAGACTCTGAAGCACTGAGTCCT | 492 | 31023348 | ASXL1_016_Myeloid_Panel |
| CGATGGGATGGGTATCCAATGCA | 493 | 31023225 | ASXL1_017_Myeloid_Panel |
| GTTGACAGTAACTGCCATTGCTGT | 494 | 31023445 | ASXL1_018_Myeloid_Panel |
| GGTGACCTTCAAAGTCAGAGGCTG | 495 | 31023541 | ASXL1_019_Myeloid_Panel |
| GTAGCTTGCCCCTAGAGAAGGT | 496 | 31023730 | ASXL1_020_Myeloid_Panel |
| GTGGGGCAGATTGGTTCCAATTGG | 497 | 31023643 | ASXL1_021_Myeloid_Panel |
| GCAACTGCATCACAAGTGGGTTAG | 498 | 31023850 | ASXL1_022_Myeloid_Panel |
| CCCTCCATCCAGTGACAAATCCCA | 499 | 31024024 | ASXL1_023_Myeloid_Panel |
| GCAGTTCTCTTCCTTTAGTTGTG | 500 | 31024088 | ASXL1_024_Myeloid_Panel |
| GCGAGCCATGGCTCTGGTCTTTTG | 501 | 31023955 | ASXL1_025_Myeloid_Panel |
| CCTCGTTTCTCATCTCCAAATGTG | 502 | 31024203 | ASXL1_026_Myeloid_Panel |
| AACCCTTCAGCGCCCCAGGC | 503 | 31024325 | ASXL1_027_Myeloid_Panel |
| GTGGGGTACAGACTCCAAGGGAAG | 504 | 31024429 | ASXL1_028_Myeloid_Panel |
| GCCGAGAACAGGAAAGCTACTGGG | 505 | 31024533 | ASXL1_029_Myeloid_Panel |
| GAAGGGGCTCAGTGAGCCTCTGGA | 506 | 31024643 | ASXL1_030_Myeloid_Panel |
| CCTCTAGCTCTCCCACCTTTC | 507 | 31024759 | ASXL1_031_Myeloid_Panel |
| GCAGCACGGTGGAAAGCATCTCG | 508 | 31024876 | ASXL1_032_Myeloid_Panel |
| GCTCTGTGTATTGTGCCTTGTGGT | 509 | 31024985 | ASXL1_033_Myeloid_Panel |
| GAGGTCAATGGATCTCACCAAAGCCA | 510 | 57484392 | GNAS_001_Myeloid_Panel |
| GGTGGTAGGAGGGCGAGCTGGCTT | 511 | 36164550 | RUNX1_001_Myeloid_Panel |
| GCGGCGGCAGGTAGGTGTGGTAG | 512 | 36164640 | RUNX1_002_Myeloid_Panel |
| GGTGACCGGCGTCGGGGAGTAGGT | 513 | 36164725 | RUNX1_003_Myeloid_Panel |
| GCGGGGGTCGGAGATGGAGGG | 514 | 36164740 | RUNX1_004_Myeloid_Panel |
| GCGGAAGTGAGTAGGAGGTTGCGGA | 515 | 36164821 | RUNX1_005_Myeloid_Panel |
| GTCCAGGAGACTAGAGGTGCATG | 516 | 36171551 | RUNX1_006_Myeloid_Panel |
| ACGCCCATTTCACCTGGACGTG | 517 | 36171668 | RUNX1_007_Myeloid_Panel |
| GAGGCACGAGGGTTGGGCGTG | 518 | 36206645 | RUNX1_008_Myeloid_Panel |
| CGGGCTTGGTCTGATCATCTAGT | 519 | 36206751 | RUNX1_009_Myeloid_Panel |
| GCGGCGCACAGCCATGAGGGT | 520 | 36206820 | RUNX1_010_Myeloid_Panel |
| GGGCTGGTACACCCTCCAGGCT | 521 | 36231754 | RUNX1_011_Myeloid_Panel |
| CATCATTGCCAGCCATCACAG | 522 | 36252824 | RUNX1_012_Myeloid_Panel |
| GCTGAGCTGAGAAATGCTACCGC | 523 | 36252939 | RUNX1_013_Myeloid_Panel |
| GCAGAGGAAGTTGGGGCTGTCGGTGCGCA | 524 | 36259124 | RUNX1_014_Myeloid_Panel |
| GGCCAGCACCTCCACCATGCTG | 525 | 36259181 | RUNX1_015_Myeloid_Panel |
| GCCAGTGACGTGACTGAGCACA | 526 | 44514751 | U2AF1_001_Myeloid_Panel |
| CATGGAATATGTCAGCAGCATGAC | 527 | 44524436 | U2AF1_002_Myeloid_Panel |
| GCATGCGTGTGGAGGAGGGGACTG | 528 | 15833892 | ZRSR2_001_Myeloid_Panel |
| GGCCAATAGTTGAAAATTACTCAC | 529 | 15833769 | ZRSR2_002_Myeloid_Panel |
| AAATTCAGGAAAAGAAACCAGCC | 530 | 15836680 | ZRSR2_003_Myeloid_Panel |
| TTTCCTCAATTGTTCCACTGC | 531 | 15838326 | ZRSR2_004_Myeloid_Panel |
| GCTTCCAGGTCTTTGGAGCAAGAG | 532 | 39911360 | BCOR_001_Myeloid_Panel |
| GTAAAATGAAAAGTGCGCCCAAC | 533 | 39911563 | BCOR_002_Myeloid_Panel |
| GTAAGGAGCTGTTAGATCTGGTGG | 534 | 39911472 | BCOR_003_Myeloid_Panel |
| CGTTCTCAACAGCATCGTGCAGAG | 535 | 39914615 | BCOR_004_Myeloid_Panel |
| CTTATGGTGCTGACCCCACC | 536 | 39914711 | BCOR_005_Myeloid_Panel |
| TCGCCCAGTCCAATGCCTTG | 537 | 39932867 | BCOR_006_Myeloid_Panel |
| CATGTGGTCAGCTTTGGAAGCATC | 538 | 39933099 | BCOR_007_Myeloid_Panel |
| CAGCAGCGGAGTTCATCATGCCCG | 539 | 39932988 | BCOR_008_Myeloid_Panel |
| CAGGGGTTTTCTTCCCCTCTGGGCCTGAG | 540 | 48649480 | GATA1_001_Myeloid_Panel |
| GCTGGAAGCTTCTCAAATGGATG | 541 | 48649716 | GATA1_002_Myeloid_Panel |
| GGTGTGGAGGACACCAGAGCAGGA | 542 | 48649600 | GATA1_003_Myeloid_Panel |
| GCAGCTCTGCCTCTTTCCGTTTTG | 543 | 53431919 | SMC1A_001_Myeloid_Panel |
| GCACTGCCTGTGGCTTACTTTC | 544 | 53432033 | SMC1A_002_Myeloid_Panel |
| GCCTTCAGGTCACTGGCCCCA | 545 | 53432127 | SMC1A_003_Myeloid_Panel |
| CCAGTACTGAGCCTGTCCAG | 546 | 53432252 | SMC1A_004_Myeloid_Panel |
| GACAAGGGCATTGCCACAAGCATA | 547 | 53432356 | SMC1A_005_Myeloid_Panel |
| CCTCACCTCCAGGTAGTCAAG | 548 | 53432571 | SMC1A_006_Myeloid_Panel |
| GCCGCATTGCCTTTGGAGGCCA | 549 | 53432460 | SMC1A_007_Myeloid_Panel |
| GCTTTTGGAAGCTGGCTCAGG | 550 | 53432798 | SMC1A_008_Myeloid_Panel |
| GGGTCAGGCCAGTGTTCAAGGG | 551 | 53432684 | SMC1A_009_Myeloid_Panel |
| GGCTATTGTGTGACCAACTTGGTC | 552 | 123181254 | STAG2_001_Myeloid_Panel |
| GGTGAAACTAATCTAACAGACACA | 553 | 123181181 | STAG2_002_Myeloid_Panel |
| GCAAGTTTGCATATTTCGTGGTGT | 554 | 123182853 | STAG2_003_Myeloid_Panel |
| AAGATGTGCCCTTCAGACTGCTT | 555 | 123184027 | STAG2_004_Myeloid_Panel |
| CCAGTCGGTTCAAGGTTAGTATTA | 556 | 123184930 | STAG2_005_Myeloid_Panel |
| GTGTCTATGACCCTTGACAAAG | 557 | 123185048 | STAG2_006_Myeloid_Panel |
| GCATATTTGCACTAATGTTCAGAT | 558 | 123185136 | STAG2_007_Myeloid_Panel |
| GAAATAAGCAGTAACAGGTGCTT | 559 | 123189974 | STAG2_008_Myeloid_Panel |
| ACCATGTCAGTCATTAGTGGAATC | 560 | 123220383 | STAG2_009_Myeloid_Panel |
| CCACCAGCTAGCAAAGAATTTCGG | 561 | 123220499 | STAG2_010_Myeloid_Panel |
| CCGGCAGCAACAGAGACCTTGAAA | 562 | 133511669 | PHF6_001_Myeloid_Panel |
| GCCACTTTAAGTCTCAAGAAATGC | 563 | 133511625 | PHF6_002_Myeloid_Panel |
| GCATTTCATCATCATCATAAAGGG | 564 | 133527622 | PHF6_003_Myeloid_Panel |
| GTATGTGACTTTCTAAGGCTGTAT | 565 | 133527501 | PHF6_004_Myeloid_Panel |
| CCACGTTTCAGCCACTTTTCAG | 566 | 133527920 | PHF6_005_Myeloid_Panel |
| GGGGAGGAAGAAAATGAAGCACGA | 567 | 133547793 | PHF6_006_Myeloid_Panel |
| GCCTGGTGTCACTAGGGCTGC | 568 | 133547896 | PHF6_007_Myeloid_Panel |
| ATGCAGGAAAATTAACATTCAGAATCC | 569 | 133549033 | PHF6_008_Myeloid_Panel |
| GACTGCAAAGTGTACATTTCTG | 570 | 133551218 | PHF6_009_Myeloid_Panel |

| | | | | | SEQ |
|---|---|---|---|---|---|
| scan_target_sequence | | | | | ID |
| CGGGGGCGGTACCTGTAGTGTGCAGGAAACTGCCACCTCAGCAGCAGCAGGCCCAGGACGGCGCTGAGGCCCAGCACTAGATGCAGAGCGGTCACCAAGGAGATCCAGGCTAGGAGACAA | | | | | 571 |
| AAAGGG | | | | | |
| GCCTTCGCCTGTCCTCATGTATTGGTCTCTCATGGCACTGTACTCTTCTTGTCCAGCTGTATCCAGTATGTCCAACAAACAGGTTTCACCATCTATAACCACTTGTTTTCTGTAAGAATC | | | | | 572 |
| CTGGGG | | | | | |
| AAATGACTGAGTACAAACTGGTGGTGGTTGGAGCAGGTGGTGTTGGGAAAAGCGCACTGACAATCCAGCTAATCCAGAACCACTTTGTAGATGAATATGATCCCACCATAGAGGTGAGGC | | | | | 573 |
| CCAGTG | | | | | |
| CCTTACACACACGCAAAATACTCCTTCAGCGGAGCGAAGAGGTGGCGGATGACTGGCACGCTCCATGACCGGCCCAGCAGTCTCTGCCTCGCCAAGCGGCTCATGTTGGAGACGTCAGTA | | | | | 574 |
| TAGTGG | | | | | |
| TTTCTCCCCCAGGGTATTTGGTTTCCCAGTCCACTATACTGACGTCTCCAACATGAGCCGCTTGGCGAGGCAGAGACTGCTGGGCCGGTCATGGAGCGTGCCAGTCATCCGCCACCTCTT | | | | | 575 |
| CGCTCC | | | | | |
| CTAGTTCAGCAAAGTGAGGACCATTACTACGAGGTCAAACTCCATAAAGCAGGGCAAAGACCAGCATTTTCCTGTCTTCATGAATGAGAAAGAGGACATCTTATGGTGCACTGAAATGGA | | | | | 576 |
| AAGGTA | | | | | |
| ACAGTCTCTCTTCTGCCTCCTAGGCCGTTGGCATCCACTGTGAATGATAAGCTGGAGCTGCAGGAGTGTCTGGAGCATGGCAGGATAGCCAAGGTCAGCTCCAGCGTCTAGAACCTCTGC | | | | | 577 |
| TGGGGG | | | | | |
| CTCATCTTCAAACCGTCTCCTGTTTTGTAGTCCAACCCTGTGATGATTGATGCCAAAGAAGTGTCAGCTGCACACAGGGCCCGCTACTTCTGGGGTAACCTTCCCGGTATGAACAGGTTG | | | | | 578 |
| GTGAAA | | | | | |
| CTGGCCAAACCAAGGTTGCTGGCTATACCTCGAGAAATCGCGAGATGTCCCTCTTGTCACTAACGCCCATGGCCACCACATTCTCAAAGAGCCAGAAGAAGGGGCGATCATCTCCCTCCT | | | | | 579 |
| TGGGCC | | | | | |
| CTTCCGACCTCTCAGAGGGCACTGGCCGGCTCTTCTTTGAGTTCTACCGCCTCCTGCATGATGCGCGGCCCAAGGAGGGAGATGATCGCCCCTTCTTCTGGCTCTTTGAGAATGTGGTGG | | | | | 580 |
| CCATGG | | | | | |
| CCTACCGTAGAGGCCCTTGCGAGCAGGGTTGACGATGGAGAGGTCATTGCAGGGACTGCCCCCAATCACCAGATCGAATGGGCCCCACTCCTGGATCTGGGAGGATAAAGGCAACGTGAT | | | | | 581 |
| GGGCCT | | | | | |
| AATGTAGCGGTCCACCTGAATGCCCAAGTCCTTCAGCACCAGGAGCCCTGCACCAGCCAGCAGACAGCACCGTTAC | | | | | 582 |
| GGACCGCTACATTGCCTCGGAGGTGTGTGAGGACTCCATCACGGTGGGCATGGTGCGGCACCAGGGGAAGATCATGTACGTCGGGGACGTCCGCAGCGTCACACAGAAGCATGTAT | | | | | 583 |
| ACCGCTGGGCCTGCATCTGACCTGTTGTGCTCACTGCTTAGGACCCTCCAAAGGTTTACCCACCTGTCCCAGCTGAGAAGAGGAAGCCCATCCGGGTGCTGTCTCTCTTTGATGGAATCG | | | | | 584 |
| CTACAG | | | | | |
| GGTCGTGGTTATTAGCGAAGAACATCTGGAGCCGGGAGGGCCAGTCCTCTCGCCGCCGCAGCAGCCCGTAGGTACCCTTGTGCCCGCACATGTAGCAGTTCCAGGGGT | | | | | 585 |
| GGCGGCGAGAGGACTGGCCCTCCCGGCTCCAGATGTTCTTCGCTAATAACCACGACCAGGAATTTGTGAGTGCTGG | | | | | 586 |
| CCTTCCAGGTGCTTTTGCGTGGAGTGTGTGGACCTCTTGGTGGGGCCGGGGGCTGCCCAGGCAGCCATTAAGGAAGA | | | | | 587 |
| CTGTCTAGAACTGCTTTCTGGAGTGTGCGTACCAGTACGACGACGACGGCTACCAGTCCTACTGCACCATCTGCTGTGGGGGCCGTGAGGTGCTCATGTGCGGAAACAACAACTGCTGCA | | | | | 588 |
| GGTGAG | | | | | |
| ACCTTGCAGTTTTGGCACATTCCTCCAACGAAGAGGGGGTGTTCCAGGGTAACATTGAGGCTCCCACAGGAGATGCAGATGTCTGGAAAGCAGAGGGAGGGGATGG | | | | | 589 |
| ACATCTGCATCTCCTGTGGGAGCCTCAATGTTACCCTGGAACACCCCCTCTTCGTTGGAGGAATGTGCCAAAACTGCAAGGTAGGAGCACACCCACCCAGGAGAGG | | | | | 590 |
| GCCCCATGCCACACTAGGAGTGCCAGAGTTCCCAGGCAACAAACTTACCCTCAATGTTCCGGCACTTCTGCCGCACCTCGTACACCAGCCGCTCTGCAAGGGGAGGAGAGCTGGCGTCAG | | | | | 591 |
| AGGAGG | | | | | |
| GAAGCAGGCCAACTACCTCTTGTGCGCTCATCAATAATCTCCTTGACCTTGGGCTTCTCCGCTGTGCTCTTCCGGGGCTTTTTGGCTGGTGGAGGTGGTGCGTAGGCAGCTGCCTCAGGT | | | | | 592 |
| TCCACC | | | | | |
| TGCGTAGGCAGCTGCCTCAGGTTCCACCCACATGTCCGTGTACACTTCTTTGTAGGGATTCTTCTCTTCTGGAGGAGGAAAGCAGGTGCCAAGGTCAGTTACAGGCTGACAGGAAACTCC | | | | | 593 |
| AGCCCC | | | | | |
| CCCCCCAGGGCCCATTCAATCATGGGCTTGTTCTGCACCTCCACGGCCTTGGCAGTGTCACTCTCATCGCTGTCGTGGCACACCGGGAACAGCTTCCCCGCGCGGCTGCTGGCCACCTGG | | | | | 594 |
| AGG | | | | | |
| CTTCCAGCCTTCTGGCCCTAAGGGCCTGGAGCCACCAGAAGGTAAATGAGGGCACCCAGCTTTCTGGGACCCCTGCCCGCCAGGCAGATCCACACCAGGGCTGGGAAAGCCATGCTTAGG | | | | | 595 |
| GAG | | | | | |
| ACATGGGCTGCTTGTTGTACGTGGCCTGGTGGAACGCACTGCAAAACGAGCTCAGCGGCATCAGCTTCTCAACACACACCTGGGGGGACAAGCCAGGCCTTGTTTGCCGAGGCTTACACT | | | | | 596 |
| TGCAAG | | | | | |
| CCGCTGAGCTCGTTTTGCAGTGCGTTCCACCAGGCCACGTACAACAAGCAGCCCATGTACCGCAAAGCCATCTACGAGGTCCTGCAGGTGAGTGTCCCTGCTGGGAGCTCGGAGGAGGAG | | | | | 597 |
| CCTAGA | | | | | |
| ACCACTGAGAATTTGCCGTCTCCGAACCACATGACCCAGCGGGTGCCTTCAGCTGCTCGGCTCCGGCCCGTCATCCACCAAGACACAATGCGGCCTGGCCACCAGGAGAAGCCCCGCAGT | | | | | 598 |
| CCTCGTGACCACTGTGTAATGATTTCTGCTCCTTGGGGCTCCAGGACGGCCGGGGCTTTGGCATTGGGGAGCTGGTGTGGGGGAAACTGCGGGGCTTCTCCTGGTGGCCAGGCCGCATT | | | | | 599 |
| GGCATTCTTGTCCCCAGCATCGGACCCCACGGGCTCAGGCGTGGTAGCCACAGTGGGGGATGCGGGGTCAGTGGGCTGCTGCACAGCAGGAGGGCTGGCCTCCTCCACCTTCTGAGACTC | | | | | 600 |
| CC | | | | | |
| CCATAAGAGGAATAAGATACCCAATAGCCTTCAAGAAAGCAGCCAAACCCTATTTTTAAATAAAAAATATATGTACTTTAGTAATTTAGATTTATGTCGCCTTAACTTTAATGAAGATAA | | | | | 601 |
| ATCAAA | | | | | |
| ATGCAGAATATGCCAACTACTATACTAGAGAAGTGATGTTAATCCTTATTCGAGAATTCCAGTCTCCTGATGAGGAAATGAAAAAAATTGTGCTGAAGGTAATTATTCCAGATTTGTTAA | | | | | 602 |
| TGTAAA | | | | | |
| TTAAAACCTGTGTTTGGTTTTGTAGGTCTTGTGGATGAGCAGCAGAAAGTTCGGACCATCAGTGCTTTGGCCATTGCTGCCTTGGCTGAAGCAGCAACTCCTTATGGTATCGAATCTTTT | | | | | 603 |
| GATTCT | | | | | |
| AGGGCAGAGGCTACAACAGCAAAAGCTCTAGCTGTTGTGTTACGGACATACTCATCCATGTTATCTATATCAGGTCTCATGGTAGAGATCATAGTAGCCAGACCAGCAGCCTAAAATGTA | | | | | 604 |
| AACAAA | | | | | |
| TTTGCTGTTGTAGCCTCTGCCCTGGGCATTCCTTCTTTATTGCCCTTCTTAAAAGCTGTGTGCAAAAGCAAGAAGTCCTGGCAAGCGAGACACACTGGTATTAAGATTGTACAACAGATA | | | | | 605 |
| GCTATT | | | | | |
| ACTTACTTGATCCCCATAAGCATGACGACCTATGATGATAGGTTTTACCCATCCACTCACAAGCCGGGGGATATTTTTGCAGATAATGGCTTCTCTGAAGACCGTGCCACCCAGAATATT | | | | | 606 |
| TCGTAT | | | | | |
| CTAGGTTAACAGGCCACTGACCATGAAGAAGGAAGGGATCCAGACTCGGAACCGGAAGATGTCCAACAAGTCCAAGAAGAGCAAGAAAGGGGCGGAGTGCTTCGAGGAGCTGTCAAAGTG | | | | | 607 |
| CA | | | | | |
| CACAGGCGTTGCAGACAGGGTCCCCGTTGGCGTTTCGGCGCCATAAGGTGGTGGTTGTCGTCTGACAATTTGCACAACA | | | | | 608 |
| CAGAAGGCCGGGAGTGTGTCAACTGTGGGGCCACAGCCACCCCTCTCTGGCGGCGGGACGGCACCGGCCACTACCTGTGCAATGCCTGTGGCCTCTACCACAAGATGAATGGGCAGAACC | | | | | 609 |
| GAC | | | | | |
| TGACATATGGCCATTTCTGTTTTCCTGTAGCAAAACCAGAAATCCTGACTTACGACAGGCTCGTGAATGGCATGCTCCAATGTGTGGCAGCAGGATTCCCAGAGCCCACAATAGATTGGT | | | | | 610 |
| ATTTTT | | | | | |
| ATGGGTACTCACGTTTCCTTTAACCACATAATTAGAATCATTCTTGATGTCTCTGGCTAGACCAAAATCACAAATCTTTGTGATCCGACCATGAGTAAGGAGGATATTTCTGGCTGCCAA | | | | | 611 |
| GTCTCT | | | | | |
| AGACTAAGTCCATTCCTGATACCATCACCTCCCATTTGCCAGACAGAACCTCTGGCTACAAAGCTCCAGAATGGAA | | | | | 612 |
| ACTCTTTCAAAAGTTATTATGGAATACCCTGTATGAAGGGAAGCCAGAATAGTCGTGTGAGTCCTGACTTTACACAAGAAA | | | | | 613 |
| TATTCCATAATAACTTTTGAAAGAGTGCCACTTGGTGTCTCCATTTACTTCTGGATGAGCTCTCTCAGGCAGTGGGCTTCCATTCTGGAGCTTTGTAGCCAGAG | | | | | 614 |
| AAATTGAAACAAGACCAAAAGGCTAATGGAGAAAGACGTAACTTCGGGGTAAGCCAAGAAAGAAATCCAGGTGAAAGCAGTCAACCAAATGTCTCCGATTTGAGTGATAAGAAAGAATCT | | | | | 615 |
| GTGAGT | | | | | |
| TCTTGTTTCAATTTCTTGATCTGAAGGAGCCCAGAGAGAGAAGGTTCACTAACTGTGCGTTTTATTCCTCCATTTTGCAAACACTTGGAATACCCTCTACTTTCTTGTGTAAAGTCAGGA | | | | | 616 |
| CTCACA | | | | | |
| AATGAGCAGGAGGGGAAAAGTGCTAATTACCATGACAAGAACATTGTATTACTTAAAAACAAGGCAGTGCTAATGCCTAATGGTGCTACAGTTTCTGCCTCTTCCG | | | | | 617 |
| TCAGAATCTGAAGCTCTGGATTTTCAGGCCCACTGCAGTTATGTGTTGAAAAACTGGTGAAATCTTTAACTGCATTTTCTTGGGCTACAGAACTCACAGATTCTTTCTTATCACTCAAAT | | | | | 618 |
| CGGAGA | | | | | |
| AATGCCATTAACAGTCAGGCTACTAATGAGTTGTCCTGTGAGATCACTCACCCATCGCATACCTCAGGGCAGATCAATTCCGCACAGACCTCTAACTCTGAGCTGCCTCCAAAGCCAGCT | | | | | 619 |
| GCAGTG | | | | | |
| TTAATGGCATTTATGTGAGATGTGGTTTTCTGCACCGCAATGGAAACACAATCTGGATAATATTGAGACAGTGTTTTTTCCAGGAGTTCACCATGTGTGTGTTCCACGGAAGAGGCAGAA | | | | | 620 |
| ACTGTA | | | | | |
| CAACTACAACAACAAAAATCAGTTTTTGAGATATGCCCATCTCCTGCAGAAAATAACATCCAGGGAACCACAAAGCTAGCGTCTGGTGAAGAATTCTGTTCAGGTT | | | | | 621 |
| CATATCTCAAAAACTGATTTTTGTTGTTGTAGTTGTTCTGGTTTCTGAAAGGAACAGGTATTTAGCATTGCAGCTAGTTTACTGGCATTATCAGCATCATCAGCATCACAGGCCTCACTC | | | | | 622 |
| ACCACT | | | | | |
| TTTTCTGCCACTACCACACCACCACCACCATCACAATTGCTTCTTTCTCCCCCTCCTCCTCTTCCACAGGTTCCTCAGCTTCCTTCAGAAGGAAAAAGCACTCTGAATGGTGGAGTTTTA | | | | | 623 |
| GAAGAA | | | | | |
| GGTAGTGGCAGAAAAGGAATCCTTAGTGAACACTGAGCTTTGCTTGAAGTAAGCACCATTCATTTCATTTTGTTTTAAATACCGTTCAGAGCTGCCACCAGGAGCTTGCAAATTGCTGCT | | | | | 624 |
| GGAACC | | | | | |
| CCATCTACACATGTATGCAGCCCTTCTCCGATGCTTTCTGAAAGGCCTCAGAATAATTGTGTGAACAGGAATGACATACAGACTGCAGGGACAATGACTGTTCCATTGTGTTCTGAGAAA | | | | | 625 |
| ACAAGA | | | | | |
| AGATGGATTAGGACTCTGGGAAGGTGGTGCCTCAGGTTTACCCTCTATTTTCACTTCCCTTAAAAGTGTTGTGTTACTTTGGTTGGGGTAGTGGTGGTGTTCTTCTAAAACTCCACCATT | | | | | 626 |
| CAGAGT | | | | | |
| GAGATTCTGAAGGGTCGAGACAAGGAGCAAACACGAGATCTTGTGCCCCCAACACAGCACTATCTGAAACCAGGATGGATTGAATTGAAGGCCCCTCGTTTTCACCAAGCGGAATCCCAT | | | | | 627 |
| CTAAAA | | | | | |
| GCCCTTCAGAATCTCTTGCTCTTTGTTTCTCATCAACTGCTGGCAGTTGTCCTGTAGCTCTCCACTGCTACCAAAAATTGGTGGGTTATGCTTGAGGTGTTCTGACATTGGTCTTGTTTTC | | | | | 628 |
| TCAGA | | | | | |
| GGGCTCCCAAGGCAAGCTTACACCCAGAAAACAACACAGCTGGAGCACAAGTCACAAATGTACCAAGTTGAAATGAATCAAGGGCAGTCCCAAGGTACAGTGGACCAACATCTCCAGTTC | | | | | 629 |
| CAAAAA | | | | | |
| AAGCTTGCCTTGGGAGCCCCCCAGGCATGTTGGAATTTCCAGTGTATTGTTTGGAGGTCATTTGATTGGAGAGATTGGGTTGATACTGAAGAATTGATGGCAGTGATGCCTCATTACGTT | | | | | 630 |
| TTAGAT | | | | | |
| ACTGAAAAACTTATGTCCCCAGTGTTGAAACAGCACTTGAATCAACAGGCTTCAGAGACTGAGCCATTTTCAAACTCACACCTTTTGCAACATAAGCCTCATAAACAGGCAGCACAAACA | | | | | 631 |
| CAACCA | | | | | |
| ACATAAGTTTTTCAGTTTGGGAATCTGCTCTTTGTTGAAAATGAAATCTAGTGCCACACAGTGACTGCACATGAGCTTTTGGTAAATGGTCTGTTTTGGAGAAGTGCACCTGGTGTGAGG | | | | | 632 |
| GTTTTT | | | | | |
| GATCATTCTTTGGCCAGACTAAAGTGGAAGAATGTTTTCATGGTGAAAATCAGTATTCAAAATCAAGCGAGTTCGAGACTCATAATGTCCAAATGGGACTGGAGGAAGTACAGAATATAA | | | | | 633 |
| ATCGTA | | | | | |
| GATCCTTCTCTTTGCTGATCATTGTTGCTTTGGGGGTGAGGAAAAGTCTGGAGTATTTCCTCTTTATTCTTTATTTGTAATTTTTGCTGCTGTTGCTGGTTTTGAGGGAGATGTGAACTC | | | | | 634 |
| TGGGAT | | | | | |
| TAGTTTCAGAGAATAAAGAACAGACTACACATCCTGAACTTTTTGCAGGAAACAAGACCCAAAACTTGCATCACATGCAATATTTTCCAAATAATGTGATCCCAAAGCAAGATCTTCTTC | | | | | 635 |
| ACAGGT | | | | | |
| ATTCTCTGAAACTAGGTGTGTATTGTTTGAACAAGAAACCTGTATTTTGCATGCACTTGATTTCATGGTCTGACTATAAGGGGAATTTCTACGATTTATATTCTGTACTTCCTCCAGTCC | | | | | 636 |
| CATTTG | | | | | |
| ATACATAACCATGCAAATGTTTTTCCTGTGCCTGACCAGGGAGGAAGTCACACTCAGACCCCTCCCCAGAAGGACACTCAAAAGCATGCTGCTCTAAGGTGGCATCTCTTACAGAAGCAA | | | | | 637 |
| GAACAG | | | | | |
| TGGTTATGTATCAAGTACCTTTGCTGAGCAAGTTGCGCAGCTTGTTGACCAGACATATCTTGGTTTCTATTTTTATATCCCTGTAGAACTGAAGCTTGTTGTGACTTCTGCTCCTGTTCT | | | | | 638 |
| TGAAAGCACCT | | | | | |
| CAGATGCACAGGCCAATTAAGGTGGAACCTGGATGCAAGCCACATGCCTGTATGCACACAGCACCACCAGAAAAAAAACATGGAAAAAGGTAACTAAGCAAGAGAATCCACCTGCAAGCT | | | | | 639 |
| GTGAT | | | | | |
| ATACAGGCATGTGGCTTGCATCCAGGTTCCACCTTAATTGGCCTGTGCATCTGACTATGGCAAGACTCAGTTTGGGGTTGCTGTGTTTGCTGCTGTTCTTGCTTCTGTAAGAGATGCCAC | | | | | 640 |
| CTTAGA | | | | | |
| AAATCACAGAAGCAAGTAAAAGTTGAAATGTCAGGGCCAGTCACAGTTTTGACTAGACAAACCACTGCTGCAGAACTTGATAGCCACACCCCAGCTTTAGAGCAGCAAACAA | | | | | 641 |
| ACATTTCAACTTTTACTTGCTTCTGTGATTTGAGAGTAAGAGCCTTATGGTCAAATAACGACTTGGCGTGAAACTGCTTCAGATGCTGCTCCATGGTCTCAATGATGCTCTTTTGCTGCA | | | | | 642 |
| CATTAT | | | | | |
| AGATACTCCTATAAAAAATTTATTGGATACACCTGTCAAGACTCAATATGATTTCCCATCTTGCAGATGTGTAGGTAAGTGCCAGAAATGTACTGAGACACATGGCGTTTATCCAGAATT | | | | | 643 |
| AGCAAATTTAT | | | | | |
| AATAAATTTTTTATAGGAGTATCTAGTAATTTGGAAGGTGACTCTATAAAATTATTGAGAACAGAAGCAGCTGTTCTTTTGGTTGGTGTCTTTTCTGAAGAAGTTGTTTGCTGCTCTAAA | | | | | 644 |
| ATAATAATCTTCTATTATCTCAACAGAGCAAATTATTGAAAAAGATGAAGGTCCTTTTTATACCCATCTAGGAGCAGGTCCTAATGTGGCAGCTATTAGAGAAATCATGGAAGAAAGGTA | | | | | 645 |
| ATTAAC | | | | | |
| GGGTTTCTTTAAGGTTTGGACAGAAGGGTAAAGCTATTAGGATTGAAAGAGTCATCTATACTGGTAAAGAAGGCAAAAGTTCTCAGGGATGTCCTATTGCTAAGTGGGTAAGTGTGACTT | | | | | 646 |
| GATAAA | | | | | |
| CTGGCCACACCTGTGAGGCTGCAGTGATTGTGATTCTCATCCTGGTGTGGGAAGGAATCCCGCTGTCTCTGGCTGACAAACTCTACTCGGAGCTTACCGAGACGCTGAGGAAATACGGCA | | | | | 647 |
| CGCTCA | | | | | |
| GGATGAGAATCACAATCACTGCAGCCTCACAGGTGTGGCCAGCTCGCTCCCGCACCAAACACAGTAGCTTCTCTTCACTGCTGCTTCTGCGAACCACCTGCGTGGATCACCATTCCATCC | | | | | 648 |
| CAAACA | | | | | |
| CAACCAAAGATTGGGCTTTCCTATCAGTGGCCGCAAAGAGGGGAGAGGCCCTGGGCTTCACTTACTCTTCATTCAAGGCACACCGGCGATTGGTGAGCGTGCCGTATTTCCTCAGCGTCT | | | | | 649 |
| CGGTAA | | | | | |
| ATAGCAATGAATTTGGTCTTTTGATTTTTCAGGAGAACTTGCGCCTGTCAGGGGCTGGATCCAGAAACCTGTGGTGCCTCCTTCTCTTTTGGTTGTTCATGGAGCATGTACTACAATGGA | | | | | 650 |
| TGTAAG | | | | | |
| TAAACAAACCTCTTTTGGGTCATCCCCAAGCAGCTTAAACTTCCTTGGGATCTTGCTTCTGGCAAACTTACATCCATTGTAGTACATGCTCCATGAACAACCAAAAGAGAAGGAGGCACC | | | | | 651 |
| ACAGGT | | | | | |
| AGGAAGAGAAACTGGAGTCTCATTTGCAAAACCTGTCCACTCTTATGGCACCAACATATAAGAAACTTGCACCTGATGCATATAATAATCAGGTAAGTTTAAATAATCATT | | | | | 652 |
| GAGTGCCGTCTGGGTCTGAAGGAAGGCCGTCCATTCTCAGGGGTCACTGCATGTTTGGACTTCTGTGCTCATGCCCACAGAGACTTGCACAACATGCAGAATGGCAGCACATTGGTAAGT | | | | | 653 |
| TGGGCT | | | | | |
| AGTGACCCCTGAGAATGGACGGCCTTCCTTCAGACCCAGACGGCACTCTGGTGCTCTGTGTTCATATTCAATCTGAAAAATAAAAGTGTGTGTGAATGCGAAAGTAGTTAGTTTTACTCT | | | | | 654 |
| TACACC | | | | | |
| CCTTTATACAAAGTCTCTGACGTGGATGAGTTTGGGAGTGTGGAAGCTCAGGAGGAGAAAAAACGGAGTGGTGCCATTCAGGTACTGAGTTCTTTTCGGCGAAAAGTCAGGATGTTAGCA | | | | | 655 |
| GAGCCA | | | | | |
| CCACACTCCCAAACTCATCCACGTCAGAGACTTTGTATAAAGGCAGAACGTGAAGCTGCTCATCCTCAGGTTTTCCTCCAAATTCTCGATTGTCTTCTCTAGTGAGAGTGCATACCTGGT | | | | | 656 |
| AGGGAA | | | | | |
| TGAAAAGCTTTCCTCCCTGGAGAACAGCTCAAATAAAAATGAAAAGGAAAAGTCAGCCCCATCACGTACAAAACAAACTGAAAACGCAAGCCAGGCTAAACAGTTGGCAGGTAAATTTAA | | | | | 657 |
| TGTAAA | | | | | |
| ACTTTTCCTTTTCATTTTTATTTGAGCTGTTCTCCAGGGAGGAAAGCTTTTCAGCTGCAGCTTTCTTGGCTTCTAGTTTCCTTTGTCGGCAAGTCTTGACTGGCTCTGCTAACATCCTGA | | | | | 658 |
| CTTTTC | | | | | |
| ACAGAACTTTTGCGACTTTCAGGACCAGTCATGCAGCAGTCCCAGCAGCCCCAGCCTCTACAGAAGCAGCCACCACAGCCCCAGCAGCAGCAGAGA | | | | | 659 |
| GGCCCAATCCAGTTAGTCCTTATCCAAACTCTTCACACACTTCAGATATCTATGGAAGCACCAGCCCTATGAACTTCTATTCCACCTCATCTCAAGCTGCAGGTTCATATTTGAATTCTT | | | | | 660 |
| CTAATC | | | | | |
| AGTTTGGATAAGGACTAACTGGATTGGGCCGTCTCATGTATGGATTGGTGGATCCAGAAGCAGAATAAGAGTTGACAGACTCTGTCTGAGGGTGATGTGGCTGCTGCTGCTGGGGTCTCT | | | | | 661 |
| GCTGCT | | | | | |
| AGTGGACAACTGCTCCCCATATCTGGGTTCCTATTCTCCCCAGTCTCAGCCGATGGATCTGTATAGGTATCCAAGCCAAGACCCTCTGTCTAAGCTCAGTCTACCACCCATCCATACACT | | | | | 662 |
| TTACCA | | | | | |
| GAACCCAGATATGGGGAGCAGTTGTCCACTGATAGGTTTCCATTGCATTGATATGATGGATATTGGGTATTCTGATTCAAAAGCCCAGGGTAAGGGTTCATGGGATTAGAAGAATTCAAA | | | | | 663 |
| TATGAA | | | | | |
| AGACCAAATGTACATCATGTAGGGAAATTGCCTCCTTATCCCACTCATGAGATGGATGGCCACTTCATGGGAGCCACCTCTAGATTACCACCCAATCTGAGCAATCCAAACATGGACTAT | | | | | 664 |
| AAAAAT | | | | | |
| TCCCTACATGATGTACATTTGGTCTAATGGTACAACTGCTGAAACCATCTCCCTGCATATTTTGGTTTCCATAACCTAAGTATTTAGATGTAAAACTCTGGCTATTTCCAAACCTTGGCT | | | | | 665 |
| GGTAAA | | | | | |
| AAACATGGACTATAAAAATGGTGAACATCATTCACCTTCTCACATAATCCATAACTACAGTGCAGCTCCGGGCATGTTCAACAGCTCTCTTCATGCCCTGCATCTCCAAAACAAGGAGAA | | | | | 666 |
| TGACAT | | | | | |
| ATGCCCTGCATCTCCAAAACAAGGAGAATGACATGCTTTCCCACACAGCTAATGGGTTATCAAAGATGCTTCCAGCTCTTAACCATGATAGAACTGCTTGTGTCCAAGGAGGCTTACACA | | | | | 667 |
| AATTAA | | | | | |
| CGAGCAGAGCTTTCTGGATCCTGACATTGGGGGAGTGGCCGTGGCTCCAACTCATGGGTCAATTCTCATTGAGTGTGCAAAGCGTGAGCTGCATGCCACAACCCCTTTAAAGAATCCCAA | | | | | 668 |
| TAGGAA | | | | | |
| CGCTCGCTGTCTGACCAGACCTCATCGTTGTCCTCTGCACCAGAAGCCACACCCTGGACTAGTGCCAATGGCTGCTTTTCCTGACCATTAGCATCACTTAATTTGTGTAAGCCTCCTTGGA | | | | | 669 |
| CACAA | | | | | |
| AAGCCAAAATGGCTGAAAAAGCCCGTGAGAAAGAGGAAGAGTGTGAAAAGTATGGCCCAGACTATGTGCCTCAGAAATCCCATGGCAAAAAAGTGAAACGGGAGCCTGCTGAGCCACATG | | | | | 670 |
| AAACTT | | | | | |
| CAGCCATTTTGGCTTCCCAAAGAGCCAAGCCATGTTTTGGCTCATTCATGCTCTTATGCTGGTAAAAGACGAGGGAGATCCTGGTGGGGTGATTCCTATTGGGATTCTTTAAAGGGGTTG | | | | | 671 |
| TGGCAT | | | | | |
| ATATATCTGTTGTAAGGCCCTGTGACCCGAGTGAAGGCATATGGAGATGTAGTTACTGTGGAGTCTGTGGTCACGGACATGGTCCTTTCGGCAAGAGACTTGATGAAACGCAGGTAAGTG | | | | | 672 |
| GGCTCT | | | | | |
| ACAGAAATGAAATAAGACGGAAAATTTTTTAACAAATTGTTTAAACTATTTTCTTAAAGAGACTTCCTCCACTGCCAGAGATCTTGAATAGCCTGGAAAAAAAAAAAAAGAAATGTGGTT | | | | | 673 |
| AAGGAA | | | | | |
| AGACAACTGTTCAAACTGATGGGACCCACTCCATCGAGATTTCACTGTAGCTAGACCAAAATCACCTATTTTTACTGTGAGGTCTTCATGAAGAAATATATCTGAGGTGTAGTAAGTAAA | | | | | 674 |
| GGAAAA | | | | | |
| TGTCAAGGGATTTCCATTTCTCTTTCGATGCCGACATACTTCAGGGCATCAGCCTGGCTGTATCTGAAACAACAGGAAGGAGATGTCCGCTGGATGGCCACCCATCCAACATGTGCTGAG | | | | | 675 |
| ACTTAA | | | | | |
| CTTTGCAGTTATGATGGTTAACGGTGATCACAGGATAGGTATTTTTGCCAAGAGAGCCATCCAGACTGGCGAAGAGCTGTTTTTTGATTACAGGTTGGTAAAGTACATTTCTAGCATGAT | | | | | 676 |
| CTCTAA | | | | | |
| ATTTACCGAATGATTTGCAAAACGAATTTTGTTACCCTTGCGGGTTGCATCCACCACAAAATCTAAAAAGAAAAAA | | | | | 677 |
| AAAAGAATTTTCTCCTGTGTCTTTCTTTTTAGATTATTTCTCAAGATGAAGCTGACAGAAGAGGGAAAGTGTATGATAAATACATGTGCAGCTTTCTGTTCAACTTGAACAATGGTATGT | | | | | 678 |
| TTCAGA | | | | | |
| TGATGTGATTGTGTTTTATTCTCTAGCATCTATTGCTGGCACCATCTGACGTGGCAGGCTGGGGGATTTTTATCAAAGATCCTGTGCAGAAAAATGAATTCATCTCAGAATACTGTGGAG | | | | | 679 |
| AGGTAA | | | | | |
| TCCACAAGTAAGACAGAGGTCAGGGTCACACTCTCGGACAGCCAGGTAGCACGGGCACTGCTTGGTGTTGCACTGTGCTTTGCAGCGGCATCCCGGAAAGCGGTTTTGACCTTCAGAGAG | | | | | 680 |
| AGGTTT | | | | | |
| CCCTGACCTCTGTCTTACTTGTGGAGCCGCTGACCATTGGGACAGTAAAAATGTGTCCTGCAAGAACTGCAGTATTCAGCGGGGCTCCAAAAAGGTGAGCAACAAGTCACTTCTGGAAGA | | | | | 681 |
| TTGTTT | | | | | |
| ATCAACCCTGTGATCATCCACGGCAGCCTTGTGACAGTTCGTGCCCTTGTGTGATAGCACAAAATTTTTGTGAAAAGTTTTGTCAATGTAGTTCAGAGTGTAAGTATTTGTTGCTTTGAT | | | | | 682 |
| GCAATT | | | | | |
| TCCCGCAGAAATTTGGTTTAATTTTCTTTGTGTTTTTGCAGGTTGTGGGCTGCACACTGCAGAAAGATACAGCTGAAAAAGGGTTAGCATCTTTCCATTCCTCTCATTATTAGCTTAACA | | | | | 683 |
| ATATCT | | | | | |
| TTATTTTGCAGCTTTTCATGCAACACCCAACACTTATAAGCGGAAGAACACAGAAACAGCTCTAGACAACAAACCTTGTGGACCACAGTGTTACCAGCATTTGGTAAGACTTAGTGCCTA | | | | | 684 |
| ATTATT | | | | | |
| TCTATGTTGGGGGTACATTCAGGAGGAAGTGCGCCTGGGAGCTGCTGTTCGGTGAGTTCTTTATATCTGACATTAACCAAGAAAAATTTAAGTAAACATGAAACAAAAATCACTTTTTTG | | | | | 685 |
| AAAACA | | | | | |
| CCAACATAGATGGACCAAATGCTAAATCTGTTCAGAGAGAGCAAAGCTTACACTCCTTTCATACGCTTTTCTGTAGGCGATGTTTTAAATATGACTGCTTCCTACATCGTAAGTGCAATT | | | | | 686 |
| ATTGTA | | | | | |
| ATGAAACTGTTTTACATAACATTCCTTATATGGGAGATGAAGTTTTAGATCAGGATGGTACTTTCATTGAAGAACTAATAAAAAATTATGATGGGAAAGTACACGGGGATAGAGGT | | | | | 687 |
| GACCTTAGAAAAAGGAGGAAAGGAGGAGAGGCAGATAATTTGGATGAATTCCTCAAAGAATTTGAAAATCCAGAGGTTCCTAGAGAGGACCAGCAACAGCAGCATCAGCAGCGTGATGTT | | | | | 688 |
| ATCGGT | | | | | |
| TAATAGCTCTTTACACGCTGTCTTACACCGCTTGTACCAGAAGACCTTAGAAAAAGGAGGAAAGGAGGAGAGGCAGATAATTTGGATGAATTCCTCAAAGAATTTGAAAATCCAGAGGTT | | | | | 689 |
| CCTAGA | | | | | |
| ATATTACCTTCAGTAGTCTGTTATTCCACAAAGGCTGAGCAGGTAAAGAAAACAGTTTTTCTACTCCTCCTGTCTCTTTCCACATCATCAATTTCTTGGTGGGCGGTGCCAGATCCAAAG | | | | | 690 |
| TAGTAACAATATCT | | | | | |
| TATCATTCCAGTTAAAGAAACAAAAGCCAAGAGGAAGAGGAAGCTAATTGTTGACAGTGTCAAAGAGTTGGATAGCAAGACAATTAGAGCCCAACTTAGTGATTATTCAGATATTGTTAC | | | | | 691 |
| TACTTT | | | | | |
| GGTGGGCCTGATAGTCCTGATTCAGTGGATCCCGTTGAACCAATGCCAACCATGACTGATCAAACAACACTTGTTCCAAATGAGGAAGAAGCATTTGCATTGGAGCCTATTGATATAACT | | | | | 692 |
| GGTAAGCATATGAA | | | | | |
| TGAGTATCTAATGACTTACAAATATCAAATCTTCATTTCTGATTTTGTGAAACACCATTTGGTTCATATGAGTAGGCCTCTGTAATGTTGGTGAGGTTGGTACATCAGAAACACCATTCG | | | | | 693 |
| TTCTGA | | | | | |
| TTTCTCACAAGCATTTGGTTTTAAATTATGGAGTATGTGTCTGTGGAGACGAGAGTAAGTAAAACTACAGGCTTTCTAATGCCTTTCTCAGAGCATCTGTTTTTGTTTATATAGAAAATT | | | | | 694 |
| CAGTTT | | | | | |
| AGGCCATTTTAAATGGAATAGACAGCATAAACAAAGTGCTAGACCACTTCCGTCGAAAAGGAATAAACCAGCATGTTCAAAATGGCTATCATGGTATTGTAATGAATAACTTTGAATGTG | | | | | 695 |
| AACCAG | | | | | |
| TCTAAAAATGCTAAATATCAAGCAAAAGCTTTAACCTGTTTCCAGCAGTGACTTCCACGCATGTGTAGAAAGCTGGTTCACATTCAAAGTTATTCATTACAATACCATGATAGCCATTTT | | | | | 696 |
| GAACAT | | | | | |
| TCTTACTCTGTTTATATTTAGGTTATTTTATCACATTGTTGATTCAGATGAAGTCAGCACGAAGATTTTAATGGAGTTTAATAAAATGAATCTTCCTGGAGAGGTTACTTTTCTGCCTCT | | | | | 697 |
| TAACAA | | | | | |
| AGCATGTGTTAGATTATGTGATAAAAAATAACATTATAAAAGAGAAATACACCTTATCACACAATGACTCACATTGGTTTCAGGATAGGCTGTATCCCTGACATCTAACTTGTTAAGAGG | | | | | 698 |
| CAGAAA | | | | | |
| ACAAAATTTCATTTTTAGGATGCTATTCCTATGATCAGCAAACTGAGGTACAATCCCAGATTTGACAAAGCTTTCAAACATGTGTTTGGAAAGACTCTTATTTGTCGTAGCATGGAAGTT | | | | | 699 |
| TCAACC | | | | | |
| TTAAATGATTTGTTAGAATTTATCTGTTTAAAGCTAAGAATTAGTATTACAAACCTTCCAAAGTAATACAGTCCATAGTGAAAGCACGGGCCAGCTGGGTTGAAACTTCCATGCTACGAC | | | | | 700 |
| AAATAA | | | | | |
| TTGTTTATAGGTGACCAAGTCAGCCATCGGGGTGCTCTAACTGGGGGTTATTATGACACAAGGAAGTCTCGACTTGAATTGCAAAAAGATGTTAGAAAAGCAGAAGAAGAACTAGGTGAA | | | | | 701 |
| CTTGAA | | | | | |
| AGTTTACGCACTTGTTTTACCTGTATGAGTCCTGGTGTGGGTCTTCAGGTGGTCGGACCGGGAGAACTTTCGCTGACAAGTTTTACACTGGAATGGTTTCACACCTAAATGGACAGAGAA | | | | | 702 |
| GGTCTA | | | | | |
| CCCCAAGGTGAGAAACCATACCAGTGTGACTTCAAGGACTGTGAACGAAGGTTTTCTCGTTCAGACCAGCTCAAAAGACACCAAAGGAGACATACAGGTTTGTAGGTTCACTTCTCATTG | | | | | 703 |
| CTGGCA | | | | | |
| CGTTTCTCACTGGTCTCAGATGCCGACCGTACAAGAGTCGGGGCTACTCCAGGCACACGTCGCACATCCTGCAGGCAGAGAGTAAGAGGAAGGGAGGCTTTAAGCCACATGTGAACATTC | | | | | 704 |
| ACGTAG | | | | | |
| AGACCAGTGAGAAACGCCCCTTCATGTGTGCTTACCCAGGCTGCAATAAGAGATATTTTAAGCTGTCCCACTTACAGATGCACAGCAGGAAGCACACTGGTAAGTGTGCCCGCTGTCCAG | | | | | 705 |
| TCTTGG | | | | | |
| ACCTGAATGCCTCTGAAGACACCGTGCGTGTGTATTCTGTATTGGGCTCCGCAGAGGATGGGCGTTGTGTGGTTATCGCTCTCGTACCCTGTGCTGTGGCTGCAAACACAAAGAAGGGAAAA | | | | | 706 |
| TATTTATTCAACTAATAGTCTTTTAATTTTTTTTAATCAAAGGAACAATATGAATTATACTGTGAGATGGGCTCCACATTCCAACTATGTAAAATATGTGCTGAAAATGATAAGGATGTA | | | | | 707 |
| AAGATT | | | | | |
| TTACATAGCTGAAAAAAGTCGCTGTTTAGATCCGTACCTGCCAGGATGTAAGACAGGATGTGCACATGAGGTGTCCACAGGGCTCAATCTTTACATCCTTATCATTTTCAGCACATATTT | | | | | 708 |
| TACATA | | | | | |
| TTACTATCTTTTGCTTCTTCTGCAGGAATCAGAAGGTCAGGGCTGTCCTTTCTGCCGATGTGAAATTAAAGGTACTGAACCCATCGTGGTAGATCCGTTTGATCCTAGAGGGAGTGGCAG | | | | | 709 |
| CCTGTT | | | | | |
| AAATAAATGTGATTTGCCTTCTAGAACAGTAGACACAAAACAGGCTCAGGACTTAGCAAGAAGTTATGGAATTCCTTTTATTGAAACATCAGCAAAGACAAGACAGGTAAGTAACACTGAA | | | | | 710 |
| ATAAA | | | | | |
| TGTGTTTCTCCCTTCTCAGGATTCCTACAGGAAGCAAGTAGTAATTGATGGAGAAACCTGTCTCTTGGATATTCTCGACACAGCAGGTCAAGAGGAGTACAGTGCAATGAGGGACCAGTA | | | | | 711 |
| CATGAG | | | | | |
| CTGAATATAAACTTGTGGTAGTTGGAGCTGGTGGCGTAGGCAAGAGTGCCTTGACGATACAGCTAATTCAGAATCATTTTGTGGACGAATATGATCCAACAATAGAGGTAAATCTTGTTT | | | | | 712 |
| TAATAT | | | | | |
| GTGATGTTCCATGTAATACTGGACCAACTCAGCCAAAGTGGCAAATTTCTCCCCTCCATACAGGTCATAGTAATCACCAGTGTTCTGAATCTTGATGTGGGTGACAGCTCCATTTCTTCT | | | | | 713 |
| AAAATA | | | | | |
| TTTCTTTCCAGACACTACAACAACAGGAGTGCAAACTTCTCTACAGCCGAAAAGAGGGTCAAAGGCAAGAAAACAAAAACAAAAATAGATATAAAAACATCCTGCCCTGTAAGTATCAAT | | | | | 714 |
| AT | | | | | |
| AAAACACTGTGAAAAGCAAAGCTTACCATGATGATATTTGCATTGATGTAATCTGAAACAGGCTCATTGGGATCACCATCGTGTAGGACAACCCTGGTATGATCAACTAGAAGAAAAAGA | | | | | 715 |
| GACCAC | | | | | |
| TGTCGCTTCTTGCCCACCAGATGACCCACCTTTCTCTCTGATGATGTCAATAAGAATATCAATCACAATGAACGTCCCTGTCCGGCCAATTCCAGCACTGCGGGCAGAAGGACAAAAAGC | | | | | 716 |
| AG | | | | | |
| TGACTGCGATATTGACGTTCCCAAAACCATCCAGATGGTGCGGTCTCAGAGGTCAGGGATGGTCCAGACAGAAGCACAGTACCGATTTATCTATATGGCGGTCCAGCATTATATTGAAAC | | | | | 717 |
| ACTACA | | | | | |
| ATAAGTAGGAAATAGCAGCCTCACATTGCCCCTGACAACATAGTTGGAATCACTCATGATATCTCGAGCCAATCCAAAGTCACATATCTTCACCACTTTCCCGTGGGTGACAAGCACGTT | | | | | 718 |
| CCTGGC | | | | | |
| TTACCTGACAGTGTGCACGCCCCCAGCAGGTTCACAATATTCTCGTGGCTTCCCAGCTGGGTCATCATCTTGAGTTCTGACATGAGTGCCTCTCTTTCAGAGCTGTCTGCTTTTTCTGTC | | | | | 719 |
| AAAGA | | | | | |
| TTTGCAGGGAAGGTACTAGGATCAGGTGCTTTTGGAAAAGTGATGAACGCAACAGCTTATGGAATTAGCAAAACAGGAGTCTCAATCCAGGTTGCCGTCAAAATGCTGAAAGGTACAGTA | | | | | 720 |
| TA | | | | | |
| CTAAATTTTCTCTTGGAAACTCCCATTTGAGATCATATTCATATTCTCTGAAATCAACGTAGAAGTACTCATTATCTGAGGAGCCGGTCACCTGTACCATCTGTAGCTGGCTTTCATACC | | | | | 721 |
| TAAATT | | | | | |
| ACAGGTGACCGGCTCCTCAGATAATGAGTACTTCTACGTTGATTTCAGAGAATATGAATATGATCTCAAATGGGAGTTTCCAAGAGAAAATTTAGAGTTTGGTAAGAATGGAATGTGCCA | | | | | 722 |
| AATGTT | | | | | |
| TTTACCTTTGCTTTTACCTTTTTGTACTTGTGACAAATTAGCAGGGTTAAAACGACAATGAAGAGGAGACAAACACCAATTGTTGCATAGAATGAGATGTTGTCTTGGATGAAAGGGAAG | | | | | 723 |
| GGGCCT | | | | | |
| AATATCACAAGAACAACTGTTGTACCTGGAGAGTTTAAAAGGATCGTCTCACAAGATGTGCCAAGGGAATTGTATGCACAGCACTTGACCAGGAACCCTTTTATGGCTTCACTCATGTTT | | | | | 724 |
| AGAGTA | | | | | |
| ATGTTTAGAGTACTGCTCGACACCCACTGTCCAAACACTTTTCTGTTAGCCTTTCTATTCCAGACTCCTTCTGTGATCTCTTCTGTGCAGCTGAAAAAAAAAATAGCAAAGAATTTAGAC | | | | | 725 |
| AGGTGA | | | | | |
| TTACTTGGGAGACTTGTCTGAACACTTCTTCCAGGTCCAAGATGGTAATGGGTATCCATCCGAGAAACAGGACGCCTGACTTGCCGATGCTTCTGCGAGCACTTGAGGTTTCCCTATAGA | | | | | 726 |
| AAAGAA | | | | | |
| AGCTCCAGTCGGGGGGTGCCCAGGTCAGTGGATCCCCTCTCCACCCTGGCCTACCTGGTCGCCATGGGCGTGCCTGCCAATGGTGATGGGCTTGGTCCAGCCAGGGACTAGGCGTGGGAT | | | | | 727 |
| GTTTTT | | | | | |
| CCAGGGACTAGGCGTGGGATGTTTTTGCAGATGATGGGCTCCCGGAAGACAGTCCCCCCCAGGATGTTCCGGATAGTTCCATTGGGACTTTTCCACATCTTCTTCAGCTTGAACTCTGTG | | | | | 728 |
| AGGACA | | | | | |
| GGGAGGCTGTCAGTGGGGAACAAGAAGTGGAGAATGTCAGTCTGAGTCAGGCCCTTCTGTCTTGAACATGAGTTTTTTATGGCGGGAGGTAGACTGACCCTTTTTGGACTTCAGGTGGCT | | | | | 729 |
| GTAGGA | | | | | |
| TCTTTTAACTCAGGTACTGTGTATATACTTACTTCTCCCCCTCCTCTGTTGCTGCAGATCCGTGGGCGTGAGCGCTTCGAGATGTTCCGAGAGCTGAATGAGGCCTTGGAACTCAAGGAT | | | | | 730 |
| GCCCAG | | | | | |
| CTCTTTCCTAGCACTGCCCAACAACACCAGCTCCTCTCCCCAGCCAAAGAAGAAACCACTGGATGGAGAATATTTCACCCTTCAGGTACTAAGTCTTGGGACCTCTTATCAAGTGGAAAG | | | | | 731 |
| TTTCCA | | | | | |
| CTCCCAGGACAGGCACAAACACGCACCTCAAAGCTGTTCCGTCCCAGTAGATTACCACTACTCAGGATAGGAAAAGAGAAGCAAGAGGCAGTAAGGAAATCAGGTCCTACCTGTCCCATT | | | | | 732 |
| TAAAAA | | | | | |
| CCTGTCCTGGGAGAGACCGGCGCACAGAGGAAGAGAATCTCCGCAAGAAAGGGGAGCCTCACCACGAGCTGCCCCCAGGGAGCACTAAGCGAGGTAAGCAAGCAGGACAAGAAGCGGTGG | | | | | 733 |
| AGGAGA | | | | | |
| GCACAGCAGGCCAGTGTGCAGGGTGGCAAGTGGCTCCTGACCTGGAGTCTTCCAGTGTGATGATGGTGAGGATGGGCCTCCGGTTCATGCCGCCCATGCAGGAACTGTTACACATGTAGT | | | | | 734 |
| T | | | | | |
| CCTGCTTGCCACAGGTCTCCCCAAGGCGCACTGGCCTCATCTTGGGCCTGTGTTATCTCCTAGGTTGGCTCTGACTGTACCACCATCCACTACAACTACATGTGTAACAGTTCCTGCATG | | | | | 735 |
| G | | | | | |
| GGGCACCACCACACTATGTCGAAAAGTGTTTCTGTCATCCAAATACTCCACACGCAAATTTCCTTCCACTCGGATAAGATGCTGAGGAGGGGCCAGACCTAAGAGCAATCAGTGAGGAAT | | | | | 736 |
| CAGAGG | | | | | |
| GAGTATTTGGATGACAGAAACACTTTTCGACATAGTGTGGTGGTGCCCTATGAGCCGCCTGAGGTCTGGTTTGCAACTGGGGTCTCTGGGAGGAGGGGTTAAGGGTGGTTGTCAGTGGCC | | | | | 737 |
| CTCCAG | | | | | |
| CACAACCTCCGTCATGTGCTGTGACTGCTTGTAGATGGCCATGGCGCGGACGCGGGTGCCGGGCGGGGGTGTGGAATCAACCCACAGCTGCACAGGGCAGGTCTTGGCCAGTTGGCAAAA | | | | | 738 |
| CATCTT | | | | | |
| GTCACAGCACATGACGGAGGTTGTGAGGCGCTGCCCCCACCATGAGCGCTGCTCAGATAGCGATGGTGAGCAGCTG | | | | | 739 |
| TTCACTTGTGCCCTGACTTTCAACTCTGTCTCCTTCCTCTTCCTACAGTACTCCCCTGCCCTCAACAAGATGTTTTGCCAACTGGCCAAGACCTGCCCTGTGCAGCTGTGGGTTGA | | | | | 740 |
| GGATACGGCCAGGCATTGAAGTCTCATGGAAGCCAGCCCCTCAGGGCAACTGACCGTGCAAGTCACAGACTTGGCTGTCCCAGAATGCAAGAAGCCCAGACGGAAACCGTAGCTGCCCTG | | | | | 741 |
| GTAGGT | | | | | |
| GTCACAGACTTGGCTGTCCCAGAATGCAAGAAGCCCAGACGGAAACCGTAGCTGCCCTGGTAGGTTTTCTGGGAAGGGACAGAAGATGACAGGGGCCAGGAGGGGGCTGGTGCAGGGGCC | | | | | 742 |
| GCCGGT | | | | | |
| GCCGCCGGTGTAGGAGCTGCTGGTGCAGGGGCCACGGGGGGAGCAGCCTCTGGCATTCTGGGAGCTTCATCTGGACCTGGGTCTTCAGTGAACCATTGTTCAATATCGTCCGGGGACA | | | | | 743 |
| GGCTGGGGGGCTGAGGACCTGGTCCTCTGACTGCTCTTTTCACCCATCTACAGTCCCCCTTGCCGTCCCAAGCAATGGATGATTTGATGCTGTCCCCGGACGATATTGAACAATGGTTCA | | | | | 744 |
| CTGAAG | | | | | |
| CTTGCAGCAGCCAGACTGCCTTCCGGGTCACTGCCATGGAGGAGCCGCAGTCAGATCCTAGCGTCGAGCCCCCTCTGAGTCAGGAAACATTTTCAGACCTATGGAAACTGTGAGTGGATC | | | | | 745 |
| CATTGG | | | | | |
| AGGTTTCAGCTGAGATAGCTCGAGAGAATGTCCAAGGTGTAGTCATACCCTCAAAAGATCCAGAACCACTTGAAGAAAATTGCGCTAAAGCCCTGACTTTAAGGATACATGATTCTTTGA | | | | | 746 |
| ATAATA | | | | | |
| AAACCTCTAAAAAATTCTCTGAGAAGGCATTGCTACGAACCAGGGCAGGTATATGGTCCTTAGAATTCACCCTTGGCCATGGATCCTCCTCCAGTGACTGAAAAATAAGAAGGTAAAACA | | | | | 747 |
| AATAAG | | | | | |
| AAATGAAAGCCCAAGAAATTGAAAGAACCCCTCCAACAAACTTTAAAAGGACATTAGAAGAGTCCAATTCTGGCCCCCTGATGAAGAAGCATAGACGAAATGGCTTAAGTCGAAGTAGTG | | | | | 748 |
| GTGCTC | | | | | |
| TGGCCAGGAGTTGACATCTTCAAATTTTTTTGGTCCATGACAGTGTTTGTTGAATTAGTAGGCACAAGGCCAATTGGAAGGCTATTATTCAAAGAATCATGTATCCTTAAAGTCAGGGCT | | | | | 749 |
| TTAGCG | | | | | |
| CCAGAAGAAAATTGGAAATCCTTTACTTCATCAACACAGGAAAACTGTTTGTGTTTGCTGAAATGCATCTGGGAAATGAGGTTTTTCCAAACTTAGGATATAAGAGGGCTTTTTAAATTT | | | | | 750 |
| GGTGCC | | | | | |
| AAAGGATTTCCAATTTTCTTCTGGCCCCTAAGTCTGCGTCGCATGGTGAGTTTAACAGAGTTCTTTCGCTGTGAGGTTGTGGGGAGACTTGCAGGCTGAGCACCACTACTTCGACTTAAGC | | | | | 751 |
| CATTT | | | | | |
| TCCGGGGGGCGGCCGTAGCGCGCCATTTGCACCCGC | | | | | 752 |
| GTCCCCTGTGAGCGAGTCCCACAGTGAGGAGACGATCCCCAGCGACAGCGGCATTGGGACAGACAACAACAGCACTTCTGACCAAGCGGAGAAGAGCTCAGAATCCCGAAGGAGGTACTC | | | | | 753 |
| TTTTGA | | | | | |
| GAGGAGGAGGAGGCAGAGGACAAGGAGGATGATGAGGACAAAGATGAGGATGAGGAGGATGAGGAGGACAAGGAGGAAGATGAGGAGGAAGATGTCCCCGGCCAGGCCAAGGACGAGCTG | | | | | 754 |
| TAGAGA | | | | | |
| GTCCTCTGCCTCCTCCTCCTCTTTGCGTTTCTTGTCTTCTTCCTCCTCCTTAAGCCTCTGCTCCTCGTCCTGTTTGTCCTTCATTTGTTTCTCTGCTGCCTGCAGGCAGAGCACACACCT | | | | | 755 |
| CAGGGC | | | | | |
| ACCAGTGACAATGACCGCCTGCGCAAGCGGGTGGAACAGCTGAGCCGCGAACTGGACACGCTGCGGGGCATCTTCCGCCAGCTGCCAGAGAGCTCCTTGGTCAAGGCCATGG | | | | | 756 |
| CAAGAACAGCAACGAGTACCGGGTGCGGCGCGAGCGCAACAACATCGCGGTGCGCAAGAGCCGCGACAAGGCCAAGCAGCGCAACGTGGAGACGCAGCAGAAGGTGCTGGAGCTGACCAG | | | | | 757 |
| TGACAA | | | | | |
| GTGCGAGGGCGGCGGCGGCGGCGGCGGCTGGTAAGGGAAGAGGCCGGCCAGCGCCAGCTGCTTGGCTTCATCCTCCTC | | | | | 758 |
| GGCCCCACGGGCGGCGGCGGCGGCGGCGACTTTGACTACCCGGGCGCGCCCGCGGGCCCCGGCGGCGCCG | | | | | 759 |
| TGCCGGCTGTGCTGGAACAGGTCGGCCAGGAACTCGTCGTTGAAGGCGGCCGGGTCGATGTAGGC | | | | | 760 |
| GCGCGGGGCCCGCGCCCCGGGGAAAGCCGAAGGCGGCGCTGCTGGGCGCGTGCGGGGGGCTCT | | | | | 761 |
| ATTTTGATTTGCAGGCTGGGTTTGACCAAAGAAGAGTCATTGCAGCAGAACGTGGGCCAGGAGGAGGCTGAAATCAAAAGTGGCTTGTGTGTCCCAGGAGAATCAGTGCGTATACAGCGT | | | | | 762 |
| GGTCCA | | | | | |
| AAGGAATCTGTACAAAAAACAGGAGTCAGAACAAGCAGGGGTTGCTAAGGATGCAAAATCTGTGGCCTCAGATGTTCCCCTCTACAAGGATGGGGAGGCTAAGACTGACCCAGCAGGGCT | | | | | 763 |
| GAGCAG | | | | | |
| TGTTCTGACTCCTGTTTTTTGTACAGATTCCTTCTGGCTCTGGTTCGGAGATCTGGCCGAGAGCGTTTCTTAAAATGCCCATCTCGCTGTCGGGTGGCTGGACCACGCTGTATACGCACT | | | | | 764 |
| GATTCT | | | | | |
| GTGGCTTCTCGGATCCAGGCTGAGCCAGACAACTTGGCACGTGCCTCTGCATCTCCAGACAGAATTCCTAGCCTGCCTCAGGAAACTGTGGATCAGGAACCCAAGGATCAGAAGAGGAAA | | | | | 765 |
| TCCTTT | | | | | |
| ATGCAGAGGCACGTGCCAAGTTGTCTGGCTCAGCCTGGATCCGAGAAGCCACAGACTCAACTGGGAATTCGGGACCCTCCAGGTCGGGTGCTGCAGAGGATGTGCCTGGCAGATGGGGAC | | | | | 766 |
| TGCTCA | | | | | |
| AACCCAAGGATCAGAAGAGGAAATCCTTTGAGCAGGCGGCCTCTGCATCCTTTCCCGAAAAGAAGCCCCGGCTTGAAGATCGTCAGTCCTTTCGTAACACAATTGAAAGTGTTCACACCG | | | | | 767 |
| AAAAGC | | | | | |
| TAAAACTATTTTCTAATTCTTTTTTTGCAGATTCAACTTTCACGTATCAAACCACCCTGGGTGGTTAAAGGTCAGCCCACTTACCAGATATGCCCCCGGATCATCCCCACCACGGAGTCC | | | | | 768 |
| TCCTGC | | | | | |
| CCACTGCCATAGAGAGGCGGCCACCACTGCCATCGGAGGGGGGGGTGGCCCGGGTGGAGGTGGCGGCGGGGCCACCGATGAGGGAGGTGGCAGAGGCAGCAGCA | | | | | 769 |
| CCTCTCTATGGCAGTGGTGACCTCTCGCCCCTCGGACCTGCAGAGCACGGGCTTTAATGTCTGCGAGGGTCCTGGCGCCAGTCCAACCCCGGCAGGAGGACTCCGT | | | | | 770 |
| AGGAGAGAGGACCTGCCTTCTCTGAGAAAGGAGGAAAGCTGCCTACTACAGAGGGCTACAGTTGGACTCACAGATGGGCTAGGAGATGCCTCCCAACTCCCCGTTGCTCCCACTGGGGAC | | | | | 771 |
| CAGCCA | | | | | |
| CAGCTTTCCTCCTTTCTCAGAGAAGGCAGGTCCTCTCTCCTAGCTCTGGACATGGCAGTTCCGGCCTGGGTATGCTCCCCATTTAGAGGATAAGGCGGCAGTAGTTGTGTTCGCTGTAGA | | | | | 772 |
| TCTGAC | | | | | |
| GGAAAGTGATGATGAGGAGCAAGGACCCACCGTTCCTGCAGACAATGGTCCCATTCTGTCTCTAGTGGGAGATGATACATTAGAGAAAGGAACTGGCCAAGCTCTTGACAGTCATCCCAC | | | | | 773 |
| TATGAA | | | | | |
| GTCCTTGCTCCTCATCATCACTTTCCCAGGAAGTGGTGCCAGACTCACATTCAGTTCTAACATCCGGATGCAACTGAGGCTGCTCCACTAATCTCTCAGCTACTGAGGTTTGGGAGGACA | | | | | 774 |
| GTAGGG | | | | | |
| CGAATTAGGGCTTGGTGGCTCATGCCCTCCTATGAGGGAAAGTGATACTAGACAAGAAAACTTGAAAACCAAGGCTCTCGTTTCTAACAGTTCTTTGCATTGGATACCCATCCCATCGAA | | | | | 775 |
| TGATGA | | | | | |
| CATGAGCCACCAAGCCCTAATTCGTCATCAAATGCTCTGTTCTGCAGGCAATCAGTCGGTGAGGATTCAGGTGTGGAACTGGGGGTCACATTTACAGGATCCTTCATAGTGGGATGACTG | | | | | 776 |
| TCAAGA | | | | | |
| ACAGCTGAGGAGGGTCTAGATCCTCTTGACAGCCTTACTTCACTCTGGACTGTGCCATCTCGAGGAGGCAGTGACAGCAATGGCAGTTACTGTCAACAGGTGGACATTGAAAAGCTGAAA | | | | | 777 |
| ATCAAC | | | | | |
| TGTCAAATCCCCAGGCAATGCAGGAGGGGTGGGAGCAGCTTTCTCCCACTCCTCTCCAACCTGGGGCTCAACAGATGGTATGTGTTCTCTGGATTCTGGTTTGGGCTGTTTCACTACCTC | | | | | 778 |
| ATCATT | | | | | |
| TAGTGTCAGCCTCACTGCTGTCCTCCGTGAGGTGACCTTCAAAGTCAGAGGCTGTATCCGTGGACTCACCGTGAGGACTCAGTGCTTCAGAGTCTCCGTTGATTTTCAGCTTTTCAATGT | | | | | 779 |
| CCACCT | | | | | |
| GCATGTCACCATTCACCTTGGACAGTGGGGCAGATTGGTTCCAATTGGGTTTCTCATCCTTGTCCACCGAAGATCCCTTTGTCACTGCAGCTTCTCTAGTGTCAGCCTCACTGCTGTCCT | | | | | 780 |
| CCGTGA | | | | | |
| TCCGCCAAAAGATCCCAGATTCCCTACTGCTGGCCAGTACTGAGTACCAGCCAAGAGCCGTGTGCCTGTCCATGCCTGGGTCCTCAGTGGAGGCCACTAACCCACTTGTGATGCAGTTGC | | | | | 781 |
| TGCAGG | | | | | |
| TACTGGCCAGCAGTAGGGAATCTGGGATCTTTTGGCGGACCGCACATACTCGAGACACCCAGCTCTGAGGAGCAACCATCCCATCTGTCCTTGTAACCAGACGCATGTCACCATTCACCT | | | | | 782 |
| TGGACA | | | | | |
| CATGTAAAGATCCCATGCGTAGCGAGCCATGGCTCTGGTCTTTTGTAAGTGGTACTTGTGGGGATTCTGACATGCTGTCATCGTGGGCTGGTGGAAGAACCTTCTCTAGGGGCAAGCTAC | | | | | 783 |
| CCTGCA | | | | | |
| TAAGGGCTTTGAAGGAGCCTCTTCTGCCAGATAGCTGTGAAACAGGCACTGGTCTTGCCAGGATTGAGGCCACCCAGGCTCCTGGAGCACCCCAAAAGAATTGCAAGGCAGTCCCAAGTT | | | | | 784 |
| TTGACT | | | | | |
| TGAGGCCACCCAGGCTCCTGGAGCACCCCAAAAGAATTGCAAGGCAGTCCCAAGTTTTGACTCCCTCCATCCAGTGACAAATCCCATTACATCCTCTAGGAAACTGGAAGAAATGGATTC | | | | | 785 |
| CAAAGA | | | | | |
| CAAGACCAGTGCCTGTTTCACAGCTATCTGGCAGAAGAGGCTCCTTCAAAGCCCTTAAAGAACTGGGGCTGCTTCCATCAACCATGCCACTGTTTTTTCCAAGACCATGTAAAGATCCCA | | | | | 786 |
| TGCGTA | | | | | |
| GATTCCAAAGAGCAGTTCTCTTCCTTTAGTTGTGAAGATCAGAAGGAAGTCCGTGCTATGTCACAGGACAGTAATTCAAATGCTGCTCCAGGAAAGAGCCCAGGAGATCTTACTACCTCG | | | | | 787 |
| AGAACA | | | | | |
| AACACCTCGTTTCTCATCTCCAAATGTGATCTCCTTTGGTCCAGAGCAGACAGGTCGGGCCCTGGGTGATCAGAGCAATGTTACAGGCCAAGGGAAGAAGCTTTTTGGCTCTGGGAATGT | | | | | 788 |
| GGCTGC | | | | | |
| TTGGCTCTGGGAATGTGGCTGCAACCCTTCAGCGCCCCAGGCCTGCGGACCCGATGCCTCTTCCTGCTGAGATCCCTCCAGTTTTTCCCAGTGGGAAGTTGGGACCAAGCACAAACTCCA | | | | | 789 |
| TGTCTG | | | | | |
| CCAAGCACAAACTCCATGTCTGGTGGGGTACAGACTCCAAGGGAAGACTGGGCTCCAAAGCCACATGCCTTTGTTGGCAGCGTCAAGAATGAGAAGACTTTTGTGGGGGGTCCTCTTAAG | | | | | 790 |
| GCAAAT | | | | | |
| TCCTCTTAAGGCAAATGCCGAGAACAGGAAAGCTACTGGGCATAGTCCCCTGGAACTGGTGGGTCACTTGGAAGGGATGCCCTTTGTCATGGACTTGCCCTTCTGGAAATTACCCCGAGA | | | | | 791 |
| GCCAGG | | | | | |
| GAGAGCCAGGGAAGGGGCTCAGTGAGCCTCTGGAGCCTTCTTCTCTCCCCTCCCAACTCAGCATCAAGCAGGCATTTTATGGGAAGCTTTCTAAACTCCAACTGAGTTCCACCAGCTTTA | | | | | 792 |
| ATTATT | | | | | |
| TTAATTATTCCTCTAGCTCTCCCACCTTTCCCAAAGGCCTTGCTGGAAGTGTGGTGCAGCTGAGCCACAAAGCAAACTTTGGTGCGAGCCACAGTGCATCACTTTCCTTGCAAATGTTCA | | | | | 793 |
| CTGACA | | | | | |
| GCAAATGTTCACTGACAGCAGCACGGTGGAAAGCATCTCGCTCCAGTGTGCGTGCAGCCTGAAAGCCATGATCATGTGCCAAGGCTGCGGTGCGTTCTGTCACGATGACTGTATTGGACC | | | | | 794 |
| CTCAAA | | | | | |
| GTTATATAAAAAGGTAACAGTTGGCTTACTGGAAGTTGACTTTGTCCACCTGGAACTTGGTCTCAAAGATTCCAGAAGTCAGGACACGGCAGCGAAGCAGGTCCTGAAACAAAATT | | | | | 795 |
| AGCAGCGCGGAGCCGGTGGAGGCGTTGGTGCAGGGCGGCAGGATGCGCGGCGGCGAGCGCTCGCCGCCCACCATGGAGAACTGGTAGGAGCCGGCCGAGGCGCCGTAGTACA | | | | | 796 |
| CCGGCCGAGGCGCCGTAGTACAGGTGGTAGGAGGGCGAGCTGGCTTGGAACGGGCCTCCCTGCGCTTGCGACGAGCCGGGGTAGG | | | | | 797 |
| GCGGCGGCAGGTAGGTGTGGTAGCGCGTGGCCGAGCCCATGGCCGACATGCCGATGCCGATGCCCGA | | | | | 798 |
| TGTGGTAGCGCGTGGCCGAGCCCATGGCCGACATGCCGATGCCGATGCCCGAGGTGACCGGCGTCGGGGAGTAGGTGAAGGCGCCTGGATAGTGCAT | | | | | 799 |
| CGCCTGGATAGTGCATGCGGGGGTCGGAGATGGAGGGCAGCGCGGGGAACTGGCGCGGGTCGCTGAACGCTGTCAGGTCGGGTGCCGCTGCAGGGCGGGCAAGAGAACGGA | | | | | 800 |
| CACCCAGCAACGCCCATTTCACCTGGACGTGCCAGCGGCATGACAACCCTCTCTGCAGAACTTTCCAGTCGACTCTCAAGTAAGCCACTTGAAAACACATTCTTTGCAGCTGAGCTGGGG | | | | | 801 |
| TGGAAG | | | | | |
| TTTACATATAATTGACCTTTCTGATTCTCTTCAGATACAAGGCAGATCCAACCATCCCCACCGTGGTCCTACGATCAGTCCTACCAATACCTGGGATCCATTGCCTCTCCTTCTGTGCAC | | | | | 802 |
| CCAGCA | | | | | |
| CCAGTTGTGGGTGGTGGCCCAGGTGCAGGAGAGGCGGGCAGTGGGCTCCATCTGGTACTTACCCTGCATCTGACTCTGAGGCTGAGGGTTAAAGGCAGTGGAGTGGTTCAGG | | | | | 803 |
| TTCAGGGAGGCACGAGGGTTGGGCGTGGGGGCTGGGTGGTGTGGGCTGACCCTCATGGCTGTGCGCCGCAGCTGCTCCAGTTCACTGAGCCGCTCGGAAAAGGACAAGCTCC | | | | | 804 |
| CCCATCCCCTCCCCTCCCTGCTCCCCACAATAGGACATCGGCAGAAACTAGATGATCAGACCAAGCCCGGGAGCTTGTCCTTTTCCGAGCGGCTCAGTGAACTGGAGCAGCT | | | | | 805 |
| ACAGGGAAAAGCTTCACTCTGACCATCACTGTCTTCACAAACCCACCGCAAGTCGCCACCTACCACAGAGCCATCAAAATCACAGTGGATGGGCCCCGAGAACCTCGAAGTAAGTGCATC | | | | | 806 |
| CACTTG | | | | | |
| TTTCAAGCATAGTTTTGACAGATAACGTACCTCTTCCACTTCGACCGACAAACCTGAGGTCATTAAATCTTGCAACCTGGTTCTTCATGGCTGCGGTAGCATTTCTCAGCTCAGCCGAGT | | | | | 807 |
| AGTTTT | | | | | |
| AAGTGTATGTATAACATCCCTGATGTCTGCATTTGTCCTTTGACTGGTGTTTAGGTGGTGGCCCTAGGGGATGTTCCAGATGGCACTCTGGTCACTGTGATGGCTGGCAATGATGAAAAC | | | | | 808 |
| TACTCG | | | | | |
| CTCTCCGGGCCAGTACCTTGAAAGCGATGGGCAGGGTCTTGTTGCAGCGCCAGTGCGTAGGCAGCACGGA | | | | | 809 |
| TAGGCAGCACGGAGCAGAGGAAGTTGGGGCTGTCGGTGCGCACCAGCTCGCCCGGGTGGTC | | | | | 810 |
| AAAAGGCTGTGATTGACTTGAATAACCGTTGGTTTAATGGACAGCCGATCCACGCCGAGCTGTCACCCGTGACGGACTTCAGAGAAGCCTGCTGCCGTCAGTATGAGATGGGGTGAGTGA | | | | | 811 |
| GGAGTG | | | | | |
| TCGGTTTATTGTGCAACCGAGAGCACCTGTCTCCATGACGACATGCTCCAATTTTGAAATAAAATGAACAGTTGACTCTGTAAGGGAAAATGAGAGCTGATTATTTTGCTGGGAAGATAT | | | | | 812 |
| CAAACA | | | | | |
| ATGACTATGACCCTGACGCAAGCCTGGAGTACAGCGAGGAAGAAACCTACCAACAGTTCCTAGACTTCTATGAGGATGTGTTGCCCGAGTTCAAGAACGTGGGGAAAGTGATTCAGTTCA | | | | | 813 |
| AGGTGG | | | | | |
| CATCCCTCCTGCACTGCTCCATTCCAAACGTCGTAAACATGCTCTTAATAAGAAGGGTAGGACTGGATGTTGGGAAATTATGTTTACGTGAACATCTGCGTATGAAAGAAAAGCTTAAAT | | | | | 814 |
| TCACTA | | | | | |
| TGTGACAAATCAGGAAGACATCCACAAGCAGAATACTTACGACTGGTACTGAACATATACATTGCCCCTCAGGTGAGGTTCCAAATTGCAGCTGACCTAGGGAGCAATGAAGAATGCTAT | | | | | 815 |
| CATAAA | | | | | |
| AAAGGGAAGAAGAATGCCAAGCAGCCCTTTCTCTGTTTAACGGACGATGGTATGCAGGACGACAGCTGCAGTGTGAATTCTGCCCCGTGACCCGGTGGAAAATGGCGATTTGTGGTAAAA | | | | | 816 |
| GACAAAGTGATGATTTT | | | | | |
| GCTCACCAGTAGTTGTCTGAGGCCAGATCACTGGGGTGGAGCCACTCTACAGAGGAGCCCAGCAGAGTCTGAATTTCGTTCGTGAATTCCACCAGATCTAACAGCTCCTTACTTTCAGGG | | | | | 817 |
| TTGAAG | | | | | |
| AATTTCCACGTTTGGAAAATTGCAGCGAAATATGCGGGAGGACATTTTCAATTTCTTAAGGACATCCGAAAGCAGTAGCCAGTTTCGTGGCCTACAAAACAGAAAGGAAAATGCTTTGAG | | | | | 818 |
| TTTCCA | | | | | |
| GCATATTTCGCTGCAATTTTCCAAACGTGGAAATTGTCACCATTGCAGAGGCAGAATTTTATCGGCAGGTTTCTGCAAGTCTCTTGTTCTCTTGCTCCAAAGACCTGGAAGCCTTCAACC | | | | | 819 |
| CTGAAA | | | | | |
| TCATACCTGTTAAGAACTTTTCCATAAGTTCACTGTGGGTCATTTTCATGATGGTTCTACCTGAGTACGTAGCCAAGGTGGGGTCAGCACCATAAGAGAGAAGTAGTCGGACAATTTCCA | | | | | 820 |
| AGTGAT | | | | | |
| AAAGGAGACAACTGCTTTATAAGCAAGTGTGGCTTGCATGAGCATAATTCTATTGCGCCTCTCTTCCTAGGCCTCTGCACGATGCTGTTGAGAACGATCACTTGGAAATTGTCCGACTAC | | | | | 821 |
| TTCTCT | | | | | |
| CATTGGCATTGGGGGCGGGTGATGCGGAGGCTGGGCGGCCTGCACTCGACACTGACCCTGAAACGTTAGTGATGACAGCATCGGTGCCGCCCATGCGCGGGCATGATGAACTCCGCTGCT | | | | | 822 |
| GTGGTA | | | | | |
| TTCAGATCTATAGATAGCACAACCATTTCCTGGAGGAGATAGTGTTTCTTTCGGAATCTCACTTCCGGAGAGCACTAAGCCACTTCCAGCCCTGCTGTGAACCAGGACCGTGGGAGCCAT | | | | | 823 |
| CTTTTT | | | | | |
| ATCTATAGATCTGAAATCATCAGCACTGCTCCCTCATCCTGGGTGGTGCCCGGGCCAAGTCCTAACGAAGAGAACAATGGCAAAAGCATGTCGCTGAAAAACAAGGCATTGGACTGGGCG | | | | | 824 |
| ATACCA | | | | | |
| CCCCTTCTGTCCTCGCAGGTTAATCCCCAGAGGCTCCATGGAGTTCCCTGGCCTGGGGTCCCTGGGGACCTCAGAGCCCCTCCCCCAGTTTGTGGATCCTGCTCTGGTGTCCTCCACACC | | | | | 825 |
| AGAAT | | | | | |
| AGGCCTACAGACACTCCCCAGGTAACTCCATTGAGTGGCTGTCTTGGCATTGGCTGAGTGCTGTTGGGGTTGCCATGGAGATCCTTGGCTAGGTCAGAATACCACTGTGAGGATATCTCA | | | | | 826 |
| GAAATG | | | | | |
| CTGTAGGCCTCAGCGTCCCTGTAGTAGGCCAGTGCCGCAGCTGCAGCGGTGGCTGTGCTCGGGGCAGTGGAGGAAGCTGCTGCATCCAAGCCCTCAGGCCCAGAGGGGAAGAAAACCCCT | | | | | 827 |
| GATTCT | | | | | |
| AACAGGGCTGAAGGCCAGGCCCCACCTGCAGATTCAGGGCTAGATGTCGTGTCTTGGTCTGTTCTAGGTCACTCTGGGAGTACTTGAGCCGCATCTGCAGTCCATGGGCCTGAGACTGCA | | | | | 828 |
| CCTGAC | | | | | |
| ACTGCACCTGACGCAGCTCTGCCTCTTTCCGTTTTGCCTTCATCTGCTCCTGAAGGGAACAAGAAAGAAGGGCTAGGTGGTAAGGTGGTGGCTGACCTAGGCTTAGGACTCCCACTGCTA | | | | | 829 |
| AAAACA | | | | | |
| ACCTAGGCTTAGGACTCCCACTGCTAAAAACAGCACTGCCTGTGGCTTACTTTCAGCTCCTCTGTCAAGCGCTCCTTCTTCTCTTTCAACTTGTCTACTGCTTTCTCATCCCAGCGCCGT | | | | | 830 |
| GCCTTG | | | | | |
| GGCCTTCAGGTCACTGGCCCCACCAGAGATCACTCCTGACTTCTGGAATAGGGTTCCATCCAGTGCCACTGTCTACACACAGCAGGGGGAAGAGAGAAGAGGGGGAGAAGCTGAACAAAT | | | | | 831 |
| GAATCT | | | | | |
| GAGAAGCTGAACAAATGAATCTCCAGTACTGAGCCTGTCCAGCTCCAGCCTGGGCAAGGGAATCCACACCTTGTGGCGCTGGTGGCCTCCAAAGGCAATGCGGCGGGCATCTTCCACGTT | | | | | 832 |
| GTCACA | | | | | |
| CCAGCTCCCGGAGTTTCTCATCTGTAGGCTTCACCTGTGGGGAGAAGCTCAGTCAGTGGCAGAACACAAACAGGGAGTACTAGAGGAGGGGCCCTTGAACACTGGCCTGACCCAATCCCCA | | | | | 833 |
| ACAAG | | | | | |
| AACTCCGGGAGCTGAAGGGGGCCAAGCTAGTGATTGATGTGATTCGCTATGAGCCACCTCATATCAAAAAGGCCCTGCAGTATGCTTGTGGCAATGCCCTTGTCTGTGACAACGTGGAAG | | | | | 834 |
| ATGCCC | | | | | |
| TAATGGCATCCATGTTCTTGCCCAAAACCTTGGTTACAGCAATCTGATACTTCTTTTGTGTGGGCTGGCATAGGTCAATGAGGCGGCCGTACTGAGTAAAGTAGGAAGGGAAAACTGAAG | | | | | 835 |
| TATGAA | | | | | |
| TGGATGCCATTATTGTGGACTCGGAGAAGACAGGCCGGGACTGTATTCAGTATATCAAGGAGCAGCGTGGGGAGCCTGAGACCTTCTTGCCTCTTGACTACCTGGAGGTGAGGCTTGTTG | | | | | 836 |
| GGGATT | | | | | |
| AATATGGATAATACACAAAGACAATATGAAGCAGAACGGAATAAAATGATTGGAAAACGAGCCAATGAGAGGCTAGAACTCCTGCTACAAAAGCGGAAAGAGGTAAACTTTTATATTGAA | | | | | 837 |
| TATTTA | | | | | |
| CCAATCATTTTATTCCGTTCTGCTTCATATTGTCTTTGTGTATTATCCATATTAATGCTAAGATTTAGTGCCACATTCACCAAAGCTGTCATCAACTTCATAGCTATTTTTAAAGAGTAA | | | | | 838 |
| AAGTAT | | | | | |
| AGCTTCAGGAAAATCAAGATGAAATAGAAAATATGATGAATGCAATATTTAAAGGAGTGTTTGTACATAGATACCGGTAAGTTGTGACAGTTTTTTTCATAAATAGCATTATGTAATTTC | | | | | 839 |
| TACTCA | | | | | |
| CCCATTCAGTGATGCGATAGCTGAAATTCGAGCTATTTGCATTGAAGAGATTGGCATTTGGATGAAGATGTATAGTGATGCCTTTCTTAATGACAGTTATTTAAAATATGTTGGTTGGAC | | | | | 840 |
| TATGCATGATAAGGT | | | | | |
| TTTTAATGCATTGTCTCATCTTTTTTTTTTTTTTTTTTTAGCAAGGTGAAGTAAGACTCAAATGTCTTACTGCTCTACAAGGGCTTTATTATAACAAAGAGCTTAATTCCAAACTGGAAC | | | | | 841 |
| TTTTTA | | | | | |
| ACTTTTTACCAGTCGGTTCAAGGTTAGTATTACTTAAGAATTTACAAATAACTTGTCATTTGCAAACACTTTTCTCTTGCTTTCCTTTTAAAAATATTTTAATTTTTTTGTCCTTAGGAT | | | | | 842 |
| AGAATT | | | | | |
| TAAAAATATTTTAATTTTTTTGTCCTTAGGATAGAATTGTGTCTATGACCCTTGACAAAGAATATGATGTTGCAGTACAAGCAATAAAATTACTCACTCTTGTTTTACAGTAAGTATGTA | | | | | 843 |
| TTTGTT | | | | | |
| ATATATAGATTTACTTTTTGTAGAGAAATTCTCCAGCTGCTACTGCTACTGGCCGGTGAGCTGAATAAACCAGATGATAGACATTTTCACAATCTTCTGCAGTGAGAACTTCTTCACTAC | | | | | 844 |
| TCCTAA | | | | | |
| GATTTCTTTTCAAGGTATGTTTACTTGGAAAAGTTCATGACCTTTCAGATGTCACTCCGAAGAGAGGATGTGTGGCTTCCACTGATGTCTTACCGAAATTCTTTGCTAGCTGGTGGTGAT | | | | | 845 |
| GATGAC | | | | | |
| TTACTGAGTGAAAGCTGCATGCCCTCAACCACTTTCCGTTTTCCTGTAGATGGTTTTGATTTTTTACTCCGTACTGTTGACCCCCGGCTGCTGATTCCACTAATGACTGACATGGTGTCA | | | | | 846 |
| TCATCA | | | | | |
| AAAAAAGGGCCTACAAGACAGCGCAAATGTGGCTTTTGTAAGTCAAATAGAGACAAGGAATGTGGACAGTTACTAATATCTGAAAACCAGAAGGTGGCAGCGCACCATAAGTGCATGGTA | | | | | 847 |
| AGTATA | | | | | |
| CAGATATTAGTAACTGTCCACATTCCTTGTCTCTATTTGACTTACAAAAGCCACATTTGCGCTGTCTTGTAGGCCCTTTTTTCTGTTCAACTGAGCTTGACATGATTTTTAAATTGCCTT | | | | | 848 |
| TAGAAT | | | | | |
| ATAAAGCTCAAATACGAGAGAAACCTTCACAAGGAATTTACATGTAATTATTTAACTTCTCTTTAAGTTTTTTTTTTAACAATAATACTTTCTTTGGGCTTTTGTAAAAATTTTTCCCAT | | | | | 849 |
| TTCAAA | | | | | |
| TTTATCATGCAATGCACAGTGGTAGTGGTATGTCCTGTGACATGTTTTCACATCACAACCAATTGTTGCTCCAGGACAATGGCACAAAGAACACATCTACAGCAAAACGACAAAATAATA | | | | | 850 |
| TTATTA | | | | | |
| ATTTCTTCATTTTTTATAGGGTCTATTGCCGAAAACACAAGAAAACTGCACATAACTCCGAAGGTACATCATTTAG | | | | | 851 |
| AATACCGATAGCATATTTTCATGATTTTTTTTAAGTTCGTTTTTTCTTTTACATTTGCAGAGAGATAGGTCTCCACACAGAAGCAGCCCTAGTGACACCAGGCCTAAATGTGGATTTTGC | | | | | 852 |
| CATGTA | | | | | |
| TTGGGCTTAAAAGAACCATGCTTACCATGCACTTATAATGGGCAGCTGCCTTCTTGGCATTAAATATATGCAGTTTTCCTCGTGCTTCATTTTCTTCCTCCCCTACATGGCAAAATCCAC | | | | | 853 |
| ATTTAG | | | | | |
| GACCATTCTTTTTCCTCGTTTAATCTCCTGAAGTACAGTTTTAATATCAAAGTCTCCAAATTCTGCTCTTGATGTTGTTGTGAGCTGGACTGTGCCAGAAGAAAACAACTAGAGAAGAAA | | | | | 854 |
| A | | | | | |
| TGTTTTCTTACTTGTAAATTCCTCGTGACATATTTTCAATGTATTTAGCTTTGTCTTGTACTCCACAGTGGTAATGGTAAGTCTTAACACAGGCTTTTATTTCACATCCAATAGTAGCAC | | | | | 855 |
| CAGGCT | | | | | |
| | | feature_ | feature_ | | |
| | SEQ | start_ | stop_ | probe_ | failure_ |
| mip_sequence | ID | position | position | strand | flags |
| TCAGACTTCGGCCCACCCNNNNCTTCAGCTTCCCGATATCCGACGGTAGTGTNNNNTGGTCCACCGCCAGTCTCCTGCCTGG | 856 | 43814931 | 43815035 | - | 0 |
| GTGTGGAGGGTAAGGGGGCAGGGNNNNCTTCAGCTTCCCGATATCCGACGGTAGTGTNNNNGATGGCAAATACACAGAGGAA | 857 | 115256486 | 115256577 | + | 0 |
| GTAGCCCGCTGACCTGATCCTGTNNNNCTTCAGCTTCCCGATATCCGACGGTAGTGTNNNNCAACAGGTTCTTGCTGGTGTG | 858 | 115258743 | 115258748 | − | 0 |
| ACTGGGAAACCAAATACCCTGGNNNNCTTCAGCTTCCCGATATCCGACGGTAGTGTNNNNTACCTCAGTTTGCCCCCATGTC | 859 | 25457148 | 25457289 | + | 0 |
| GCTGAAGGAGTATTTTGCGTGTGTNNNNCTTCAGCTTCCCGATATCCGACGGTAGTGTNNNNTCACCCTGCCCTCTCTGCCT | 860 | 25457144 | 25457294 | − | 0 |
| CCCGGGTTGTGCTGGCATCTGGCTNNNNCTTCAGCTTCCCGATATCCGACGGTAGTGTNNNNTCCTCTTTTCTCCTCTTCAT | 861 | 25458573 | 25458698 | − | 0 |
| GCAGGGAGAAGGAAGGGCAGGATNNNNCTTCAGCTTCCCGATATCCGACGGTAGTGTNNNNGTTATCCAGGTTTCTGTTGTT | 862 | 25459792 | 25459882 | − | 0 |
| GCTCCTGGGCCTGGGGGGCTGTNNNNCTTCAGCTTCCCGATATCCGACGGTAGTGTNNNNGCTGTTTCATGCTCCTCCTTGG | 863 | 25461992 | 25462092 | − | 0 |
| GCGCATCATGCAGGAGGCGGTAGANNNNCTTCAGCTTCCCGATATCCGACGGTAGTGTNNNNAGGTAGAAGCCATTAGTGAG | 864 | 25463166 | 25463483 | + | 0 |
| GCGTTAGTGACAAGAGGGACATCTNNNNCTTCAGCTTCCCGATATCCGACGGTAGTGTNNNNGCCCCAGCTGATGGCTTTCT | 865 | 25463166 | 25463483 | − | 0 |
| GCTGTCCAGGGACAGAGGCAGACANNNNCTTCAGCTTCCCGATATCCGACGGTAGTGTNNNNGTGGAGGGGACAGGATGGTA | 866 | 25463503 | 25463605 | + | 0 |
| TTGGAGCCATCTCCCTGGCACCCTNNNNCTTCAGCTTCCCGATATCCGACGGTAGTGTNNNNTCCTCACACACCTCCGAGGC | 867 | 25464533 | 25464580 | + | 0 |
| GTCCATGCTGTGGGGCGCAGNNNNCTTCAGCTTCCCGATATCCGACGGTAGTGTNNNNGCTGAAGGACTTGGGCATTCAGGT | 868 | 25464429 | 25464508 | − | 0 |
| GTGAGGGGTGCAGGCCCAAGAGGTNNNNCTTCAGCTTCCCGATATCCGACGGTAGTGTNNNNGCTGGGTGGGAGCTTGGGAC | 869 | 25466760 | 25466845 | − | 0 |
| CTTCCTTAATGGCTGCCTGGGCAGNNNNCTTCAGCTTCCCGATATCCGACGGTAGTGTNNNNCCCAGCACTCACAAATTCCT | 870 | 25467088 | 25467139 | + | 0 |
| GCCTGGGGCGCGGTCTCGAGCTNNNNCTTCAGCTTCCCGATATCCGACGGTAGTGTNNNNAAGGGTACCTACGGGCTGCTGC | 871 | 25467018 | 25467212 | − | 0 |
| CCCCTGGAACTGCTACATGTGCNNNNCTTCAGCTTCCCGATATCCGACGGTAGTGTNNNNGTCTCCTCTGCTCACTGGGTCT | 872 | 25467018 | 25467212 | − | 0 |
| GCTGTTGTGGCCTCCAGTGGTNNNNCTTCAGCTTCCCGATATCCGACGGTAGTGTNNNNGTGGTTTCTGACCCTTCCCGCTG | 873 | 25467407 | 25467525 | − | 0 |
| GGTGAGTACCACCGAAGGGCCTCTNNNNCTTCAGCTTCCCGATATCCGACGGTAGTGTNNNNTCCTGGGTGGGTGTGCTCCT | 874 | 25468119 | 25468204 | + | 0 |
| TGCCGTTGAGGCCGGCCCTTCNNNNCTTCAGCTTCCCGATATCCGACGGTAGTGTNNNNCATCCCCTCCCTCTGCTTTCCAG | 875 | 25468119 | 25468204 | − | 0 |
| CCGCGCCTGCTCCTCGGATGNNNNCTTCAGCTTCCCGATATCCGACGGTAGTGTNNNNCTTCCTAAGTGCCTCTGCTACTCT | 876 | 25468879 | 25468940 | + | 0 |
| CACATGTCCGTGTACACTTCTTTGNNNNCTTCAGCTTCCCGATATCCGACGGTAGTGTNNNNTCCTGGTGCCACCCTCTCCA | 877 | 25469025 | 25469183 | + | 0 |
| TTCCCCCACCCTCCTTACAGNNNNCTTCAGCTTCCCGATATCCGACGGTAGTGTNNNNGGGCTTTTTGGCTGGTGGAGGTGG | 878 | 25469025 | 25469183 | + | 0 |
| GTGACACGCCAGGGTTGGGGTTGTNNNNCTTCAGCTTCCCGATATCCGACGGTAGTGTNNNNTAGGGCCAGAAGGCTGGAAG | 879 | 25469487 | 25469650 | + | 0 |
| GGCTGCCAAGGCCTCCACAGAGNNNNCTTCAGCTTCCCGATATCCGACGGTAGTGTNNNNTGATTGAATGGGCCCTGGGGGG | 880 | 25469487 | 25469650 | − | 0 |
| CACCCACCCCATGCCTTGCAANNNNCTTCAGCTTCCCGATATCCGACGGTAGTGTNNNNGACCTCGTAGATGGCTTTGCGGT | 881 | 25469917 | 25470030 | + | 0 |
| GCACTAGGAGGCCTGGAAGTTGNNNNCTTCAGCTTCCCGATATCCGACGGTAGTGTNNNNGGTGTGTGTTGAGAAGCTGATG | 882 | 25469917 | 25470030 | − | 0 |
| TTCCCCCACACCAGCTCCCCAANNNNCTTCAGCTTCCCGATATCCGACGGTAGTGTNNNNCTACTGCCAAACCCCACAACTT | 883 | 25470458 | 25470621 | + | 0 |
| GTGTCTTGGTGGATGACGGGCNNNNCTTCAGCTTCCCGATATCCGACGGTAGTGTNNNNATGGGGGGATCAGGGTGGCAGGG | 884 | 25470458 | 25470621 | − | 0 |
| CGGGCCCCTGGTTTTCTTNNNNCTTCAGCTTCCCGATATCCGACGGTAGTGTNNNNTCTGGCTCGTCATCGCCTGCTTTGGT | 885 | 25470974 | 25471055 | + | 0 |
| AGGTAATTGGTGGATTTACCTTTCNNNNCTTCAGCTTCCCGATATCCGACGGTAGTGTNNNNGTAGTTGGCATATTCTGCAT | 886 | 198266442 | 198266624 | + | 0 |
| CTGGATATGTTTCATGGTTCTNNNNCTTCAGCTTCCCGATATCCGACGGTAGTGTNNNNTGGGTATCTTATTCCTCTTATGG | 887 | 198266442 | 198266624 | − | 0 |
| GTGTTAAAGCCTTTATGGAAGGGTNNNNCTTCAGCTTCCCGATATCCGACGGTAGTGTNNNNTAGGTAATGTTGGGGCATAG | 888 | 198266810 | 198266849 | − | 0 |
| GAAAGGACAGTCATGAGTTGGTNNNNCTTCAGCTTCCCGATATCCGACGGTAGTGTNNNNGGGCAATAAAGAAGGAATGCCC | 889 | 198267481 | 198267491 | + | 0 |
| CTTATGGGCTGTGCCATCTTGCCNNNNCTTCAGCTTCCCGATATCCGACGGTAGTGTNNNNCGTAACACAACAGCTAGAGCT | 890 | 198267342 | 198267373 | − | 0 |
| GGTGCCATTTGGTGATTTCCNNNNCTTCAGCTTCCCGATATCCGACGGTAGTGTNNNNCAAAATCACATTATTGCCAACATG | 891 | 209113112 | 209113210 | + | 0 |
| TGCAGGAGAAGTCATCCCCCTTCNNNNCTTCAGCTTCCCGATATCCGACGGTAGTGTNNNNTCTGAAAACTGGTGGTTGCCT | 892 | 128200060 | 128200163 | − | 0 |
| GGTGCCGGCTCTTCTGGCGGNNNNCTTCAGCTTCCCGATATCCGACGGTAGTGTNNNNCATTGTGCAGCTTGTAGTAGAGGC | 893 | 128200661 | 128200789 | + | 0 |
| CACTCATCAAGCCCAAGCGAAGACNNNNCTTCAGCTTCCCGATATCCGACGGTAGTGTNNNNTGTCTCTCCCTGTTCCCCTG | 894 | 128202700 | 128202851 | − | 0 |
| GTCCAGGAACTGAGCAGAGGTNNNNCTTCAGCTTCCCGATATCCGACGGTAGTGTNNNNGTTGCTGAGGTTTTCCAGCACTC | 895 | 55589745 | 55589869 | + | 0 |
| GTGAATACACTATTAGGTTGGAGGNNNNCTTCAGCTTCCCGATATCCGACGGTAGTGTNNNNGCAGGACTGTCAAGCAGAGA | 896 | 55599285 | 55599349 | − | 0 |
| GCCCACTGCCTGAGAGAGCTCATNNNNCTTCAGCTTCCCGATATCCGACGGTAGTGTNNNNACCAACCATGTTGAGGGCAAC | 897 | 106155099 | 106155185 | + | 0 |
| GTAGAGGGTATTCCAAGTGTTTGCNNNNCTTCAGCTTCCCGATATCCGACGGTAGTGTNNNNAAATGGAGACACCAAGTGGC | 898 | 106155144 | 106155319 | + | 0 |
| GTTCTGTCTGGCAAATGGGAGGTGNNNNCTTCAGCTTCCCGATATCCGACGGTAGTGTNNNNTGGCTTCCCTTCATACAGGG | 899 | 106155144 | 106155319 | − | 0 |
| TCTGTAGCCCAAGAAAATGCAGNNNNCTTCAGCTTCCCGATATCCGACGGTAGTGTNNNNCTCTGGGCTCCTTCAGATCAAG | 900 | 106155044 | 106158550 | + | 0 |
| CGACTATTCTGGCTTCCCTTCNNNNCTTCAGCTTCCCGATATCCGACGGTAGTGTNNNNGTCTTTCTCCATTAGCCTTTTGG | 901 | 106155044 | 106158550 | − | 0 |
| TGGAACACACACATGGTGAACTCCTGNNNNCTTCAGCTTCCCGATATCCGACGGTAGTGTNNNNCCAGAGCTTCAGATTCTG | 902 | 106155646 | 106155733 | + | 0 |
| CATTTGGTTGACTGCTTTCACCTGNNNNCTTCAGCTTCCCGATATCCGACGGTAGTGTNNNNACTTTTCCCCTCCTGCTCAT | 903 | 106155044 | 106158550 | − | 0 |
| GTGAGTGAGGCCTGTGATGCTGATNNNNCTTCAGCTTCCCGATATCCGACGGTAGTGTNNNNAGAAAACCACATCTCACATA | 904 | 106155044 | 106158550 | + | 0 |
| GCACCATTAGGCATTAGCACTGCCNNNNCTTCAGCTTCCCGATATCCGACGGTAGTGTNNNNACTCATTAGTAGCCTGACTG | 905 | 106155044 | 106158550 | − | 0 |
| CCAGCAGCAATTTGCAAGCNNNNCTTCAGCTTCCCGATATCCGACGGTAGTGTNNNNTACCTGTTCCTTTCAGAAACCAGAA | 906 | 106156091 | 106156165 | + | 0 |
| GCAGCTGGCTTTGGAGGCAGCTNNNNCTTCAGCTTCCCGATATCCGACGGTAGTGTNNNNGTTATTTTCTGCAGGAGATGGG | 907 | 106155044 | 106158550 | − | 0 |
| CACCACCACTACCCCAACCAAANNNNCTTCAGCTTCCCGATATCCGACGGTAGTGTNNNNCTCAGTGTTCACTAAGGATTCC | 908 | 106155044 | 106158550 | + | 0 |
| TGAACAGAATTCTTCACCANNNNCTTCAGCTTCCCGATATCCGACGGTAGTGTNNNNGCAATTGTGATGGTGGTGGTGGTGT | 909 | 106156165 | 106156274 | − | 0 |
| CCAATGTCAGAACACCTCAAGCNNNNCTTCAGCTTCCCGATATCCGACGGTAGTGTNNNNACCACCTTCCCAGAGTCCTAAT | 910 | 106155044 | 106158550 | + | 0 |
| GCTTTTTCCTTCTGAAGGAAGCTGNNNNCTTCAGCTTCCCGATATCCGACGGTAGTGTNNNNGAAGGGCTGCATACATGTGT | 911 | 106155044 | 106158550 | − | 0 |
| CGTAATGAGGCATCACTGCCATCANNNNCTTCAGCTTCCCGATATCCGACGGTAGTGTNNNNTGATGAGAAACAAAGAGCAA | 912 | 106155044 | 106158550 | + | 0 |
| CCACAATGGAACAGTCATTGTCCCNNNNCTTCAGCTTCCCGATATCCGACGGTAGTGTNNNNCGTGTTTGCTCCTTGTCTCG | 913 | 106155044 | 106158550 | − | 0 |
| CCCTCACACCAGGTGCACTTCNNNNCTTCAGCTTCCCGATATCCGACGGTAGTGTNNNNCTGGAAATTCCAACATGCCTGGG | 914 | 106155044 | 106158550 | + | 0 |
| GGGATTCCGCTTGGTGAAAACGANNNNCTTCAGCTTCCCGATATCCGACGGTAGTGTNNNNGCTGTGTTGTTTTCTGGGTGT | 915 | 106155044 | 106158550 | − | 0 |
| TCCCAGAGTTCACATCTCCCTCANNNNCTTCAGCTTCCCGATATCCGACGGTAGTGTNNNNCAACAAAGAGCAGATTCCCAA | 916 | 106155044 | 106158550 | + | 0 |
| GGAACTGGAGATGTTGGTCCACTGNNNNCTTCAGCTTCCCGATATCCGACGGTAGTGTNNNNGTGCTGTTTCAACACTGGGG | 917 | 106155044 | 106158550 | − | 0 |
| GAAATTCCCCTTATAGTCAGACCATGNNNNCTTCAGCTTCCCGATATCCGACGGTAGTGTNNNNATGATCAGCAAAGAGAAG | 918 | 106157293 | 106157510 | + | 0 |
| GGTTGTGTTTGTGCTGCCTGTTTATGNNNNCTTCAGCTTCCCGATATCCGACGGTAGTGTNNNNTTAGTCTGGCCAAAGAAT | 919 | 106157294 | 106157418 | − | 0 |
| GCTTTCAAGAACAGGAGCAGAAGNNNNCTTCAGCTTCCCGATATCCGACGGTAGTGTNNNNCTTGTTCAAACAATACACACC | 920 | 106155044 | 106158550 | + | 0 |
| GACATTATGAGTCTCGAACTCGCTNNNNCTTCAGCTTCCCGATATCCGACGGTAGTGTNNNNGGATGTGTAGTCTGTTCTTT | 921 | 106155044 | 106158550 | − | 0 |
| CAGCAAACACAGCAACCCCAAACNNNNCTTCAGCTTCCCGATATCCGACGGTAGTGTNNNNCTTGCTCAGCAAAGGTACTTG | 922 | 106155044 | 106158550 | + | 0 |
| GTGAAGAAGATCTTGCTTTGGGNNNNCTTCAGCTTCCCGATATCCGACGGTAGTGTNNNNAGGCACAGGAAAAACATTTGCA | 923 | 106157742 | 106157861 | − | 0 |
| AATGTGCAGCAAAAGAGCATCATTGANNNNCTTCAGCTTCCCGATATCCGACGGTAGTGTNNNNACTGAGTCTTGCCATAGT | 924 | 106158077 | 106158150 | + | 0 |
| GCAGCATGCTTTTGAGTGTCCNNNNCTTCAGCTTCCCGATATCCGACGGTAGTGTNNNNTGTTTTCTGGTGGTGCTGTGTGC | 925 | 106155044 | 106158550 | − | 0 |
| CTTCTTCAGAAAAGACACCAACCNNNNCTTCAGCTTCCCGATATCCGACGGTAGTGTNNNNGACCATAAGGCTCTTACTCTC | 926 | 106158273 | 106158480 | + | 0 |
| CACAGCTTGCAGGTGGATTCTCNNNNCTTCAGCTTCCCGATATCCGACGGTAGTGTNNNNGTCAAAACTGTGACTGGCCCTG | 927 | 106155044 | 106158550 | − | 0 |
| CTTCAGATATGGGATTTTCCTTCTNNNNCTTCAGCTTCCCGATATCCGACGGTAGTGTNNNNGAGTCACCTTCCAAATTACT | 928 | 106158273 | 106158480 | + | 0 |
| GCTGGGGTGTGGCTATCAAGTTCTNNNNCTTCAGCTTCCCGATATCCGACGGTAGTGTNNNNGAGTCTTGACAGGTGTATCC | 929 | 106158273 | 106158480 | − | 0 |
| GCAAAGGCACAGGGCAGATTAACGNNNNCTTCAGCTTCCCGATATCCGACGGTAGTGTNNNNTAGTATAATTGAGGTCTAAA | 930 | 106162468 | 106162605 | + | 0 |
| GCCTTTGGTCTTAAATCTTGGGNNNNCTTCAGCTTCCCGATATCCGACGGTAGTGTNNNNTTGTATGTGTGTGTGTTTCTGT | 931 | 106163988 | 106164088 | + | 0 |
| CCAATCGCCGGTGTGCCTTGAATNNNNCTTCAGCTTCCCGATATCCGACGGTAGTGTNNNNTGTGTTTGGTGCGGGAGCGAG | 932 | 106164823 | 106164875 | + | 0 |
| CCCCCCACCCCAACCAAAACAAAANNNNCTTCAGCTTCCCGATATCCGACGGTAGTGTNNNNCGGGATTCCTTCCCACACCA | 933 | 106164725 | 106164940 | − | 0 |
| GCTCCGAGTAGAGTTTGTCAGCCNNNNCTTCAGCTTCCCGATATCCGACGGTAGTGTNNNNCACGCTGAACTCTCTTCCTTT | 934 | 106164725 | 106164940 | − | 0 |
| TTTGCCAGAAGCAAGATCCCNNNNCTTCAGCTTCCCGATATCCGACGGTAGTGTNNNNTGCACAGCCTATATAATGCTATCC | 935 | 106180773 | 106180933 | + | 0 |
| TTCTGGATCCAGCCCCTGACAGGCNNNNCTTCAGCTTCCCGATATCCGACGGTAGTGTNNNNAGCGATTATACATCAGGAAG | 936 | 106180773 | 106180933 | − | 0 |
| GGCAGCAATTGTAACAACTTACTTGNNNNCTTCAGCTTCCCGATATCCGACGGTAGTGTNNNNAGAATTATTCACTTTATAC | 937 | 106182914 | 106183008 | + | 0 |
| GAGGACAGCTTAGCAGCTGTTGAGNNNNCTTCAGCTTCCCGATATCCGACGGTAGTGTNNNNAATATGAACACAGAGCACCA | 938 | 106190761 | 106190911 | + | 0 |
| GAAAACTCACTAGTATTTAGACCNNNNCTTCAGCTTCCCGATATCCGACGGTAGTGTNNNNAGCACAGAAGTCCAAACATGC | 939 | 106190761 | 106190911 | − | 0 |
| GTCAAGACTTGCCGACAAAGGANNNNCTTCAGCTTCCCGATATCCGACGGTAGTGTNNNNGGATGAGCAGCTTCACGTTCTG | 940 | 106193714 | 106194080 | + | 0 |
| GTAAGACATTACAGCCTCAACTACNNNNCTTCAGCTTCCCGATATCCGACGGTAGTGTNNNNTTTTTTCTCCTCCTGAGCTT | 941 | 106193714 | 106194080 | − | 0 |
| GCATTTGTAGATAAATGTGTTGTGNNNNCTTCAGCTTCCCGATATCCGACGGTAGTGTNNNNGAAGCCAAGAAAGCTGCAGC | 942 | 106193714 | 106194080 | + | 0 |
| GCCGAAAAGAACTCAGTACCTGNNNNCTTCAGCTTCCCGATATCCGACGGTAGTGTNNNNTGTTTTGTACGTGATGGGGCTG | 943 | 106193918 | 106193967 | − | 0 |
| CCCCAGCAGCAGCAGCCACANNNNCTTCAGCTTCCCGATATCCGACGGTAGTGTNNNNTTTCTGTTCTCTCTTACCCTGTCC | 944 | 106196203 | 106196292 | + | 0 |
| CCATGAACCCTTACCCTGGGCTTNNNNCTTCAGCTTCCCGATATCCGACGGTAGTGTNNNNCCACCAATCCATACATGAGAC | 945 | 106196203 | 106197683 | + | 0 |
| GCTGGGGCTGTGGTGGCTGCTTCTNNNNCTTCAGCTTCCCGATATCCGACGGTAGTGTNNNNGATATCTGAAGTGTGTGAAG | 946 | 106196203 | 106197683 | − | 0 |
| GCCAAGGTTTGGAAATAGCCAGAGNNNNCTTCAGCTTCCCGATATCCGACGGTAGTGTNNNNCAATGCAATGGAAACCTATC | 947 | 106196203 | 106197683 | + | 0 |
| CCTGCAGCTTGAGATGAGGTGGANNNNCTTCAGCTTCCCGATATCCGACGGTAGTGTNNNNGGCTGAGACTGGGGAGAATAG | 948 | 106196203 | 106197683 | − | 0 |
| GGTGAACATCATTCACCTTCTCACNNNNCTTCAGCTTCCCGATATCCGACGGTAGTGTNNNNGTTTCAGCAGTTGTACCATT | 949 | 106196203 | 106197683 | + | 0 |
| GTGTATGGATGGGTGGTAGACTGNNNNCTTCAGCTTCCCGATATCCGACGGTAGTGTNNNNGAGTGGGATAAGGAGGCAATT | 950 | 106196203 | 106197683 | − | 10 |
| GCTTTCCCACACAGCTAATGGGTNNNNCTTCAGCTTCCCGATATCCGACGGTAGTGTNNNNACCACCCAATCTGAGCAATCC | 951 | 106196203 | 106197683 | + | 0 |
| GTGATGCTAATGGTCAGGAAAAGCNNNNCTTCAGCTTCCCGATATCCGACGGTAGTGTNNNNCATGTTCAACAGCTCTCTTC | 952 | 106196203 | 106197683 | + | 0 |
| TCACCCCACCAGGATCTCCCNNNNCTTCAGCTTCCCGATATCCGACGGTAGTGTNNNNCAACGATGAGGTCTGGTCAGACAG | 953 | 106196203 | 106197683 | + | 0 |
| GCAGTTCTATCATGGTTAAGAGCTGGNNNNCTTCAGCTTCCCGATATCCGACGGTAGTGTNNNNTCAGGATCCAGAAAGCTC | 954 | 106197125 | 106197218 | − | 0 |
| CAGAGCCCACTTACCTGCGTTTCANNNNCTTCAGCTTCCCGATATCCGACGGTAGTGTNNNNACATGGCTTGGCTCTTTGGG | 955 | 106196203 | 106197683 | + | 0 |
| GCAGCTCACGCTTTGCACACNNNNCTTCAGCTTCCCGATATCCGACGGTAGTGTNNNNCTTCCTCTTTCTCACGGGCTTTTT | 956 | 106196203 | 106197683 | − | 0 |
| GAAGTTTCATGTGGCTCAGCAGGCNNNNCTTCAGCTTCCCGATATCCGACGGTAGTGTNNNNACAAAAGGGGGTGATATCAT | 957 | 106196203 | 106197683 | − | 0 |
| CCACAACACTTCATAGACATCANNNNCTTCAGCTTCCCGATATCCGACGGTAGTGTNNNNGGACAGCCAGATATCAACTGTT | 958 | 170837531 | 170837568 | − | 0 |
| CAGTAGATCTCATTTTCCTATCAGNNNNCTTCAGCTTCCCGATATCCGACGGTAGTGTNNNNACCATCCACAAAATGGATCC | 959 | 140453092 | 140453191 | + | 0 |
| CACAACAAAGCCTGCTGAAGATAGNNNNCTTCAGCTTCCCGATATCCGACGGTAGTGTNNNNAGGGGGGAGGAGGTAGCAGA | 960 | 148504736 | 148504801 | + | 10 |
| GGGCTTTTTCTACTGGATTGTGNNNNCTTCAGCTTCCCGATATCCGACGGTAGTGTNNNNGCTCACTGACACCAGTGTGTCT | 961 | 148506160 | 148506251 | − | 0 |
| GTAAGCACAGCCCAGTGAATNNNNCTTCAGCTTCCCGATATCCGACGGTAGTGTNNNNCCTACCTTTTGCATAGCAGTTTGG | 962 | 148506400 | 148506485 | + | 0 |
| GCTGGGAGGCAGTGAGTTCCTNNNNCTTCAGCTTCCCGATATCCGACGGTAGTGTNNNNGTCAGGCTTGATCACCTTTATCC | 963 | 148507415 | 148507487 | − | 0 |
| GGCACTGATAACCTGTATTCAGGTNNNNCTTCAGCTTCCCGATATCCGACGGTAGTGTNNNNTTTTCACCCTCCTTTTTTGA | 964 | 148508716 | 148508816 | − | 0 |
| GGAGGTTCTTCACTCATCACCNNNNCTTCAGCTTCCCGATATCCGACGGTAGTGTNNNNTTACTGTCCCAATGGTCAGCGGC | 965 | 148511048 | 148511235 | + | 0 |
| GTAGTTAGCTATTTAGTGATGCAANNNNCTTCAGCTTCCCGATATCCGACGGTAGTGTNNNNCTGGCTGTCCGAGAGTGTGA | 966 | 148511048 | 148511235 | − | 0 |
| GCATGAGAACTAAATAGGTCTTTGNNNNCTTCAGCTTCCCGATATCCGACGGTAGTGTNNNNCTCTAACCATGTTTACAACT | 967 | 148511999 | 148512138 | − | 0 |
| GCTTTGTTTTCATTTGTTTTAGNNNNCTTCAGCTTCCCGATATCCGACGGTAGTGTNNNNGCTCTCTGTTGGATTTGTAGCT | 968 | 148512570 | 148512669 | − | 0 |
| GCAGAGGGTACTTGAGAGGACTTTNNNNCTTCAGCTTCCCGATATCCGACGGTAGTGTNNNNTATTAGATTCTTTGTTTCAT | 969 | 148516685 | 148516783 | − | 0 |
| GTTTCTAAAAGGTTTCCATGTGTTNNNNCTTCAGCTTCCCGATATCCGACGGTAGTGTNNNNCAGATTTAGCATTTGGTCCA | 970 | 148523544 | 148523730 | + | 0 |
| CGTTTTCATTTTCTATCTTTGTTGNNNNCTTCAGCTTCCCGATATCCGACGGTAGTGTNNNNACTTCCTCCTGAATGTACCC | 971 | 148523544 | 148523730 | − | 0 |
| GAGCCATATGCTTCTTCTCTTGGNNNNCTTCAGCTTCCCGATATCCGACGGTAGTGTNNNNTTTTTTTCTTTTAGGTGGAAG | 972 | 148526828 | 148526931 | − | 0 |
| TTGTTCTGTTGGAATAGTTCCTTGNNNNCTTCAGCTTCCCGATATCCGACGGTAGTGTNNNNTTACACCGCTTGTACCAGAA | 973 | 117864785 | 117864951 | − | 0 |
| GAGGACCAGCAACAGCAGCATNNNNCTTCAGCTTCCCGATATCCGACGGTAGTGTNNNNGAAATTTTTCTTTTTTTTTTTTT | 974 | 117864785 | 117864951 | − | 0 |
| GAATAATCACTAAGTTGGGCTCTNNNNCTTCAGCTTCCCGATATCCGACGGTAGTGTNNNNGAAGTAAAAATTCTGCAAACT | 975 | 117866482 | 117866595 | + | 0 |
| GGATCTGGCACCGCCCACCANNNNCTTCAGCTTCCCGATATCCGACGGTAGTGTNNNNCAGGAAAATGATCTTATTTTTTAT | 976 | 117866604 | 117866713 | − | 0 |
| CCTTTAGATTTATACAGCATNNNNCTTCAGCTTCCCGATATCCGACGGTAGTGTNNNNTTTTTTTTTTTTTTTTTTACAGTG | 977 | 117868403 | 117868530 | − | 0 |
| AGACTAGAAGGTTTGATTTATCTGNNNNCTTCAGCTTCCCGATATCCGACGGTAGTGTNNNNAAGGACAAAAAAGACAGTAA | 978 | 5069950 | 5070055 | − | 0 |
| CAGGATCACAGCTAGGTGTCAGTGNNNNCTTCAGCTTCCCGATATCCGACGGTAGTGTNNNNAGCAAGTATGATGAGCAAGC | 979 | 5073739 | 5073781 | + | 0 |
| CTTTCTACACATGCGTGGAAGTCNNNNCTTCAGCTTCCCGATATCCGACGGTAGTGTNNNNTTTTCATTTCTGACAACTTAC | 980 | 112350168 | 112350333 | + | 0 |
| GCTGGTTTATTCCTTTTCGACGGNNNNCTTCAGCTTCCCGATATCCGACGGTAGTGTNNNNTAAGCTATTCTTATTCCCTCT | 981 | 112350168 | 112350333 | − | 0 |
| GTTAGATGTCAGGGATACAGCCNNNNCTTCAGCTTCCCGATATCCGACGGTAGTGTNNNNCCTGAGTACCAATAAAGATTTG | 982 | 112350747 | 112350893 | + | 0 |
| AGTAACCTCTCCAGGAAGATTCATNNNNCTTCAGCTTCCCGATATCCGACGGTAGTGTNNNNGTGTATAGTGCCTGGGCACT | 983 | 112350747 | 112350893 | − | 0 |
| CAGCTGGCCCGTGCTTTCACTANNNNCTTCAGCTTCCCGATATCCGACGGTAGTGTNNNNCTGTGTTGTGATCTCTCTGTTG | 984 | 112352828 | 112352984 | + | 0 |
| GAGTCTTTCCAAACACATGTTTGNNNNCTTCAGCTTCCCGATATCCGACGGTAGTGTNNNNCGCCCAGCCAGATTATATGTT | 985 | 112352828 | 112352984 | − | 0 |
| GCAAAGCTCAATGAAAACCTGCGCNNNNCTTCAGCTTCCCGATATCCGACGGTAGTGTNNNNACAGACCTATTACATATGTT | 986 | 112356153 | 112356245 | + | 0 |
| GCCTCGGCCCTAACAATGTGGGCANNNNCTTCAGCTTCCCGATATCCGACGGTAGTGTNNNNGAAAAATAAATGTGAAGAAA | 987 | 32413516 | 32413615 | + | 0 |
| GCTGTGTTCCCTTGGGCTAGGGTTNNNNCTTCAGCTTCCCGATATCCGACGGTAGTGTNNNNTCAAATAGAATATGTGTCTT | 988 | 32414197 | 32414308 | − | 0 |
| GTCTTGAGGGAGAGTGAGCACTGGNNNNCTTCAGCTTCCCGATATCCGACGGTAGTGTNNNNGGTAAGCACACATGAAGGGG | 989 | 32417797 | 32417956 | + | 0 |
| GCAAACATGGTTCAAGAGCTCCTTNNNNCTTCAGCTTCCCGATATCCGACGGTAGTGTNNNNTCTTGTACGGTCGGCATCTG | 990 | 32417797 | 32417956 | − | 0 |
| AGGCTCAGTGTGGCTCACAGTCGCNNNNCTTCAGCTTCCCGATATCCGACGGTAGTGTNNNNTGGGGCCTGTCTGTGTGCTC | 991 | 32421491 | 32421591 | + | 0 |
| GAGCCCTGTGGACACCTCATGNNNNCTTCAGCTTCCCGATATCCGACGGTAGTGTNNNNCTGTTAACATTTATAATTGCAGT | 992 | 119148874 | 119149011 | + | 0 |
| GTTGGAATGTGGAGCCCATCTCACNNNNCTTCAGCTTCCCGATATCCGACGGTAGTGTNNNNTACCGAATTTTCCAAGGTTA | 993 | 119148874 | 119149011 | − | 0 |
| GAGGCAAGGAGCAGAGGGAGCTCCNNNNCTTCAGCTTCCCGATATCCGACGGTAGTGTNNNNAGTATTTTCAGATGCATCTG | 994 | 119149218 | 119149335 | + | 0 |
| CACAGATCTGTTTTCTGCAAAATCNNNNCTTCAGCTTCCCGATATCCGACGGTAGTGTNNNNGTACCTATGGTCCTAGTAGG | 995 | 25378562 | 25378647 | − | 0 |
| GACTGGGGAGGGCTTTCTTTGTGTNNNNCTTCAGCTTCCCGATATCCGACGGTAGTGTNNNNTGCACTGTAATAATCCAGAC | 996 | 25380275 | 25380285 | − | 0 |
| GCATATTACTGGTGCAGGACCATTNNNNCTTCAGCTTCCCGATATCCGACGGTAGTGTNNNNATAAGGCCTGCTGAAAATGA | 997 | 25398211 | 25398286 | − | 0 |
| GTCCATTGGAAAGGGAGGCAANNNNCTTCAGCTTCCCGATATCCGACGGTAGTGTNNNNCCATTCTTCTCTTTTAATTGCCC | 998 | 112888139 | 112888229 | − | 0 |
| TCCGCTCAGTAATAGTCACTCTNNNNCTTCAGCTTCCCGATATCCGACGGTAGTGTNNNNCACGTAATAATATTGACTTTTC | 999 | 112910747 | 112910847 | + | 0 |
| GGTCACATAAGTCCTGGACTGCTTNNNNCTTCAGCTTCCCGATATCCGACGGTAGTGTNNNNGGCTAGAAATGTATGGTCAG | 1000 | 112915440 | 112915540 | − | 10 |
| GGACAACAGAATCATTCATGGGGGNNNNCTTCAGCTTCCCGATATCCGACGGTAGTGTNNNNCATAAACTAAAAACAGAAAC | 1001 | 112926228 | 112926328 | − | 0 |
| GCGCAGGATTGAAGAAGAGCAGGTNNNNCTTCAGCTTCCCGATATCCGACGGTAGTGTNNNNGATGTTTCCTTCGTAGGTGT | 1002 | 112926848 | 112926919 | + | 0 |
| GGCCAGGTCTCTGTGAACACACTGNNNNCTTCAGCTTCCCGATATCCGACGGTAGTGTNNNNCACAAAATAGCCGTATAAAA | 1003 | 28592612 | 28592705 | + | 0 |
| AAGGAGCATTAAAAATGTAAAACTCAAGTNNNNCTTCAGCTTCCCGATATCCGACGGTAGTGTNNNNTTTTCGTGGAAGTGGG | 1004 | 28602314 | 28602426 | + | 0 |
| GTGGAAGGACAGCAACAAAGATGCNNNNCTTCAGCTTCCCGATATCCGACGGTAGTGTNNNNGCACGTACTCACCATTTGTC | 1005 | 28608022 | 28608130 | − | 0 |
| GCTTCAGAGATGAAATGATGAGTCNNNNCTTCAGCTTCCCGATATCCGACGGTAGTGTNNNNCATTCCATTCTTACCAAACT | 1006 | 28608200 | 28608360 | + | 0 |
| TCTGCAGCATTTCTTTTCCATTGGNNNNCTTCAGCTTCCCGATATCCGACGGTAGTGTNNNNGAAAGCCAGCTACAGATGGT | 1007 | 28608200 | 28608360 | − | 0 |
| GCAACAAAAGAGTGTCACTCAGCGNNNNCTTCAGCTTCCCGATATCCGACGGTAGTGTNNNNAGAGGAAAGAATAATGAATT | 1008 | 28608437 | 28608546 | + | 0 |
| CTGCTCGACACCCACTGTCCAAANNNNCTTCAGCTTCCCGATATCCGACGGTAGTGTNNNNGTGGGGAATTCCTGATGGTGG | 1009 | 28609629 | 28609815 | + | 0 |
| GCTGTCATCAGATTGGAAGTTAGGNNNNCTTCAGCTTCCCGATATCCGACGGTAGTGTNNNNACCCTTTTATGGCTTCACTC | 1010 | 28609629 | 28609815 | + | 0 |
| CGTGTGAAATAAGCTCACTGGCTGNNNNCTTCAGCTTCCCGATATCCGACGGTAGTGTNNNNAAGAGGCATCAATGTCCTTA | 1011 | 28610070 | 28610185 | + | 0 |
| GCAGATGATGGGCTCCCGGAAGACNNNNCTTCAGCTTCCCGATATCCGACGGTAGTGTNNNNAAGAGGATGGCTAGGCGAGG | 1012 | 90631835 | 90631946 | + | 0 |
| GAGATAATAGTGGTCCCACTGCAGNNNNCTTCAGCTTCCCGATATCCGACGGTAGTGTNNNNTGGTGATGGGCTTGGTCCAG | 1013 | 90631835 | 90631946 | + | 0 |
| GACAGAAGCAGGGAGGAGAGATGNNNNCTTCAGCTTCCCGATATCCGACGGTAGTGTNNNNAGGGGAGGGAGAGATGGGGGT | 1014 | 7572925 | 7573012 | + | 0 |
| GCTGGGAAGGAGCCAGGGGGGANNNNCTTCAGCTTCCCGATATCCGACGGTAGTGTNNNNCAAACAATTGTAACTTGAACCA | 1015 | 7573973 | 7574036 | − | 0 |
| GTCTAACACTCAAAATGCCGNNNNCTTCAGCTTCCCGATATCCGACGGTAGTGTNNNNTTCACTTTTATCACCTTTCCTTGC | 1016 | 7576850 | 7576934 | − | 0 |
| ACCAGGCTCCATCTACTCCCNNNNCTTCAGCTTCCCGATATCCGACGGTAGTGTNNNNTTCTCTTCCTCTGTGCGCCGGTCT | 1017 | 7577014 | 7577158 | + | 0 |
| CCAAGGGTGCAGTTATGCCTCAGNNNNCTTCAGCTTCCCGATATCCGACGGTAGTGTNNNNGCTTTGAGGTGCGTGTTTGTG | 1018 | 7577014 | 7577158 | − | 0 |
| GTAGTGGATGGTGGTACAGTCAGANNNNCTTCAGCTTCCCGATATCCGACGGTAGTGTNNNNAGAGGCAAGCAGAGGCTGGG | 1019 | 7577495 | 7577613 | + | 0 |
| GCGGCATGAACCGGAGGCCCATNNNNCTTCAGCTTCCCGATATCCGACGGTAGTGTNNNNAAAAAAAAAAAAAAAGGCCTCC | 1020 | 7577495 | 7577613 | − | 0 |
| CCTGGGGACCCTGGGCAACCANNNNCTTCAGCTTCCCGATATCCGACGGTAGTGTNNNNAACCAGACCTCAGGCGGCTCATA | 1021 | 7578168 | 7578295 | + | 0 |
| GTGAGCAGTAGGGGGGCTTTCNNNNCTTCAGCTTCCCGATATCCGACGGTAGTGTNNNNGAGTGGAAGGAAATTTGCGTGTG | 1022 | 7578115 | 7578211 | − | 0 |
| GTTGAGGGCAGGGGAGTACTGTAGGANNNNCTTCAGCTTCCCGATATCCGACGGTAGTGTNNNNATGGTGGGGGCAGCGCCT | 1023 | 7578364 | 7578560 | + | 0 |
| GGGCTGGAGAGACGACAGGGCTGGTTNNNNCTTCAGCTTCCCGATATCCGACGGTAGTGTNNNNCATGGCCATCTACAAGCA | 1024 | 7578364 | 7578560 | − | 0 |
| TTCCACACCCCCGCCCGGNNNNCTTCAGCTTCCCGATATCCGACGGTAGTGTNNNNCTGCCGTCTTCCAGTTGCTTTATCTG | 1025 | 7578364 | 7578560 | − | 0 |
| TTTCTGGGAAGGGACAGAAGATGANNNNCTTCAGCTTCCCGATATCCGACGGTAGTGTNNNNCAAACAAAAGAAATGCAGGG | 1026 | 7579308 | 7579600 | + | 0 |
| GTAGGAGCTGCTGGTGCAGGGGCNNNNCTTCAGCTTCCCGATATCCGACGGTAGTGTNNNNTCAGGGCAACTGACCGTGCAA | 1027 | 7579308 | 7579600 | + | 0 |
| GCATCAAATCATCCATTGCTTGGGNNNNCTTCAGCTTCCCGATATCCGACGGTAGTGTNNNNAGGAGGGGGCTGGTGCAGGG | 1028 | 7579443 | 7579523 | + | 0 |
| ACCCAGGTCCAGATGAAGCTCCCNNNNCTTCAGCTTCCCGATATCCGACGGTAGTGTNNNNTGGGGACCTGGAGGGCTGGGG | 1029 | 7579308 | 7579600 | − | 0 |
| AAGGGCAGGCCCACCACCCCNNNNCTTCAGCTTCCCGATATCCGACGGTAGTGTNNNNTCATGCTGGATCCCCACTTTTCCT | 1030 | 7579812 | 7579915 | − | 0 |
| GCCTTCCAATTGGCCTTGTGCCNNNNCTTCAGCTTCCCGATATCCGACGGTAGTGTNNNNGCCTTCTCAGAGAATTTTTTAG | 1031 | 58740352 | 58740920 | + | 0 |
| AAAAAATTTATCCCAGAACTCAACNNNNCTTCAGCTTCCCGATATCCGACGGTAGTGTNNNNCTCTCGAGCTATCTCAGCTG | 1032 | 58740352 | 58740920 | − | 0 |
| AGCCTGCAAGTCTCCCCACAACCNNNNCTTCAGCTTCCCGATATCCGACGGTAGTGTNNNNTGAAGATGTCAACTCCTGGCC | 1033 | 58740352 | 58740920 | + | 0 |
| CAATTTTCTTCAAGTGGTTCTGGNNNNCTTCAGCTTCCCGATATCCGACGGTAGTGTNNNNTCAATTTCTTGGGCTTTCATT | 1034 | 58740352 | 58740920 | − | 0 |
| GATGTTGAACTTTTTTTAAGGGGANNNNCTTCAGCTTCCCGATATCCGACGGTAGTGTNNNNATGCGACGCAGACTTAGGGG | 1035 | 58740352 | 58740920 | + | 0 |
| CGTCTATGCTTCTTCATCAGGGGNNNNCTTCAGCTTCCCGATATCCGACGGTAGTGTNNNNGTTTTCCTGTGTTGATGAAGT | 1036 | 58740352 | 58740920 | − | 0 |
| AGCTCGCGGCCGTCCAGCACNNNNCTTCAGCTTCCCGATATCCGACGGTAGTGTNNNNGGTCCCCGGCGGCTGTGGTGTGAG | 1037 | 74732956 | 74732961 | + | 0 |
| TTTCTGCTCCCTGGACAACCCGGNNNNCTTCAGCTTCCCGATATCCGACGGTAGTGTNNNNCTCCCTAAAGGAAATCACGCT | 1038 | 42531868 | 42531969 | + | 0 |
| GGCCTGCCTCCAGGGCTGGACTGANNNNCTTCAGCTTCCCGATATCCGACGGTAGTGTNNNNAAGAAGACAAGAAACGCAAA | 1039 | 13054525 | 13054730 | + | 0 |
| CTTGCCCCCTGCCAGCCCTGNNNNCTTCAGCTTCCCGATATCCGACGGTAGTGTNNNNATCTTTGTCCTCATCATCCTCCTT | 1040 | 13054525 | 13054730 | − | 0 |
| GCAACTGCGCGTGAGGCGCGCGGCTGTNNNNCTTCAGCTTCCCGATATCCGACGGTAGTGTNNNNGAAGGTGCTGGAGCTG | 1041 | 33792300 | 33792469 | − | 0 |
| TGACCGCCTGCGCAAGCGGGTGGANNNNCTTCAGCTTCCCGATATCCGACGGTAGTGTNNNNAAGGCCAAGAAGTCGGTGGA | 1042 | 33792244 | 33793326 | − | 0 |
| GCGGGGCTCCTGCTTGATCACCAGCGNNNNCTTCAGCTTCCCGATATCCGACGGTAGTGTNNNNGCGGGTGCGGGTGCGG | 1043 | 33792674 | 33792865 | + | 0 |
| TCATGCCCGGGGGAGCGCACGGGCNNNNCTTCAGCTTCCCGATATCCGACGGTAGTGTNNNNGGCCAAGGCGGCCGTG | 1044 | 33792970 | 33793010 | − | 0 |
| GCTGATGTCGATGGACGTCTCGTGCTCGCNNNNCTTCAGCTTCCCGATATCCGACGGTAGTGTNNNNCTTGGCCTTCTCCTGC | 1045 | 33793025 | 33793090 | + | 0 |
| GCAGGTGGCTGCTCATCGGGGGCCGCNNNNCTTCAGCTTCCCGATATCCGACGGTAGTGTNNNNGGTGGGGGGGGAGGCT | 1046 | 33793243 | 33793300 | + | 0 |
| GCCACCCGACAGCGAGATGGGCANNNNCTTCAGCTTCCCGATATCCGACGGTAGTGTNNNNGGCCTGAAACTGATGGCTGTG | 1047 | 31021087 | 31021727 | + | 0 |
| TCCCCATCTGCCAGGCACATCCNNNNCTTCAGCTTCCCGATATCCGACGGTAGTGTNNNNCAGATCTCCGAACCAGAGCCAG | 1048 | 31021087 | 31021727 | + | 0 |
| CCTGGGACACACAAGCCACTNNNNCTTCAGCTTCCCGATATCCGACGGTAGTGTNNNNTTTGCATCCTTAGCAACCCCTGCT | 1049 | 31021087 | 31021727 | − | 0 |
| GAGCAGGCGGCCTCTGCATCCTTTNNNNCTTCAGCTTCCCGATATCCGACGGTAGTGTNNNNCCGAATTCCCAGTTGAGTCT | 1050 | 31021087 | 31021727 | + | 0 |
| GCCCTGCTGGGTCAGTCTTAGNNNNCTTCAGCTTCCCGATATCCGACGGTAGTGTNNNNCAGGCTAGGAATTCTGTCTGGAG | 1051 | 31021087 | 31021727 | − | 0 |
| CACAGCCCACTAAAGAGGAGCCCANNNNCTTCAGCTTCCCGATATCCGACGGTAGTGTNNNNTCAGGAAACTGTGGATCAGG | 1052 | 31021087 | 31021727 | + | 0 |
| CGGGGTTGGACTGGCGCCAGGANNNNCTTCAGCTTCCCGATATCCGACGGTAGTGTNNNNTAGGTCAGATCACCCAGTCAGT | 1053 | 31022233 | 31025152 | + | 0 |
| GTGGTGATGGTGGTGAGGCCTGTGGNNNNCTTCAGCTTCCCGATATCCGACGGTAGTGTNNNNGAGGGGCGAGAGGTCA | 1054 | 31022435 | 31022452 | + | 0 |
| GGTGGGGATGATCCGGGGGCATATNNNNCTTCAGCTTCCCGATATCCGACGGTAGTGTNNNNTCCGATGGCAGTGGTGGCCG | 1055 | 31022322 | 31022412 | − | 0 |
| TGCCAGGCCTTGCCCCTACTGTCNNNNCTTCAGCTTCCCGATATCCGACGGTAGTGTNNNNGGAACTGCCATGTCCAGAGCT | 1056 | 31022233 | 31025152 | + | 0 |
| GTACACTTTCCAGGGGTGCTCGGGNNNNCTTCAGCTTCCCGATATCCGACGGTAGTGTNNNNCTGTAGCCCTCTGTAGTAGG | 1057 | 31022233 | 31025152 | − | 0 |
| GGATCCTGTAAATGTGACCCCCNNNNCTTCAGCTTCCCGATATCCGACGGTAGTGTNNNNGTGAGTCTGGCACCACTTCCTG | 1058 | 31022233 | 31025152 | + | 0 |
| GCAAGGCCTGGCATGGCTGGTNNNNCTTCAGCTTCCCGATATCCGACGGTAGTGTNNNNACCATTGTCTGCAGGAACGGTGG | 1059 | 31022233 | 31025152 | − | 0 |
| GGTAGTGAAACAGCCCAAACCAGNNNNCTTCAGCTTCCCGATATCCGACGGTAGTGTNNNNGCAGAACAGAGCATTTGATGA | 1060 | 31022233 | 31025152 | + | 0 |
| GCTTGGCCAGTTCCTTTCTCTNNNNCTTCAGCTTCCCGATATCCGACGGTAGTGTNNNNTATCACTTTCCCTCATAGGAGGG | 1061 | 31022233 | 31025152 | − | 0 |
| GGAGACTCTGAAGCACTGAGTCCTNNNNCTTCAGCTTCCCGATATCCGACGGTAGTGTNNNNCTGCATTGCCTGGGGATTTG | 1062 | 31022233 | 31025152 | + | 0 |
| CGATGGGATGGGTATCCAATGCANNNNCTTCAGCTTCCCGATATCCGACGGTAGTGTNNNNAGGATCTAGACCCTCCTCAGC | 1063 | 31022233 | 31025152 | − | 0 |
| GTTGACAGTAACTGCCATTGCTGTNNNNCTTCAGCTTCCCGATATCCGACGGTAGTGTNNNNCTTTGTCACTGCAGCTTCTC | 1064 | 31022233 | 31025152 | − | 0 |
| GGTGACCTTCAAAGTCAGAGGCTGNNNNCTTCAGCTTCCCGATATCCGACGGTAGTGTNNNNATCTGTCCTTGTAACCAGAC | 1065 | 31022233 | 31025152 | − | 0 |
| GTAGCTTGCCCCTAGAGAAGGTNNNNCTTCAGCTTCCCGATATCCGACGGTAGTGTNNNNTGGGTGTCTCGAGTATGTGCGG | 1066 | 31022233 | 31025152 | + | 0 |
| GTGGGGCAGATTGGTTCCAATTGGNNNNCTTCAGCTTCCCGATATCCGACGGTAGTGTNNNNGGCTCTTGGCTGGTACTCAG | 1067 | 31022233 | 31025152 | − | 0 |
| GCAACTGCATCACAAGTGGGTTAGNNNNCTTCAGCTTCCCGATATCCGACGGTAGTGTNNNNGCCACTGTTTTTTCCAAGAC | 1068 | 31022233 | 31025152 | − | 0 |
| CCCTCCATCCAGTGACAAATCCCANNNNCTTCAGCTTCCCGATATCCGACGGTAGTGTNNNNTGGAAGCAGCCCCAGTTCTT | 1069 | 31022233 | 31025152 | + | 10 |
| GCAGTTCTCTTCCTTTAGTTGTGNNNNCTTCAGCTTCCCGATATCCGACGGTAGTGTNNNNAGGCACTGGTCTTGCCAGGAT | 1070 | 31022233 | 31025152 | + | 0 |
| GCGAGCCATGGCTCTGGTCTTTTGNNNNCTTCAGCTTCCCGATATCCGACGGTAGTGTNNNNCTGGGTGGCCTCAATCCTGG | 1071 | 31022233 | 31025152 | − | 0 |
| CCTCGTTTCTCATCTCCAAATGTGNNNNCTTCAGCTTCCCGATATCCGACGGTAGTGTNNNNCTAGGAAACTGGAAGAAATG | 1072 | 31022233 | 31025152 | + | 0 |
| AACCCTTCAGCGCCCCAGGCNNNNCTTCAGCTTCCCGATATCCGACGGTAGTGTNNNNCCCAGGAGATCTTACTACCTCGAG | 1073 | 31022233 | 31025152 | + | 0 |
| GTGGGGTACAGACTCCAAGGGAAGNNNNCTTCAGCTTCCCGATATCCGACGGTAGTGTNNNNAGGCCAAGGGAAGAAGCTTT | 1074 | 31022233 | 31025152 | + | 0 |
| GCCGAGAACAGGAAAGCTACTGGGNNNNCTTCAGCTTCCCGATATCCGACGGTAGTGTNNNNTTCCCAGTGGGAAGTTGGGA | 1075 | 31022233 | 31025152 | + | 0 |
| GAAGGGGCTCAGTGAGCCTCTGGANNNNCTTCAGCTTCCCGATATCCGACGGTAGTGTNNNNGAGAAGACTTTTGTGGGGGG | 1076 | 31022233 | 31025152 | + | 0 |
| CCTCTAGCTCTCCCACCTTTCNNNNCTTCAGCTTCCCGATATCCGACGGTAGTGTNNNNCTTGCCCTTCTGGAAATTACCCC | 1077 | 31022233 | 31025152 | + | 0 |
| GCAGCACGGTGGAAAGCATCTCGNNNNCTTCAGCTTCCCGATATCCGACGGTAGTGTNNNNTCCAACTGAGTTCCACCAGCT | 1078 | 31022233 | 31025152 | + | 0 |
| GCTCTGTGTATTGTGCCTTGTGGTNNNNCTTCAGCTTCCCGATATCCGACGGTAGTGTNNNNCACAGTGCATCACTTTCCTT | 1079 | 31022233 | 31025152 | + | 0 |
| GAGGTCAATGGATCTCACCAAAGCCANNNNCTTCAGCTTCCCGATATCCGACGGTAGTGTNNNNCAAGAAACCATGATCTCT | 1080 | 57484402 | 57484481 | − | 0 |
| GGTGGTAGGAGGGCGAGCTGGCTTNNNNCTTCAGCTTCCCGATATCCGACGGTAGTGTNNNNGGTTCGGGAGGCTGGGGTTG | 1081 | 36164474 | 36164925 | + | 0 |
| GCGGCGGCAGGTAGGTGTGGTAGNNNNCTTCAGCTTCCCGATATCCGACGGTAGTGTNNNNACCATGGAGAACTGGTAGGAG | 1082 | 36164474 | 36164925 | + | 0 |
| GGTGACCGGCGTCGGGGAGTAGGTNNNNCTTCAGCTTCCCGATATCCGACGGTAGTGTNNNNTTGCGACGAGCCGGGGTAGG | 1083 | 36164474 | 36164925 | + | 0 |
| GCGGGGGTCGGAGATGGAGGGNNNNCTTCAGCTTCCCGATATCCGACGGTAGTGTNNNNGGGGTAGGGCGGCGGCAGGTAGG | 1084 | 36164474 | 36164925 | + | 0 |
| GCGGAAGTGAGTAGGAGGTTGCGGANNNNCTTCAGCTTCCCGATATCCGACGGTAGTGTNNNNGTCGGGGAGTAGGTGAAGG | 1085 | 36164474 | 36164925 | + | 0 |
| GTCCAGGAGACTAGAGGTGCATGNNNNCTTCAGCTTCCCGATATCCGACGGTAGTGTNNNNTCCATTGCCTCTCCTTCTGTG | 1086 | 36171591 | 36171765 | − | 0 |
| ACGCCCATTTCACCTGGACGTGNNNNCTTCAGCTTCCCGATATCCGACGGTAGTGTNNNNCATTTTTTAAATCCCACCCCAC | 1087 | 36171591 | 36171765 | − | 0 |
| GAGGCACGAGGGTTGGGCGTGNNNNCTTCAGCTTCCCGATATCCGACGGTAGTGTNNNNGGGAAGGTGTGTGCACATGGGGG | 1088 | 36206704 | 36206900 | + | 0 |
| CGGGCTTGGTCTGATCATCTAGTNNNNCTTCAGCTTCCCGATATCCGACGGTAGTGTNNNNGGGTTAAAGGCAGTGGAGTGG | 1089 | 36206704 | 36206900 | + | 0 |
| GCGGCGCACAGCCATGAGGGTNNNNCTTCAGCTTCCCGATATCCGACGGTAGTGTNNNNTCTTCCCTCCCTCCTTCCCTCCC | 1090 | 36206704 | 36206900 | − | 0 |
| GGGCTGGTACACCCTCCAGGCTNNNNCTTCAGCTTCCCGATATCCGACGGTAGTGTNNNNTTTTGTTCTCTATCGTGTCCCC | 1091 | 36231767 | 36231880 | − | 0 |
| CATCATTGCCAGCCATCACAGNNNNCTTCAGCTTCCCGATATCCGACGGTAGTGTNNNNTGGGTTTGTTGCCATGAAACGTG | 1092 | 36252844 | 36253026 | + | 0 |
| GCTGAGCTGAGAAATGCTACCGCNNNNCTTCAGCTTCCCGATATCCGACGGTAGTGTNNNNATCACTACACAAATGCCCTAA | 1093 | 36252844 | 36253026 | − | 0 |
| GCAGAGGAAGTTGGGGCTGTCGGTGCGCANNNNCTTCAGCTTCCCGATATCCGACGGTAGTGTNNNNCTGTCCTCCCACCACC | 1094 | 36259157 | 36259187 | + | 0 |
| GGCCAGCACCTCCACCATGCTGNNNNCTTCAGCTTCCCGATATCCGACGGTAGTGTNNNNGTCTTGTTGCAGCGCCAGTGCG | 1095 | 36259192 | 36259400 | + | 0 |
| GCCAGTGACGTGACTGAGCACANNNNCTTCAGCTTCCCGATATCCGACGGTAGTGTNNNNTTTCGCCGTGAGGAAGATGCGG | 1096 | 44514777 | 44514876 | − | 0 |
| CATGGAATATGTCAGCAGCATGACNNNNCTTCAGCTTCCCGATATCCGACGGTAGTGTNNNNCAAACAAACCTGGCTAAACG | 1097 | 44524444 | 44524480 | + | 0 |
| GCATGCGTGTGGAGGAGGGGACTGNNNNCTTCAGCTTCCCGATATCCGACGGTAGTGTNNNNAATGGAGCAGTGCAGGAGGG | 1098 | 15833798 | 15834016 | + | 0 |
| GGCCAATAGTTGAAAATTACTCACNNNNCTTCAGCTTCCCGATATCCGACGGTAGTGTNNNNGCTTGCGTCAGGGTCATAGT | 1099 | 15833798 | 15834016 | − | 0 |
| AAATTCAGGAAAAGAAACCAGCCNNNNCTTCAGCTTCCCGATATCCGACGGTAGTGTNNNNAACAGAAACAGAACAACAAAC | 1100 | 15836703 | 15836772 | − | 0 |
| TTTCCTCAATTGTTCCACTGCNNNNCTTCAGCTTCCCGATATCCGACGGTAGTGTNNNNGCATATCATTTGATTTTTGGTTT | 1101 | 15838328 | 15838442 | + | 0 |
| GCTTCCAGGTCTTTGGAGCAAGAGNNNNCTTCAGCTTCCCGATATCCGACGGTAGTGTNNNNGTACATGGTGGGTCCAGCTT | 1102 | 39911360 | 39911656 | + | 0 |
| GTAAAATGAAAAGTGCGCCCAACNNNNCTTCAGCTTCCCGATATCCGACGGTAGTGTNNNNTTCTGCCTCTGCAATGGTGAC | 1103 | 39911360 | 39911656 | + | 0 |
| GTAAGGAGCTGTTAGATCTGGTGGNNNNCTTCAGCTTCCCGATATCCGACGGTAGTGTNNNNGAAATTGAAAATGTCCTCCC | 1104 | 39911360 | 39911656 | − | 0 |
| CGTTCTCAACAGCATCGTGCAGAGNNNNCTTCAGCTTCCCGATATCCGACGGTAGTGTNNNNAGACCATTTCTTGAACTTTG | 1105 | 39914617 | 39914770 | + | 0 |
| CTTATGGTGCTGACCCCACCNNNNCTTCAGCTTCCCGATATCCGACGGTAGTGTNNNNTGCCCAGCTTTGCCTGTTGCTTTT | 1106 | 39914617 | 39914770 | − | 0 |
| TCGCCCAGTCCAATGCCTTGNNNNCTTCAGCTTCCCGATATCCGACGGTAGTGTNNNNTCCTGCTGGTTTTGGTGCCATCTG | 1107 | 39931589 | 39934460 | + | 0 |
| CATGTGGTCAGCTTTGGAAGCATCNNNNCTTCAGCTTCCCGATATCCGACGGTAGTGTNNNNGAGGGAGCAGTGCTGATGAT | 1108 | 39931589 | 39934460 | + | 0 |
| CAGCAGCGGAGTTCATCATGCCCGNNNNCTTCAGCTTCCCGATATCCGACGGTAGTGTNNNNCTCCAGGAAATGGTTGTGCT | 1109 | 39931589 | 39934460 | − | 0 |
| CAGGGGTTTTCTTCCCCTCTGGGCCTGAGNNNNCTTCAGCTTCCCGATATCCGACGGTAGTGTNNNNTTTCTGTGTCTGAGGA | 1110 | 48649515 | 48649604 | + | 0 |
| GCTGGAAGCTTCTCAAATGGATGNNNNCTTCAGCTTCCCGATATCCGACGGTAGTGTNNNNTGGCCTACTACAGGGACGCTG | 1111 | 48649516 | 48649738 | + | 0 |
| GGTGTGGAGGACACCAGAGCAGGANNNNCTTCAGCTTCCCGATATCCGACGGTAGTGTNNNNTGGAGTTACCTGGGGAGTGT | 1112 | 48649516 | 48649738 | − | 0 |
| GCAGCTCTGCCTCTTTCCGTTTTGNNNNCTTCAGCTTCCCGATATCCGACGGTAGTGTNNNNTGTGTGGATGGCCTTTGGAG | 1113 | 53431940 | 53432902 | + | 0 |
| GCACTGCCTGTGGCTTACTTTCNNNNCTTCAGCTTCCCGATATCCGACGGTAGTGTNNNNATCTGCAGTCCATGGGCCTGAG | 1114 | 53431940 | 53432902 | + | 0 |
| GCCTTCAGGTCACTGGCCCCANNNNCTTCAGCTTCCCGATATCCGACGGTAGTGTNNNNGCTAGGTGGTAAGGTGGTGGCTG | 1115 | 53431940 | 53432902 | + | 0 |
| CCAGTACTGAGCCTGTCCAGNNNNCTTCAGCTTCCCGATATCCGACGGTAGTGTNNNNTTTCTCATCCCAGCGCCGTGCCTT | 1116 | 53431940 | 53432902 | + | 0 |
| GACAAGGGCATTGCCACAAGCATANNNNCTTCAGCTTCCCGATATCCGACGGTAGTGTNNNNGGGGGAAGAGAGAAGAGGGG | 1117 | 53431940 | 53432902 | + | 0 |
| CCTCACCTCCAGGTAGTCAAGNNNNCTTCAGCTTCCCGATATCCGACGGTAGTGTNNNNATCAATCACTAGCTTGGCCCCCT | 1118 | 53431940 | 53432902 | + | 0 |
| GCCGCATTGCCTTTGGAGGCCANNNNCTTCAGCTTCCCGATATCCGACGGTAGTGTNNNNCAGGTGAAGCCTACAGATGAGA | 1119 | 53431940 | 53432902 | − | 0 |
| GCTTTTGGAAGCTGGCTCAGGNNNNCTTCAGCTTCCCGATATCCGACGGTAGTGTNNNNGCCTGTCTTCTCCGAGTCCACAA | 1120 | 53431940 | 53432902 | + | 0 |
| GGGTCAGGCCAGTGTTCAAGGGNNNNCTTCAGCTTCCCGATATCCGACGGTAGTGTNNNNACCAAGGTTTTGGGCAAGAACA | 1121 | 53431940 | 53432902 | − | 0 |
| GGCTATTGTGTGACCAACTTGGTCNNNNCTTCAGCTTCCCGATATCCGACGGTAGTGTNNNNTGGCACTAAATCTTAGCATT | 1122 | 123181202 | 123181359 | + | 0 |
| GGTGAAACTAATCTAACAGACACANNNNCTTCAGCTTCCCGATATCCGACGGTAGTGTNNNNGCCTCTCATTGGCTCGTTTT | 1123 | 123181202 | 123181359 | − | 0 |
| GCAAGTTTGCATATTTCGTGGTGTNNNNCTTCAGCTTCCCGATATCCGACGGTAGTGTNNNNGAAATGATGTGTTTTTTTAC | 1124 | 123182853 | 123182930 | + | 0 |
| AAGATGTGCCCTTCAGACTGCTTNNNNCTTCAGCTTCCCGATATCCGACGGTAGTGTNNNNTTATGCATCGTTTTTCCTTCC | 1125 | 123184036 | 123184161 | + | 0 |
| CCAGTCGGTTCAAGGTTAGTATTANNNNCTTCAGCTTCCCGATATCCGACGGTAGTGTNNNNGAAGTTGAAAATACATAGAG | 1126 | 123184969 | 123185250 | + | 0 |
| GTGTCTATGACCCTTGACAAAGNNNNCTTCAGCTTCCCGATATCCGACGGTAGTGTNNNNAAGAGCTTAATTCCAAACTGGA | 1127 | 123184969 | 123185250 | + | 0 |
| GCATATTTGCACTAATGTTCAGATNNNNCTTCAGCTTCCCGATATCCGACGGTAGTGTNNNNCTTTTCTCTTGCTTTCCTTT | 1128 | 123184969 | 123185250 | + | 0 |
| GAAATAAGCAGTAACAGGTGCTTNNNNCTTCAGCTTCCCGATATCCGACGGTAGTGTNNNNGCCTTAGAAAATGAGTAACAG | 1129 | 123189975 | 123190088 | − | 0 |
| ACCATGTCAGTCATTAGTGGAATCNNNNCTTCAGCTTCCCGATATCCGACGGTAGTGTNNNNGTATCAAAGCTAACAGTTTC | 1130 | 123220395 | 123220624 | + | 0 |
| CCACCAGCTAGCAAAGAATTTCGGNNNNCTTCAGCTTCCCGATATCCGACGGTAGTGTNNNNAAAGAGAATAAATTATATCC | 1131 | 123220395 | 123220624 | − | 0 |
| CCGGCAGCAACAGAGACCTTGAAANNNNCTTCAGCTTCCCGATATCCGACGGTAGTGTNNNNTGTCAAGCTCAGTTGAACAG | 1132 | 133511647 | 133511790 | + | 0 |
| GCCACTTTAAGTCTCAAGAAATGCNNNNCTTCAGCTTCCCGATATCCGACGGTAGTGTNNNNCGCTGCCACCTTCTGGTTTT | 1133 | 133511647 | 133511790 | − | 0 |
| GCATTTCATCATCATCATAAAGGGNNNNCTTCAGCTTCCCGATATCCGACGGTAGTGTNNNNCTACCACTGTGCATTGCATG | 1134 | 133527529 | 133527670 | + | 0 |
| GTATGTGACTTTCTAAGGCTGTATNNNNCTTCAGCTTCCCGATATCCGACGGTAGTGTNNNNGGTTTCTCTCGTATTTGAGC | 1135 | 133527529 | 133527670 | − | 0 |
| CCACGTTTCAGCCACTTTTCAGNNNNCTTCAGCTTCCCGATATCCGACGGTAGTGTNNNNTTAGTTTGCTTACTAATTTTTG | 1136 | 133527932 | 133527989 | + | 0 |
| GGGGAGGAAGAAAATGAAGCACGANNNNCTTCAGCTTCCCGATATCCGACGGTAGTGTNNNNGAAATGTTAAGTAAGCTTGA | 1137 | 133547847 | 133548010 | + | 0 |
| GCCTGGTGTCACTAGGGCTGCNNNNCTTCAGCTTCCCGATATCCGACGGTAGTGTNNNNAAACAAACAACAAAAAAACAAAA | 1138 | 133547847 | 133548010 | − | 0 |
| ATGCAGGAAAATTAACATTCAGAATCCNNNNCTTCAGCTTCCCGATATCCGACGGTAGTGTNNNNTGTTGCATAACAAATATAAAAACTACA | 1139 | 133549044 | 133549153 | − | 0 |
| GACTGCAAAGTGTACATTTCTGNNNNCTTCAGCTTCCCGATATCCGACGGTAGTGTNNNNGTTTAGGGAAATAGACAACTGT | 1140 | 133551219 | 133551319 | − | 0 |

### REFERENCE LIST

1. Diepen S Van, Katz J N, Albert N M, Henry T D, Jacobs A K, Kapur N K, Kilic A, Menon V, Ohman E M, Sweitzer N K, Thiele H, Washam J B, Cohen M G. Contemporary Management of Cardiogenic Shock: A Scientific Statement from the American Heart Association. Circulation.
2. Helgestad O K L, Josiassen J, Hassager C, Jensen L O, Holmvang L, Sørensen A, Frydland M, Lassen A T, Udesen N L J, Schmidt H, Ravn H B, Møller J E. Temporal trends in incidence and patient characteristics in cardiogenic shock following acute myocardial infarction from 2010 to 2017: a Danish cohort study. Eur J Heart Fail 2019; 21; (11):1370-1378.
3. Jentzer J C, Lawler P R, Diepen S Van, Henry T D, Menon V, Baran D A, Džavík V, Barsness G W, Holmes D R, Kashani K B. Systemic Inflammatory Response Syndrome Is Associated With Increased Mortality Across the Spectrum of Shock Severity in Cardiac Intensive Care Patients. Circ Cardiovasc Qual Outcomes 2020; 13; (12):1033-1045.
4. Andrié R P, Becher U M, Frommold R, Tiyerili V, Schrickel J W, Nickenig G, Schwab J O. Interleukin-6 is the strongest predictor of 30-day mortality in patients with cardiogenic shock due to myocardial infarction. Crit Care 2012; 16; (4).
5. Jaiswal S, Fontanillas P, Flannick J, Manning A, Grauman P V., Mar B G, Lindsley R C, Mermel C H, Burtt N, Chavez A, Higgins J M, Moltchanov V, Kuo F C, Kluk M J, Henderson B, Kinnunen L, Koistinen H A, Ladenvall C, Getz G, Correa A, Banahan B F, Gabriel S, Kathiresan S, Stringham H M, McCarthy M I, Boehnke M, Tuomilehto J, Haiman C, Groop L, Atzmon G, Wilson J G, Neuberg D, Altshuler D, Ebert B L. Age-related clonal hematopoiesis associated with adverse outcomes. N Engl J Med 2014; 371; (26):2488-2498.
6. Acuna-Hidalgo R, Sengul H, Steehouwer M, Vorst M van de, Vermeulen S H, Kiemeney L A L M, Veltman J A, Gilissen C, Hoischen A. Ultra-sensitive sequencing identifies high prevalence of clonal hematopoiesis-associated mutations throughout adult life. Am J Hum Genet 2017; 101; (1):50-64.
7. Young A L, Challen G A, Birmann B M, Druley T E. Clonal haematopoiesis harbouring AML-associated mutations is ubiquitous in healthy adults. Nat Commun 2016; 7:12484.
8. Genovese G, Kähler A K, Handsaker R E, Lindberg J, Rose S A, Bakhoum S F, Chambert K, Mick E, Neale B M, Fromer M, Purcell S M, Svantesson O, Landen M, Hoglund M, Lehmann S, Gabriel S B, Moran J L, Lander E S, Sullivan P F, Sklar P, Grönberg H, Hultman C M, McCarroll S A. Clonal hematopoiesis and blood-cancer risk inferred from blood DNA sequence. N Engl J Med 2014; 371; (26):2477-2487.
9. Dorsheimer L, Assmus B, Rasper T, Ortmann C A, Ecke A, Abou-El-Ardat K, Schmid T, Brune B, Wagner S, Serve H, Hoffmann J, Seeger F, Dimmeler S, Zeiher A M, Rieger M A. Association of mutations contributing to clonal hematopoiesis with prognosis in chronic ischemic heart failure. JAMA Cardiol 2019; 4; (1):25-33.
10. Jaiswal S, Natarajan P, Silver A J, Gibson C J, Bick A G, Shvartz E, McConkey M, Gupta N, Gabriel S, Ardissino D, Baber U, Mehran R, Fuster V, Danesh J, Frossard P, Saleheen D, Melander O, Sukhova G K, Neuberg D, Libby P, Kathiresan S, Ebert B L. Clonal hematopoiesis and risk of atherosclerotic cardiovascular disease. N Engl J Med 2017; 377; (2):111-121.
11. Jaiswal S, Libby P. Clonal haematopoiesis: connecting ageing and inflammation in cardiovascular disease. Nature Reviews Cardiology.
12. Yu B, Roberts M B, Raffield L M, Zekavat S M, Nguyen NQH, Biggs M L, Brown M R, Griffin G, Desai P, Correa A, Morrison A C, Shah A M, Niroula A, Uddin M M, Honigberg M C, Ebert B L, Psaty B M, Whitsel E A, Manson JAE, Kooperberg C, Bick A G, Ballantyne C M, Reiner A P, Natarajan P, Eaton C B. Supplemental association of clonal hematopoiesis with incident heart failure. J Am Coll Cardiol 2021; 78; (1):42-52.
13. Sano S, Oshima K, Wang Y, Katanasaka Y, Sano M, Walsh K. CRISPR-mediated gene editing to assess the roles of TET2 and DNMT3A in clonal hematopoiesis and cardiovascular disease. Circ Res 2018; 123; (3):335-341.
14. Baran D A, Grines C L, Bailey S, Burkhoff D, Hall S A, Henry T D, Hollenberg S M, Kapur N K, O'Neill W, Ornato J P, Stelling K, Thiele H, Diepen S, Naidu S S. SCAI clinical expert consensus statement on the classification of cardiogenic shock. Catheter Cardiovasc Interv 2019; 94; (1):29-37.
15. Medeiros J J F, Capo-Chichi J-M, Shlush L I, Dick J E, Arruda A, Minden M D, Abelson S. SmMIP-tools: a computational toolset for processing and analysis of single-molecule molecular inversion probes derived data 2. bioRxiv 2021:2021.06.03.446993.
16. Pascual-Figal D A, Bayes-Genis A, Diez-Diez M, Hernández-Vicente Á, Vázquez-Andrés D, la Barrera J de, Vazquez E, Quintas A, Zuriaga M A, Asensio-López M C, Dopazo A, Sánchez-Cabo F, Fuster J J. Clonal hematopoiesis and risk of progression of heart failure with reduced left ventricular ejection fraction. J Am Coll Cardiol 2021; 77; (14):1747-1759.
17. Watson C J, Papula A L, Poon GYP, Wong W H, Young A L, Druley T E, Fisher D S, Blundell J R. The evolutionary dynamics and fitness landscape of clonal hematopoiesis. Science (80-) 2020; 367; (6485):1449-1454.
18. Abdel-Wahab O, Adli M, LaFave L M, Gao J, Hricik T, Shih A H, Pandey S, Patel J P, Chung Y R, Koche R, Perna F, Zhao X, Taylor J E, Park C Y, Carroll M, Melnick A, Nimer S D, Jaffe J D, Aifantis I, Bernstein B E, Levine R L. ASXL1 mutations promote myeloid transformation through loss of PRC2-mediated gene repression. Cancer Cell 2012; 22; (2):180-193.
19. Assmus B, Cremer S, Kirschbaum K, Culmann D, Kiefer K, Dorsheimer L, Rasper T, Abou-El-Ardat K, Herrmann E, Berkowitsch A, Hoffmann J, Seeger F, Mas-Peiro S, Rieger M A, Dimmeler S, Zeiher A M. Clonal haematopoiesis in chronic ischaemic heart failure: prognostic role of clone size for DNMT3A- and TET2-driver gene mutations. Eur Heart J 2021; 42; (3):257-265.
20. Geppert A, Dorninger A, Delle-Karth G, Zorn G, Heinz G, Huber K. Plasma concentrations of interleukin-6, organ failure, vasopressor support, and successful coronary revascularization in predicting 30-day mortality of patients with cardiogenic shock complicating acute myocardial infarction. Crit Care Med 2006; 34; (8):2035-2042.
21. Heeschen C, Dimmeler S, Hamm C W, Brand M J van den, Boersma E, Zeiher A M, Simoons M L. Soluble CD40 ligand in acute coronary syndromes. N Engl J Med 2003; 348; (12):1104-1111.
22. Shami A, Edsfeldt A, Bengtsson E, Nilsson J, Shore A C, Natali A, Khan F, Lutgens E, Gonçalves I. Soluble CD40 levels in plasma are associated with cardiovascular disease and in carotid plaques with a vulnerable phenotype. J Stroke 2021; 23; (3):367-376.
23. Milner J D, Orekov T, Ward J M, Cheng L, Torres-Velez F, Junttila I, Sun G, Buller M, Morris S C, Finkelman F D, Paul W E. Sustained IL-4 exposure leads to a novel pathway for hemophagocytosis, inflammation, and tissue macrophage accumulation. Blood 2010; 116; (14):2476-2483.
24. Qin Y, Zhang C. The regulatory role of IFN-γ on the proliferation and differentiation of hematopoietic stem and progenitor cells. Stem cell Rev reports 2017; 13; (6):705-712.
25. Baldridge M T, King K Y, Boles N C, Weksberg D C, Goodell M A. Quiescent haematopoietic stem cells are activated by IFN-gamma in response to chronic infection. Nature 2010; 465; (7299):793-797.
26. Agarwal P, Li H, Choi K, Hueneman K, He J, Welner R S, Starczynowski D T, Bhatia R. TNF-α-induced alterations in stromal progenitors enhance leukemic stem cell growth via CXCR2 signaling. Cell Rep 2021; 36; (2):109386.
27. Abegunde S O, Buckstein R, Wells R A, Rauh M J. An inflammatory environment containing TNFα favors Tet2-mutant clonal hematopoiesis. Exp Hematol 2018; 59:60-65.
28. Broek I Vande, Asosingh K, Vanderkerken K, Straetmans N, Camp B Van, Riet I Van. Chemokine receptor CCR2 is expressed by human multiple myeloma cells and mediates migration to bone marrow stromal cell-produced monocyte chemotactic proteins MCP-1, -2 and -3. Br J Cancer 2003; 88; (6):855-862.
29. Bick A G, Pirruccello J P, Griffin G K, Gupta N, Gabriel S, Saleheen D, Libby P, Kathiresan S, Natarajan P. Genetic interleukin 6 signaling deficiency attenuates cardiovascular risk in clonal hematopoiesis. Circulation 2020; 141; (2):124-131.
s1. Mehra M R, Canter C E, Hannan M M, et al. The 2016 International Society for Heart Lung Transplantation listing criteria for heart transplantation: A 10-year update. J Heart Lung Transplant. 2016; 35(1):1-23. doi:10.1016/j.healun.2015.10.023
s2. Organ Transplant|US Organ Donation System|UNOS. Accessed Jan. 27, 2022. https://unos.org/transplant/
s3. Khush K K, Cherikh W S, Chambers D C, et al. The International Thoracic Organ Transplant Registry of the International Society for Heart and Lung Transplantation: Thirty-sixth adult heart transplantation report—2019; focus theme: Donor and recipient size match. J Heart Lung Transplant. 2019; 38(10):1056-1066. doi:10.1016/J.HEALUN.2019.08.004
s4. Costanzo M R, Force T, Rosa M, et al. The International Society of Heart and Lung Transplantation Guidelines for the care of heart transplant recipients. J Heart Lung Transplant. 2010; 29(8):914-956. doi:10.1016/j.healun.2010.05.034
s5. Kittleson M M, Kobashigawa J A. Long-term care of the heart transplant recipient. Curr Opin Organ Transplant. 2014; 19(5):515-524. doi:10.1097/MOT.0000000000000117
s6. Giarraputo A, Barison I, Fedrigo M, et al. A changing paradigm in heart transplantation: An integrative approach for invasive and non-invasive allograft rejection monitoring. Biomolecules. 2021; 11(2):1-17. doi:10.3390/biom11020201
s7. Jaiswal S, Fontanillas P, Flannick J, et al. Age-related clonal hematopoiesis associated with adverse outcomes. N Engl J Med. 2014; 371(26):2488-2498. doi:10.1056/NEJMoa1408617
s8. Jaiswal S, Natarajan P, Silver A J, et al. Clonal hematopoiesis and risk of atherosclerotic cardiovascular disease. N Engl J Med. 2017; 377(2):111-121. doi:10.1056/NEJMoa1701719
s9. Bhattacharya R, Zekavat S M, Haessler J, et al. Clonal hematopoiesis is associated with higher risk of stroke. Stroke. 2020; 53(3):788-797. doi:10.1161/strokeaha.121.037388
s10. Dorsheimer L, Assmus B, Rasper T, et al. Association of mutations contributing to clonal hematopoiesis with prognosis in chronic ischemic heart failure. JAMA Cardiol. 2019; 4(1):25-33. doi:10.1001/jamacardio.2018.3965
s11. Bolton K L, Koh Y, Foote M B, et al. Clonal hematopoiesis is associated with risk of severe Covid-19. Nat Commun. 2021; 12(1):5975. doi:10.1038/s41467-021-26138-6
s12. Genovese G, Kähler A K, Handsaker R E, et al. Clonal hematopoiesis and blood-cancer risk inferred from blood DNA sequence. N Engl J Med. 2014; 371(26):2477-2487. doi:10.1056/NEJMoa1409405
s13. Dawoud A A Z, Gilbert R D, Tapper W J, Cross N C P. Clonal myelopoiesis promotes adverse outcomes in chronic kidney disease. Leukemia. 2022; 36(2):507-515. doi:10.1038/s41375-021-01382-3
s14. Pascual-Figal D A, Bayes-Genis A, Diez-Diez M, et al. Clonal hematopoiesis and risk of progression of heart failure with reduced left ventricular ejection fraction. J Am Coll Cardiol. 2021; 77(14):1747-1759. doi:10.1016/j.jacc.2021.02.028
s15. Sano S, Oshima K, Wang Y, Katanasaka Y, Sano M, Walsh K. CRISPR-mediated gene editing to assess the roles of TET2 and DNMT3A in clonal hematopoiesis and cardiovascular disease. Circ Res. 2018; 123(3):335-341. doi:10.1161/CIRCRESAHA.118.313225
s16. Medeiros J J F, Capo-Chichi J-M, Shlush L I, et al. SmMIP-tools: a computational toolset for processing and analysis of single-molecule molecular inversion probes-derived data. Bioinformatics. 2022; 38(8):2088-2095. doi:10.1093/bioinformatics/btac081
s17. Jaiswal S, Libby P. Clonal haematopoiesis: connecting ageing and inflammation in cardiovascular disease. Nat Rev Cardiol. 2020; 17(3):137-144. doi:10.1038/s41569-019-0247-5
s18. Sano S, Oshima K, Wang Y, et al. TET2-mediated clonal hematopoiesis accelerates heart failure through a mechanism involving the IL-1β/NLRP3 inflammasome. J Am Coll Cardiol. 2018; 71(8):875-886. doi:10.1016/j.jacc.2017.12.037
19. Sano S, Wang Y, Yura Y, et al. JAK2-V617F-Mediated clonal hematopoiesis accelerates pathological remodeling in murine heart failure. JACC Basic Transl Sci. 2019; 4(6):684-697. doi: 10.1016/j.jacbts.2019.05.013
s20. Sano S, Wang Y, Ogawa H, et al. TP53-mediated therapy-related clonal hematopoiesis contributes to doxorubicin-induced cardiomyopathy by augmenting a neutrophil-mediated cytotoxic response. JCI Insight. 2021; 6 (13): e146076. doi: 10.1172/jci.insight.146076
s21. Yura Y, Miura-Yura E, Katanasaka Y, et al. The Cancer Therapy-Related Clonal Hematopoiesis Driver Gene Ppm1d Promotes Inflammation and Non-Ischemic Heart Failure in Mice. Circ Res. 2021; 129(6):684-698. doi: 10.1161/CIRCRESAHA.121.319314
s22. Fuster J J, MacLauchlan S, Zuriaga M A, et al. Clonal hematopoiesis associated with TET2 deficiency accelerates atherosclerosis development in mice. Science. 2017; 355(6327): 842-847. doi: 10.1126/science.aag1381
s23. Nikolova A P, Kobashigawa J A. Cardiac Allograft Vasculopathy: The enduring enemy of cardiac transplantation. Transplantation. 2019; 103(7): 1338-1348. doi: 10.1097/TP.0000000000002704
s24. Chih S, Chong A Y, Mielniczuk L M, Bhatt D L, Beanlands R S B. Allograft vasculopathy: the achilles' heel of heart transplantation. J Am Coll Cardiol. 2016; 68 (1):80-91. doi:10.1016/J.JACC.2016.04.033
s25. Lee F, Nair V, Chih S. Cardiac allograft vasculopathy: Insights on pathogenesis and therapy. Clin Transplant. 2020; 34(3):e13794. doi:10.1111/CTR.13794
s26. Fahmy N M, Yamani M H, Starling R C, et al. Chemokine and chemokine receptor gene expression indicates acute rejection of human cardiac transplants. Transplantation. 2003; 75(1):72-78. doi:10.1097/00007890-200301150-00013
s27. Tarazón E, Corbacho-Alonso N, G. Barderas M, et al. Plasma CD5L and non-invasive diagnosis of acute heart rejection. J Heart Lung Transplant. 2020; 39(3):257-266. doi:10.1016/J.HEALUN.2019.11.004
s28. Jones I K A, Orloff S, Burg J M, et al. Blocking the IL-1 receptor reduces cardiac transplant ischemia and reperfusion injury and mitigates CMV-accelerated chronic rejection. Am J Transplant. 2021; 21(1):44-59. doi:10.1111/ajt.16149
s29. Alyaydin E, Welp H, Reinecke H, Tuleta I. Predisposing factors for late mortality in heart transplant patients. Cardiol J. 2021; 28(5):746-757. doi:10.5603/CJ.A2020.0011
s30. Newell L F, Dunlap J, Gatter K, et al. Graft-versus-host disease after liver transplantation is associated with bone marrow failure, hemophagocytosis, and DNMT3A mutations. Am J Transplant. 2021; 21(12):3894-3906.

1. A method of predicting the risk of a disease condition of a solid organ in a patient, the method comprising:
a) receiving a sample from the patient containing hematopoietic stem cells;
b) sequencing the sample to detect a degree of clonal hematopoiesis;
c) comparing the degree of clonal hematopoiesis in the patient to a control degree; and
d) determining the patient is at an elevated risk of the disease condition if the degree of clonal hematopoiesis in the patient is higher than the control degree in a statistically significant manner.

2. The method of claim 1, wherein the degree of clonal hematopoiesis is measured using a variant allele frequency of mutations determined to be associated with clonal hematopoiesis.

3. The method of claim 2, wherein the variant allele frequency (VAF) is ≥2%.

4. The method of claim 3 wherein the VAF is ≥5%.

5. The method of claim 1, wherein the following genes are sequenced in the sample: TET2, DNMT3A, and ASXL1, and optionally one or more of, but preferably all of, BCOR, BRAF, CALR, CBL, CEBPA, EZH2, FLT3A, GATA1, GATA2, GNAS, IDH1, IDH2, JAK2, KIT, KRAS, MPL, NRAS, PHF6, PPM1D, PTPN11, RAD21, RUNX1, SETBP1, SF3B1, SMC1A, SMC3, SRSF2, STAG2, TP53, U2AF1, WT1, and ZRSR2.

6. The method of claim 1, wherein the sequencing is performed using single-molecule molecular inversion probes (smMIPs).

7. The method of claim 2, wherein the mutations associated with clonal hematopoiesis are detectable by the probes listed in Table A.

8. The method of claim 2, wherein the mutations associated with clonal hematopoiesis are detected using a library comprising at least 50%, 60%, 70%, 80%, 90%, 95%, 98% or 99% of the probes listed in Table A.

9. (canceled)

10. (canceled)

11. (canceled)

12. The method of claim 1, wherein the solid organ is a heart.

13. The method of claim 12, wherein the disease condition is cardiogenic shock.

14. The method of claim 13, wherein an elevated risk of cardiogenic shock is associated with an elevated risk of death.

15. The method of claim 13, further comprising treating or preventatively treating the patient for cardiogenic shock.

16. The method of claim 12, wherein the disease condition is an adverse outcome after orthotopic heart transplant (OHT).

17. The method of claim 16, wherein the adverse outcome is an elevated risk of mortality and/or elevated risk of cardiac allograft vasculopathy.

18. The method of claim 17, further comprising treating or preventatively treating the patient for cardiac allograft vasculopathy.

19. The method of claim 12, wherein the disease condition is hypertrophic cardiomyopathy.

20. The method of claim 19, further comprising treating or preventatively treating the patient for hypertrophic cardiomyopathy.

21. The method of claim 1, wherein the solid organ is a lung.

22. The method of claim 21, wherein the disease condition is an adverse outcome, including death, after lung transplant.

23. The method of claim 22, further comprising treating or preventatively treating the patient for the adverse outcome after lung transplant.

24. A kit comprising a library of probes library comprising at least 50%, 60%, 70%, 80%, 90%, 95%, 98% or 99% of the probes listed in Table A.

25. (canceled)

26. (canceled)

27. (canceled)

28. A method of predicting the benefit of immunotherapy in a patient with melanoma, the method comprising:
a) receiving a sample from the patient containing hematopoietic stem cells;
b) sequencing the sample to detect a degree of clonal hematopoiesis in TET2;
c) comparing the degree of TET2 clonal hematopoiesis in the patient to a control degree; and
d) determining the patient would benefit from immunotherapy if the degree of TET2 clonal hematopoiesis in the patient is higher than the control degree in a statistically significant manner.

29. The method of claim 28, further comprising treating the patient with immunotherapy.

30. A method of predicting metastatic risk in a patient with a non-hematological cancer, the method comprising:
a) receiving a sample from the patient containing hematopoietic stem cells;
b) sequencing the sample to detect a degree of clonal hematopoiesis in TET2;
c) comparing the degree of TET2 clonal hematopoiesis in the patient to a control degree; and
d) determining the patient is at a lower risk of metastasis if the degree of TET2 clonal hematopoiesis in the patient is higher than the control degree in a statistically significant manner.

31. The method of claim 30, further comprising treating the patient with a treatment and monitoring regimen reflective of a low risk of metastasis.
